(19) United States
(12) Patent Application Publication
(10) Pub. No.: US 2025/0301902 A1
(43) Pub. Date: Sep. 25, 2025

(54) COMPOUND FOR ORGANIC ELECTRONIC ELEMENT, ORGANIC ELECTRONIC ELEMENT USING THE SAME, AND AN ELECTRONIC DEVICE THEREOF

(71) Applicant: DUK SAN NEOLUX CO., LTD., Cheonan-si (KR)

(72) Inventors: Hyo Min JIN, Cheonan-si (KR); Hyun Ju SONG, Cheonan-si (KR); Ji young KIM, Cheonan-si (KR); Se Hoon LEE, Cheonan-si (KR); Hyun Ji OH, Cheonan-si (KR)

(73) Assignee: DUK SAN NEOLUX CO., LTD., Cheonan-si (KR)

(21) Appl. No.: 19/231,353

(22) Filed: Jun. 6, 2025

Related U.S. Application Data
(63) Continuation-in-part of application No. 18/180,625, filed on Mar. 8, 2023.
Application No. 18/180,625 is a continuation-in-part of application No. 17/212,886, filed on Mar. 25, 2021, now Pat. No. 11,678,577.
Application No. 17/212,886 is a continuation of application No. 17/096,790, filed on Nov. 12, 2020, now Pat. No. 11,063,226.

(30) Foreign Application Priority Data
Oct. 26, 2020 (KR) 10-2020-0139441

(51) Int. Cl. H10K 85/60; C07D 251/24; C09K 11/06; H10K 50/11; H10K 50/15; H10K 50/16; H10K 50/18; H10K 101/00; H10K 101/10

(52) U.S. Cl. CPC H10K 85/636; C07D 251/24; C09K 11/06; H10K 85/626; H10K 85/631; H10K 85/653; H10K 85/654; H10K 85/6572; H10K 85/6574; H10K 85/6576; C07B 2200/05; C09K 2211/1018; H10K 50/11; H10K 50/15; H10K 50/16; H10K 50/18; H10K 2101/10; H10K 2101/90

(57) ABSTRACT

Provided are a compound of Formula 1 that can improve the luminescence efficiency, stability, and lifespan of an organic electronic element, a composition comprising the same, an organic electronic element employing the same, and an electronic device thereof.

### BACKGROUND

### Technical Field

The present invention relates to compounds for organic electronic elements, organic electronic elements using the same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Here, in order to increase the efficiency and stability of the organic electronic element, the organic material layer is often composed of a multi-layered structure composed of different materials, and for example, may include a hole injection layer, a hole transport layer, an emitting layer, an electron transport layer, an electron injection layer etc.

A material used as an organic material layer in an organic electronic element may be classified into a light emitting material and a charge transport material, such as a hole injection material, a hole transport material, an electron transport material, an electron injection material etc. depending on its function. And the light emitting material can be classified into a high molecular weight type and a low molecular weight type according to the molecular weight, and it can be classified into a fluorescent material derived from a singlet excited state of an electron and a phosphorescent material derived from a triplet excited state of an electron depending on the light emission mechanism. Also, the light emitting material may be divided into blue, green, and red light emitting materials and yellow and orange light emitting materials necessary for realizing a better natural color according to the emission color.

However, when only one material is used as a light emitting material, due to intermolecular interaction, the maximum emission wavelength shifts to a longer wavelength, and there are problems in that the color purity is lowered or the device efficiency is reduced due to the emission attenuation effect, therefore in order to increase color purity and increase luminescence efficiency through energy transfer, a host/dopant system may be used as a light emitting material. The principle is that when a small amount of a dopant having a smaller energy band gap than that of the host forming the emitting layer is mixed in the emitting layer, excitons generated in the emitting layer are transported to the dopant to emit light with high efficiency. Here, since the wavelength of the host moves to the wavelength band of the dopant, light having a desired wavelength can be obtained according to the type of dopant used.

Currently, the portable display market is a large-area display, and the size thereof is increasing, and thus, more power consumption than the power consumption required for the existing portable display is required. Therefore, power consumption has become a very important factor for a portable display having a limited power supply such as a battery, and the problem of efficiency and lifetime must also be solved.

Efficiency, lifetime and driving voltage are related to each other, and when the efficiency is increased, the driving voltage is relatively decreased, and as the driving voltage is decreased, crystallization of organic materials due to Joule heating generated during driving decreases, and consequently, the lifetime tends to increase. However, the efficiency cannot be maximized simply by improving the organic material layer. This is because, when the energy level and T1 value between each organic material layer, and the intrinsic properties (mobility, interfacial properties, etc.) of materials are optimally combined, long lifetime and high efficiency can be achieved at the same time.

Therefore, while delaying the penetration and diffusion of metal oxide from the anode electrode (ITO) into the organic layer, which is one of the causes of shortening the lifetime of the organic electronic element, it should have stable characteristics against Joule heating generated during device driving, and OLED devices are mainly formed by a deposition method, and it is necessary to develop a material that can withstand a long time during deposition, that is, a material with strong heat resistance.

That is, in order to fully exhibit the excellent characteristics of an organic electronic element, it should be preceded that the material constituting the organic material layer in the device, such as a hole injection material, a hole transport material, a light emitting material, an electron transport material, an electron injection material, etc., is supported by a stable and efficient material. But the development of a stable and efficient organic material layer material for an organic electronic element has not yet been sufficiently made. Therefore, the development of new materials is continuously required, and in particular, the development of a host material for the emitting layer is urgently needed.

### BRIEF DESCRIPTION OF THE INVENTION

### Summary

In order to solve the problems of the above-mentioned background technology, the present invention has discovered a compound with a novel structure, and also discovered that when the compound is applied to an organic electronic element, the luminescence efficiency, stability, and lifetime of the element can be greatly improved.

Accordingly, the purpose of the present invention is to provide a novel compound, an organic electronic element using the same, and an electronic device thereof.

### Technical Solution

The present invention provides a compound represented by Formula 1.

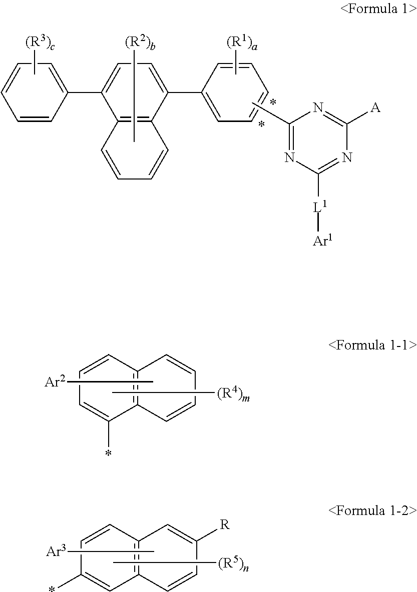

In another aspect, the present invention provides a composition for an organic electronic element comprising a mixture of a compound represented by Formula 1 and a compound represented by Formula 2 or Formula 3.

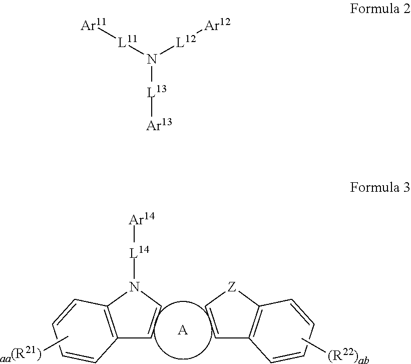

In another aspect, the present invention provides an organic electronic element comprising the compound represented by Formula 1 or the composition for the organic electronic element, and an electronic device thereof.

### Effects of the Invention

By using the compound according to the present invention, high luminescence efficiency, low driving voltage and high heat resistance of the element can be achieved, and color purity and lifetime of the element can be greatly improved.

### BRIEF DESCRIPTION OF THE DRAWINGS

FIG. 1 to FIG. 3 are exemplary views of an organic electroluminescent device according to the present invention.

[Description of the numerals]

| | |
|---|---|
| 100, 200, 300: organic electronic | 110: the first electrode |
| element | |
| 120: hole injection layer | 130: hole transport layer |
| 140: emitting layer | 150: electron transport layer |
| 160: electron injection layer | 170: second electrode |
| 180: light efficiency enhancing Layer | 210: buffer layer |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

### DETAILED DESCRIPTION OF THE INVENTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, 1 to 30 carbon atoms, 1 to 25 carbon atoms, 1 to 18 carbon atoms, or 1 to 12 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, 2 to 30 carbon atoms, 2 to 25 carbon atoms, 2 to 18 carbon atoms, 2 to 12 carbon atoms, but is not limited thereto, and includes a linear or a branched chain group.

Unless otherwise stated, the term “cycloalkyl”, as used herein, means alkyl forming a ring having 3 to 60 carbon atoms, 3 to 30 carbon atoms, 3 to 25 carbon atoms, 3 to 18 carbon atoms, 3 to 12 carbon atoms, but is not limited thereto.

Unless otherwise stated, the term “alkoxyl group”, “alkoxy group” or “alkyloxy group”, as used herein, means an alkyl group bonded to oxygen radical, but is not limited thereto, and has 1 to 60 carbon atoms, 1 to 30 carbon atoms, 1 to 25 carbon atoms, 1 to 18 carbon atoms, or 1 to 12 carbon atoms.

Unless otherwise stated, the term “aryloxyl group” or “aryloxy group”, as used herein, means an aryl group bonded to oxygen radical, but is not limited thereto, and has 6 to 60 carbon atoms, 6 to 30 carbon atoms, 6 to 25 carbon atoms, 6 to 18 carbon atoms, or 6 to 12 carbon atoms.

Unless otherwise specified, the terms “aryl group” and “arylene group” used in the present invention have 6 to 60 carbon atoms, 6 to 30 carbon atoms, 6 to 25 carbon atoms, 6 to 18 carbon atoms, or 6 to 12 carbon atoms, respectively, but are not limited thereto. In the present invention, an aryl group or arylene group refers to an aromatic group of a single ring or multiple rings, and includes an aromatic ring formed by combining adjacent substituents or participating in a reaction. For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, 2 to 30 carbon atoms, 2 to 25 carbon atoms, 2 to 18 carbon atoms, 2 to 12 carbon atoms, and includes any one of a single ring or multiple ring, and may include heteroaliphatic ring and heteroaromatic ring. Also, the heterocyclic group may also be formed in conjunction with an adjacent group.

Unless otherwise stated, the term “heteroatom”, as used herein, represents at least one of N, O, S, P, or Si.

Also, “heterocyclic group” refers to a single ring containing heteroatoms, a ring aggregate, multiple fused ring systems, spiro compounds, etc. Additionally, compounds containing heteroatom groups such as SO₂, P═O, etc., such as the compounds below, instead of carbon forming a ring, may also be included in the heterocyclic group. For example, “heterocyclic group” includes the following compound.

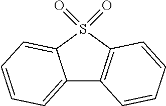 [SMILES: O=S1(=O)c2ccccc2-c2ccccc21]

The term “aliphatic ring group” used in the present invention refers to cyclic hydrocarbons excluding aromatic hydrocarbons, and includes single rings, ring aggregates, fused multiple ring systems, spiro compounds, etc., and means a ring having 3 to 60 carbon atoms, 3 to 30 carbon atoms, 3 to 25 carbon atoms, 3 to 18 carbon atoms, 3 to 12 carbon atoms, but is not limited thereto. For example, even when benzene, an aromatic ring, and cyclohexane, a non-aromatic ring, are fused, it is an aliphatic ring.

Unless otherwise stated, the term “fluorenyl group”, “fluorenylene group” or “fluorentriyl group” as used herein, means a monovalent, divalent or trivalent functional group, in which R, R′ and R″ are all hydrogen in the following structures, and the term “substituted fluorenyl group”, “substituted fluorenylene group” or “substituted fluorentriyl group” means that at least one of the substituents R, R′ and R″ is a substituent other than hydrogen, and include those in which R and R′ are bonded to each other to form a spiro compound together with the carbon to which they are bonded. In this specification, fluorenyl group, fluorenylene group, and fluorenetriyl group may all be referred to as fluorene groups, regardless of valence.

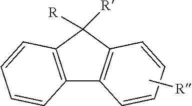

The term “spiro compound”, as used herein, has a ‘spiro union’, and a spiro union means a connection formed by 2 rings sharing only one atom. Wherein, the atoms shared between the 2 rings are called ‘spiro atoms’, and depending on the number of spiro atoms contained in a compound, they are called ‘monospiro-’, ‘dispiro-’, and ‘trispiro-’ compounds, respectively.

Unless otherwise stated, the term “aliphatic” as used herein means an aliphatic hydrocarbon having 1 to 60 carbon atoms, 1 to 30 carbon atoms, 1 to 25 carbon atoms, 1 to 18 carbon atoms or 1 to 12 carbon atoms, and “aliphatic ring” means an aliphatic hydrocarbon ring having 3 to 60 carbon atoms, 3 to 30 carbon atoms, 3 to 25 carbon atoms, 3 to 18 carbon atoms or 3 to 12 carbon atoms.

Unless otherwise stated, the term “ring”, as used herein, means an aliphatic ring having 3 to 60 carbon atoms, 3 to 30 carbon atoms, 3 to 25 carbon atoms, 3 to 18 carbon atoms or 3 to 12 carbon atoms; or an aromatic ring having 6 to 60 carbon atoms, 6 to 30 carbon atoms, 6 to 25 carbon atoms, 6 to 18 carbon atoms, or 6 to 12 carbon atoms; or a heterocyclic having 2 to 60 carbon atoms, 2 to 30 carbon atoms, 2 to 25 carbon atoms, 2 to 18 carbon atoms, 2 to 12 carbon atoms, or a fused ring formed by the combination thereof, and includes a saturated or unsaturated ring.

Other hetero compounds or hetero radicals other than the above-mentioned hetero compounds include, but are not limited thereto, one or more heteroatoms.

Also, unless expressly stated, as used herein, “substituted” in the term “substituted or unsubstituted” means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

In this specification, the ‘group name’ corresponding to the aryl group, arylene group, heterocyclic group, etc., as examples of each symbol and its substituent, may be written as the ‘name of the group reflecting the valence’, but is written as the ‘parent compound name’. For example, in the case of ‘phenanthrene’, a type of aryl group, the name of the group may be written by distinguishing the valence, such as the monovalent ‘group’ is ‘phenanthryl’ and the divalent group is ‘phenanthrylene’, but may be written as ‘phenanthrene’, which is the name of the parent compound, regardless of the valence. Similarly, in the case of pyrimidine, it can be written as ‘pyrimidine’ regardless of the valence, or it can be written as the ‘name of the group’ of the valence, such as pyrimidineyl group in the case of monovalent group, pyrimidineylene in the case of divalent group, etc. Additionally, in this specification, when describing compound names or substituent names, numbers or alphabets indicating positions may be omitted. For example, pyrido[4,3-d]pyrimidine to pyridopyrimidine, benzofuro[2,3-d]pyrimidine to benzofuropyrimidine, 9,9-dimethyl-9H-fluorene can be described as dimethylfluorene, etc. Therefore, both benzo[g]quinoxaline and benzo[f]quinoxaline can be described as benzoquinoxaline.

Also, unless there is an explicit explanation, the formula used in the present invention is the same as the definition of the substituent by the exponent definition of the following formula.

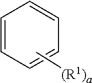

Here, when a is an integer of 0, the substituent R¹ is absent, when a is an integer of 1, the sole substituent R¹ is linked to any one of the carbon constituting the benzene ring, when a is an integer of 2 or 3, each is combined as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

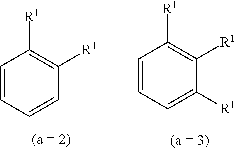

Unless otherwise expressly stated, the terms “ortho”, “meta”, and “para” used in the present invention refer to the substitution positions of all substituents, and the ortho position refers to a compound in which the position of the substituent is immediately adjacent, for example, when benzene is used, it means 1 or 2 position, and the meta position is the next substitution position of the neighbor substitution position, when benzene as an example stands for 1 or 3 position, and the para position is the next substitution position of the meta position, which means 1 and 4 position when benzene is taken as an example. A more detailed example of the substitution position is as follows, and it can be confirmed that the ortho-, and meta-position are substituted by non-linear type and para-positions are substituted by linear type.

### [Example of Ortho-Position]

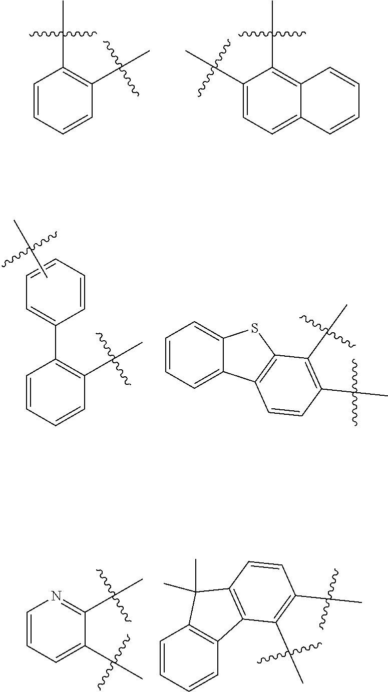

### [Example of Meta-Position]

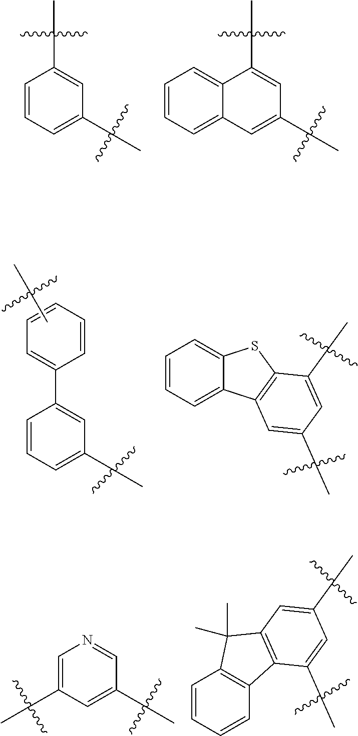

### [Example of Para-Position]

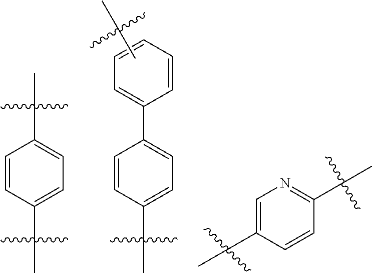

As used herein, the term “composition” is intended to be interpreted broadly, comprising compounds as well as solutions, dispersions, liquids and solid mixtures (mixture, admixture). The composition of the present invention may comprise the compound of the present invention alone, or the compounds are comprised in a combination of 2 or more different types, or the compounds may be comprised in combinations of 2 or more types with other compounds. In other words, the composition may comprise a compound corresponding to Formula 1 alone, a mixture of 2 or more compounds of Formula 1, or a mixture of a compound of Formula 1 and a compound not corresponding to the present invention. Wherein, the compound not corresponding to the present invention may be a single compound, and may be 2 or more types of compounds. Here, when the compound is comprised in a combination of 2 or more types of other compounds, the other compounds may be already known compounds of each organic material layer, or may be compounds to be developed in the future. Wherein, the compound contained in the organic material layer may consist of only the same type of compound, but may also be a mixture of 2 or more types of different compounds represented by Formula 1.

Hereinafter, a compound according to an aspect of the present invention, a composition for an organic electronic element and an organic electronic element comprising the same will be described.

The present invention provides a compound represented by Formula 1.

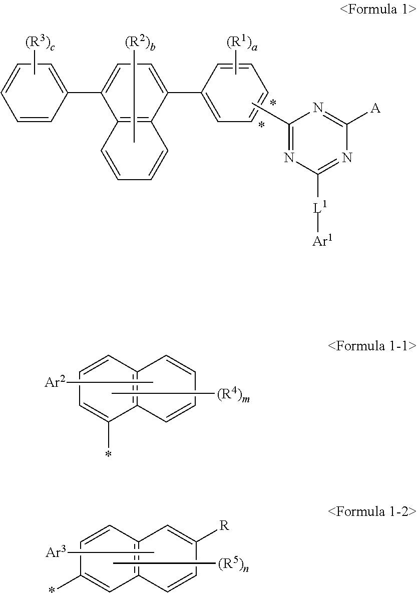

wherein:
A is represented by Formula 1-1 or Formula 1-2,
R¹, R² and R³ are the same or different from each other and are independently hydrogen; or deuterium;
a is an integer of 0 to 4, b is an integer of 0 to 6, c is an integer of 0 to 5
wherein R¹, R² and R³ do not be bonded to each other to form a ring,
L¹ is a single bond; or a C₆-C₆₀ arylene group;
Wherein when L¹ is an arylene group, preferably a C₆-C₆₀ arylene group, more preferably a C₆-C₂₅ arylene group, such as phenyl, biphenyl, terphenyl, naphthalene, phenanthrene, chrysene, etc.
Ar¹ is a C₆-C₆₀ aryl group; or a C₂-C₆₀ heterocyclic group including at least one heteroatom of O, N, S, Si and P;
Wherein when Ar¹ is an aryl group, preferably a C₆-C₂₅ aryl group, a C₆-C₁₈ aryl group or a C₆-C₁₂ aryl group, such as phenyl, biphenyl, terphenyl, naphthalene, phenanthrene, chrysene, etc.
Wherein when Ar¹ is a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidine, quinoline, pyrimidoindole, 5-phenyl-5H-pyrimido[5,4-b]indole, quinazoline, quinoxaline, benzoquinazoline, carbazole, dibenzoquinazoline, benzofuran, benzothiophene, dibenzofuran, dibenzothiophene, benzothienopyrimidine, benzofuropyrimidine, phenothiazine, phenylphenothiazine, naphthobenzofuran, naphthobenzothiophene, etc.
* indicates the binding position,
R⁴ and R⁵ are the same or different from each other and are independently hydrogen; deuterium; or a C₆-C₆₀ aryl group;
Wherein R⁴ and R⁵ do not form a ring by bonding with adjacent substituents,
Wherein when R⁴ and R⁵ are an aryl group, preferably a C₆-C₂₅ aryl group, a C₆-C₁₈ aryl group or a C₆-C₁₂ aryl group, such as phenyl, biphenyl, naphthalene, etc.
R is hydrogen; or deuterium;
m is an integer of 0 to 6, and n is an integer of 0 to 5.
Ar² and Ar³ are independently a C₆-C₆₀ aryl groups, identically or differently from each other, preferably a C₆-C₂₅ aryl group, a C₆-C₁₈ aryl group, a C₆-C₁₂ aryl group, such as phenyl, biphenyl, terphenyl, naphthalene, phenanthrene, chrysene, etc.
wherein the aryl group, arylene group and heterocyclic group may be substituted with one or more substituents selected from the group consisting of deuterium; a C₁-C₂₀ alkyl group; a C₆-C₂₀ aryl group; a C₆-C₂₀ aryl group substituted with deuterium; and a C₃-C₆₀ aliphatic ring, additionally, the hydrogens of these substituents may be further substituted with one or more deuterium atoms.

Also, Formula 1 is represented by Formula 2-1, 2-2, 2-3 or 2-4.

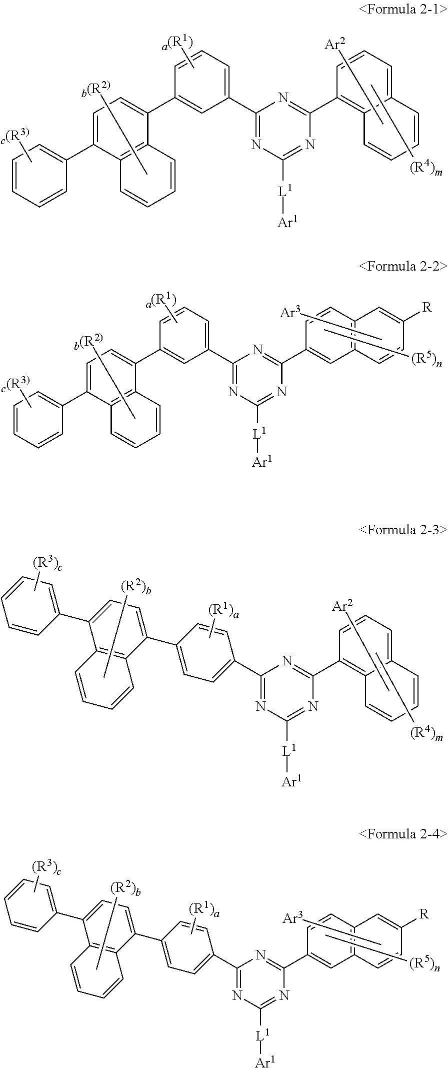

wherein:
L¹, Ar¹, Ar², Ar³, R, R¹, R², R³, R⁴, R⁵, a, b, c, m and n are the same as defined above.

Also, in Formula 1, Ar¹ is represented by any one of Formulas (A-1) to (A-5):

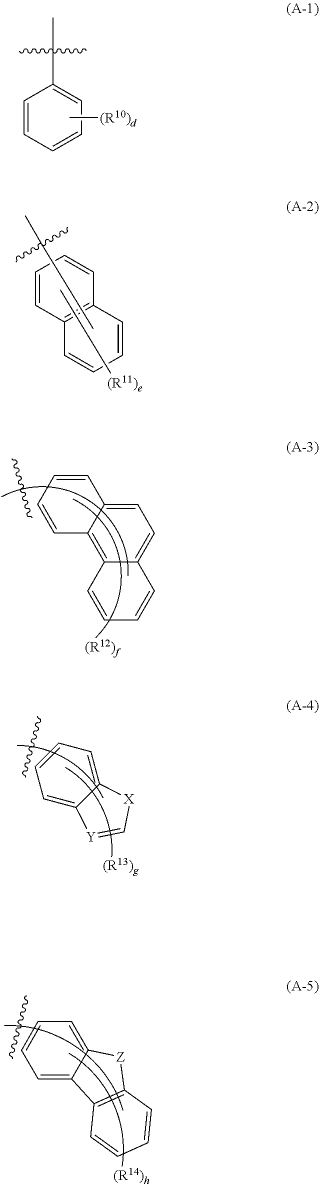

wherein:
R¹⁰, R¹¹, R¹², R¹³ and R¹⁴ are the same or different from each other and are independently selected from the group consisting of hydrogen; deuterium; a C₆-C₂₀ aryl group; or a C₆-C₂₀ aryl group substituted with deuterium;
an adjacent plurality of R¹⁰s or a plurality of R¹¹s or a plurality of R¹²s or a plurality of R¹³s or a plurality of R¹⁴s may be bonded to each other to form a ring,
d is an integer of 0 to 5,
e and h are integers from 0 to 7, f is an integer of 0 to 9, g is an integer of 0 to 4,
X and Z are independently O or S,
Y is N or C,
[symbol] indicates a position to be bonded.

L¹ is represented by any one of Formulas (L-1) to (L-19):

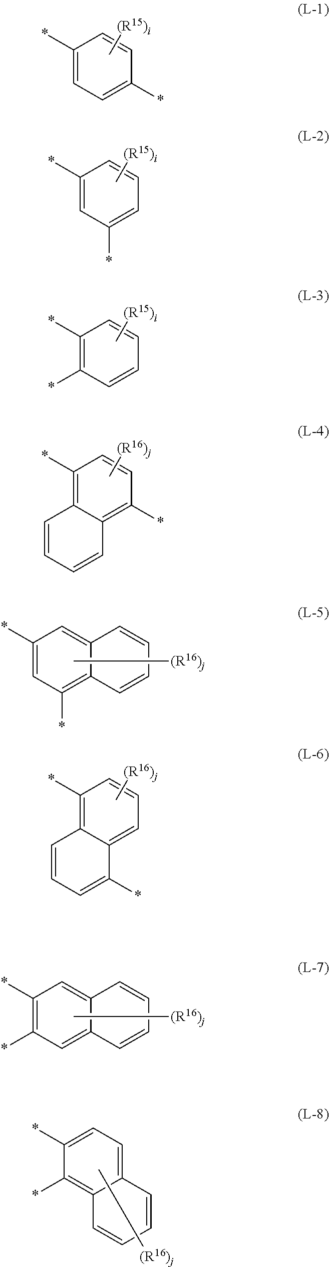

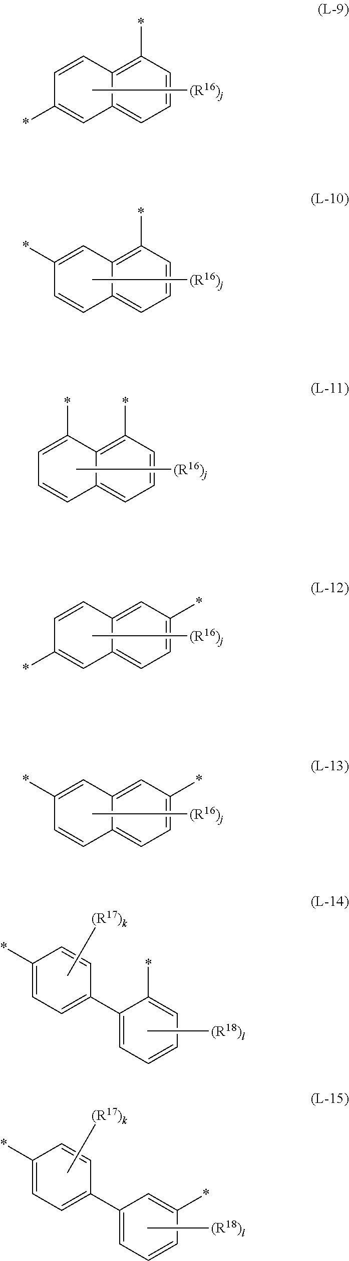

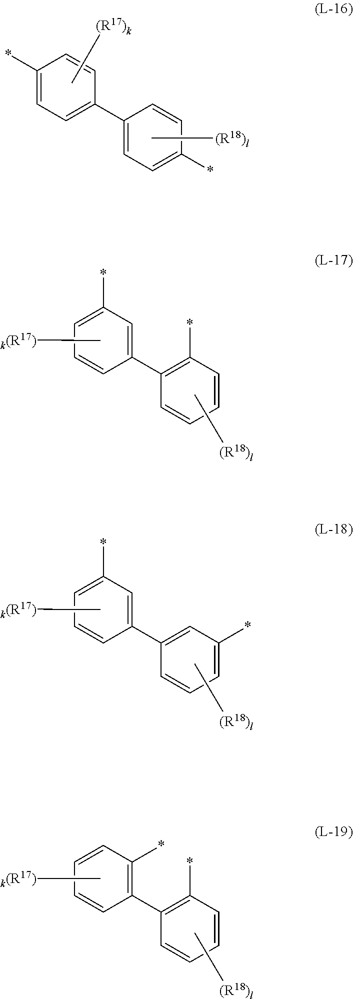

Wherein:
R¹⁵, R¹⁶, R¹⁷ and R¹⁸ are the same or different from each other and are independently hydrogen; or deuterium;
i, k and l are integer of 0 to 4, independently of each other, j is an integer of 0 to 6, and
* indicates the position to be bonded.

As another example, the compound represented by Formula 1 comprises at least one deuterium.

Specifically, the compound represented by Formula 1 may be a compound represented by any one of the compounds below, but is not limited thereto.

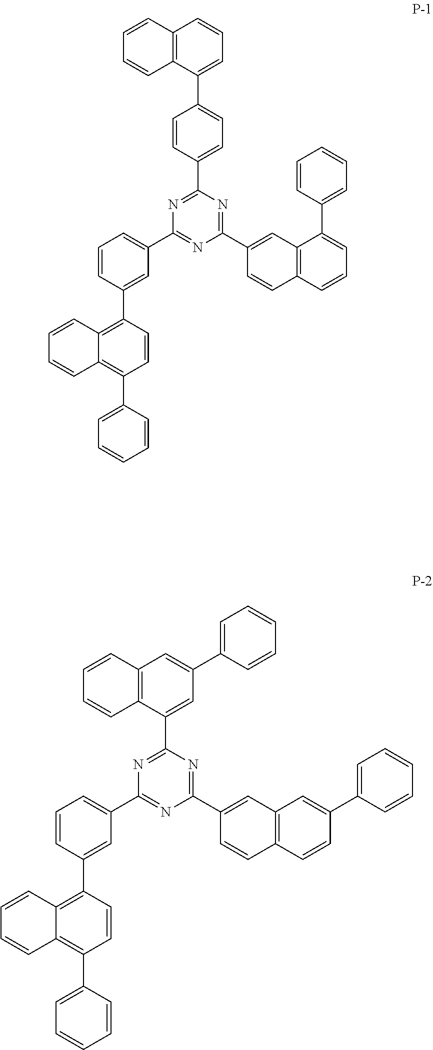 [SMILES: c1ccc(-c2cccc3ccc(-c4nc(-c5ccc(-c6cccc7ccccc67)cc5)nc(-c5cccc(-c6ccc(-c7ccccc7)c7ccccc67)c5)n4)cc23)cc1 ; c1ccc(-c2ccc3ccc(-c4nc(-c5cccc(-c6ccc(-c7ccccc7)c7ccccc67)c5)nc(-c5cc(-c6ccccc6)cc6ccccc56)n4)cc3c2)cc1]

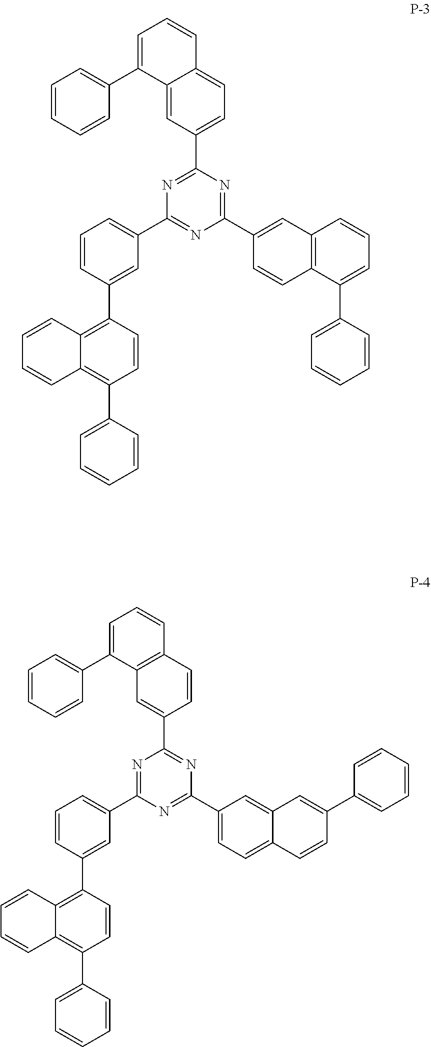 [SMILES: c1ccc(-c2cccc3cc(-c4nc(-c5cccc(-c6ccc(-c7ccccc7)c7ccccc67)c5)nc(-c5ccc6cccc(-c7ccccc7)c6c5)n4)ccc23)cc1 ; c1ccc(-c2ccc3ccc(-c4nc(-c5cccc(-c6ccc(-c7ccccc7)c7ccccc67)c5)nc(-c5ccc6cccc(-c7ccccc7)c6c5)n4)cc3c2)cc1]

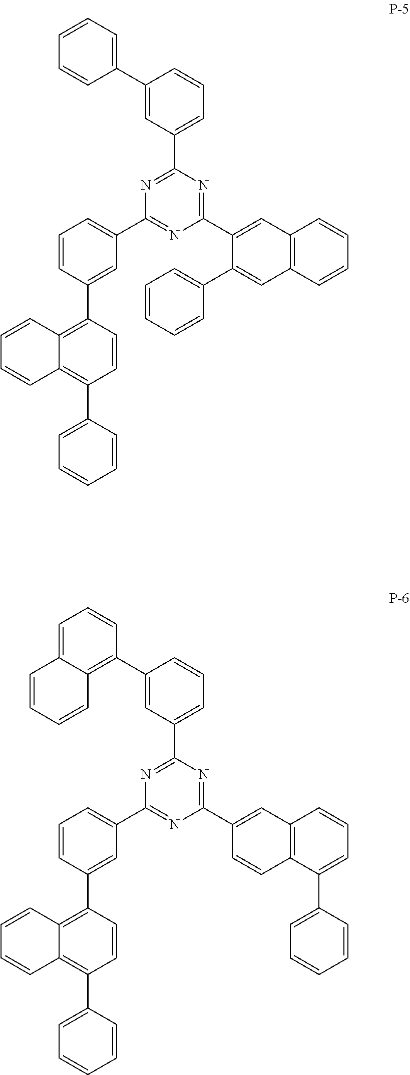 [SMILES: c1ccc(-c2cccc(-c3nc(-c4cccc(-c5ccc(-c6ccccc6)c6ccccc56)c4)nc(-c4cc5ccccc5cc4-c4ccccc4)n3)c2)cc1 ; c1ccc(-c2cccc3cc(-c4nc(-c5cccc(-c6cccc7ccccc67)c5)nc(-c5cccc(-c6ccc(-c7ccccc7)c7ccccc67)c5)n4)ccc23)cc1]

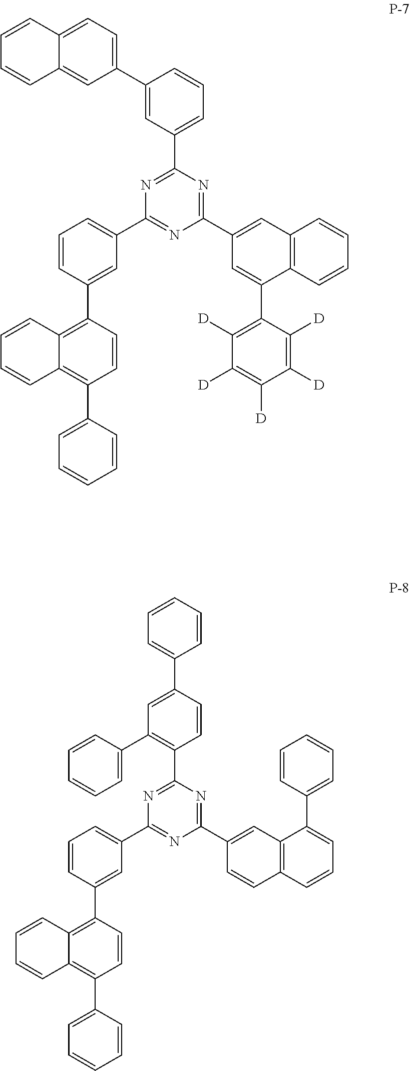 [SMILES: [2H]c1c([2H])c([2H])c(-c2cc(-c3nc(-c4cccc(-c5ccc6ccccc6c5)c4)nc(-c4cccc(-c5ccc(-c6ccccc6)c6ccccc56)c4)n3)cc3ccccc23)c([2H])c1[2H] ; c1ccc(-c2ccc(-c3nc(-c4cccc(-c5ccc(-c6ccccc6)c6ccccc56)c4)nc(-c4ccc5cccc(-c6ccccc6)c5c4)n3)c(-c3ccccc3)c2)cc1]

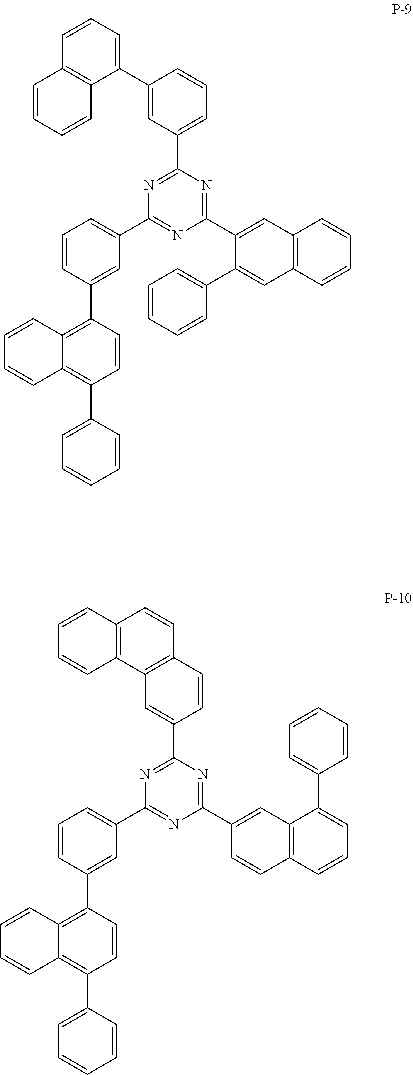 [SMILES: c1ccc(-c2cc3ccccc3cc2-c2nc(-c3cccc(-c4cccc5ccccc45)c3)nc(-c3cccc(-c4ccc(-c5ccccc5)c5ccccc45)c3)n2)cc1 ; c1ccc(-c2cccc3ccc(-c4nc(-c5cccc(-c6ccc(-c7ccccc7)c7ccccc67)c5)nc(-c5ccc6ccc7ccccc7c6c5)n4)cc23)cc1]

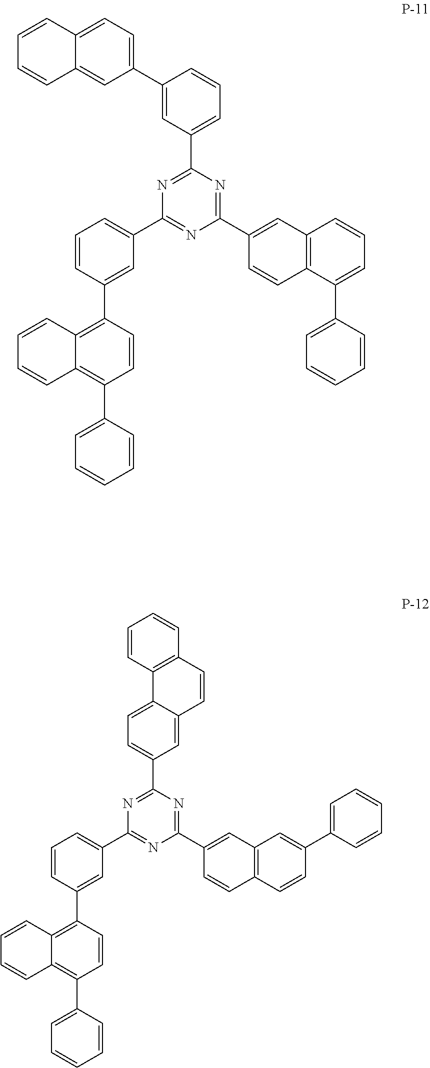 [SMILES: c1ccc(-c2cccc3cc(-c4nc(-c5cccc(-c6ccc7ccccc7c6)c5)nc(-c5cccc(-c6ccc(-c7ccccc7)c7ccccc67)c5)n4)ccc23)cc1 ; c1ccc(-c2ccc3ccc(-c4nc(-c5cccc(-c6ccc(-c7ccccc7)c7ccccc67)c5)nc(-c5ccc6c(ccc7ccccc76)c5)n4)cc3c2)cc1]

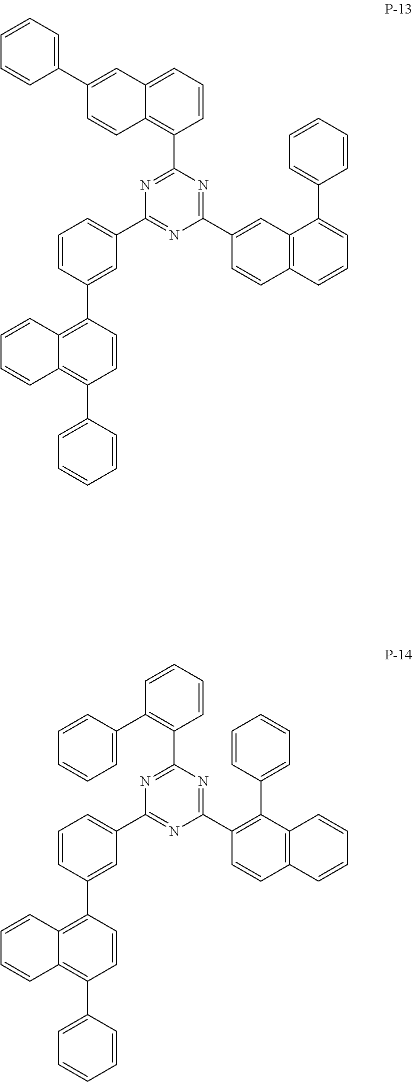 [SMILES: c1ccc(-c2ccc3c(-c4nc(-c5cccc(-c6ccc(-c7ccccc7)c7ccccc67)c5)nc(-c5ccc6cccc(-c7ccccc7)c6c5)n4)cccc3c2)cc1 ; c1ccc(-c2ccccc2-c2nc(-c3cccc(-c4ccc(-c5ccccc5)c5ccccc45)c3)nc(-c3ccc4ccccc4c3-c3ccccc3)n2)cc1]

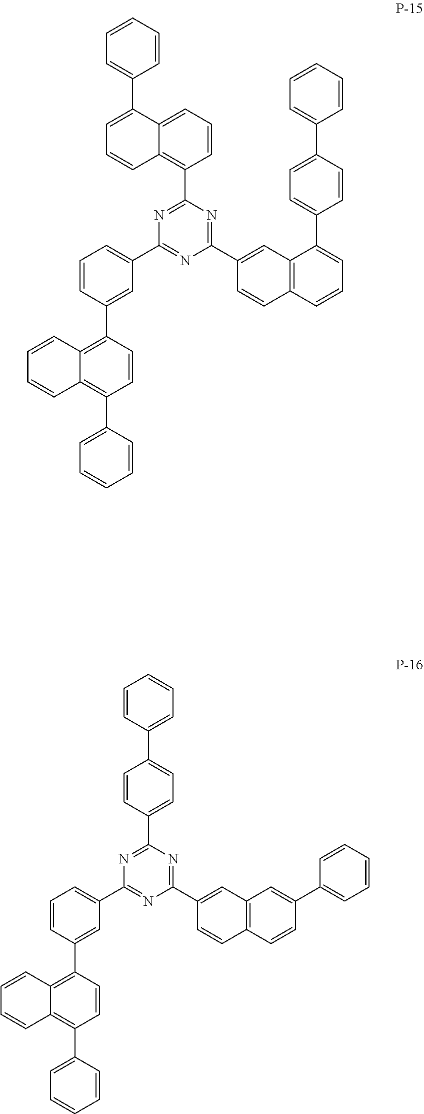 [SMILES: c1ccc(-c2ccc(-c3cccc4ccc(-c5nc(-c6cccc(-c7ccc(-c8ccccc8)c8ccccc78)c6)nc(-c6cccc7c(-c8ccccc8)cccc67)n5)cc34)cc2)cc1 ; c1ccc(-c2ccc(-c3nc(-c4cccc(-c5ccc(-c6ccccc6)c6ccccc56)c4)nc(-c4ccc5ccc(-c6ccccc6)cc5c4)n3)cc2)cc1]

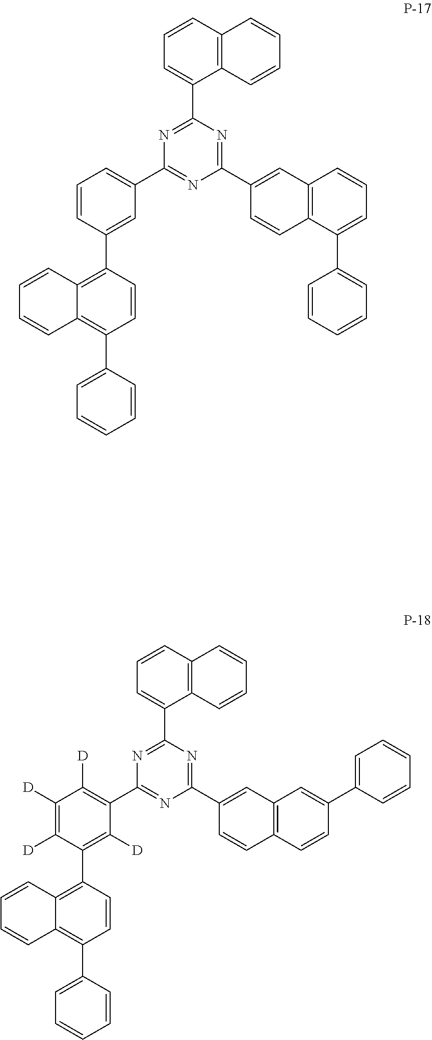 [SMILES: c1ccc(-c2cccc3cc(-c4nc(-c5cccc(-c6ccc(-c7ccccc7)c7ccccc67)c5)nc(-c5cccc6ccccc56)n4)ccc23)cc1 ; [2H]c1c([2H])c(-c2nc(-c3ccc4ccc(-c5ccccc5)cc4c3)nc(-c3cccc4ccccc34)n2)c([2H])c(-c2ccc(-c3ccccc3)c3ccccc23)c1[2H]]

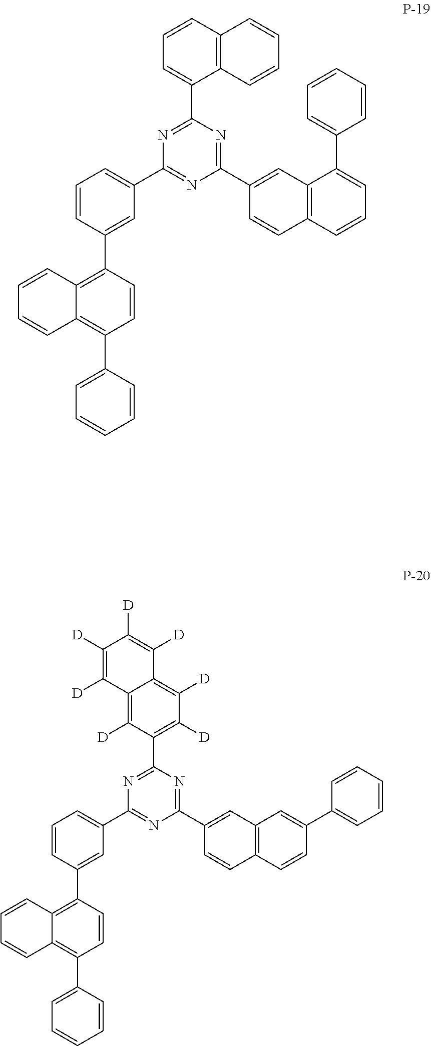 [SMILES: c1ccc(-c2cccc3ccc(-c4nc(-c5cccc(-c6ccc(-c7ccccc7)c7ccccc67)c5)nc(-c5cccc6ccccc56)n4)cc23)cc1 ; [2H]c1c([2H])c([2H])c2c([2H])c(-c3nc(-c4cccc(-c5ccc(-c6ccccc6)c6ccccc56)c4)nc(-c4ccc5ccc(-c6ccccc6)cc5c4)n3)c([2H])c([2H])c2c1[2H]]

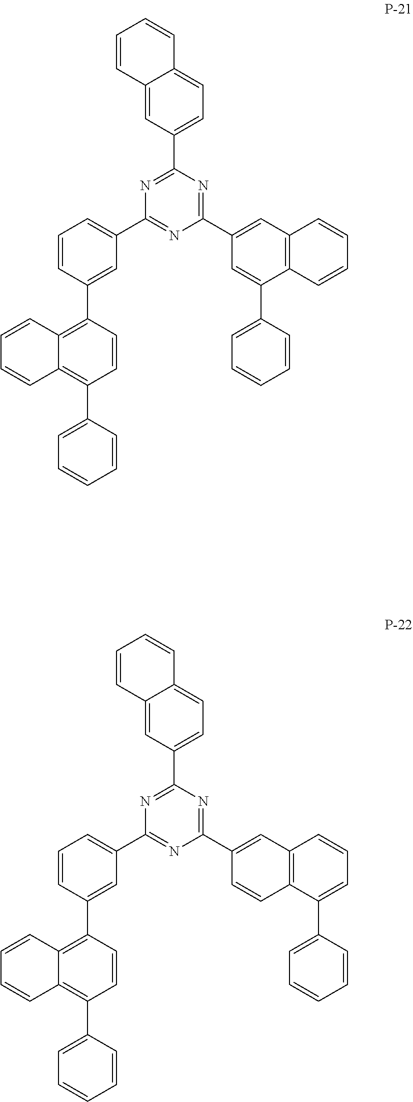 [SMILES: c1ccc(-c2cc(-c3nc(-c4cccc(-c5ccc(-c6ccccc6)c6ccccc56)c4)nc(-c4ccc5ccccc5c4)n3)cc3ccccc23)cc1 ; c1ccc(-c2cccc3cc(-c4nc(-c5cccc(-c6ccc(-c7ccccc7)c7ccccc67)c5)nc(-c5ccc6ccccc6c5)n4)ccc23)cc1]

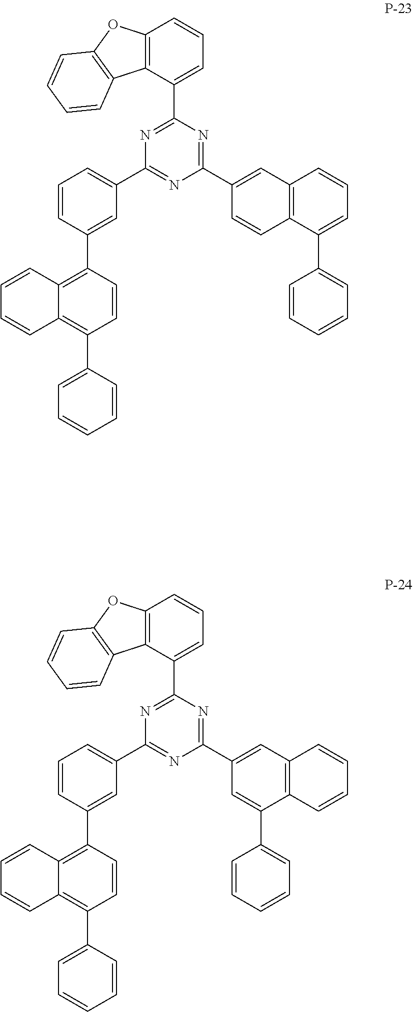 [SMILES: c1ccc(-c2cccc3cc(-c4nc(-c5cccc(-c6ccc(-c7ccccc7)c7ccccc67)c5)nc(-c5cccc6oc7ccccc7c56)n4)ccc23)cc1 ; c1ccc(-c2cc(-c3nc(-c4cccc(-c5ccc(-c6ccccc6)c6ccccc56)c4)nc(-c4cccc5oc6ccccc6c45)n3)cc3ccccc23)cc1]

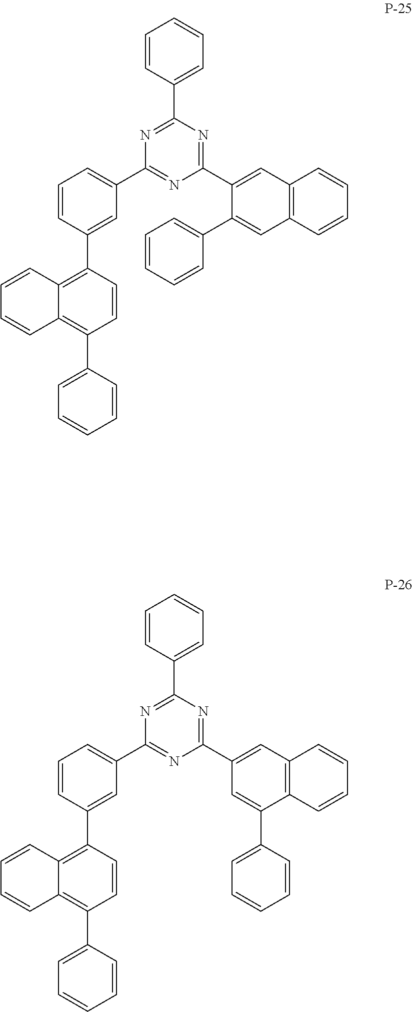 [SMILES: c1ccc(-c2nc(-c3cccc(-c4ccc(-c5ccccc5)c5ccccc45)c3)nc(-c3cc4ccccc4cc3-c3ccccc3)n2)cc1 ; c1ccc(-c2nc(-c3cccc(-c4ccc(-c5ccccc5)c5ccccc45)c3)nc(-c3cc(-c4ccccc4)c4ccccc4c3)n2)cc1]

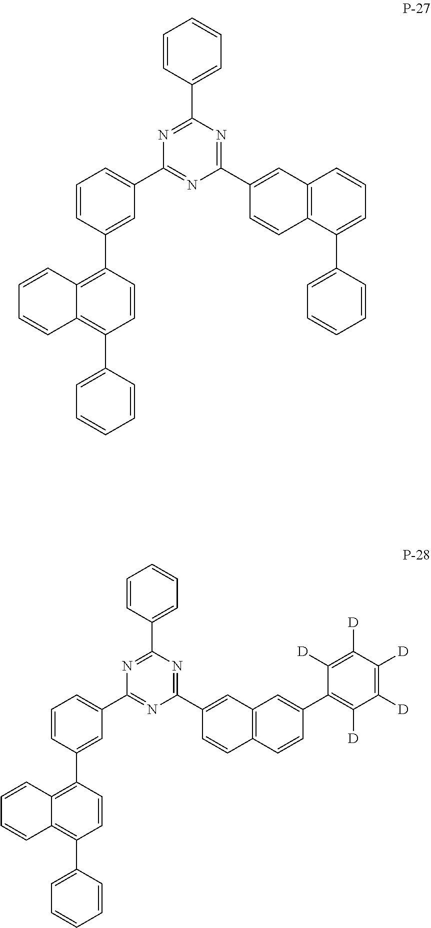 [SMILES: c1ccc(-c2nc(-c3cccc(-c4ccc(-c5ccccc5)c5ccccc45)c3)nc(-c3ccc4c(-c5ccccc5)cccc4c3)n2)cc1 ; [2H]c1c([2H])c([2H])c(-c2ccc3ccc(-c4nc(-c5ccccc5)nc(-c5cccc(-c6ccc(-c7ccccc7)c7ccccc67)c5)n4)cc3c2)c([2H])c1[2H]]

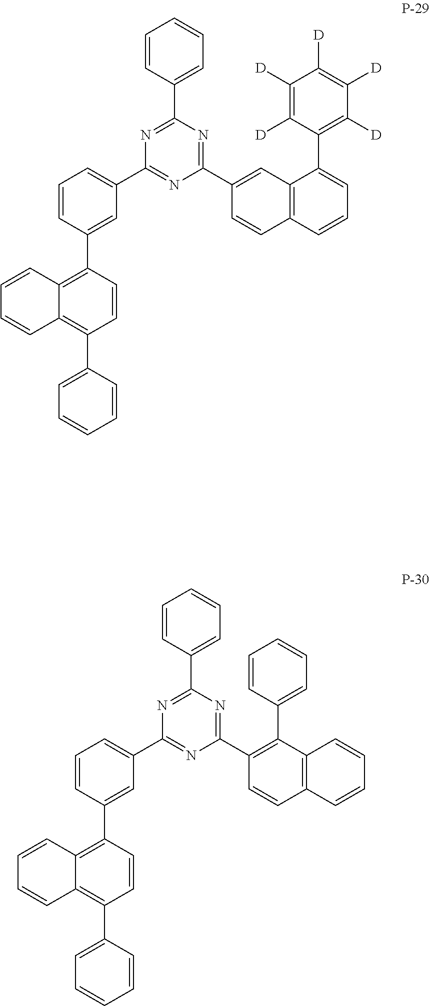 [SMILES: [2H]c1c([2H])c([2H])c(-c2cccc3ccc(-c4nc(-c5ccccc5)nc(-c5cccc(-c6ccc(-c7ccccc7)c7ccccc67)c5)n4)cc23)c([2H])c1[2H] ; c1ccc(-c2nc(-c3cccc(-c4ccc(-c5ccccc5)c5ccccc45)c3)nc(-c3ccc4ccccc4c3-c3ccccc3)n2)cc1]

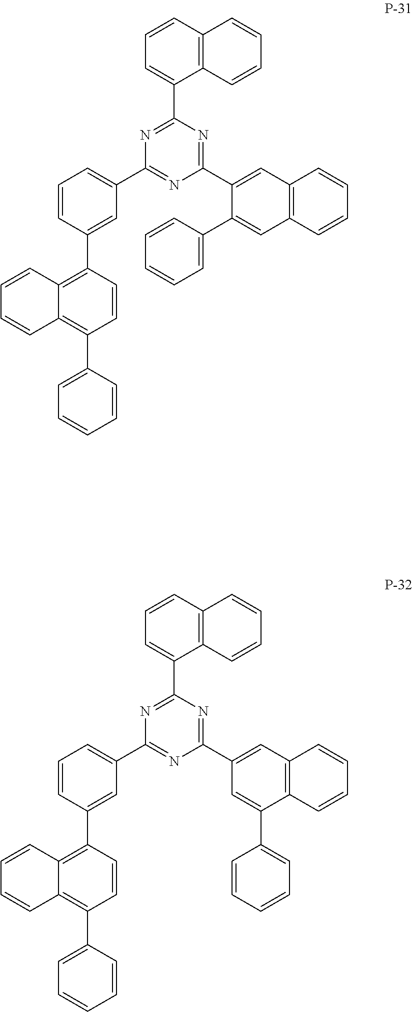 [SMILES: c1ccc(-c2cc3ccccc3cc2-c2nc(-c3cccc(-c4ccc(-c5ccccc5)c5ccccc45)c3)nc(-c3cccc4ccccc34)n2)cc1 ; c1ccc(-c2cc(-c3nc(-c4cccc(-c5ccc(-c6ccccc6)c6ccccc56)c4)nc(-c4cccc5ccccc45)n3)cc3ccccc23)cc1]

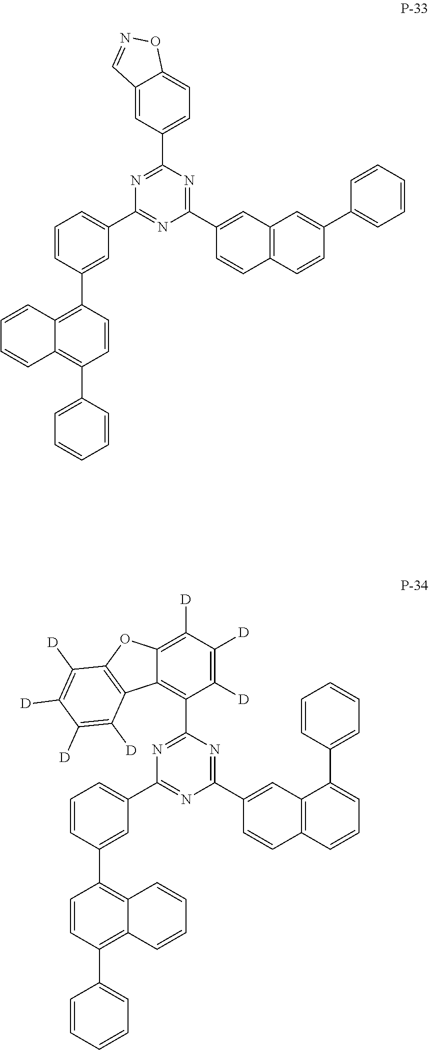 [SMILES: c1ccc(-c2ccc3ccc(-c4nc(-c5cccc(-c6ccc(-c7ccccc7)c7ccccc67)c5)nc(-c5ccc6oncc6c5)n4)cc3c2)cc1 ; [2H]c1c([2H])c([2H])c2c(oc3c([2H])c([2H])c([2H])c(-c4nc(-c5cccc(-c6ccc(-c7ccccc7)c7ccccc67)c5)nc(-c5ccc6cccc(-c7ccccc7)c6c5)n4)c32)c1[2H]]

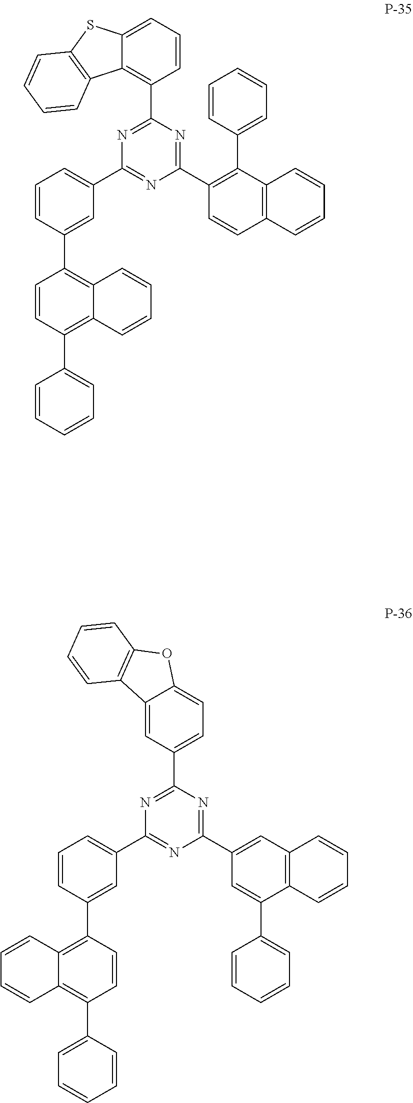 [SMILES: c1ccc(-c2c(-c3nc(-c4cccc(-c5ccc(-c6ccccc6)c6ccccc56)c4)nc(-c4cccc5sc6ccccc6c45)n3)ccc3ccccc23)cc1 ; c1ccc(-c2cc(-c3nc(-c4cccc(-c5ccc(-c6ccccc6)c6ccccc56)c4)nc(-c4ccc5oc6ccccc6c5c4)n3)cc3ccccc23)cc1]

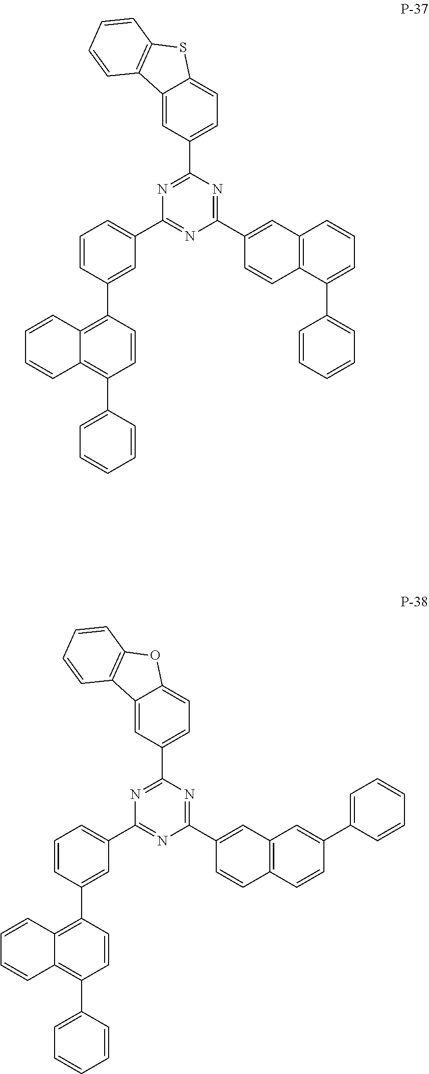 [SMILES: c1ccc(-c2cccc3cc(-c4nc(-c5cccc(-c6ccc(-c7ccccc7)c7ccccc67)c5)nc(-c5ccc6sc7ccccc7c6c5)n4)ccc23)cc1 ; c1ccc(-c2ccc3ccc(-c4nc(-c5cccc(-c6ccc(-c7ccccc7)c7ccccc67)c5)nc(-c5ccc6oc7ccccc7c6c5)n4)cc3c2)cc1]

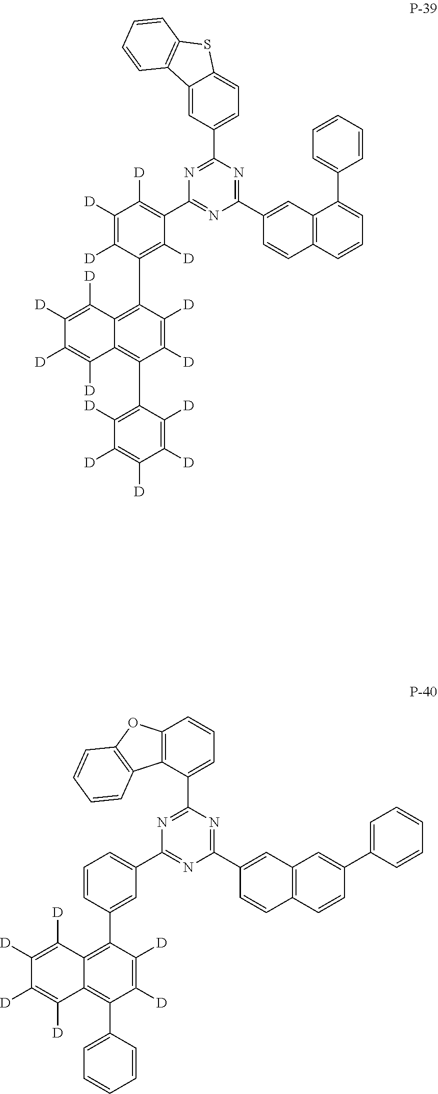 [SMILES: [2H]c1c([2H])c([2H])c(-c2c([2H])c([2H])c(-c3c([2H])c([2H])c([2H])c(-c4nc(-c5ccc6cccc(-c7ccccc7)c6c5)nc(-c5ccc6sc7ccccc7c6c5)n4)c3[2H])c3c([2H])c([2H])c([2H])c([2H])c23)c([2H])c1[2H] ; [2H]c1c([2H])c([2H])c2c(-c3cccc(-c4nc(-c5ccc6ccc(-c7ccccc7)cc6c5)nc(-c5cccc6oc7ccccc7c56)n4)c3)c([2H])c([2H])c(-c3ccccc3)c2c1[2H]]

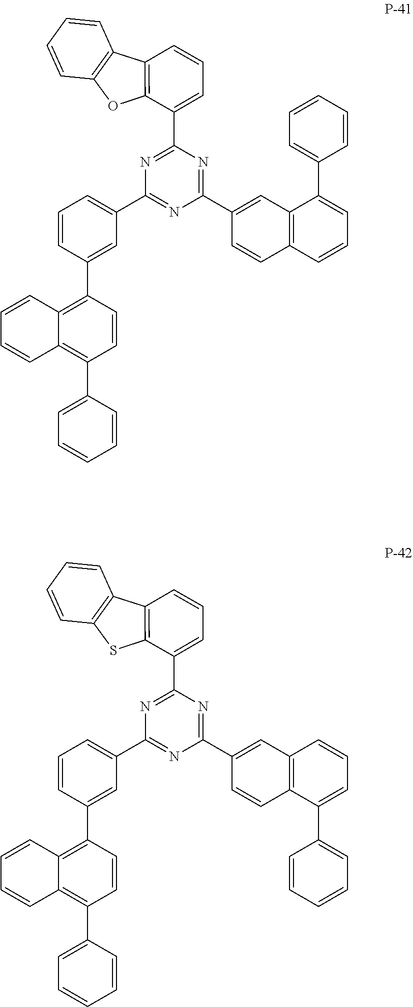 [SMILES: c1ccc(-c2cccc3ccc(-c4nc(-c5cccc(-c6ccc(-c7ccccc7)c7ccccc67)c5)nc(-c5cccc6c5oc5ccccc56)n4)cc23)cc1 ; c1ccc(-c2cccc3cc(-c4nc(-c5cccc(-c6ccc(-c7ccccc7)c7ccccc67)c5)nc(-c5cccc6c5sc5ccccc56)n4)ccc23)cc1]

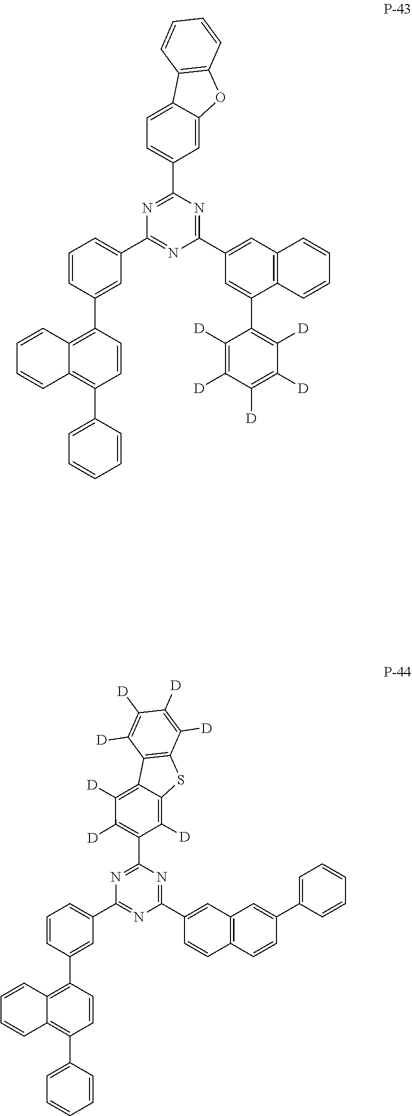 [SMILES: [2H]c1c([2H])c([2H])c(-c2cc(-c3nc(-c4cccc(-c5ccc(-c6ccccc6)c6ccccc56)c4)nc(-c4ccc5c(c4)oc4ccccc45)n3)cc3ccccc23)c([2H])c1[2H] ; [2H]c1c([2H])c([2H])c2c(sc3c([2H])c(-c4nc(-c5cccc(-c6ccc(-c7ccccc7)c7ccccc67)c5)nc(-c5ccc6ccc(-c7ccccc7)cc6c5)n4)c([2H])c([2H])c32)c1[2H]]

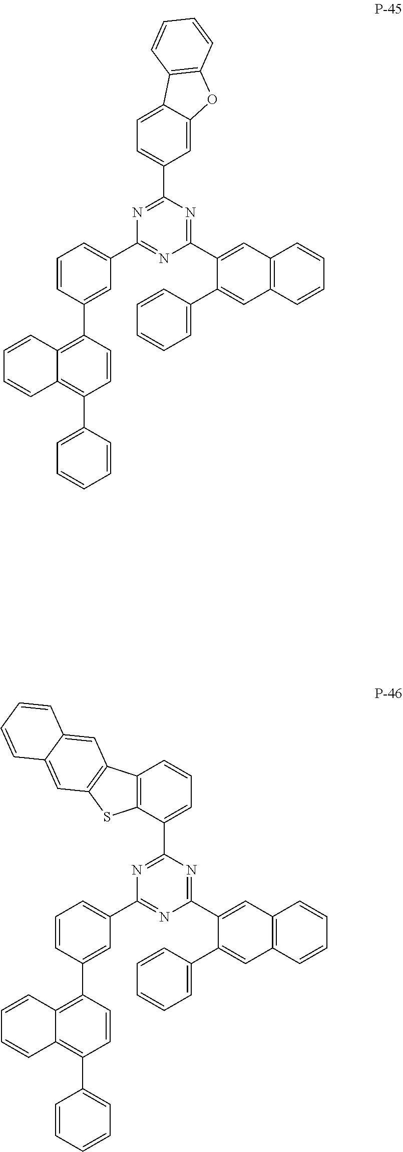 [SMILES: c1ccc(-c2cc3ccccc3cc2-c2nc(-c3cccc(-c4ccc(-c5ccccc5)c5ccccc45)c3)nc(-c3ccc4c(c3)oc3ccccc34)n2)cc1 ; c1ccc(-c2cc3ccccc3cc2-c2nc(-c3cccc(-c4ccc(-c5ccccc5)c5ccccc45)c3)nc(-c3cccc4c3sc3cc5ccccc5cc34)n2)cc1]

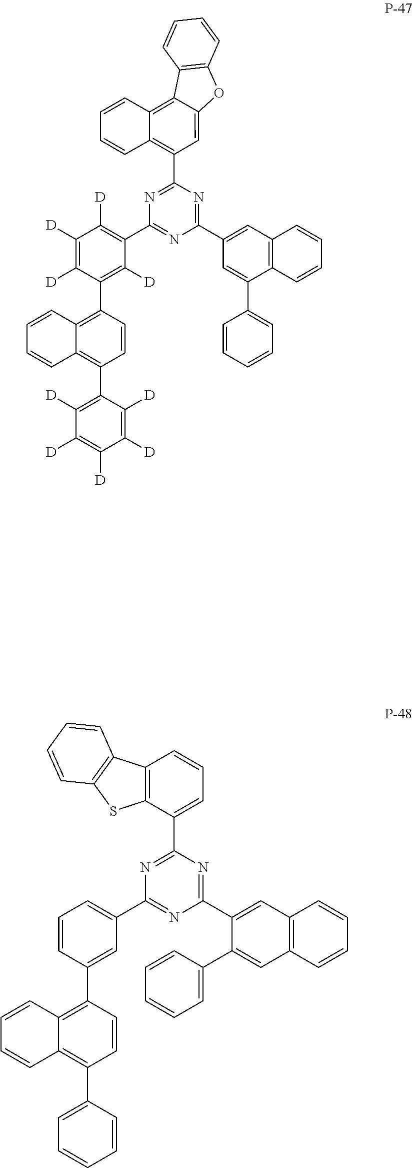 [SMILES: [2H]c1c([2H])c([2H])c(-c2ccc(-c3c([2H])c([2H])c([2H])c(-c4nc(-c5cc(-c6ccccc6)c6ccccc6c5)nc(-c5cc6oc7ccccc7c6c6ccccc56)n4)c3[2H])c3ccccc23)c([2H])c1[2H] ; c1ccc(-c2cc3ccccc3cc2-c2nc(-c3cccc(-c4ccc(-c5ccccc5)c5ccccc45)c3)nc(-c3cccc4c3sc3ccccc34)n2)cc1]

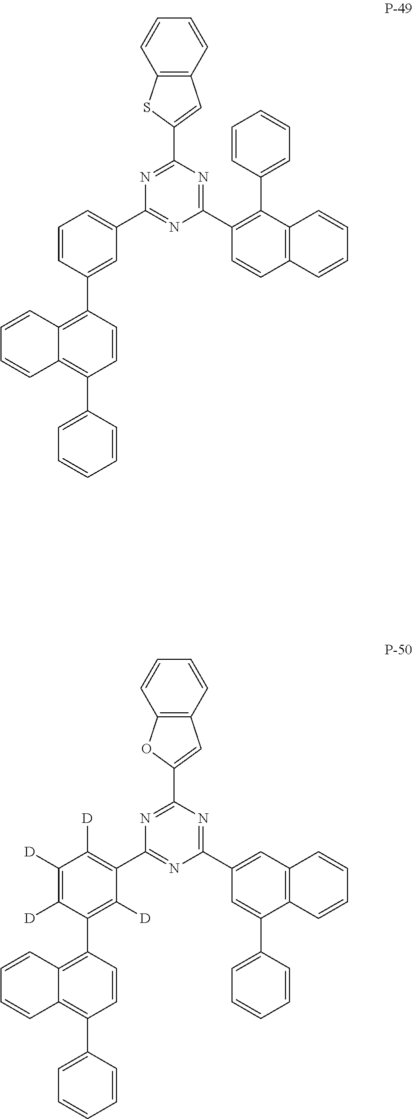 [SMILES: c1ccc(-c2c(-c3nc(-c4cccc(-c5ccc(-c6ccccc6)c6ccccc56)c4)nc(-c4cc5ccccc5s4)n3)ccc3ccccc23)cc1 ; [2H]c1c([2H])c(-c2nc(-c3cc(-c4ccccc4)c4ccccc4c3)nc(-c3cc4ccccc4o3)n2)c([2H])c(-c2ccc(-c3ccccc3)c3ccccc23)c1[2H]]

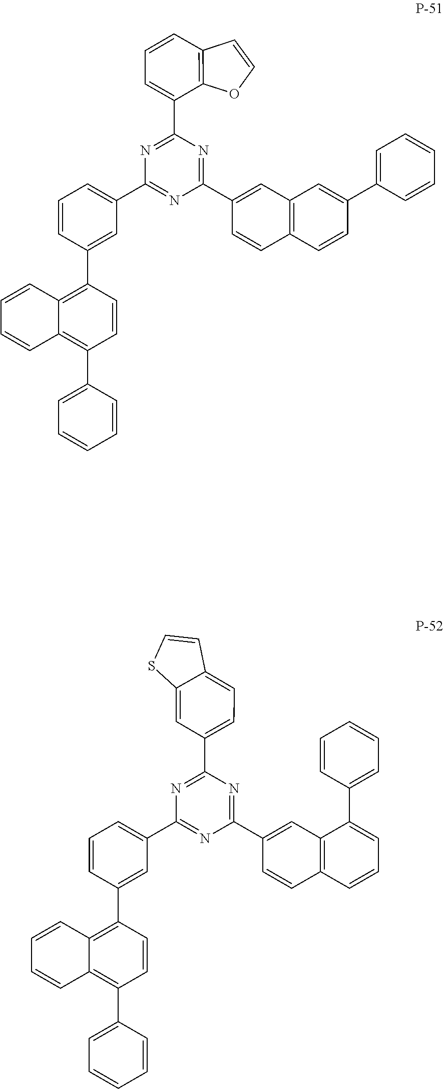 [SMILES: c1ccc(-c2ccc3ccc(-c4nc(-c5cccc(-c6ccc(-c7ccccc7)c7ccccc67)c5)nc(-c5cccc6ccoc56)n4)cc3c2)cc1 ; c1ccc(-c2cccc3ccc(-c4nc(-c5cccc(-c6ccc(-c7ccccc7)c7ccccc67)c5)nc(-c5ccc6ccsc6c5)n4)cc23)cc1]

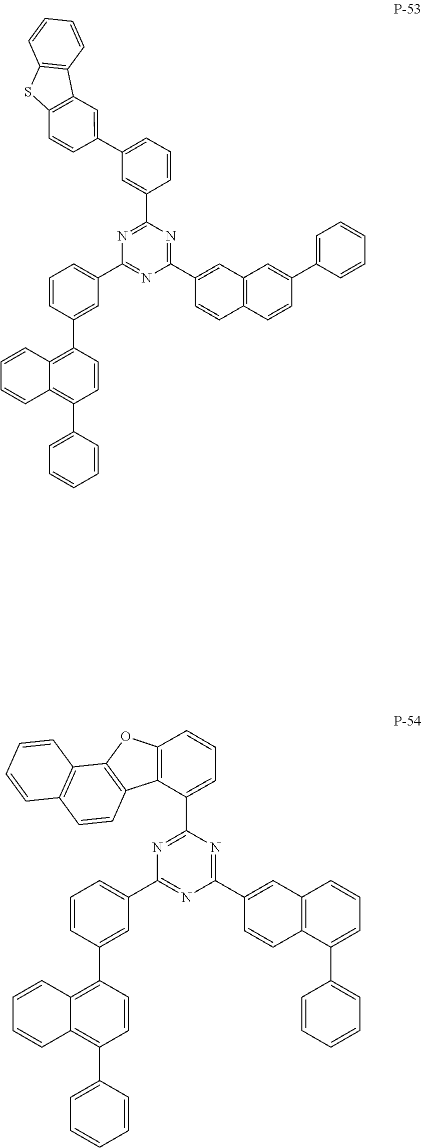 [SMILES: c1ccc(-c2ccc3ccc(-c4nc(-c5cccc(-c6ccc7sc8ccccc8c7c6)c5)nc(-c5cccc(-c6ccc(-c7ccccc7)c7ccccc67)c5)n4)cc3c2)cc1 ; c1ccc(-c2cccc3cc(-c4nc(-c5cccc(-c6ccc(-c7ccccc7)c7ccccc67)c5)nc(-c5cccc6oc7c8ccccc8ccc7c56)n4)ccc23)cc1]

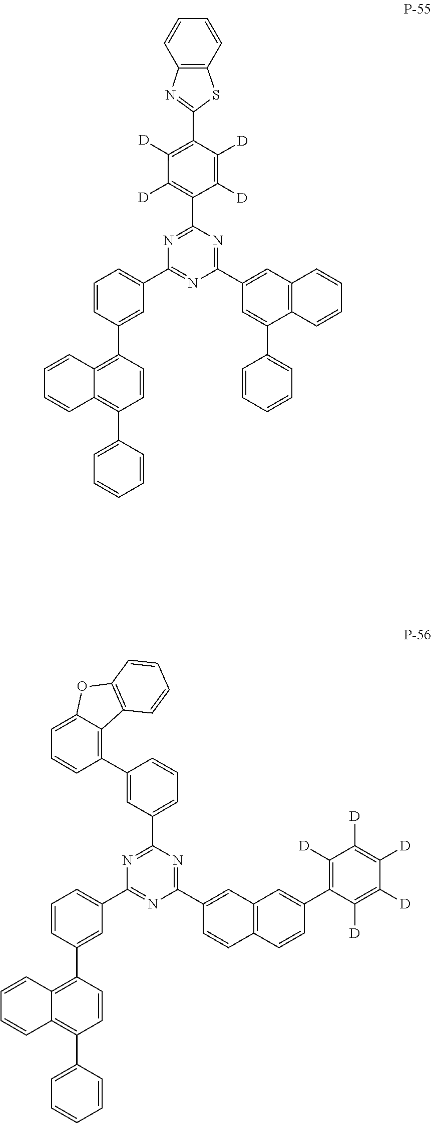 [SMILES: [2H]c1c([2H])c(-c2nc3ccccc3s2)c([2H])c([2H])c1-c1nc(-c2cccc(-c3ccc(-c4ccccc4)c4ccccc34)c2)nc(-c2cc(-c3ccccc3)c3ccccc3c2)n1 ; [2H]c1c([2H])c([2H])c(-c2ccc3ccc(-c4nc(-c5cccc(-c6ccc(-c7ccccc7)c7ccccc67)c5)nc(-c5cccc(-c6cccc7oc8ccccc8c67)c5)n4)cc3c2)c([2H])c1[2H]]

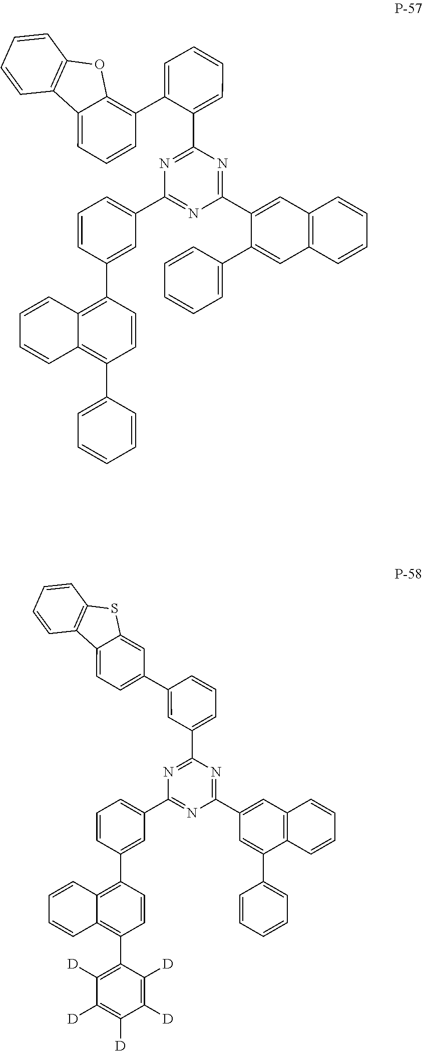 [SMILES: c1ccc(-c2cc3ccccc3cc2-c2nc(-c3cccc(-c4ccc(-c5ccccc5)c5ccccc45)c3)nc(-c3ccccc3-c3cccc4c3oc3ccccc34)n2)cc1 ; [2H]c1c([2H])c([2H])c(-c2ccc(-c3cccc(-c4nc(-c5cccc(-c6ccc7c(c6)sc6ccccc67)c5)nc(-c5cc(-c6ccccc6)c6ccccc6c5)n4)c3)c3ccccc23)c([2H])c1[2H]]

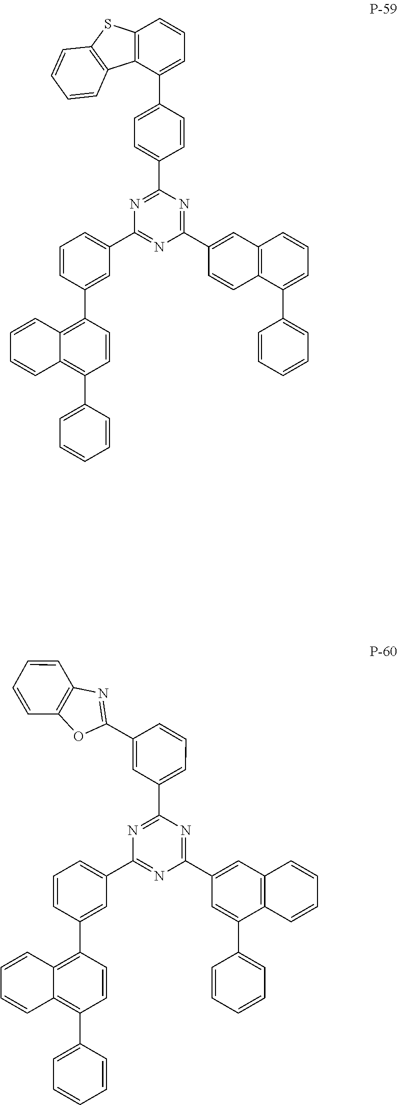 [SMILES: c1ccc(-c2cccc3cc(-c4nc(-c5ccc(-c6cccc7sc8ccccc8c67)cc5)nc(-c5cccc(-c6ccc(-c7ccccc7)c7ccccc67)c5)n4)ccc23)cc1 ; c1ccc(-c2cc(-c3nc(-c4cccc(-c5nc6ccccc6o5)c4)nc(-c4cccc(-c5ccc(-c6ccccc6)c6ccccc56)c4)n3)cc3ccccc23)cc1]

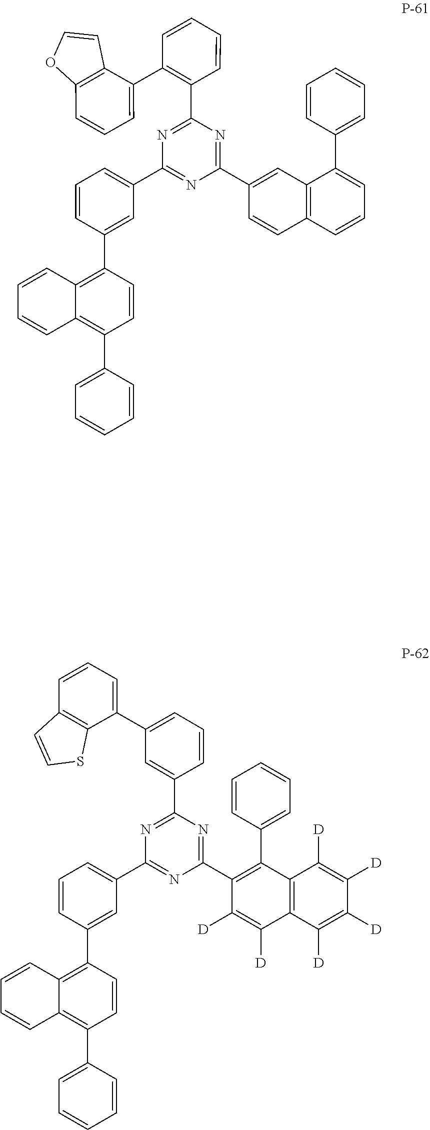 [SMILES: c1ccc(-c2cccc3ccc(-c4nc(-c5cccc(-c6ccc(-c7ccccc7)c7ccccc67)c5)nc(-c5ccccc5-c5cccc6occc56)n4)cc23)cc1 ; [2H]c1c([2H])c([2H])c2c(-c3ccccc3)c(-c3nc(-c4cccc(-c5ccc(-c6ccccc6)c6ccccc56)c4)nc(-c4cccc(-c5cccc6ccsc56)c4)n3)c([2H])c([2H])c2c1[2H]]

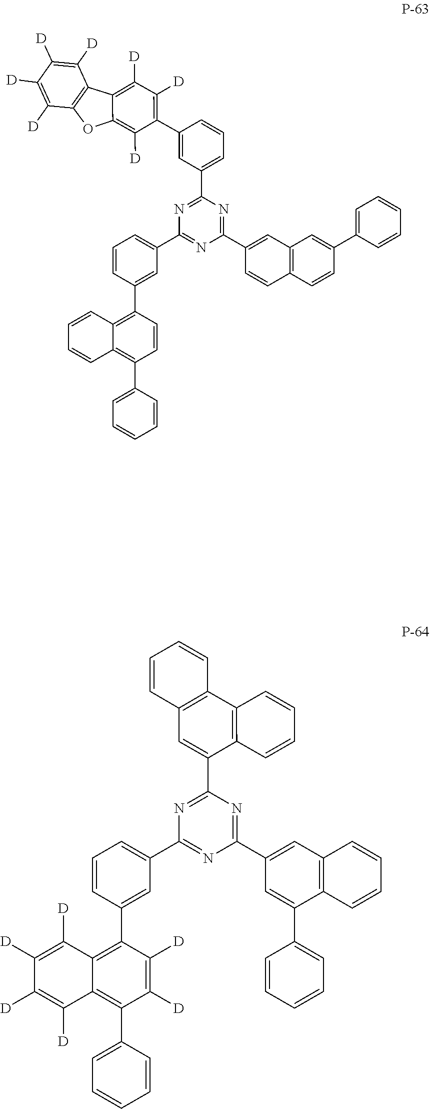 [SMILES: [2H]c1c([2H])c([2H])c2c(oc3c([2H])c(-c4cccc(-c5nc(-c6cccc(-c7ccc(-c8ccccc8)c8ccccc78)c6)nc(-c6ccc7ccc(-c8ccccc8)cc7c6)n5)c4)c([2H])c([2H])c32)c1[2H] ; [2H]c1c([2H])c([2H])c2c(-c3cccc(-c4nc(-c5cc(-c6ccccc6)c6ccccc6c5)nc(-c5cc6ccccc6c6ccccc56)n4)c3)c([2H])c([2H])c(-c3ccccc3)c2c1[2H]]

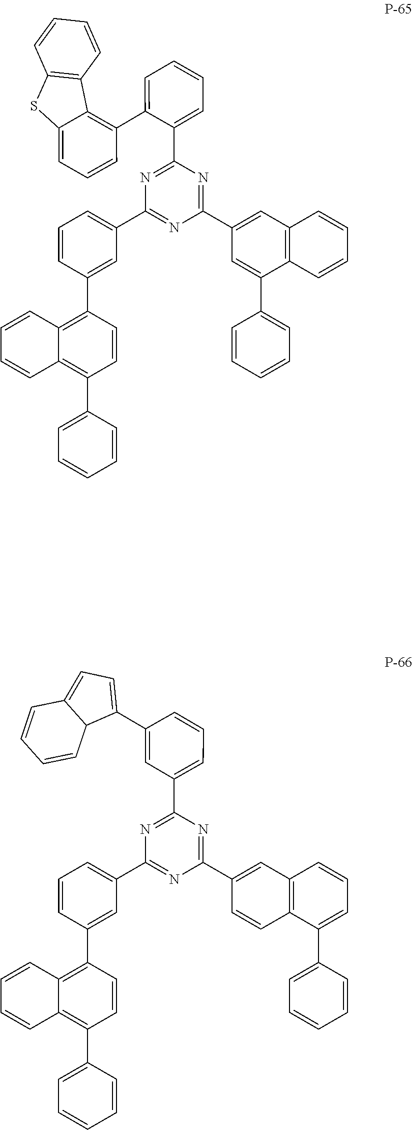 [SMILES: c1ccc(-c2cc(-c3nc(-c4cccc(-c5ccc(-c6ccccc6)c6ccccc56)c4)nc(-c4ccccc4-c4cccc5sc6ccccc6c45)n3)cc3ccccc23)cc1 ; C1=CC2=CC=C(c3cccc(-c4nc(-c5cccc(-c6ccc(-c7ccccc7)c7ccccc67)c5)nc(-c5ccc6c(-c7ccccc7)cccc6c5)n4)c3)C2C=C1]

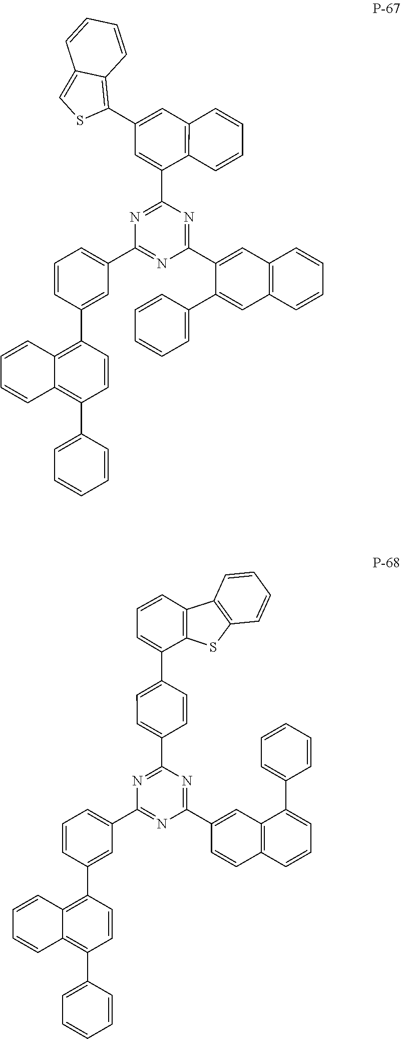 [SMILES: c1ccc(-c2cc3ccccc3cc2-c2nc(-c3cccc(-c4ccc(-c5ccccc5)c5ccccc45)c3)nc(-c3cc(-c4scc5ccccc45)cc4ccccc34)n2)cc1 ; c1ccc(-c2cccc3ccc(-c4nc(-c5ccc(-c6cccc7c6sc6ccccc67)cc5)nc(-c5cccc(-c6ccc(-c7ccccc7)c7ccccc67)c5)n4)cc23)cc1]

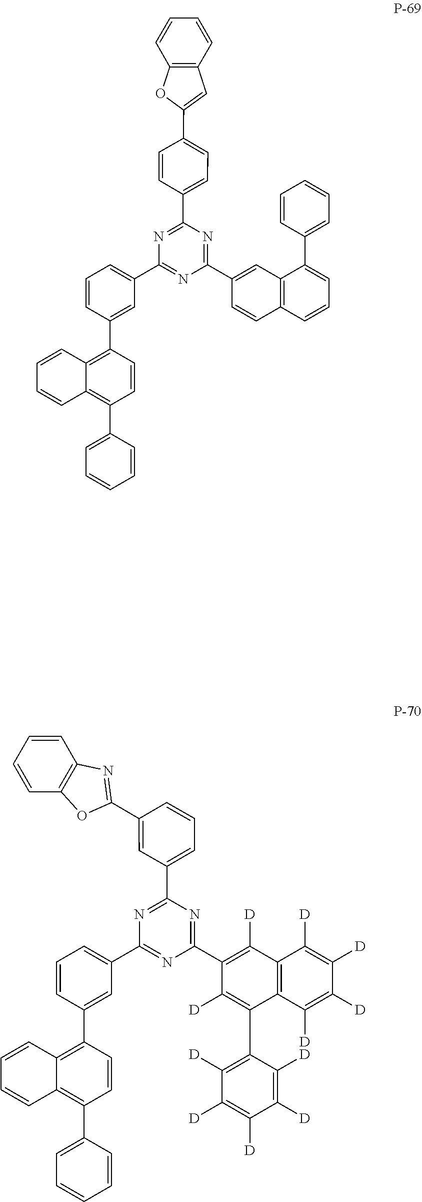 [SMILES: c1ccc(-c2cccc3ccc(-c4nc(-c5ccc(-c6cc7ccccc7o6)cc5)nc(-c5cccc(-c6ccc(-c7ccccc7)c7ccccc67)c5)n4)cc23)cc1 ; [2H]c1c([2H])c([2H])c(-c2c([2H])c(-c3nc(-c4cccc(-c5nc6ccccc6o5)c4)nc(-c4cccc(-c5ccc(-c6ccccc6)c6ccccc56)c4)n3)c([2H])c3c([2H])c([2H])c([2H])c([2H])c23)c([2H])c1[2H]]

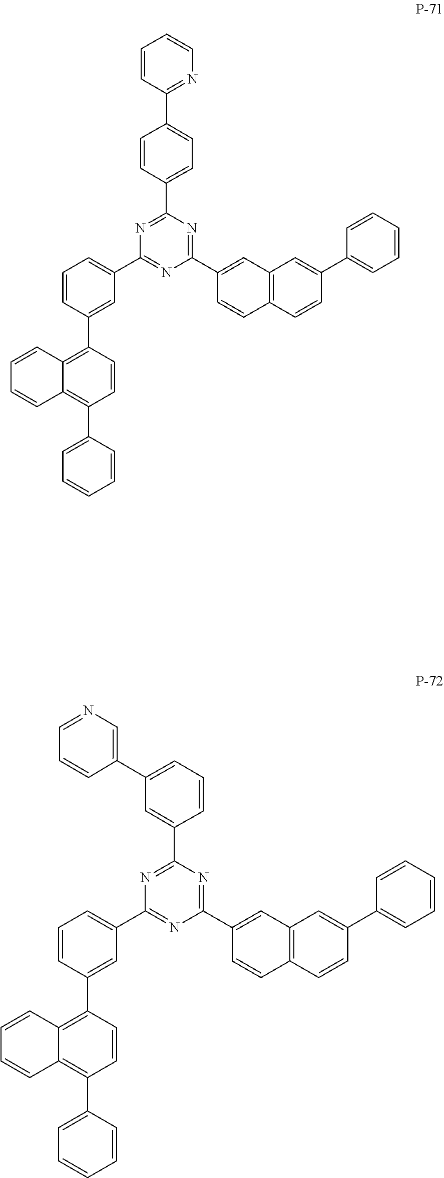 [SMILES: c1ccc(-c2ccc3ccc(-c4nc(-c5ccc(-c6ccccn6)cc5)nc(-c5cccc(-c6ccc(-c7ccccc7)c7ccccc67)c5)n4)cc3c2)cc1 ; c1ccc(-c2ccc3ccc(-c4nc(-c5cccc(-c6cccnc6)c5)nc(-c5cccc(-c6ccc(-c7ccccc7)c7ccccc67)c5)n4)cc3c2)cc1]

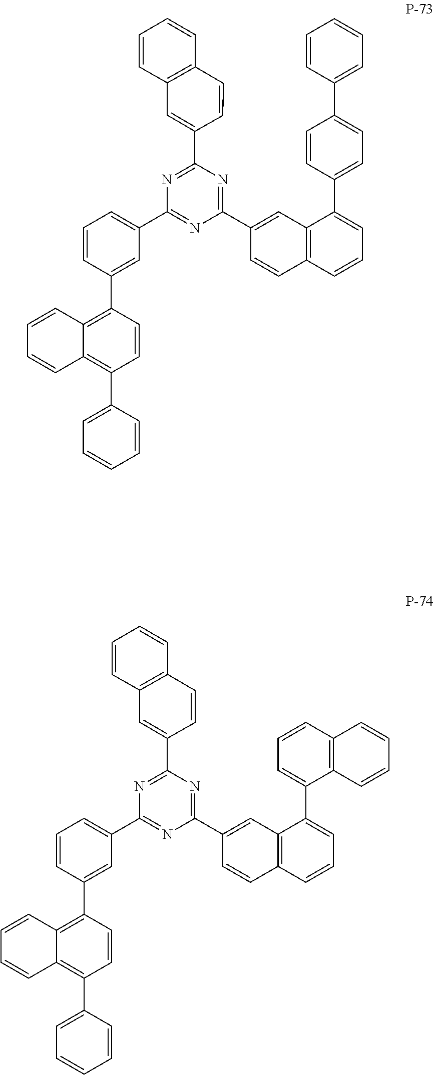 [SMILES: c1ccc(-c2ccc(-c3cccc4ccc(-c5nc(-c6cccc(-c7ccc(-c8ccccc8)c8ccccc78)c6)nc(-c6ccc7ccccc7c6)n5)cc34)cc2)cc1 ; c1ccc(-c2ccc(-c3cccc(-c4nc(-c5ccc6ccccc6c5)nc(-c5ccc6cccc(-c7cccc8ccccc78)c6c5)n4)c3)c3ccccc23)cc1]

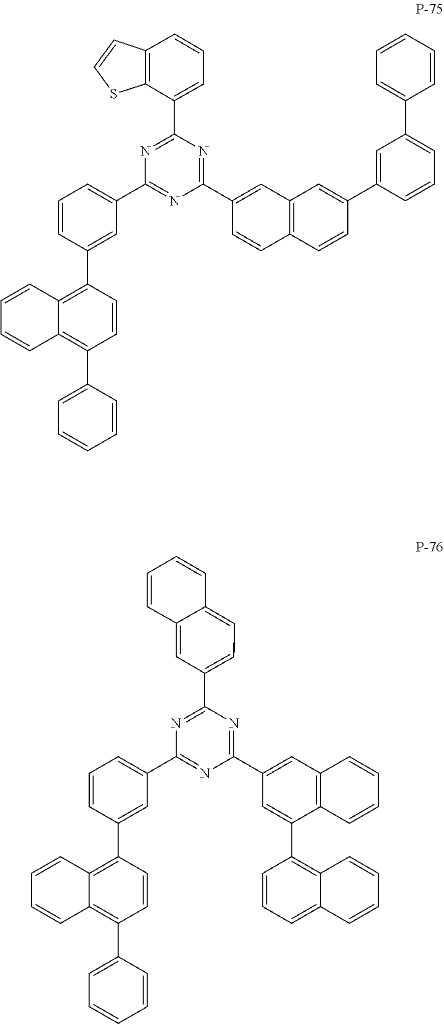 [SMILES: c1ccc(-c2cccc(-c3ccc4ccc(-c5nc(-c6cccc(-c7ccc(-c8ccccc8)c8ccccc78)c6)nc(-c6cccc7ccsc67)n5)cc4c3)c2)cc1 ; c1ccc(-c2ccc(-c3cccc(-c4nc(-c5ccc6ccccc6c5)nc(-c5cc(-c6cccc7ccccc67)c6ccccc6c5)n4)c3)c3ccccc23)cc1]

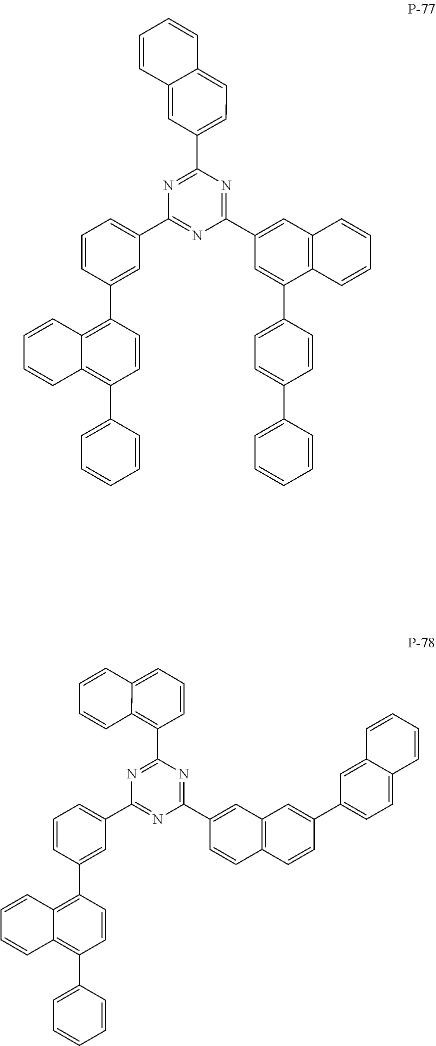 [SMILES: c1ccc(-c2ccc(-c3cc(-c4nc(-c5cccc(-c6ccc(-c7ccccc7)c7ccccc67)c5)nc(-c5ccc6ccccc6c5)n4)cc4ccccc34)cc2)cc1 ; c1ccc(-c2ccc(-c3cccc(-c4nc(-c5ccc6ccc(-c7ccc8ccccc8c7)cc6c5)nc(-c5cccc6ccccc56)n4)c3)c3ccccc23)cc1]

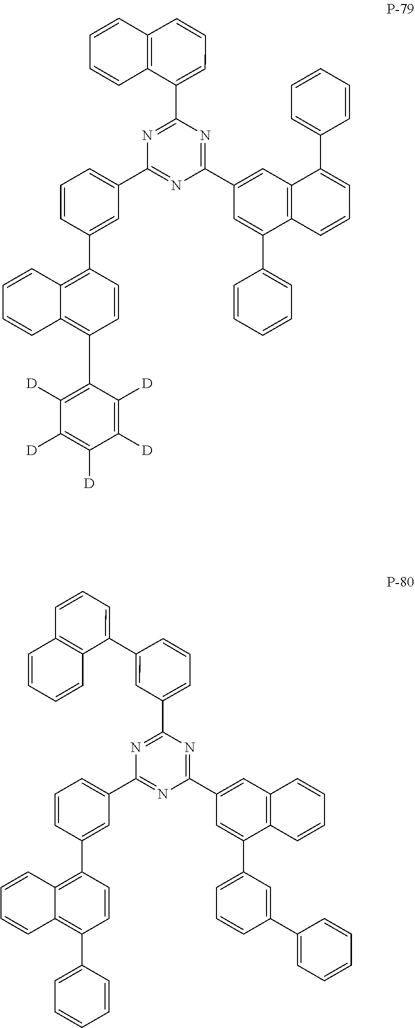 [SMILES: [2H]c1c([2H])c([2H])c(-c2ccc(-c3cccc(-c4nc(-c5cc(-c6ccccc6)c6cccc(-c7ccccc7)c6c5)nc(-c5cccc6ccccc56)n4)c3)c3ccccc23)c([2H])c1[2H] ; c1ccc(-c2cccc(-c3cc(-c4nc(-c5cccc(-c6cccc7ccccc67)c5)nc(-c5cccc(-c6ccc(-c7ccccc7)c7ccccc67)c5)n4)cc4ccccc34)c2)cc1]

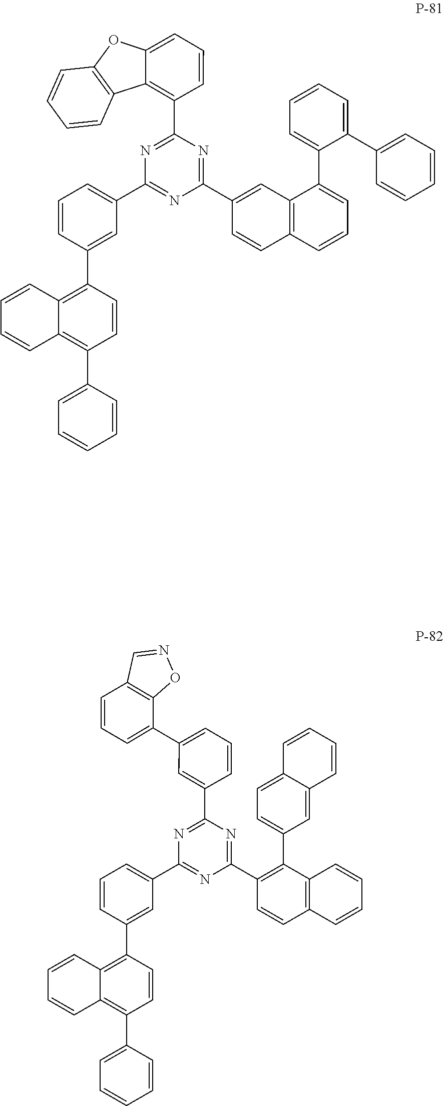 [SMILES: c1ccc(-c2ccccc2-c2cccc3ccc(-c4nc(-c5cccc(-c6ccc(-c7ccccc7)c7ccccc67)c5)nc(-c5cccc6oc7ccccc7c56)n4)cc23)cc1 ; c1ccc(-c2ccc(-c3cccc(-c4nc(-c5cccc(-c6cccc7cnoc67)c5)nc(-c5ccc6ccccc6c5-c5ccc6ccccc6c5)n4)c3)c3ccccc23)cc1]

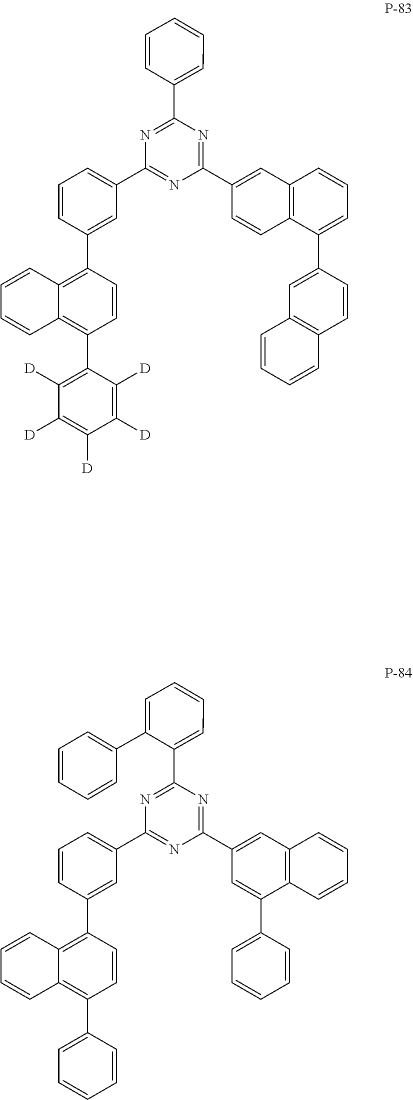 [SMILES: [2H]c1c([2H])c([2H])c(-c2ccc(-c3cccc(-c4nc(-c5ccccc5)nc(-c5ccc6c(-c7ccc8ccccc8c7)cccc6c5)n4)c3)c3ccccc23)c([2H])c1[2H] ; c1ccc(-c2ccccc2-c2nc(-c3cccc(-c4ccc(-c5ccccc5)c5ccccc45)c3)nc(-c3cc(-c4ccccc4)c4ccccc4c3)n2)cc1]

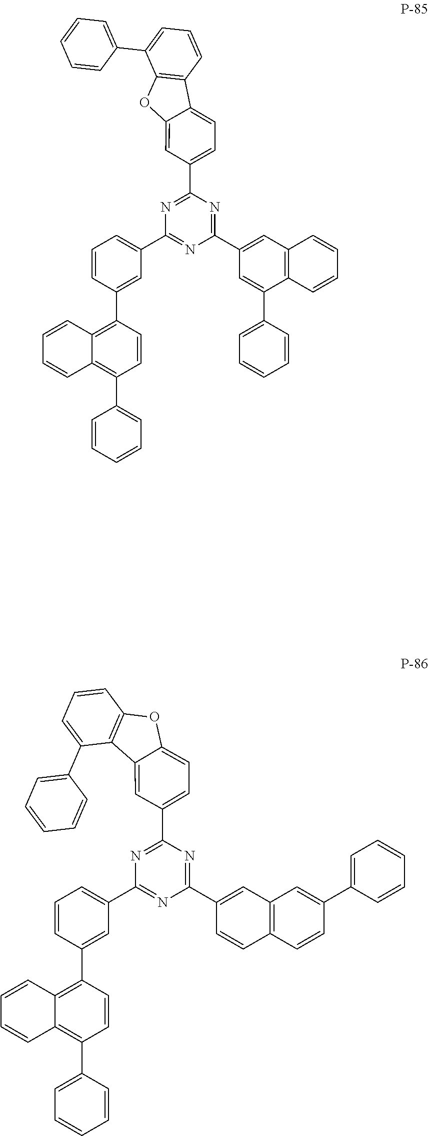 [SMILES: c1ccc(-c2cc(-c3nc(-c4cccc(-c5ccc(-c6ccccc6)c6ccccc56)c4)nc(-c4ccc5c(c4)oc4c(-c6ccccc6)cccc45)n3)cc3ccccc23)cc1 ; c1ccc(-c2ccc3ccc(-c4nc(-c5cccc(-c6ccc(-c7ccccc7)c7ccccc67)c5)nc(-c5ccc6oc7cccc(-c8ccccc8)c7c6c5)n4)cc3c2)cc1]

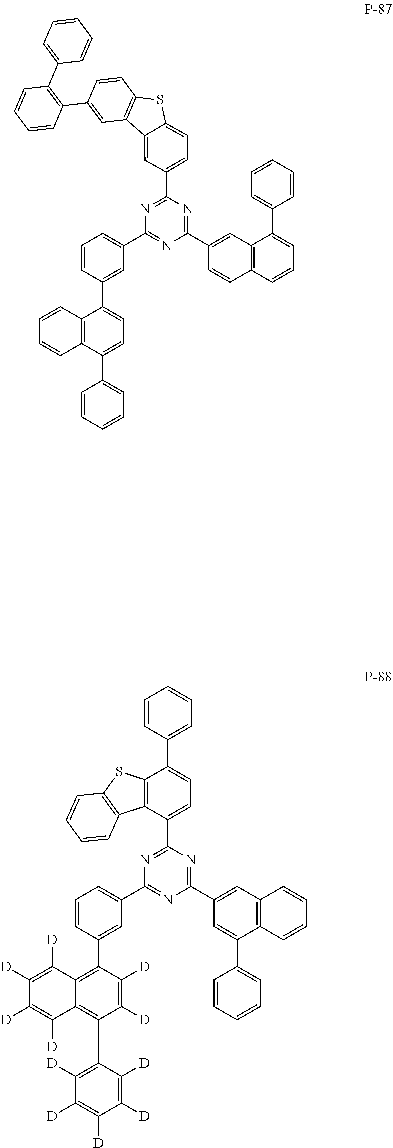 [SMILES: c1ccc(-c2ccccc2-c2ccc3sc4ccc(-c5nc(-c6cccc(-c7ccc(-c8ccccc8)c8ccccc78)c6)nc(-c6ccc7cccc(-c8ccccc8)c7c6)n5)cc4c3c2)cc1 ; [2H]c1c([2H])c([2H])c(-c2c([2H])c([2H])c(-c3cccc(-c4nc(-c5cc(-c6ccccc6)c6ccccc6c5)nc(-c5ccc(-c6ccccc6)c6sc7ccccc7c56)n4)c3)c3c([2H])c([2H])c([2H])c([2H])c23)c([2H])c1[2H]]

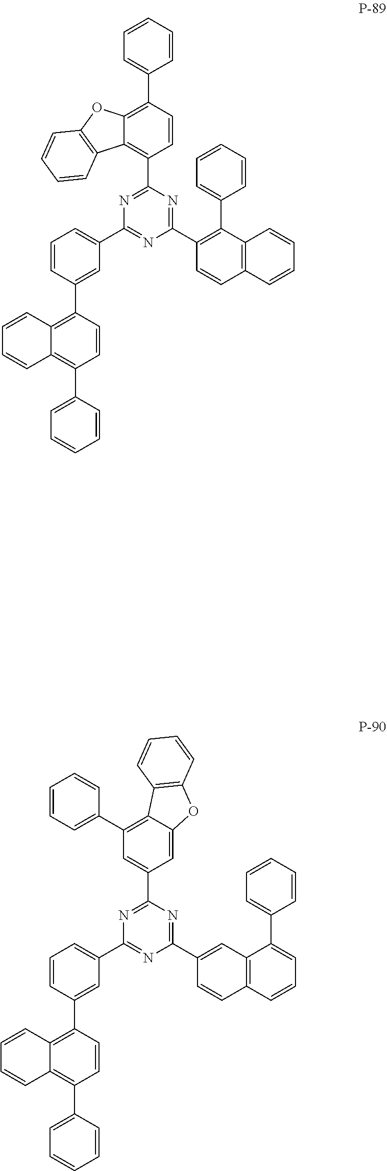 [SMILES: c1ccc(-c2c(-c3nc(-c4cccc(-c5ccc(-c6ccccc6)c6ccccc56)c4)nc(-c4ccc(-c5ccccc5)c5oc6ccccc6c45)n3)ccc3ccccc23)cc1 ; c1ccc(-c2cccc3ccc(-c4nc(-c5cccc(-c6ccc(-c7ccccc7)c7ccccc67)c5)nc(-c5cc(-c6ccccc6)c6c(c5)oc5ccccc56)n4)cc23)cc1]

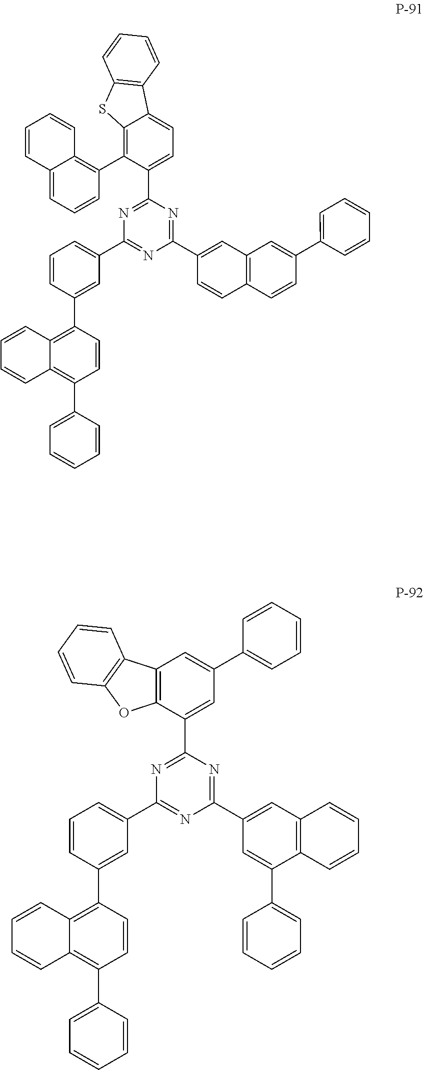 [SMILES: c1ccc(-c2ccc3ccc(-c4nc(-c5cccc(-c6ccc(-c7ccccc7)c7ccccc67)c5)nc(-c5ccc6c(sc7ccccc76)c5-c5cccc6ccccc56)n4)cc3c2)cc1 ; c1ccc(-c2cc(-c3nc(-c4cccc(-c5ccc(-c6ccccc6)c6ccccc56)c4)nc(-c4cc(-c5ccccc5)c5ccccc5c4)n3)c3oc4ccccc4c3c2)cc1]

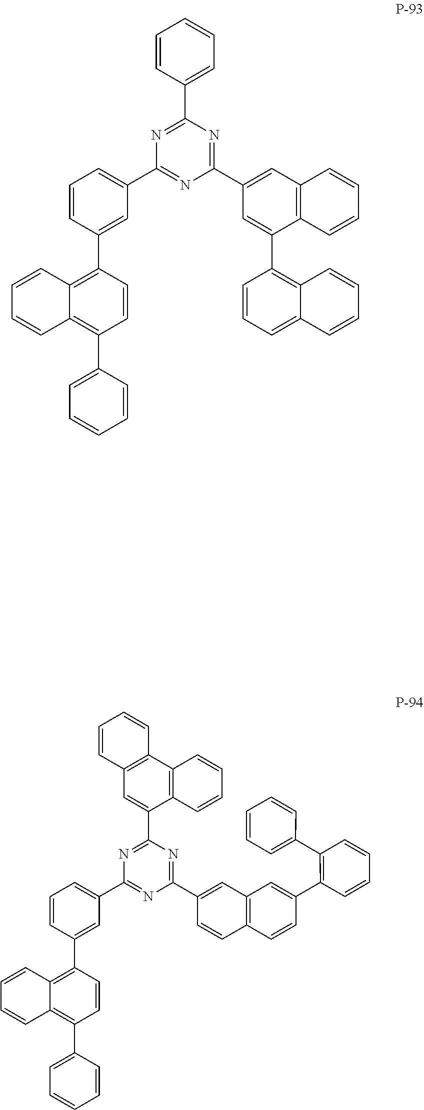 [SMILES: c1ccc(-c2nc(-c3cccc(-c4ccc(-c5ccccc5)c5ccccc45)c3)nc(-c3cc(-c4cccc5ccccc45)c4ccccc4c3)n2)cc1 ; c1ccc(-c2ccccc2-c2ccc3ccc(-c4nc(-c5cccc(-c6ccc(-c7ccccc7)c7ccccc67)c5)nc(-c5cc6ccccc6c6ccccc56)n4)cc3c2)cc1]

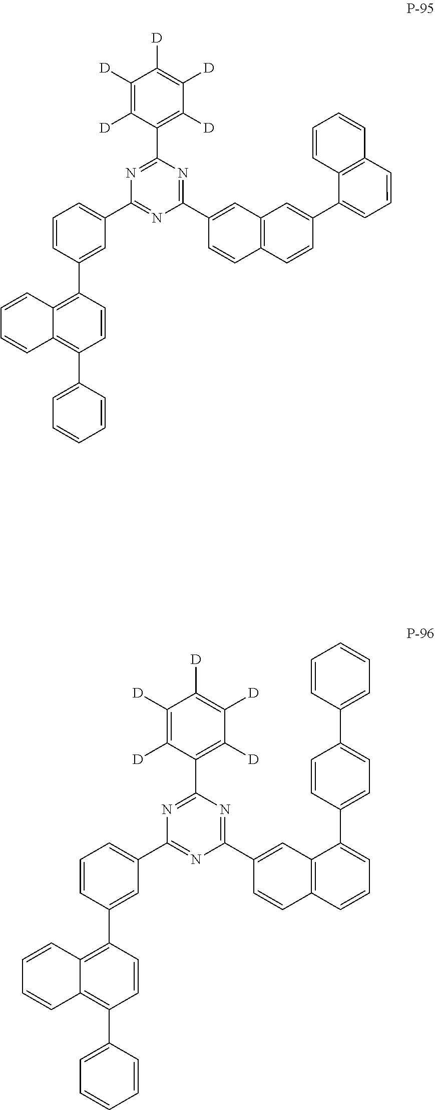 [SMILES: [2H]c1c([2H])c([2H])c(-c2nc(-c3cccc(-c4ccc(-c5ccccc5)c5ccccc45)c3)nc(-c3ccc4ccc(-c5cccc6ccccc56)cc4c3)n2)c([2H])c1[2H] ; [2H]c1c([2H])c([2H])c(-c2nc(-c3cccc(-c4ccc(-c5ccccc5)c5ccccc45)c3)nc(-c3ccc4cccc(-c5ccc(-c6ccccc6)cc5)c4c3)n2)c([2H])c1[2H]]

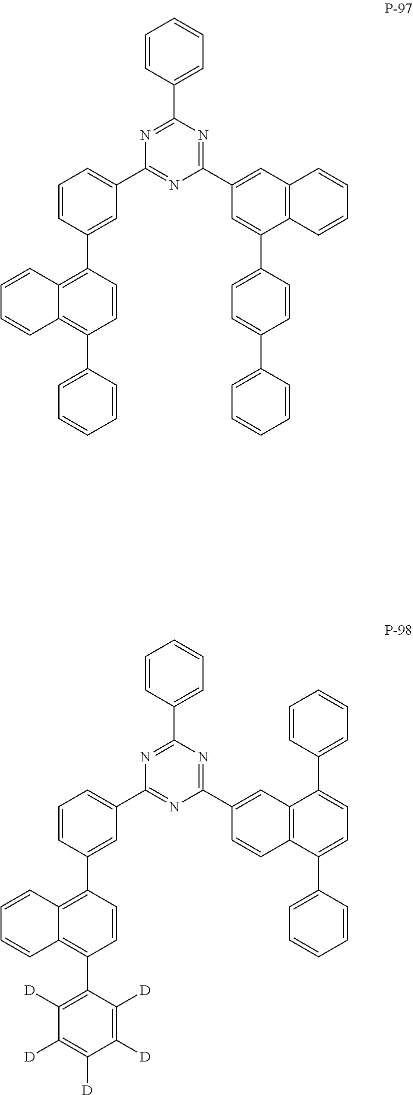 [SMILES: c1ccc(-c2ccc(-c3cc(-c4nc(-c5ccccc5)nc(-c5cccc(-c6ccc(-c7ccccc7)c7ccccc67)c5)n4)cc4ccccc34)cc2)cc1 ; [2H]c1c([2H])c([2H])c(-c2ccc(-c3cccc(-c4nc(-c5ccccc5)nc(-c5ccc6c(-c7ccccc7)ccc(-c7ccccc7)c6c5)n4)c3)c3ccccc23)c([2H])c1[2H]]

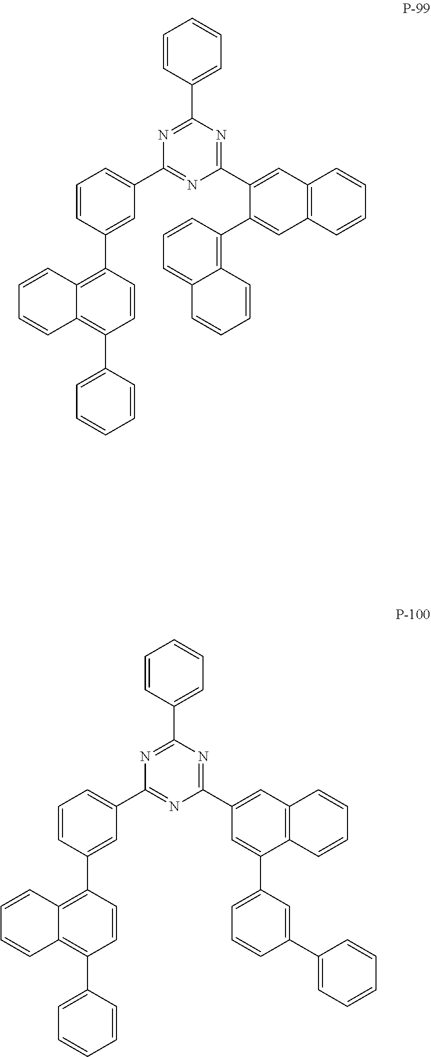 [SMILES: c1ccc(-c2nc(-c3cccc(-c4ccc(-c5ccccc5)c5ccccc45)c3)nc(-c3cc4ccccc4cc3-c3cccc4ccccc34)n2)cc1 ; c1ccc(-c2cccc(-c3cc(-c4nc(-c5ccccc5)nc(-c5cccc(-c6ccc(-c7ccccc7)c7ccccc67)c5)n4)cc4ccccc34)c2)cc1]

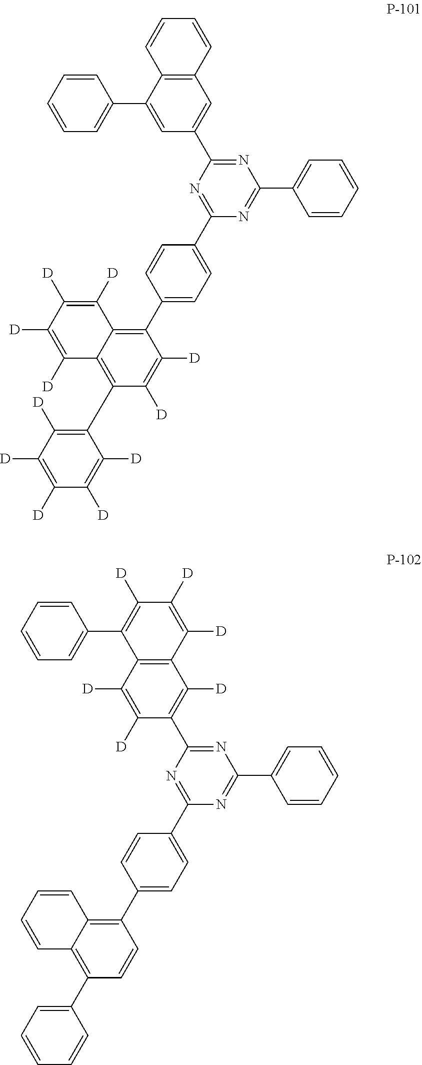 [SMILES: [2H]c1c([2H])c([2H])c(-c2c([2H])c([2H])c(-c3ccc(-c4nc(-c5ccccc5)nc(-c5cc(-c6ccccc6)c6ccccc6c5)n4)cc3)c3c([2H])c([2H])c([2H])c([2H])c23)c([2H])c1[2H] ; [2H]c1c([2H])c(-c2ccccc2)c2c([2H])c([2H])c(-c3nc(-c4ccccc4)nc(-c4ccc(-c5ccc(-c6ccccc6)c6ccccc56)cc4)n3)c([2H])c2c1[2H]]

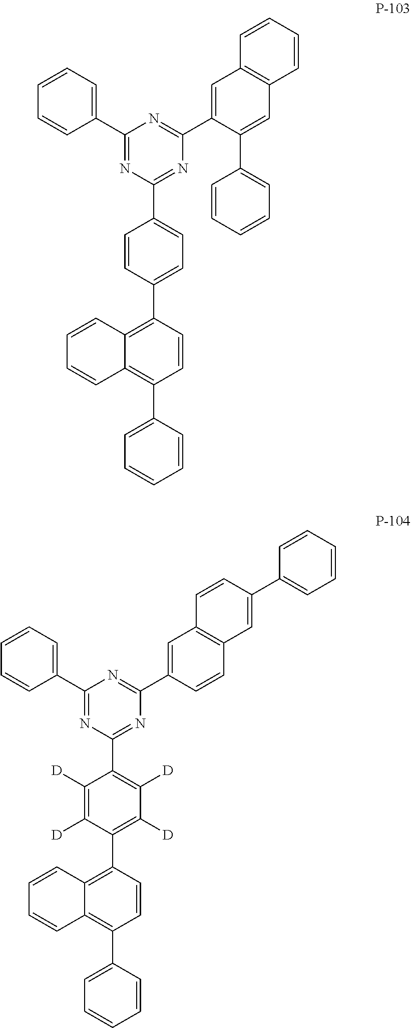 [SMILES: c1ccc(-c2nc(-c3ccc(-c4ccc(-c5ccccc5)c5ccccc45)cc3)nc(-c3cc4ccccc4cc3-c3ccccc3)n2)cc1 ; [2H]c1c([2H])c(-c2ccc(-c3ccccc3)c3ccccc23)c([2H])c([2H])c1-c1nc(-c2ccccc2)nc(-c2ccc3cc(-c4ccccc4)ccc3c2)n1]

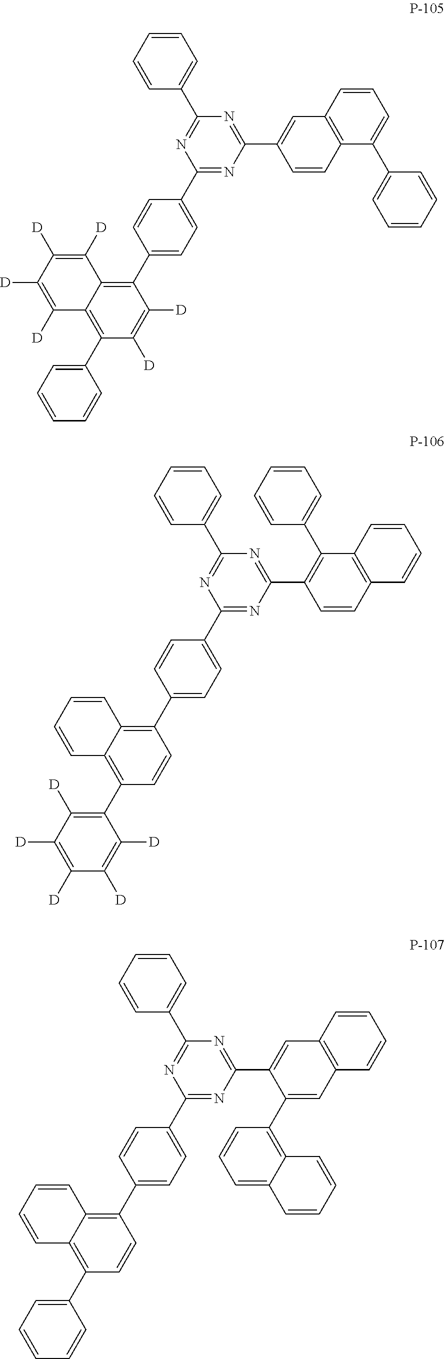 [SMILES: [2H]c1c([2H])c([2H])c2c(-c3ccc(-c4nc(-c5ccccc5)nc(-c5ccc6c(-c7ccccc7)cccc6c5)n4)cc3)c([2H])c([2H])c(-c3ccccc3)c2c1[2H] ; [2H]c1c([2H])c([2H])c(-c2ccc(-c3ccc(-c4nc(-c5ccccc5)nc(-c5ccc6ccccc6c5-c5ccccc5)n4)cc3)c3ccccc23)c([2H])c1[2H] ; c1ccc(-c2nc(-c3ccc(-c4ccc(-c5ccccc5)c5ccccc45)cc3)nc(-c3cc4ccccc4cc3-c3cccc4ccccc34)n2)cc1]

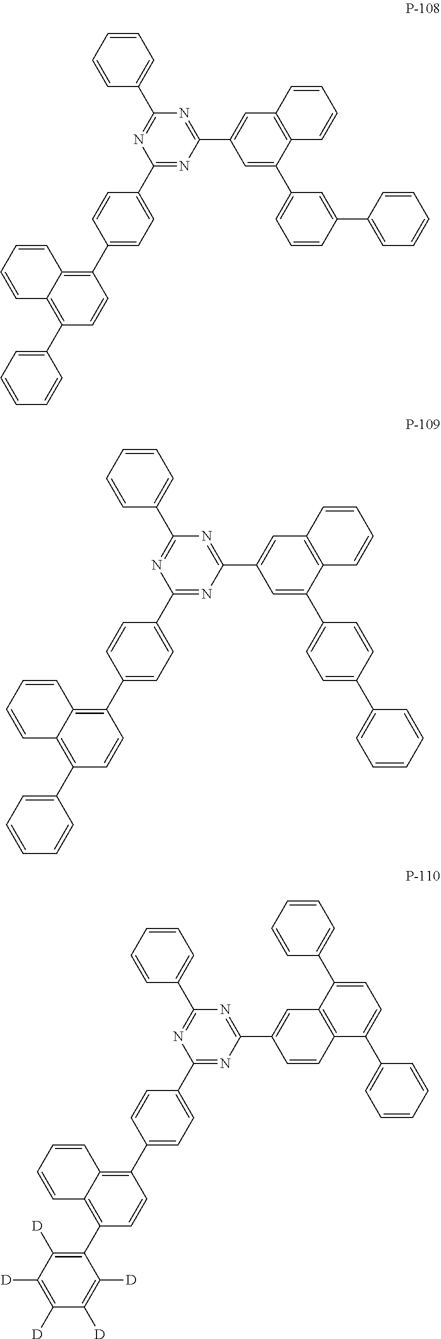 [SMILES: c1ccc(-c2cccc(-c3cc(-c4nc(-c5ccccc5)nc(-c5ccc(-c6ccc(-c7ccccc7)c7ccccc67)cc5)n4)cc4ccccc34)c2)cc1 ; c1ccc(-c2ccc(-c3cc(-c4nc(-c5ccccc5)nc(-c5ccc(-c6ccc(-c7ccccc7)c7ccccc67)cc5)n4)cc4ccccc34)cc2)cc1 ; [2H]c1c([2H])c([2H])c(-c2ccc(-c3ccc(-c4nc(-c5ccccc5)nc(-c5ccc6c(-c7ccccc7)ccc(-c7ccccc7)c6c5)n4)cc3)c3ccccc23)c([2H])c1[2H]]

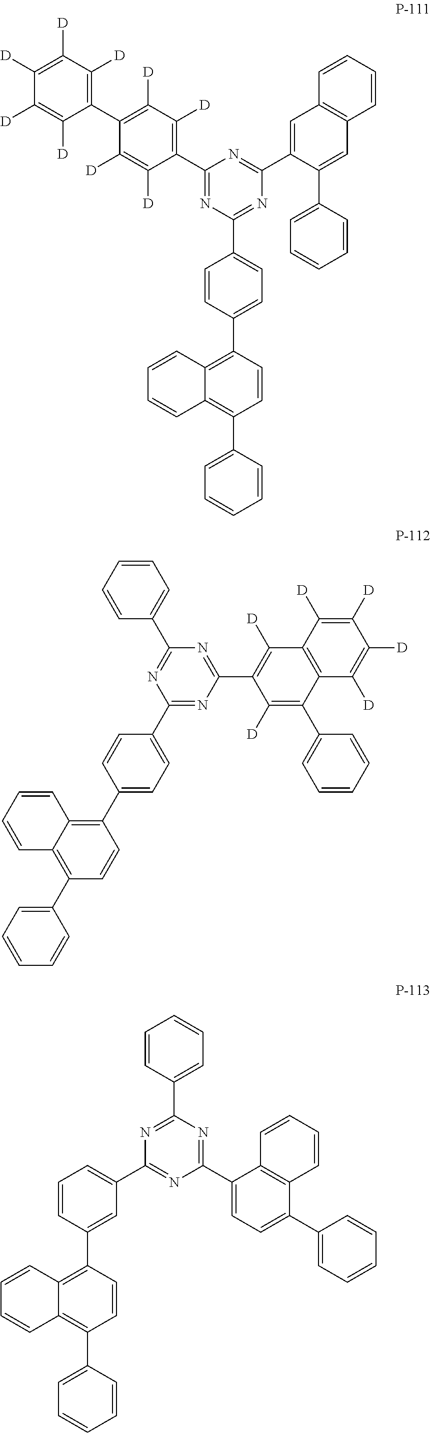 [SMILES: [2H]c1c([2H])c([2H])c(-c2c([2H])c([2H])c(-c3nc(-c4ccc(-c5ccc(-c6ccccc6)c6ccccc56)cc4)nc(-c4cc5ccccc5cc4-c4ccccc4)n3)c([2H])c2[2H])c([2H])c1[2H] ; [2H]c1c([2H])c([2H])c2c(-c3ccccc3)c([2H])c(-c3nc(-c4ccccc4)nc(-c4ccc(-c5ccc(-c6ccccc6)c6ccccc56)cc4)n3)c([2H])c2c1[2H] ; c1ccc(-c2nc(-c3cccc(-c4ccc(-c5ccccc5)c5ccccc45)c3)nc(-c3ccc(-c4ccccc4)c4ccccc34)n2)cc1]

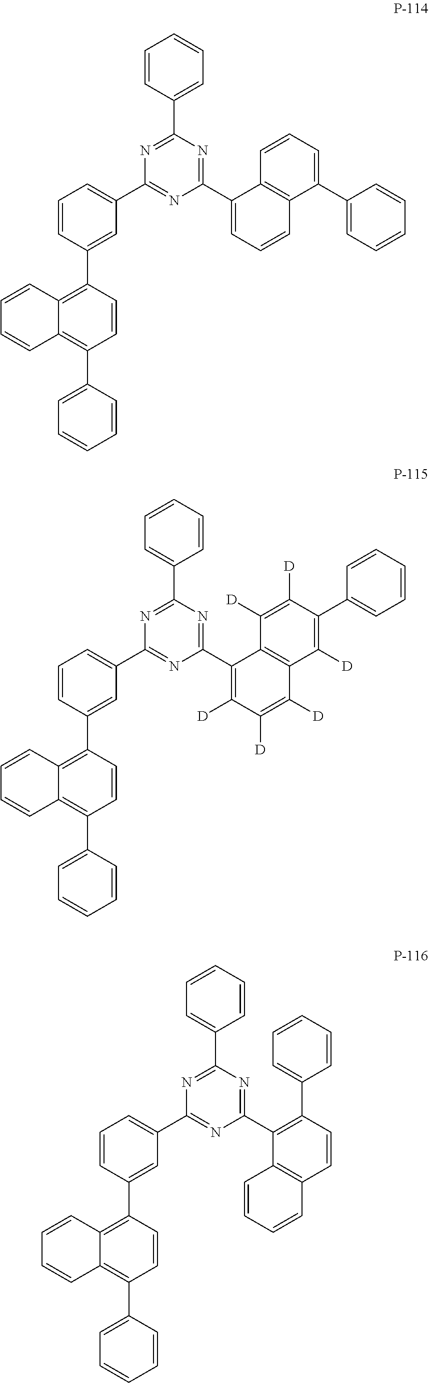 [SMILES: c1ccc(-c2nc(-c3cccc(-c4ccc(-c5ccccc5)c5ccccc45)c3)nc(-c3cccc4c(-c5ccccc5)cccc34)n2)cc1 ; [2H]c1c([2H])c(-c2nc(-c3ccccc3)nc(-c3cccc(-c4ccc(-c5ccccc5)c5ccccc45)c3)n2)c2c([2H])c([2H])c(-c3ccccc3)c([2H])c2c1[2H] ; c1ccc(-c2nc(-c3cccc(-c4ccc(-c5ccccc5)c5ccccc45)c3)nc(-c3c(-c4ccccc4)ccc4ccccc34)n2)cc1]

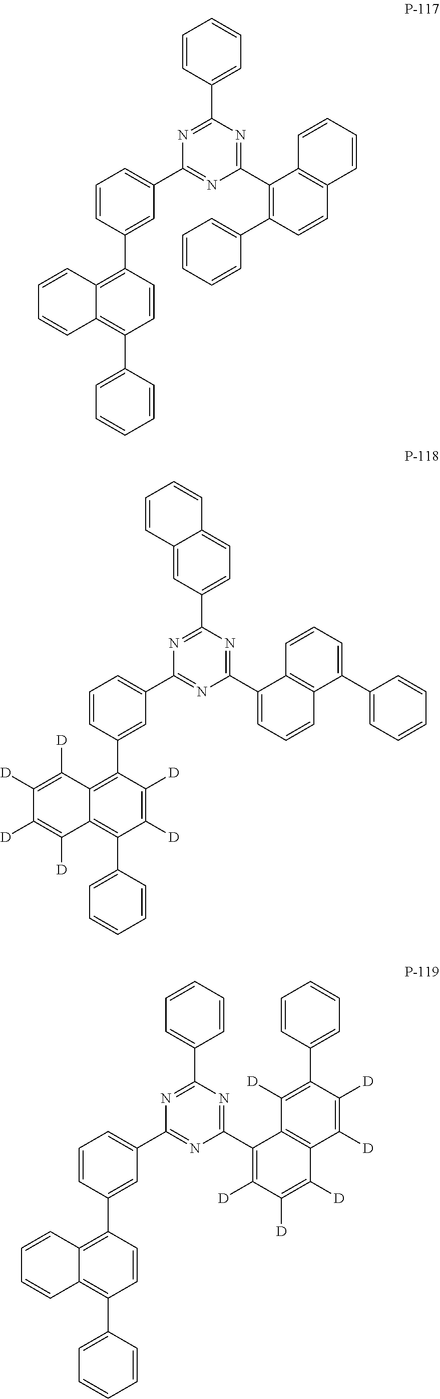 [SMILES: c1ccc(-c2nc(-c3cccc(-c4ccc(-c5ccccc5)c5ccccc45)c3)nc(-c3c(-c4ccccc4)ccc4ccccc34)n2)cc1 ; [2H]c1c([2H])c([2H])c2c(-c3cccc(-c4nc(-c5ccc6ccccc6c5)nc(-c5cccc6c(-c7ccccc7)cccc56)n4)c3)c([2H])c([2H])c(-c3ccccc3)c2c1[2H] ; [2H]c1c([2H])c(-c2nc(-c3ccccc3)nc(-c3cccc(-c4ccc(-c5ccccc5)c5ccccc45)c3)n2)c2c([2H])c(-c3ccccc3)c([2H])c([2H])c2c1[2H]]

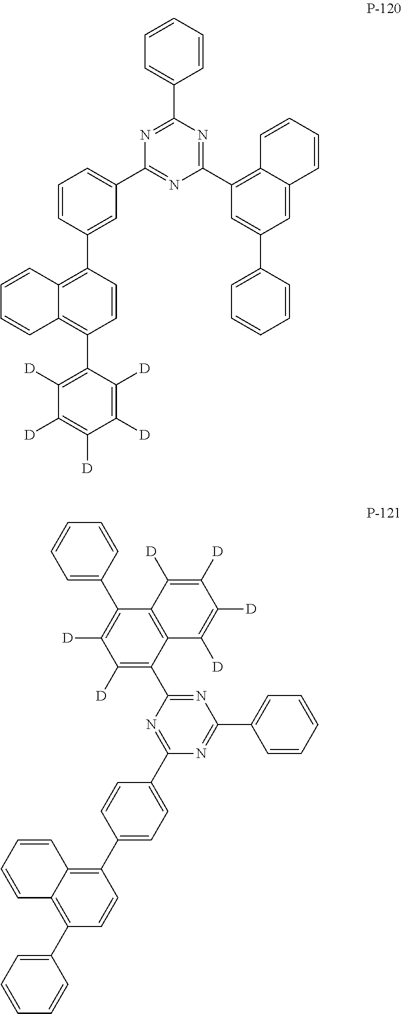 [SMILES: [2H]c1c([2H])c([2H])c(-c2ccc(-c3cccc(-c4nc(-c5ccccc5)nc(-c5cc(-c6ccccc6)cc6ccccc56)n4)c3)c3ccccc23)c([2H])c1[2H] ; [2H]c1c([2H])c([2H])c2c(-c3nc(-c4ccccc4)nc(-c4ccc(-c5ccc(-c6ccccc6)c6ccccc56)cc4)n3)c([2H])c([2H])c(-c3ccccc3)c2c1[2H]]

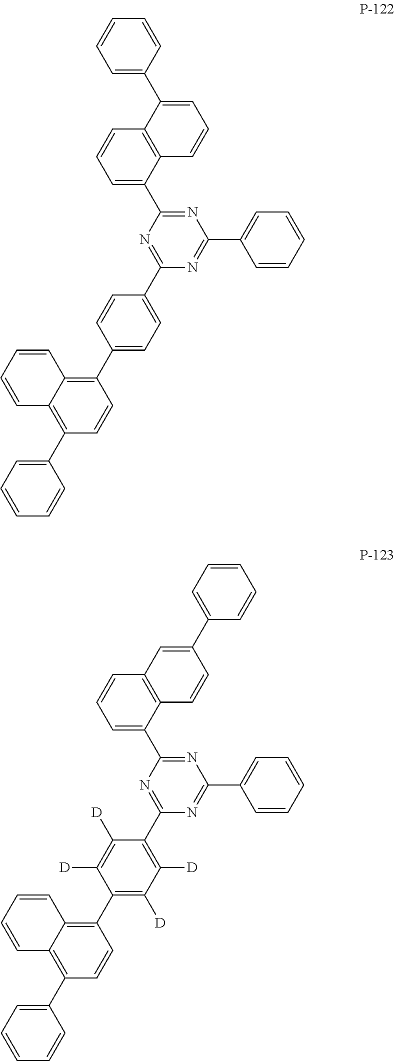 [SMILES: c1ccc(-c2nc(-c3ccc(-c4ccc(-c5ccccc5)c5ccccc45)cc3)nc(-c3cccc4c(-c5ccccc5)cccc34)n2)cc1 ; [2H]c1c([2H])c(-c2ccc(-c3ccccc3)c3ccccc23)c([2H])c([2H])c1-c1nc(-c2ccccc2)nc(-c2cccc3cc(-c4ccccc4)ccc23)n1]

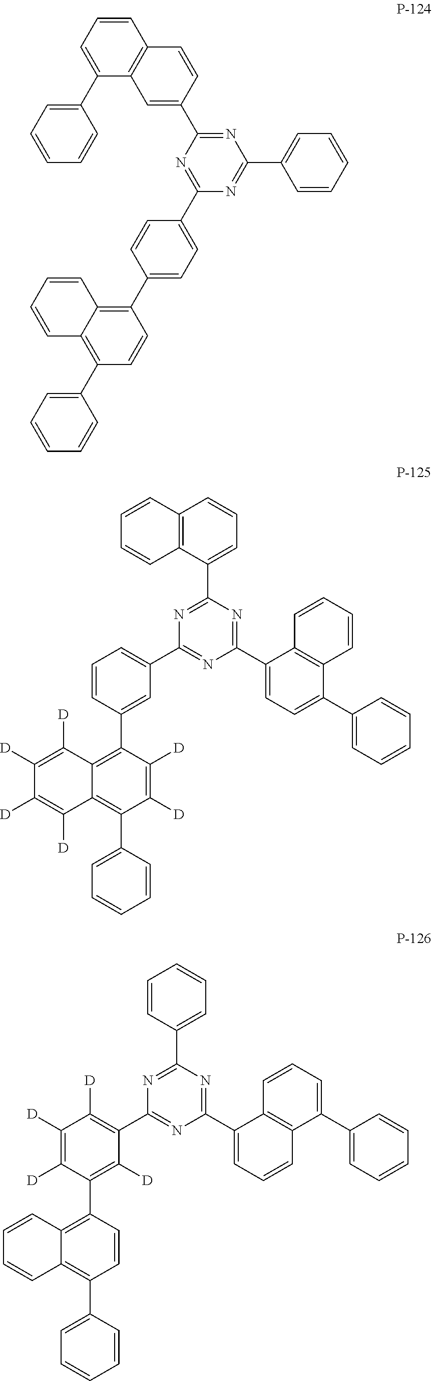 [SMILES: c1ccc(-c2nc(-c3ccc(-c4ccc(-c5ccccc5)c5ccccc45)cc3)nc(-c3ccc4cccc(-c5ccccc5)c4c3)n2)cc1 ; [2H]c1c([2H])c([2H])c2c(-c3cccc(-c4nc(-c5cccc6ccccc56)nc(-c5ccc(-c6ccccc6)c6ccccc56)n4)c3)c([2H])c([2H])c(-c3ccccc3)c2c1[2H] ; [2H]c1c([2H])c(-c2nc(-c3ccccc3)nc(-c3cccc4c(-c5ccccc5)cccc34)n2)c([2H])c(-c2ccc(-c3ccccc3)c3ccccc23)c1[2H]]

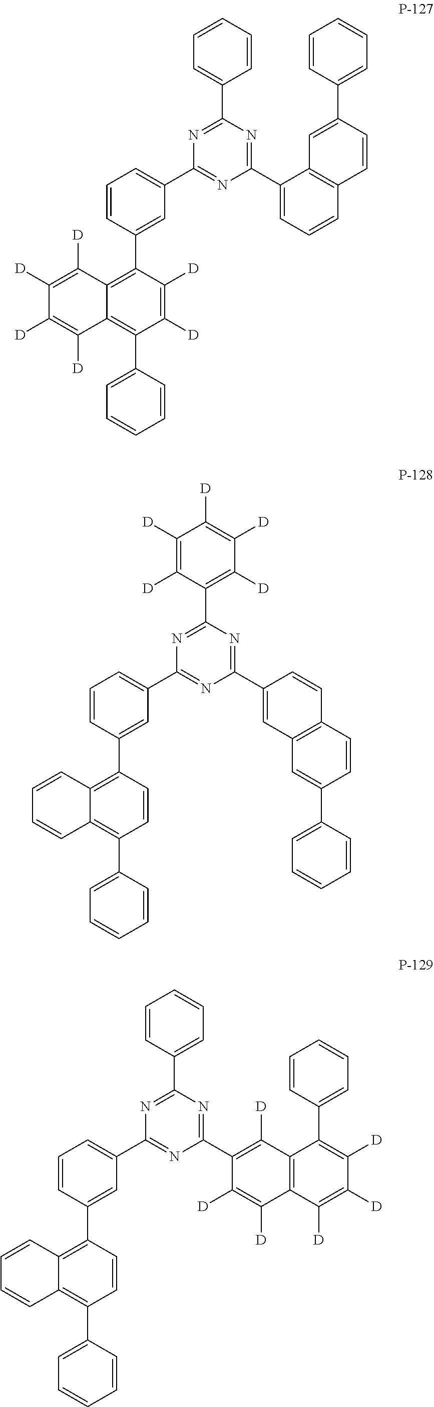 [SMILES: [2H]c1c([2H])c([2H])c2c(-c3cccc(-c4nc(-c5ccccc5)nc(-c5cccc6ccc(-c7ccccc7)cc56)n4)c3)c([2H])c([2H])c(-c3ccccc3)c2c1[2H] ; [2H]c1c([2H])c([2H])c(-c2nc(-c3cccc(-c4ccc(-c5ccccc5)c5ccccc45)c3)nc(-c3ccc4ccc(-c5ccccc5)cc4c3)n2)c([2H])c1[2H] ; [2H]c1c([2H])c(-c2ccccc2)c2c([2H])c(-c3nc(-c4ccccc4)nc(-c4cccc(-c5ccc(-c6ccccc6)c6ccccc56)c4)n3)c([2H])c([2H])c2c1[2H]]

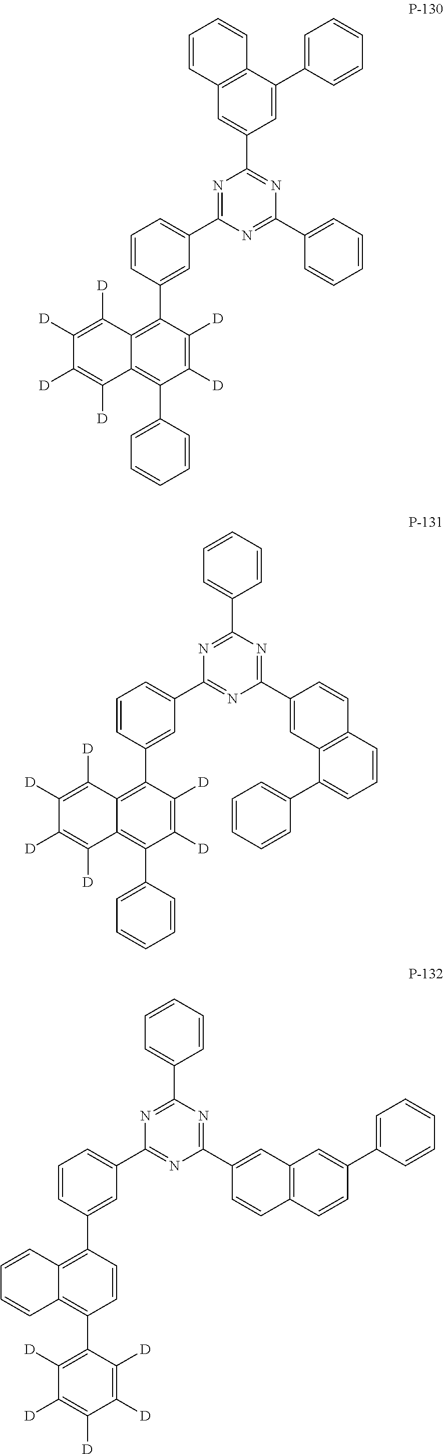 [SMILES: [2H]c1c([2H])c([2H])c2c(-c3cccc(-c4nc(-c5ccccc5)nc(-c5cc(-c6ccccc6)c6ccccc6c5)n4)c3)c([2H])c([2H])c(-c3ccccc3)c2c1[2H] ; [2H]c1c([2H])c([2H])c2c(-c3cccc(-c4nc(-c5ccccc5)nc(-c5ccc6cccc(-c7ccccc7)c6c5)n4)c3)c([2H])c([2H])c(-c3ccccc3)c2c1[2H] ; [2H]c1c([2H])c([2H])c(-c2ccc(-c3cccc(-c4nc(-c5ccccc5)nc(-c5ccc6ccc(-c7ccccc7)cc6c5)n4)c3)c3ccccc23)c([2H])c1[2H]]

In another aspect, the present invention provides a composition for an organic electronic element comprising a mixture of a compound represented by Formula 1 and a compound represented by Formula 2 or 3.

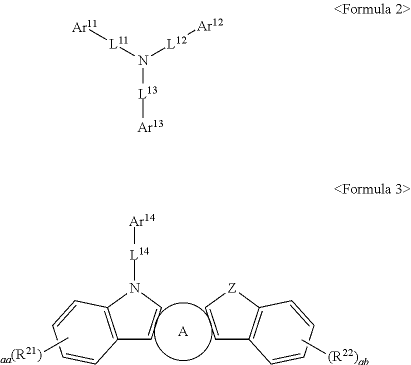

wherein:
L¹¹, L¹², L¹³ and L¹⁴ are each independently selected from the group consisting of single bond; a C₆-C₆₀ arylene group; a fluorenylene group; a C₂-C₆₀ heterocyclic group including at least one heteroatom of O, N, S, Si and P; and a fused ring group of a C₃-C₆₀ aliphatic ring and a C₆-C₆₀ aromatic ring;
wherein when L¹¹, L¹², L¹³ and L¹⁴ are an arylene group, preferably a C₆-C₃₀ arylene group, more preferably a C₆-C₂₅ arylene group, a C₆-C₁₈ arylene group or a C₆-C₁₂ arylene group, such as phenylene, biphenylene, naphthylene, terphenylene, anthracenylene, phenanthrenylen, etc.
wherein when L¹¹, L¹², L¹³ and L¹⁴ are a heterocyclic group, preferably a C₂-C₆₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidine, quinoline, pyrimidoindole, 5-phenyl-5H-pyrimido[5,4-b]indole, quinazoline, quinoxaline, benzoquinazoline, carbazole, dibenzoquinazoline, benzofuran, benzothiophene, dibenzofuran, dibenzothiophene, benzothienopyrimidine, benzofuropyrimidine, phenothiazine, phenylphenothiazine, naphthobenzofuran, naphthobenzothiophene, etc.
wherein when L¹¹, L¹², L¹³ and L¹⁴ are a fused ring group, preferably a fused ring group of a C₃-C₃₀ aliphatic ring and a C₆-C₃₀ aromatic ring, more preferably a fused ring group of a C₃-C₂₅ aliphatic ring and a C₆-C₂₅ aromatic ring.
Ar¹¹, Ar¹² and Ar¹³ are each independently selected from the group consisting of a C₆-C₆₀ aryl group; a fluorenyl group; a C₂-C₆₀ heterocyclic group including at least one heteroatom of O, N, S, Si and P; a fused ring group of a C₃-C₆₀ aliphatic ring and a C₆-C₆₀ aromatic ring; a C₃-C₆₀ aliphatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group;
wherein when Ar¹¹, Ar¹² and Ar¹³ are an aryl group, preferably a C₆-C₃₀ aryl group, more preferably a C₆-C₂₅ aryl group, a C₆-C₁₈ aryl group or a C₆-C₁₂ aryl group, such as phenyl, biphenyl, terphenyl, naphthalene, phenanthrene, chryshen, etc.
wherein when Ar¹¹, Ar¹² and Ar¹³ are a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidine, quinoline, pyrimidoindole, 5-phenyl-5H-pyrimido[5,4-b]indole, quinazoline, quinoxaline, benzoquinazoline, carbazole, dibenzoquinazoline, benzofuran, benzothiophene, dibenzofuran, dibenzothiophene, benzothienopyrimidine, benzofuropyrimidine, phenothiazine, phenylphenothiazine, naphthobenzofuran, naphthobenzothiophene, etc.
wherein when Ar¹¹, Ar¹² and Ar¹³ are a fused ring group, preferably a fused ring group of a C₃-C₃₀ aliphatic ring and a C₆-C₃₀ aromatic ring, more preferably a fused ring group of a C₃-C₂₅ aliphatic ring and a C₆-C₂₅ aromatic ring.
wherein when Ar¹¹, Ar¹² and Ar¹³ are an aliphatic ring group, preferably a C₃-C₃₀ aliphatic ring group, more preferably a C₅-C₂₅ aliphatic ring group, a C₃-C₁₈ aliphatic ring group, and a C₃-C₁₂ aliphatic ring group, and specifically, cyclobutane, cyclopentane, cyclohexane, bicycloheptane, adamantyl, etc.
wherein when Ar¹¹, Ar¹² and Ar¹³ are an alkyl group, preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.
wherein when Ar¹¹, Ar¹² and Ar¹³ are an alkoxyl group, preferably a C₁-C₂₅ alkoxyl group, a C₁-C₁₈ alkoxyl group or a C₁-C₁₂ alkoxyl group.
wherein when Ar¹¹, Ar¹² and Ar¹³ are an aryloxy group, preferably a C₆-C₂₅ aryloxy group, a C₆-C₁₈ an aryloxy group or a C₆-C₁₂ aryloxyl group.
Ar¹⁴ is each independently selected from the group consisting of a C₆-C₆₀ aryl group; a fluorenyl group; a C₂-C₆₀ heterocyclic group including at least one heteroatom of O, N, S, Si and P; and a fused ring group of a C₃-C₆₀ aliphatic ring and a C₆-C₆₀ aromatic ring;
wherein when Ar¹⁴ is an aryl group, preferably a C₆-C₃₀ aryl group, more preferably a C₆-C₂₅ aryl group, a C₆-C₁₈ aryl group or a C₆-C₁₂ aryl group, such as phenyl, biphenyl, terphenyl, naphthalene, phenanthrene, chryshen, etc.
wherein when Ar¹⁴ is a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidine, quinoline, pyrimidoindole, 5-phenyl-5H-pyrimido[5,4-b]indole, quinazoline, quinoxaline, benzoquinazoline, carbazole, dibenzoquinazoline, benzofuran, benzothiophene, dibenzofuran, dibenzothiophene, benzothienopyrimidine, benzofuropyrimidine, phenothiazine, phenylphenothiazine, naphthobenzofuran, naphthobenzothiophene, etc.
wherein when Ar¹⁴ is a fused ring group, preferably a fused ring group of a C₃-C₃₀ aliphatic ring and a C₆-C₃₀ aromatic ring, more preferably a fused ring group of an C₃-C₂₅ aliphatic ring and an C₆-C₂₅ aromatic ring.
Z is O, S, C(R⁵¹)(R⁵²) or NR⁵³,
wherein R⁵¹, R⁵² and R⁵³ are the same as the definition of Ar¹¹, or R⁵¹ and R⁵² may be bonded to each other to form a spiro,
Ring A is a C₆-C₂₀ aryl group,
R²¹ and R²² are each the same or different, and each independently selected from the group consisting of hydrogen; deuterium; halogen; cyano group; a C₆-C₆₀ aryl group; a fluorenyl group; a C₂-C₆₀ heterocyclic group including at least one hetero atom of O, N, S, Si and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group; or an adjacent plurality of R²¹s or plurality of R²²s may be bonded to each other to form a ring,
wherein when R²¹ and R²² are an aryl group, preferably a C₆-C₃₀ aryl group, more preferably a C₆-C₂₅ aryl group, a C₆-C₁₈ aryl group or a C₆-C₁₂ aryl group, such as phenyl, biphenyl, terphenyl, naphthalene, phenanthrene, chryshen, etc.
wherein when R²¹ and R²² are a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidine, quinoline, pyrimidoindole, 5-phenyl-5H-pyrimido[5,4-b]indole, quinazoline, quinoxaline, benzoquinazoline, carbazole, dibenzoquinazoline, benzofuran, benzothiophene, dibenzofuran, dibenzothiophene, benzothienopyrimidine, benzofuropyrimidine, phenothiazine, phenylphenothiazine, naphthobenzofuran, naphthobenzothiophene, etc.
wherein when R²¹ and R²² are a fused ring group, preferably a fused ring group of a C₃-C₃₀ aliphatic ring and an C₆-C₃₀ aromatic ring, more preferably a fused ring group of an C₃-C₂₅ aliphatic ring and an C₆-C₂₅ aromatic ring.
wherein when R²¹ and R²² are an alkyl group, preferably a C₁-C₃₀ alkyl group, more preferably a C₁-C₂₅ alkyl group, a C₁-C₁₈ alkyl group or a C₁-C₁₂ alkyl group, such as a methyl group, ethyl group, propyl group, isopropyl group, butyl group, t-butyl group, pentyl group, etc.
wherein when R²¹ and R²² are an alkoxyl group, preferably a C₁-C₂₅ alkoxyl group, a C₁-C₁₈ alkoxyl group or a C₁-C₁₂ alkoxyl group.
wherein when R²¹ and R²² are an aryloxy group, preferably a C₆-C₂₅ aryloxy group, a C₆-C₁₈ aryloxy group or a C₆-C₁₂ aryloxy group.
aa and ab are each independently an integer of 0 to 4,
wherein the aryl group, arylene group, heterocyclic group, fluorenyl group, fluorenylene group, fused ring group, aliphatic ring group, alkyl group, alkenyl group, alkynyl group, alkoxy group and aryloxy group may be substituted with one or more substituents selected from the group consisting of deuterium; halogen; silane group; siloxane group; boron group; germanium group; cyano group; nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic ring; a C₇-C₂₀ arylalkyl group; a C₈-C₂₀ arylalkenyl group; a C₇-C₂₀ alkylaryl group; and -L′-N(R^{a})(R^{b}); also the hydrogen of these substituents may be further substituted with one or more deuterium, and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.
Wherein L′ is selected from the group consisting of a single bond; a C₆-C₆₀ arylene group; a fluorenylene group; a C₂-C₆₀ heterocyclic group including at least one heteroatom of O, N, S, Si and P; and a C₃-C₆₀ aliphatic ring;
Wherein when L′ is an arylene group, preferably a C₆-C₃₀ arylene group, more preferably a C₆-C₂₅ arylene group, a C₆-C₁₈ arylene group or a C₆-C₁₂ arylene group, such as phenylene, biphenylene, naphthylene, terphenylene, anthracenylene, phenanthrenylene, etc.
wherein when L′ is a heterocyclic group, preferably a C₂-C₃₀ heterocyclic group, more preferably a C₂-C₂₅ heterocyclic group, a C₂-C₁₈ heterocyclic group, or a C₂-C₁₂ heterocyclic group, such as pyrazine, thiophene, pyridine, pyrimidine, quinoline, pyrimidoindole, 5-phenyl-5H-pyrimido[5,4-b]indole, quinazoline, quinoxaline, benzoquinazoline, carbazole, dibenzoquinazoline, benzofuran, benzothiophene, dibenzofuran, dibenzothiophene, benzothienopyrimidine, benzofuropyrimidine, phenothiazine, phenylphenothiazine, naphthobenzofuran, naphthobenzothiophene, etc.
wherein when L′ is an aliphatic ring, preferably a C₃-C₃₀ aliphatic ring; more preferably a C₃-C₂₅ aliphatic ring; a C₃-C₁₈ aliphatic ring; or a C₃-C₁₂ aliphatic ring; and specifically, cyclobutane, cyclopentane, cyclohexane, bicycloheptane, adamantyl, etc.
R^{a} and R^{b} are the same as the definition of Ar¹¹,

Preferably, the composition for an organic electronic element may be used as a host for an emitting layer.

Formula 2 may be represented by any one of Formulas 2-1 to 2-3.

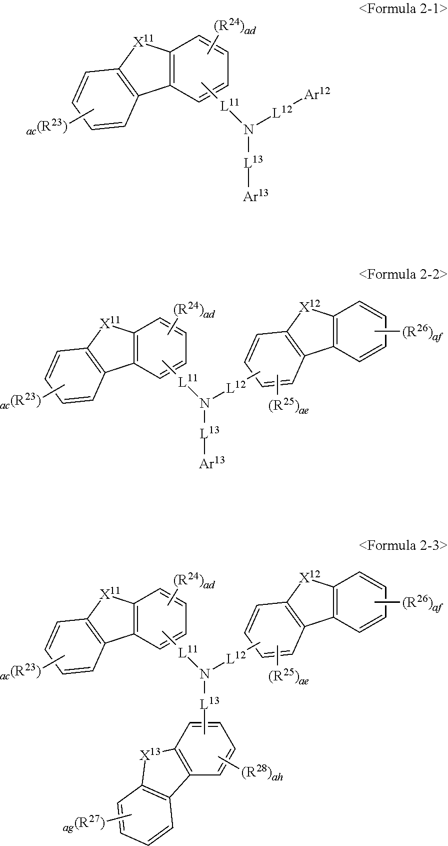

wherein:
Ar¹², Ar¹³, L¹¹, L¹² and L¹³ are the same as defined in Formula 2,
X¹¹, X¹² and X¹³ are the same as the definition of Z in Formula 3,
R²³, R²⁴, R²⁵, R²⁶, R²⁷ and R²⁸ are each the same or different, and each independently selected from the group consisting of hydrogen; deuterium; halogen; silane group; siloxane group; boron group; germanium group; cyano group; nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; C₆-C₂₀ aryl group substituted with deuterium; a fluorenyl group; a C₂-C₂₀ heterocyclic group; a C₅-C₂₀ aliphatic group; a C₇-C₂₀ arylalkyl group; a C₈-C₂₀ arylalkenyl group; and a C₇-C₂₀ alkylaryl group; or an adjacent plurality of R²³s or plurality of R²⁴s or plurality of R²⁵s or plurality of R²⁶s or plurality of R²⁷s or plurality of R²⁸s may be bonded to each other to form a ring,
ac, af and ag are independently an integer of 0 to 4, and ad, ae and ah are independently an integer of 0 to 3.

Formula 3 may be represented by any one of Formulas 3-1 to 3-6.

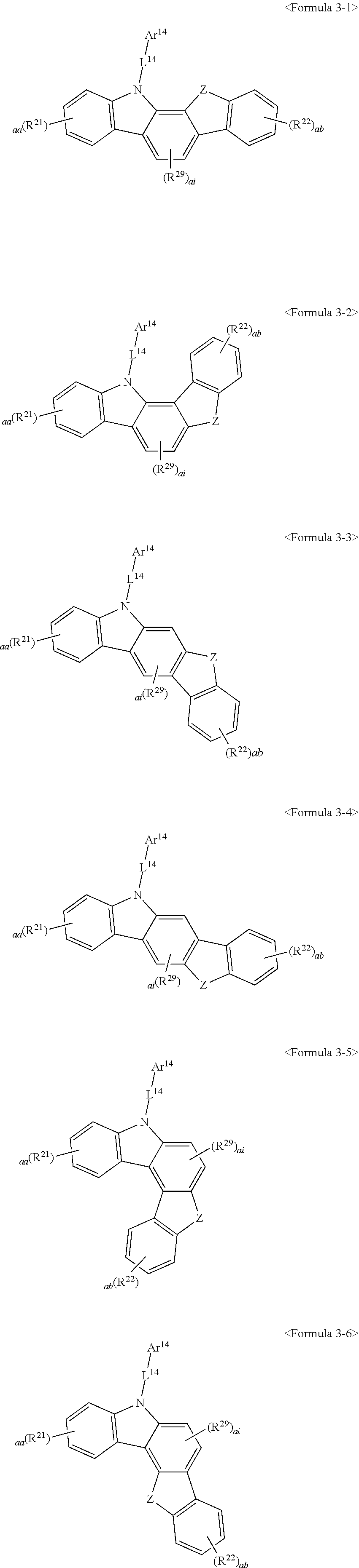

Wherein:
Z, Ar¹⁴, L¹⁴, R²¹, R²², aa and ab are the same as defined in Formula 3,
R²⁹ is the same as the definition of R²¹, or an adjacent plurality of R²⁹s may be bonded to each other to form a ring,
ai is an integer of 0 to 2.

Formula 3 may be represented by any one of the following Formulas 3-7 to 3-9.

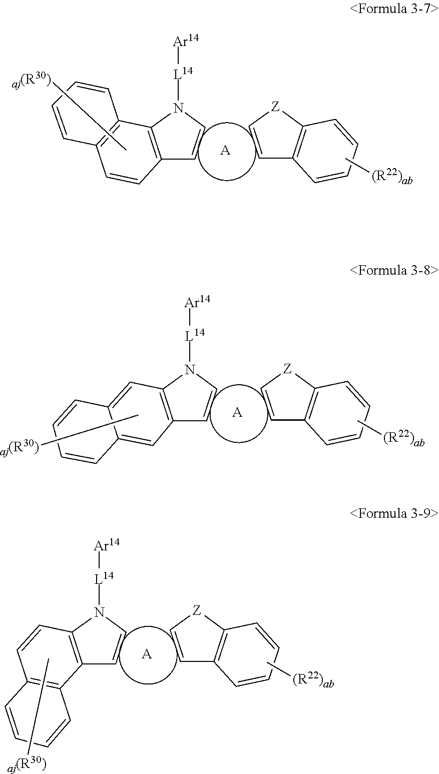

wherein:
Z, Ring A, Ar¹⁴, L¹⁴, R²² and ab are the same as defined in Formula 3,
R³⁰ is the same as the definition of R²¹, or an adjacent plurality of R³⁰s may be bonded to each other to form a ring,
aj is an integer of 0 to 6.

Formula 3 may be represented by any one of Formulas 3-10 to 3-12.

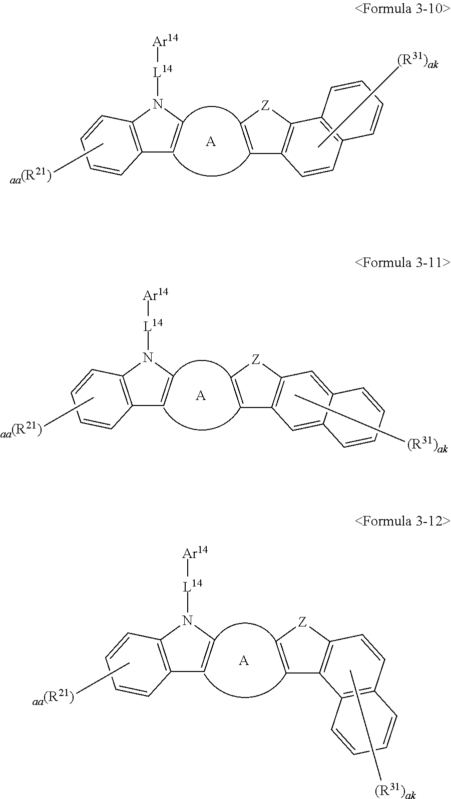

wherein:
Z, Ring A, Ar¹⁴, L¹⁴, R²² and ab are the same as defined in Formula 3,
R³¹ is the same as the definition of R²¹, or an adjacent plurality of R³¹s may be bonded to each other to form a ring,
ak is an integer of 0 to 6.

Formula 3 may be represented by any one of Formulas 3-13 to 3-18.

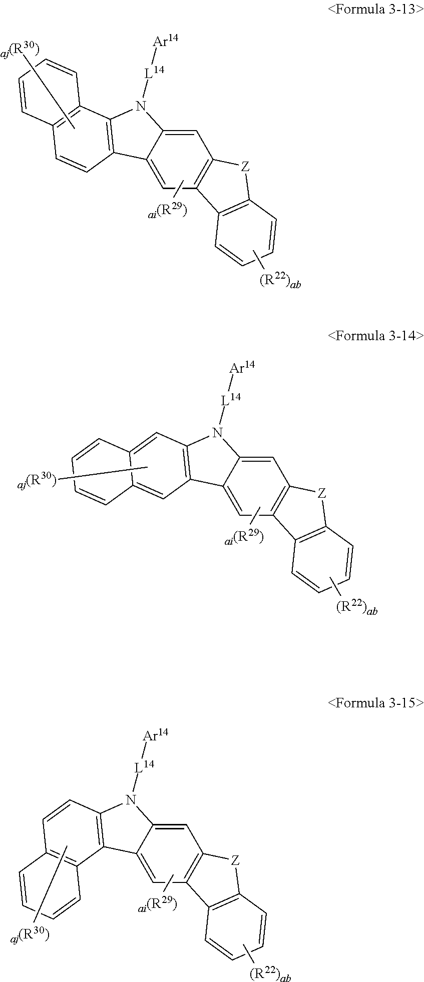

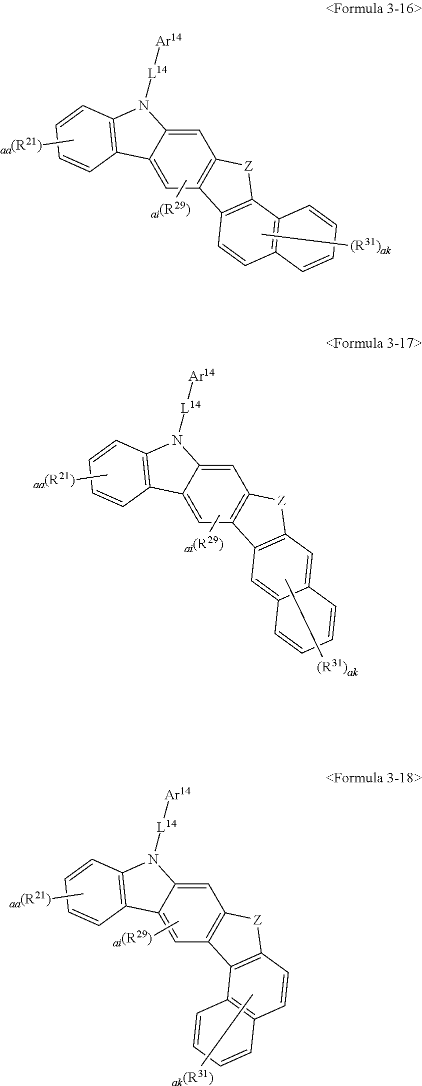

wherein:
Z, Ar¹⁴, L¹⁴, R²¹, R²², aa and ab are the same as defined in Formula 3,
R²⁹, R³⁰ and R³¹ are the same as the definition of R²¹, or an adjacent plurality of R³⁰s or an adjacent plurality of R³¹s may be bonded to each other to form a ring,
ai is an integer of 0 to 2, and aj and ak are integer of 0 to 6, independently of each other.

Formula 3 may be represented by Formula 3-19.

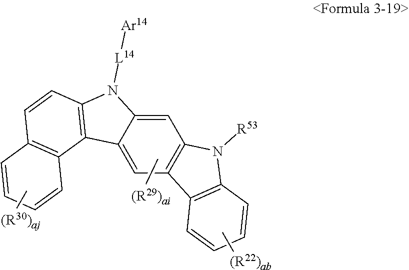

wherein:
Ar¹⁴, L¹⁴, R²², R⁵³ and ab are the same as defined in Formula 3,
R²⁹ and R³⁰ are the same as the definition of R²¹, or an adjacent plurality of R³⁰s may be bonded to each other to form a ring,
ai is an integer of 0 to 2, and aj is an integer of 0 to 6.

Specifically, the compound represented by Formula 2 may be a compound represented by any one of the compounds N-1 to N-128, but is not limited thereto.

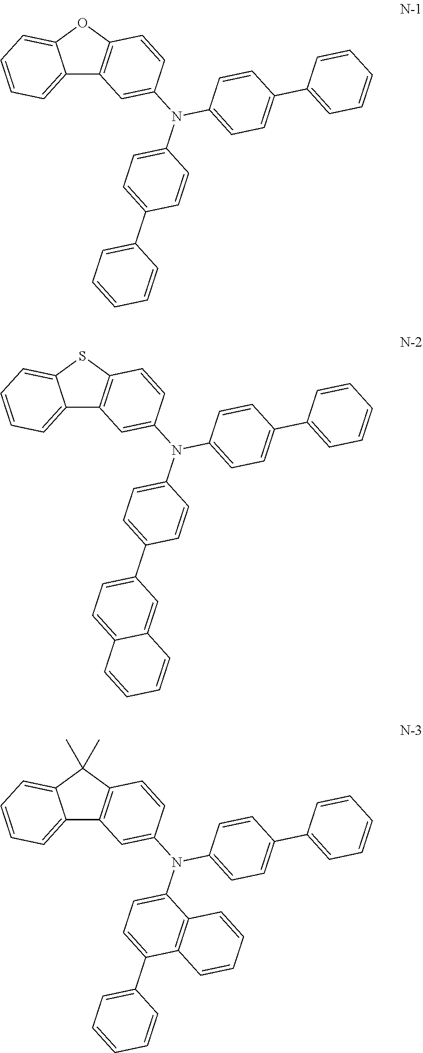 [SMILES: c1ccc(-c2ccc(N(c3ccc(-c4ccccc4)cc3)c3ccc4oc5ccccc5c4c3)cc2)cc1 ; c1ccc(-c2ccc(N(c3ccc(-c4ccc5ccccc5c4)cc3)c3ccc4sc5ccccc5c4c3)cc2)cc1 ; CC1(C)c2ccccc2-c2cc(N(c3ccc(-c4ccccc4)cc3)c3ccc(-c4ccccc4)c4ccccc34)ccc21]

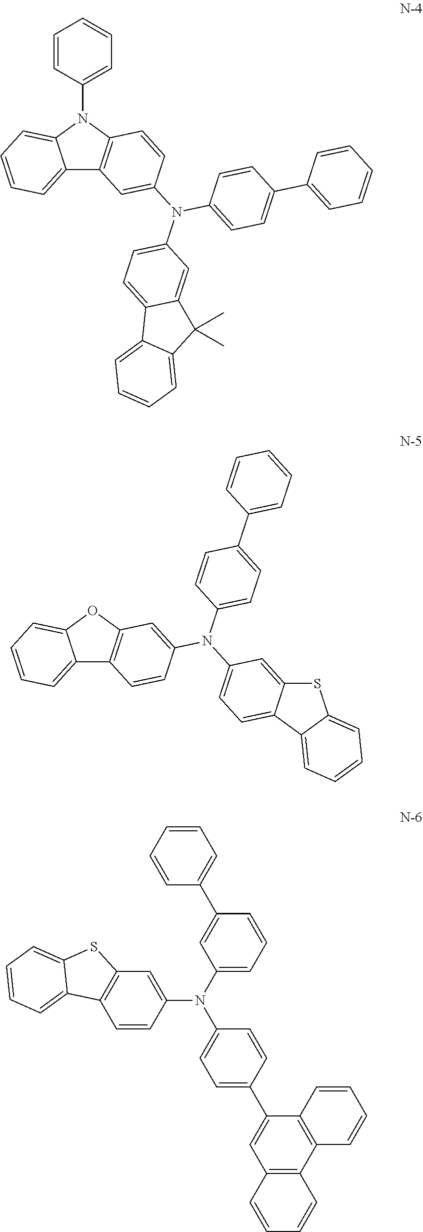 [SMILES: CC1(C)c2ccccc2-c2ccc(N(c3ccc(-c4ccccc4)cc3)c3ccc4c(c3)c3ccccc3n4-c3ccccc3)cc21 ; c1ccc(-c2ccc(N(c3ccc4c(c3)oc3ccccc34)c3ccc4c(c3)sc3ccccc34)cc2)cc1 ; c1ccc(-c2cccc(N(c3ccc(-c4cc5ccccc5c5ccccc45)cc3)c3ccc4c(c3)sc3ccccc34)c2)cc1]

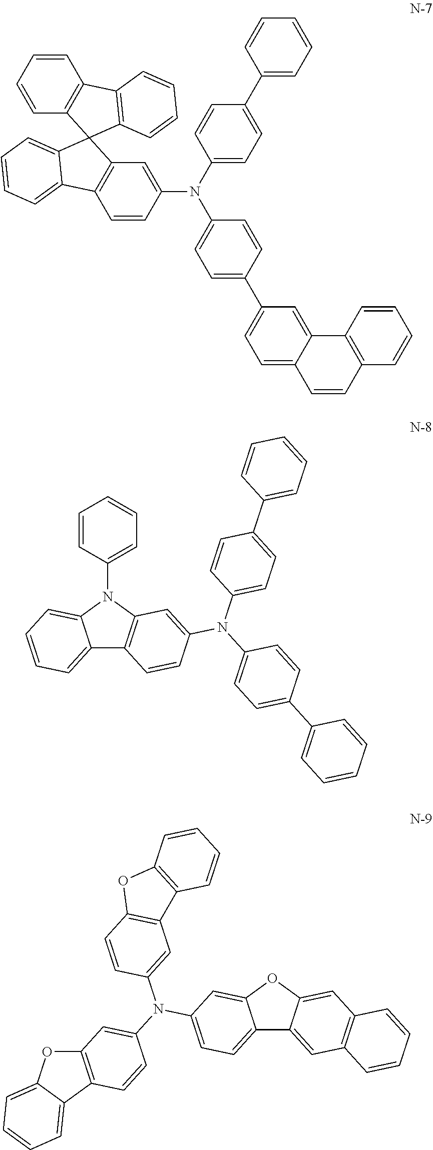 [SMILES: c1ccc(-c2ccc(N(c3ccc(-c4ccc5ccc6ccccc6c5c4)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3ccc4c5ccccc5n(-c5ccccc5)c4c3)cc2)cc1 ; c1ccc2cc3c(cc2c1)oc1cc(N(c2ccc4c(c2)oc2ccccc24)c2ccc4oc5ccccc5c4c2)ccc13]

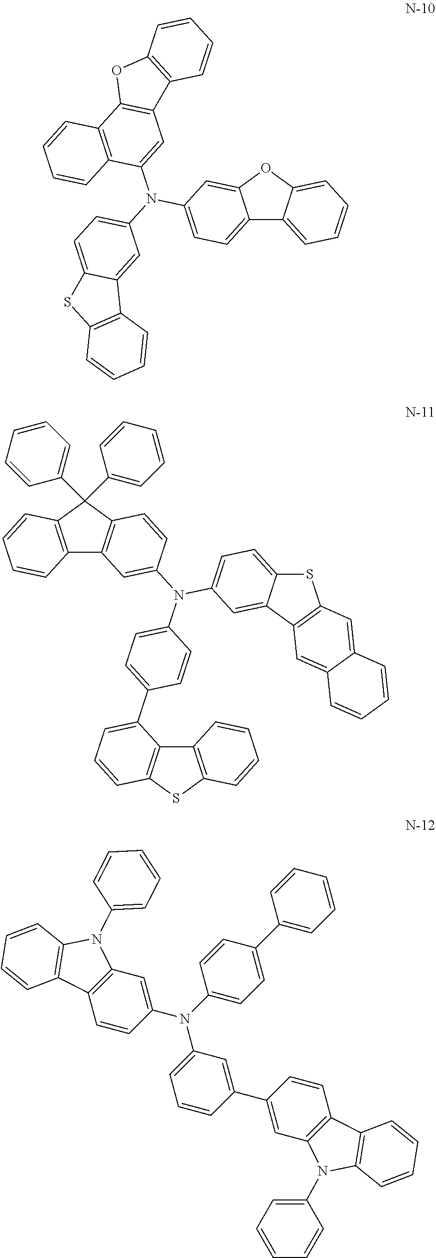

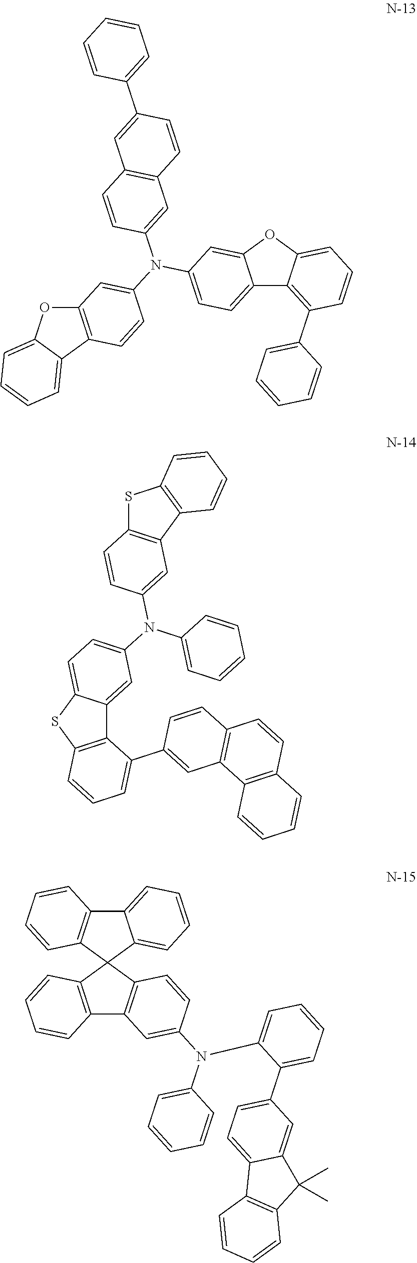 [SMILES: c1ccc(-c2ccc3cc(N(c4ccc5c(c4)oc4ccccc45)c4ccc5c(c4)oc4cccc(-c6ccccc6)c45)ccc3c2)cc1 ; c1ccc(N(c2ccc3sc4ccccc4c3c2)c2ccc3sc4cccc(-c5ccc6ccc7ccccc7c6c5)c4c3c2)cc1 ; CC1(C)c2ccccc2-c2ccc(-c3ccccc3N(c3ccccc3)c3ccc4c(c3)-c3ccccc3C43c4ccccc4-c4ccccc43)cc21]

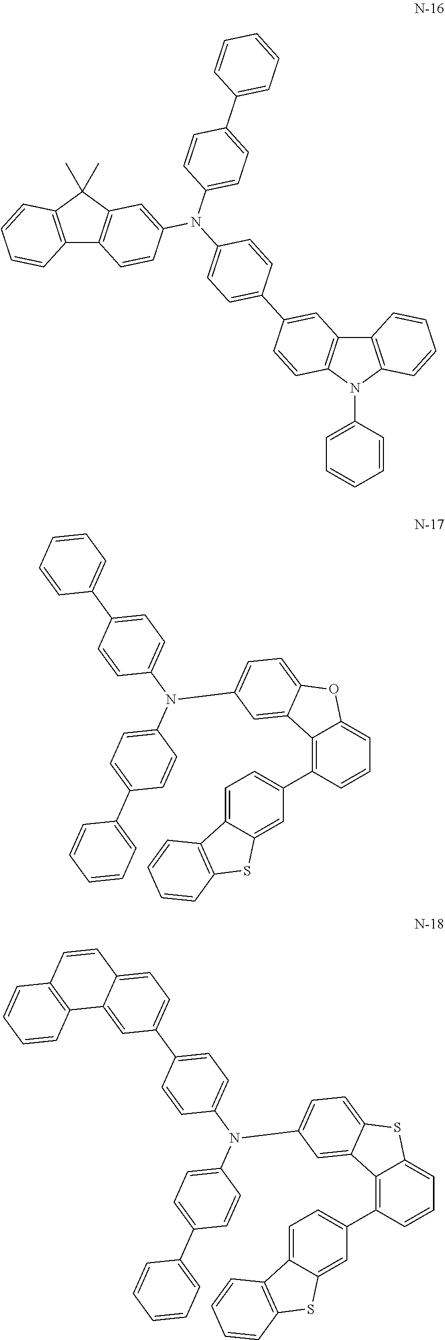 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccccc4)cc3)c3ccc4oc5cccc(-c6ccc7c(c6)sc6ccccc67)c5c4c3)cc2)cc1 ; c1ccc(-c2ccc(N(c3ccc(-c4ccc5ccc6ccccc6c5c4)cc3)c3ccc4sc5cccc(-c6ccc7c(c6)sc6ccccc67)c5c4c3)cc2)cc1]

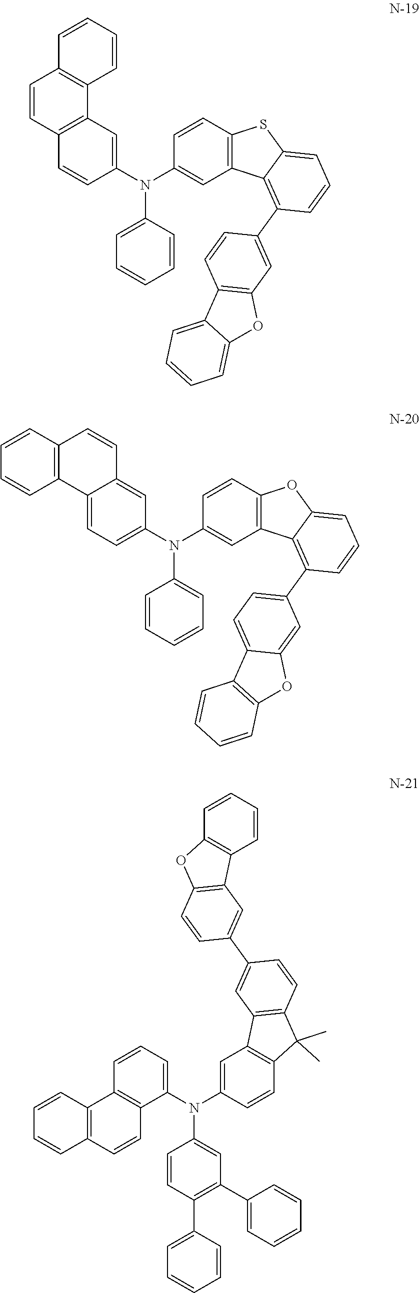 [SMILES: c1ccc(N(c2ccc3ccc4ccccc4c3c2)c2ccc3sc4cccc(-c5ccc6c(c5)oc5ccccc56)c4c3c2)cc1 ; c1ccc(N(c2ccc3c(ccc4ccccc43)c2)c2ccc3oc4cccc(-c5ccc6c(c5)oc5ccccc56)c4c3c2)cc1 ; CC1(C)c2ccc(-c3ccc4oc5ccccc5c4c3)cc2-c2cc(N(c3ccc(-c4ccccc4)c(-c4ccccc4)c3)c3cccc4c3ccc3ccccc34)ccc21]

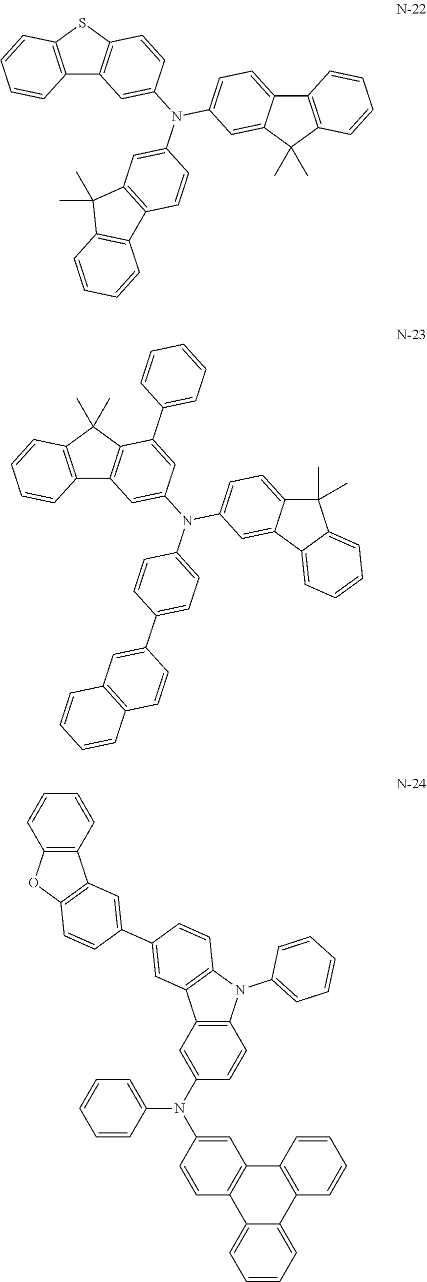

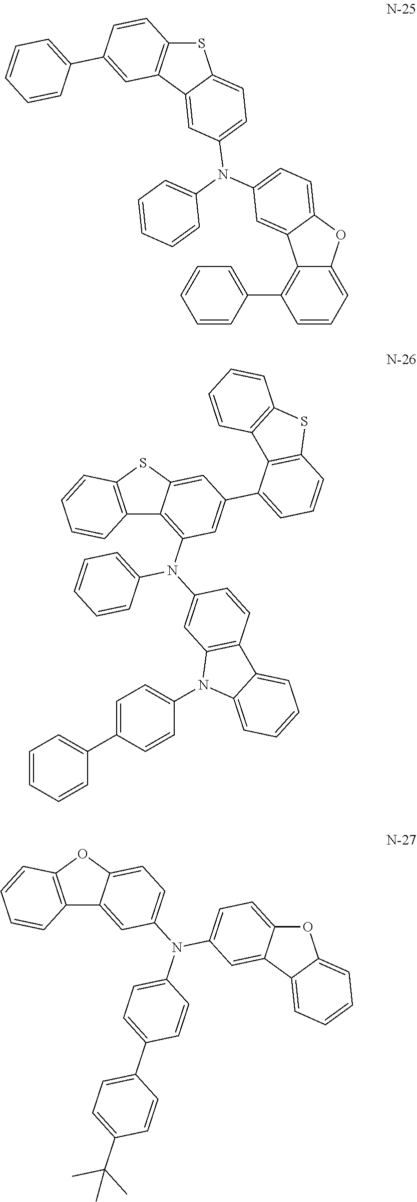

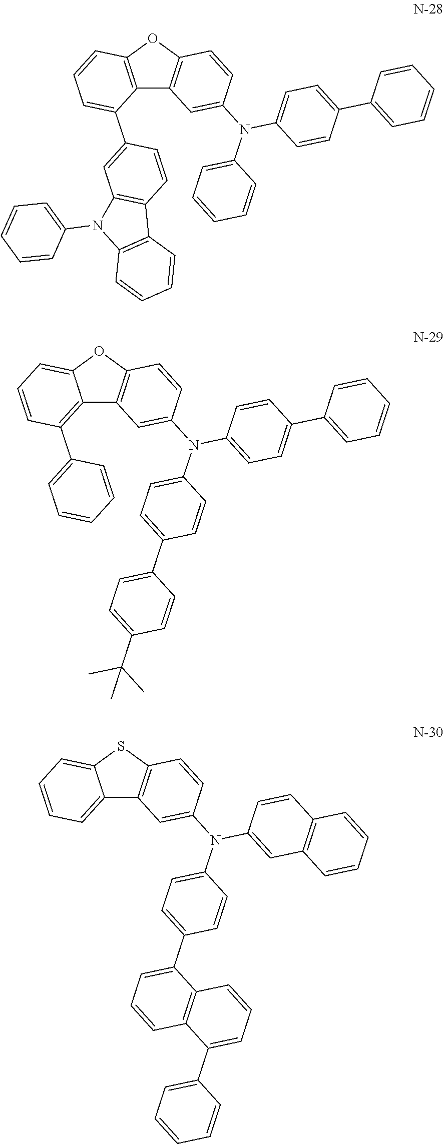 [SMILES: c1ccc(-c2ccc(N(c3ccccc3)c3ccc4oc5cccc(-c6ccc7c8ccccc8n(-c8ccccc8)c7c6)c5c4c3)cc2)cc1 ; CC(C)(C)c1ccc(-c2ccc(N(c3ccc(-c4ccccc4)cc3)c3ccc4oc5cccc(-c6ccccc6)c5c4c3)cc2)cc1 ; c1ccc(-c2cccc3c(-c4ccc(N(c5ccc6ccccc6c5)c5ccc6sc7ccccc7c6c5)cc4)cccc23)cc1]

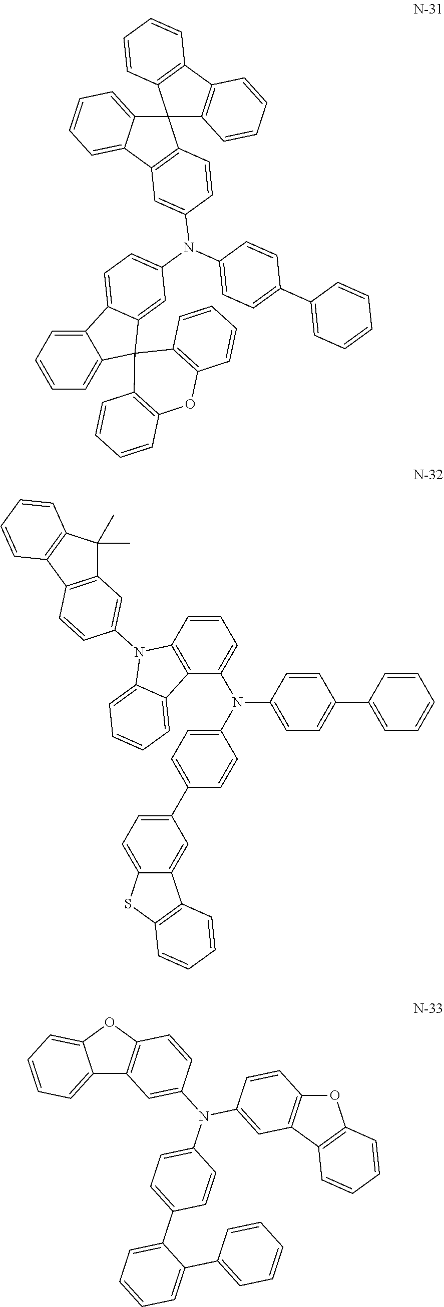 [SMILES: c1ccc(-c2ccc(N(c3ccc4c(c3)-c3ccccc3C43c4ccccc4-c4ccccc43)c3ccc4c(c3)C3(c5ccccc5Oc5ccccc53)c3ccccc3-4)cc2)cc1 ; CC1(C)c2ccccc2-c2ccc(-n3c4ccccc4c4c(N(c5ccc(-c6ccccc6)cc5)c5ccc(-c6ccc7sc8ccccc8c7c6)cc5)cccc43)cc21 ; c1ccc(-c2ccccc2-c2ccc(N(c3ccc4oc5ccccc5c4c3)c3ccc4oc5ccccc5c4c3)cc2)cc1]

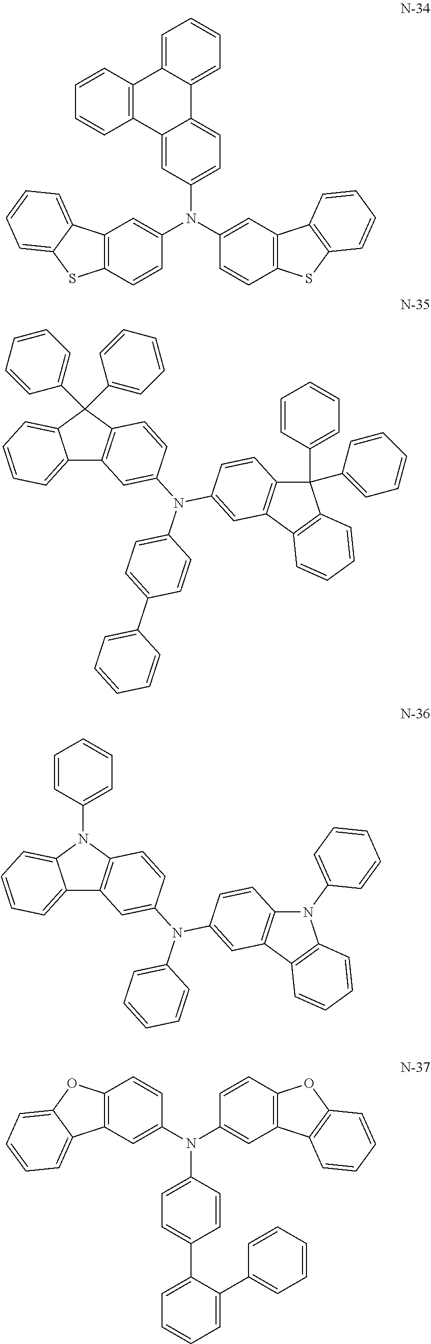

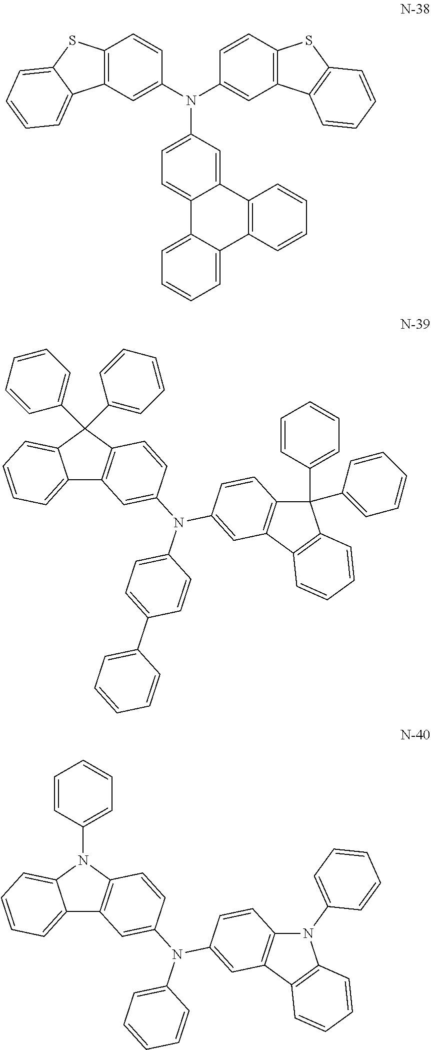 [SMILES: c1ccc2c(c1)sc1ccc(N(c3ccc4sc5ccccc5c4c3)c3ccc4c5ccccc5c5ccccc5c4c3)cc12 ; c1ccc(-c2ccc(N(c3ccc4c(c3)-c3ccccc3C4(c3ccccc3)c3ccccc3)c3ccc4c(c3)-c3ccccc3C4(c3ccccc3)c3ccccc3)cc2)cc1 ; c1ccc(N(c2ccc3c(c2)c2ccccc2n3-c2ccccc2)c2ccc3c(c2)c2ccccc2n3-c2ccccc2)cc1]

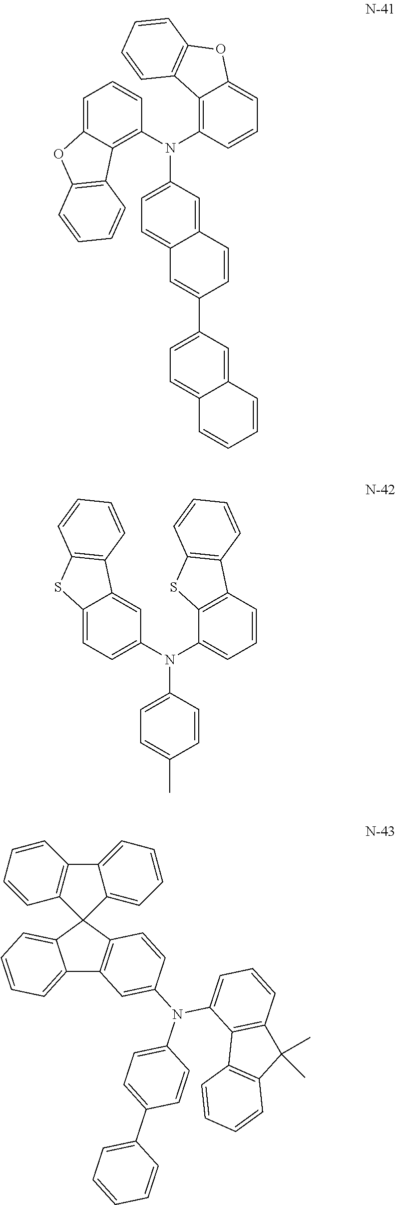 [SMILES: c1ccc2cc(-c3ccc4cc(N(c5cccc6oc7ccccc7c56)c5cccc6oc7ccccc7c56)ccc4c3)ccc2c1 ; Cc1ccc(N(c2ccc3sc4ccccc4c3c2)c2cccc3c2sc2ccccc23)cc1 ; CC1(C)c2ccccc2-c2c(N(c3ccc(-c4ccccc4)cc3)c3ccc4c(c3)-c3ccccc3C43c4ccccc4-c4ccccc43)cccc21]

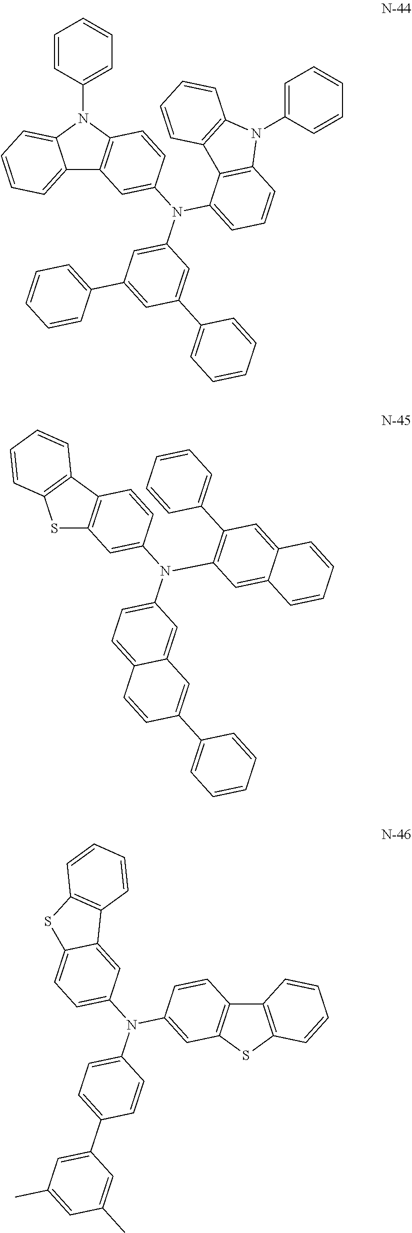 [SMILES: c1ccc(-c2cc(-c3ccccc3)cc(N(c3ccc4c(c3)c3ccccc3n4-c3ccccc3)c3cccc4c3c3ccccc3n4-c3ccccc3)c2)cc1 ; c1ccc(-c2ccc3ccc(N(c4ccc5c(c4)sc4ccccc45)c4cc5ccccc5cc4-c4ccccc4)cc3c2)cc1 ; Cc1cc(C)cc(-c2ccc(N(c3ccc4c(c3)sc3ccccc34)c3ccc4sc5ccccc5c4c3)cc2)c1]

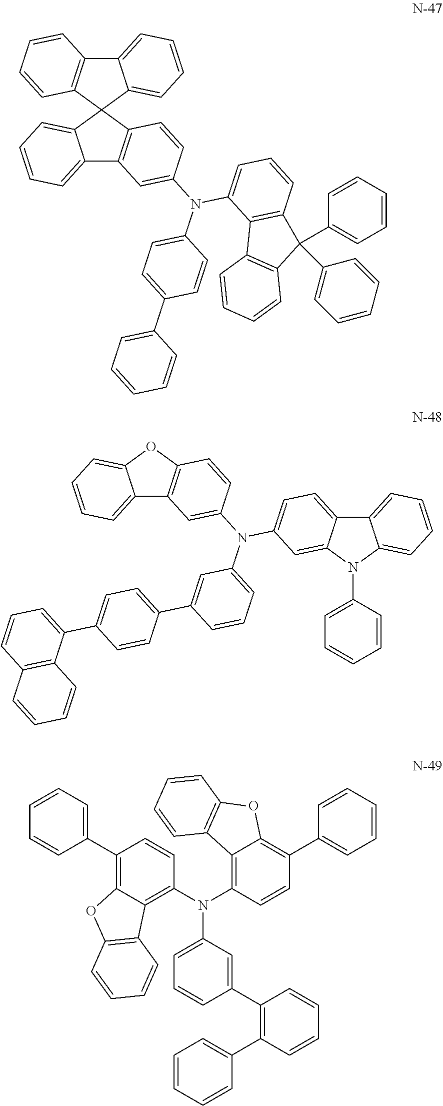 [SMILES: c1ccc(-c2ccc(N(c3ccc4c(c3)-c3ccccc3C43c4ccccc4-c4ccccc43)c3cccc4c3-c3ccccc3C4(c3ccccc3)c3ccccc3)cc2)cc1 ; c1ccc(-n2c3ccccc3c3ccc(N(c4cccc(-c5ccc(-c6cccc7ccccc67)cc5)c4)c4ccc5oc6ccccc6c5c4)cc32)cc1 ; c1ccc(-c2ccccc2-c2cccc(N(c3ccc(-c4ccccc4)c4oc5ccccc5c34)c3ccc(-c4ccccc4)c4oc5ccccc5c34)c2)cc1]

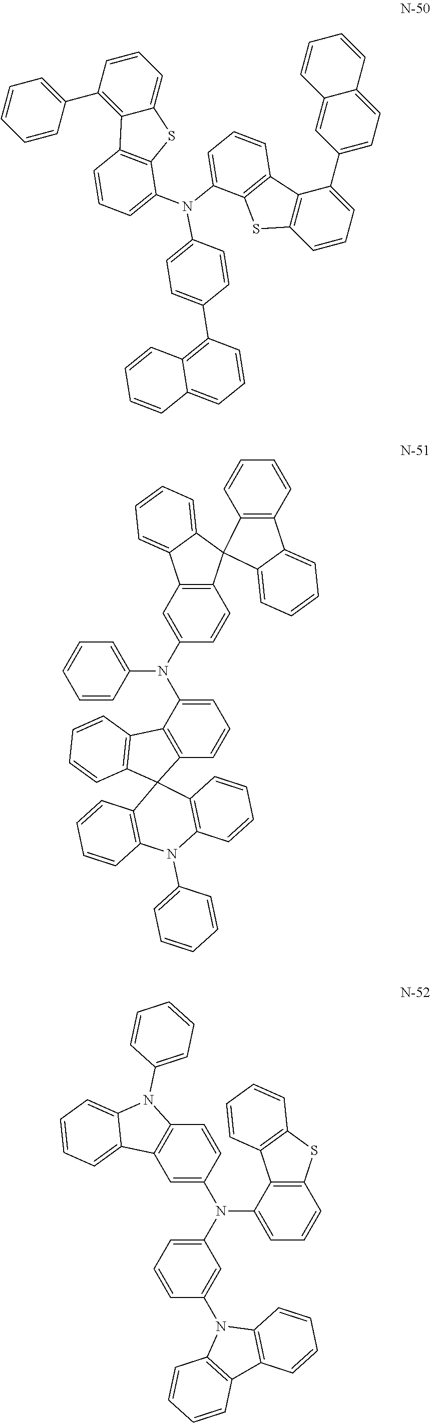 [SMILES: c1ccc(-c2cccc3sc4c(N(c5ccc(-c6cccc7ccccc67)cc5)c5cccc6c5sc5cccc(-c7ccc8ccccc8c7)c56)cccc4c23)cc1 ; c1ccc(N(c2ccc3c(c2)-c2ccccc2C32c3ccccc3-c3ccccc32)c2cccc3c2-c2ccccc2C32c3ccccc3N(c3ccccc3)c3ccccc32)cc1 ; c1ccc(-n2c3ccccc3c3cc(N(c4cccc(-n5c6ccccc6c6ccccc65)c4)c4cccc5sc6ccccc6c45)ccc32)cc1]

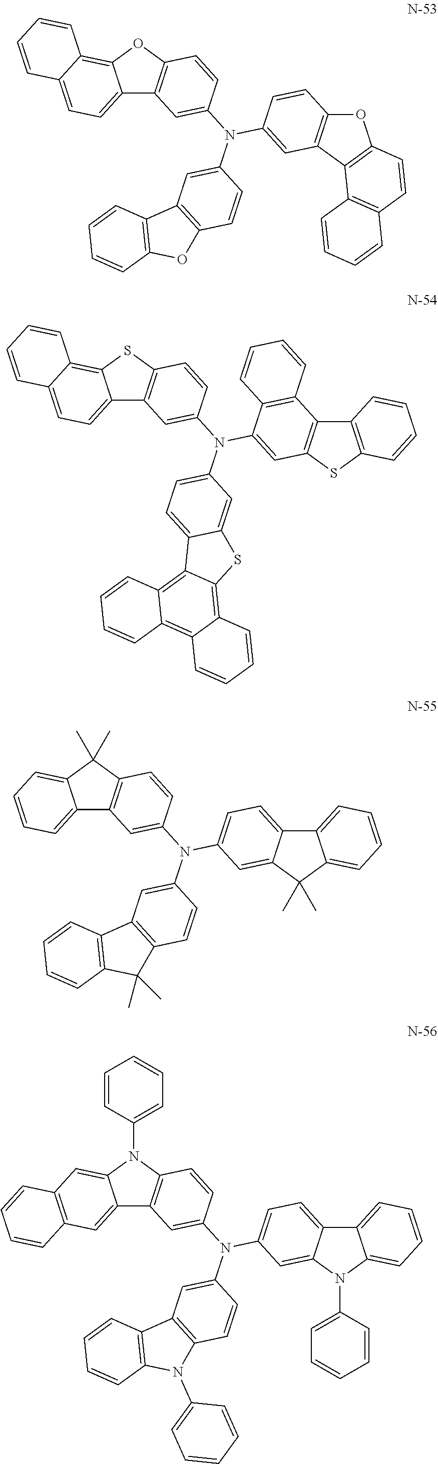 [SMILES: c1ccc2c(c1)ccc1c3cc(N(c4ccc5oc6ccccc6c5c4)c4ccc5oc6ccc7ccccc7c6c5c4)ccc3oc21 ; c1ccc2c(c1)ccc1c3cc(N(c4ccc5c(c4)sc4c6ccccc6c6ccccc6c54)c4cc5sc6ccccc6c5c5ccccc45)ccc3sc21 ; CC1(C)c2ccccc2-c2cc(N(c3ccc4c(c3)-c3ccccc3C4(C)C)c3ccc4c(c3)C(C)(C)c3ccccc3-4)ccc21 ; c1ccc(-n2c3ccccc3c3cc(N(c4ccc5c(c4)c4cc6ccccc6cc4n5-c4ccccc4)c4ccc5c6ccccc6n(-c6ccccc6)c5c4)ccc32)cc1]

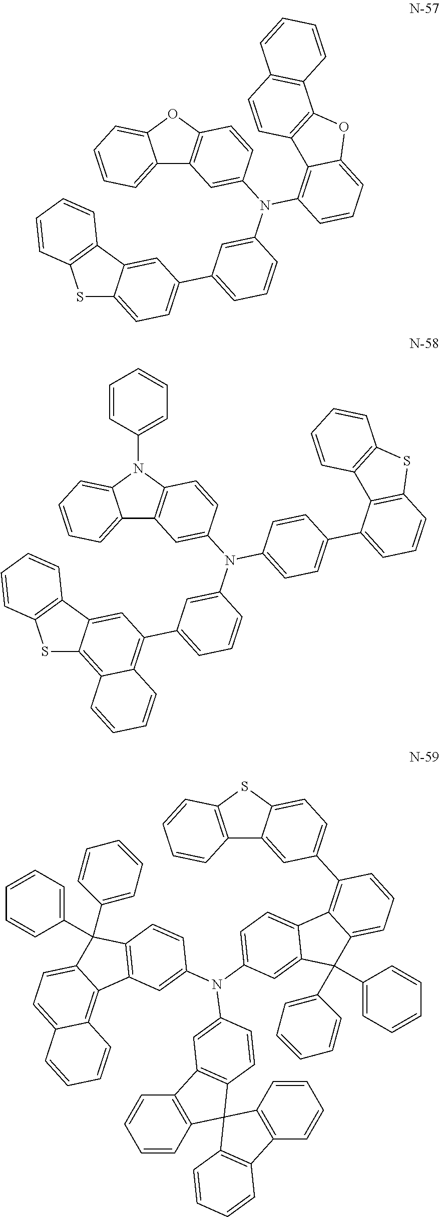 [SMILES: c1cc(-c2ccc3sc4ccccc4c3c2)cc(N(c2ccc3oc4ccccc4c3c2)c2cccc3oc4c5ccccc5ccc4c23)c1 ; c1ccc(-n2c3ccccc3c3cc(N(c4ccc(-c5cccc6sc7ccccc7c56)cc4)c4cccc(-c5cc6c7ccccc7sc6c6ccccc56)c4)ccc32)cc1 ; c1ccc(C2(c3ccccc3)c3cc(N(c4ccc5c(c4)-c4c(ccc6ccccc46)C5(c4ccccc4)c4ccccc4)c4ccc5c(c4)-c4ccccc4C54c5ccccc5-c5ccccc54)ccc3-c3c(-c4ccc5sc6ccccc6c5c4)cccc32)cc1]

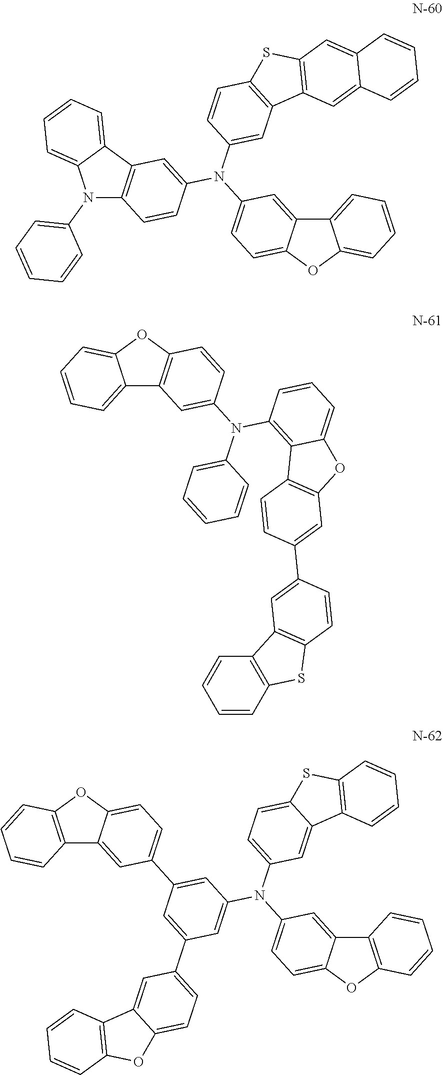 [SMILES: c1ccc(-n2c3ccccc3c3cc(N(c4ccc5oc6ccccc6c5c4)c4ccc5sc6cc7ccccc7cc6c5c4)ccc32)cc1 ; c1ccc(N(c2ccc3oc4ccccc4c3c2)c2cccc3oc4cc(-c5ccc6sc7ccccc7c6c5)ccc4c23)cc1 ; c1ccc2c(c1)oc1ccc(-c3cc(-c4ccc5oc6ccccc6c5c4)cc(N(c4ccc5oc6ccccc6c5c4)c4ccc5sc6ccccc6c5c4)c3)cc12]

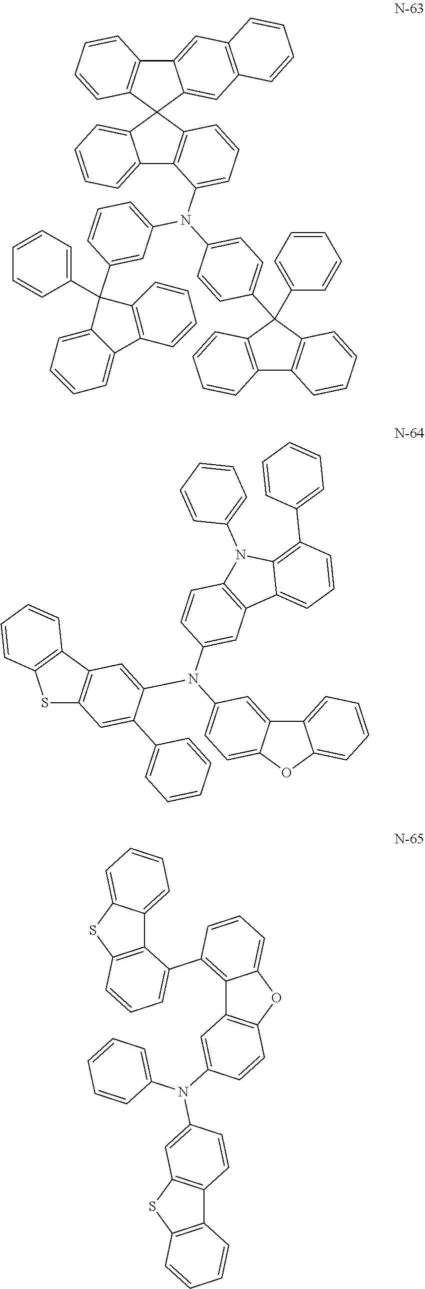 [SMILES: c1ccc(C2(c3ccc(N(c4cccc(C5(c6ccccc6)c6ccccc6-c6ccccc65)c4)c4cccc5c4-c4ccccc4C54c5ccccc5-c5cc6ccccc6cc54)cc3)c3ccccc3-c3ccccc32)cc1 ; c1ccc(-c2cc3sc4ccccc4c3cc2N(c2ccc3oc4ccccc4c3c2)c2ccc3c(c2)c2cccc(-c4ccccc4)c2n3-c2ccccc2)cc1 ; c1ccc(N(c2ccc3c(c2)sc2ccccc23)c2ccc3oc4cccc(-c5cccc6sc7ccccc7c56)c4c3c2)cc1]

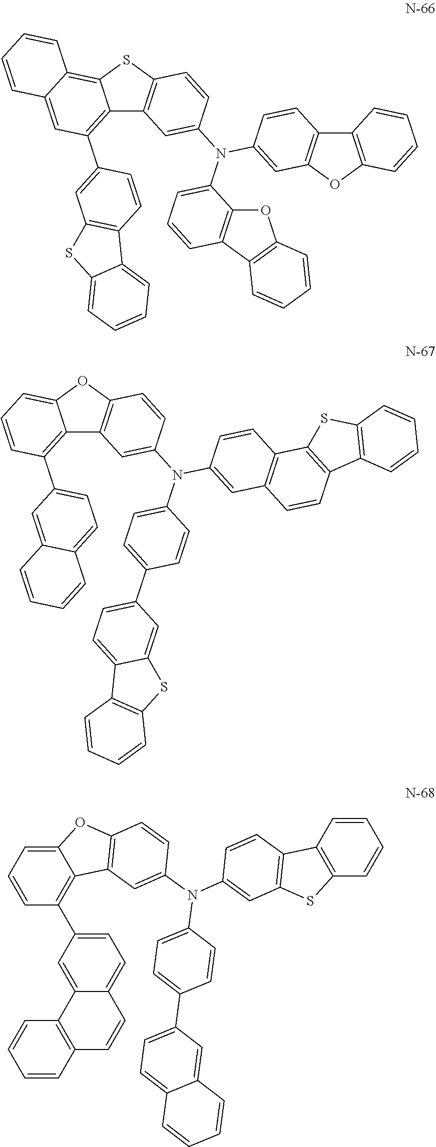 [SMILES: c1ccc2c(c1)cc(-c1ccc3c(c1)sc1ccccc13)c1c3cc(N(c4ccc5c(c4)oc4ccccc45)c4cccc5c4oc4ccccc45)ccc3sc21 ; c1ccc2cc(-c3cccc4oc5ccc(N(c6ccc(-c7ccc8c(c7)sc7ccccc78)cc6)c6ccc7c(ccc8c9ccccc9sc78)c6)cc5c34)ccc2c1 ; c1ccc2cc(-c3ccc(N(c4ccc5c(c4)sc4ccccc45)c4ccc5oc6cccc(-c7ccc8ccc9ccccc9c8c7)c6c5c4)cc3)ccc2c1]

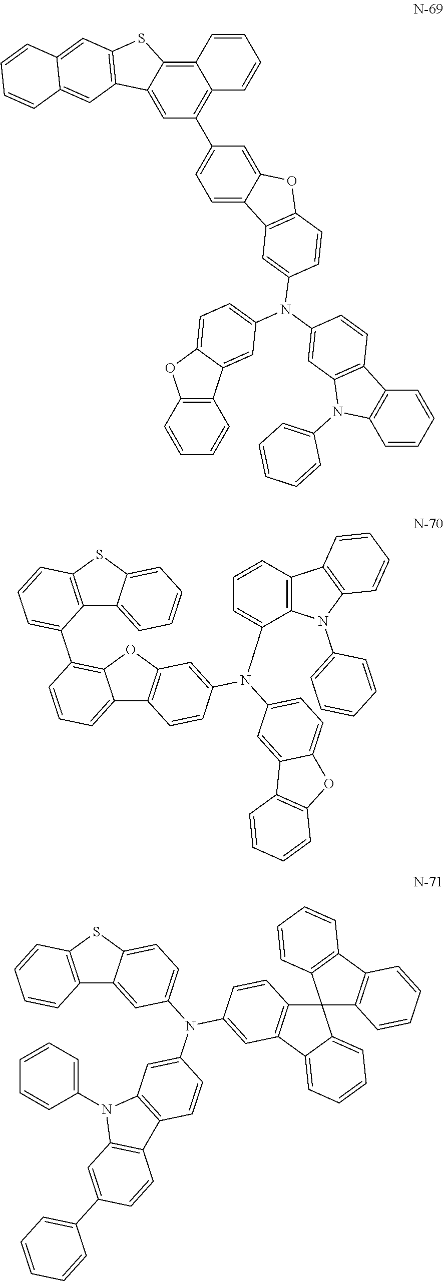 [SMILES: c1ccc(-n2c3ccccc3c3ccc(N(c4ccc5oc6ccccc6c5c4)c4ccc5oc6cc(-c7cc8c9cc%10ccccc%10cc9sc8c8ccccc78)ccc6c5c4)cc32)cc1 ; c1ccc(-n2c3ccccc3c3cccc(N(c4ccc5c(c4)oc4c(-c6cccc7sc8ccccc8c67)cccc45)c4ccc5oc6ccccc6c5c4)c32)cc1 ; c1ccc(-c2ccc3c4ccc(N(c5ccc6c(c5)-c5ccccc5C65c6ccccc6-c6ccccc65)c5ccc6sc7ccccc7c6c5)cc4n(-c4ccccc4)c3c2)cc1]

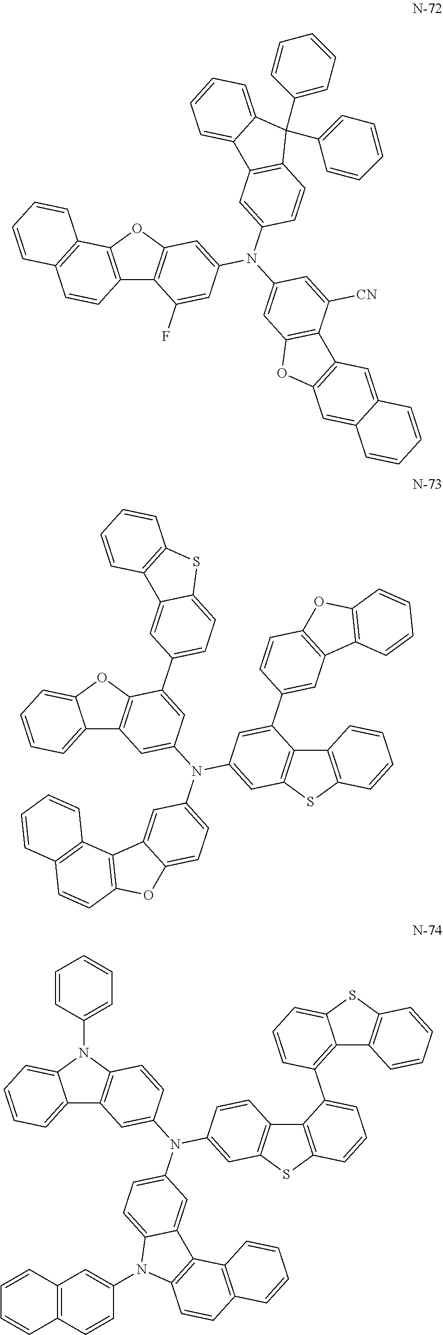 [SMILES: N#Cc1cc(N(c2ccc3c(c2)-c2ccccc2C3(c2ccccc2)c2ccccc2)c2cc(F)c3c(c2)oc2c4ccccc4ccc23)cc2oc3cc4ccccc4cc3c12 ; c1ccc2c(c1)ccc1oc3ccc(N(c4cc(-c5ccc6oc7ccccc7c6c5)c5c(c4)sc4ccccc45)c4cc(-c5ccc6sc7ccccc7c6c5)c5oc6ccccc6c5c4)cc3c12 ; c1ccc(-n2c3ccccc3c3cc(N(c4ccc5c(c4)sc4cccc(-c6cccc7sc8ccccc8c67)c45)c4ccc5c(c4)c4c6ccccc6ccc4n5-c4ccc5ccccc5c4)ccc32)cc1]

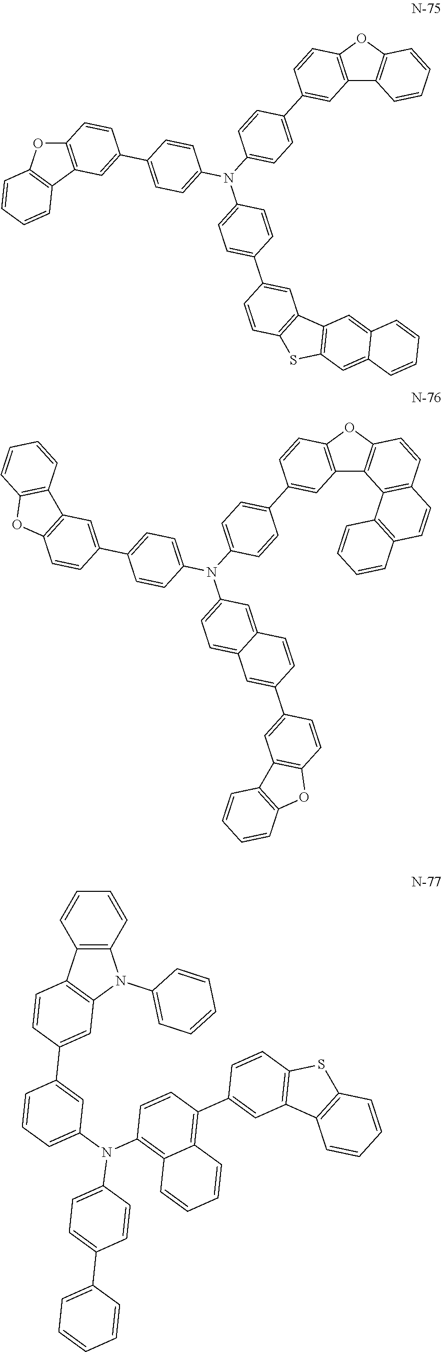 [SMILES: c1ccc2cc3c(cc2c1)sc1ccc(-c2ccc(N(c4ccc(-c5ccc6oc7ccccc7c6c5)cc4)c4ccc(-c5ccc6oc7ccccc7c6c5)cc4)cc2)cc13 ; c1ccc2c(c1)ccc1ccc3oc4ccc(-c5ccc(N(c6ccc(-c7ccc8oc9ccccc9c8c7)cc6)c6ccc7cc(-c8ccc9oc%10ccccc%10c9c8)ccc7c6)cc5)cc4c3c12 ; c1ccc(-c2ccc(N(c3cccc(-c4ccc5c6ccccc6n(-c6ccccc6)c5c4)c3)c3ccc(-c4ccc5sc6ccccc6c5c4)c4ccccc34)cc2)cc1]

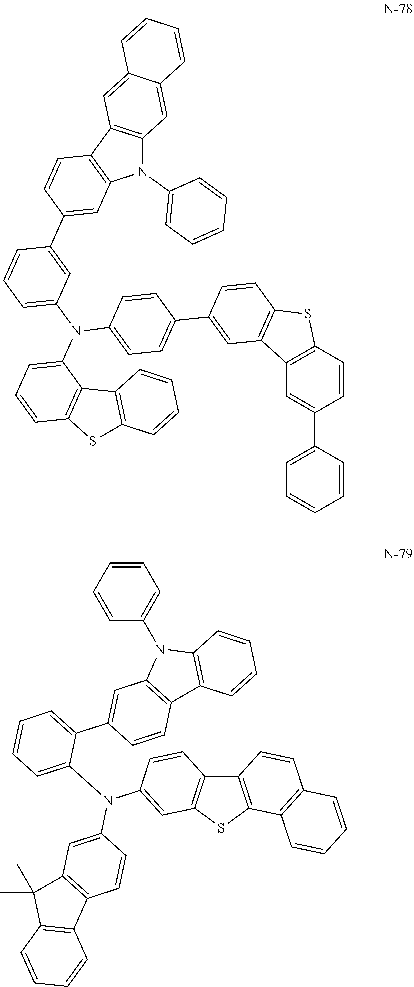 [SMILES: c1ccc(-c2ccc3sc4ccc(-c5ccc(N(c6cccc(-c7ccc8c9cc%10ccccc%10cc9n(-c9ccccc9)c8c7)c6)c6cccc7sc8ccccc8c67)cc5)cc4c3c2)cc1 ; CC1(C)c2ccccc2-c2ccc(N(c3ccc4c(c3)sc3c5ccccc5ccc43)c3ccccc3-c3ccc4c5ccccc5n(-c5ccccc5)c4c3)cc21]

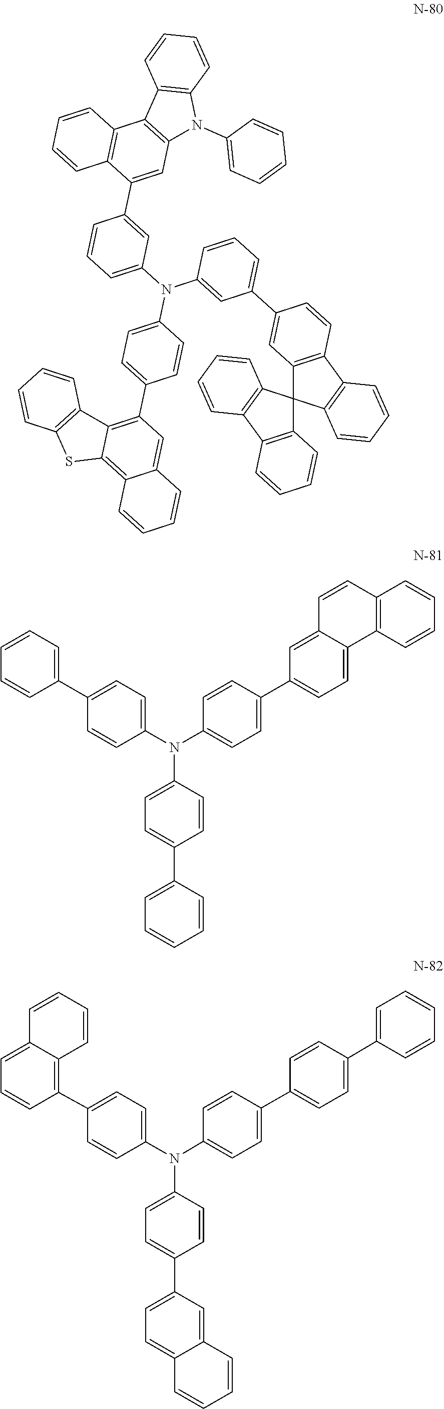 [SMILES: c1ccc(-n2c3ccccc3c3c4ccccc4c(-c4cccc(N(c5ccc(-c6cc7ccccc7c7sc8ccccc8c67)cc5)c5cccc(-c6ccc7c(c6)C6(c8ccccc8-c8ccccc86)c6ccccc6-7)c5)c4)cc32)cc1 ; c1ccc(-c2ccc(N(c3ccc(-c4ccccc4)cc3)c3ccc(-c4ccc5c(ccc6ccccc65)c4)cc3)cc2)cc1 ; c1ccc(-c2ccc(-c3ccc(N(c4ccc(-c5ccc6ccccc6c5)cc4)c4ccc(-c5cccc6ccccc56)cc4)cc3)cc2)cc1]

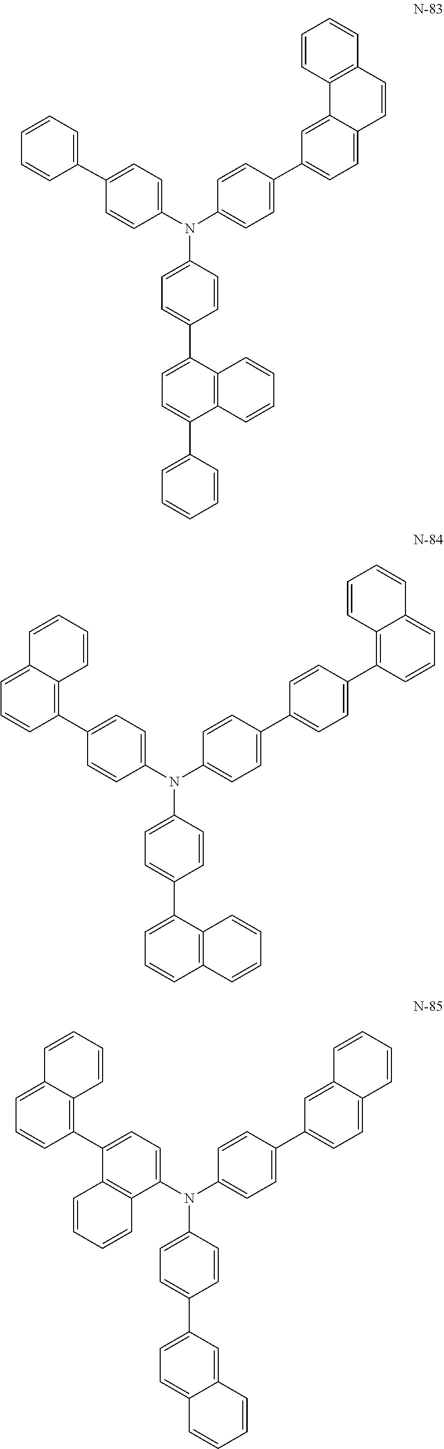 [SMILES: c1ccc(-c2ccc(N(c3ccc(-c4ccc5ccc6ccccc6c5c4)cc3)c3ccc(-c4ccc(-c5ccccc5)c5ccccc45)cc3)cc2)cc1 ; c1ccc2c(-c3ccc(-c4ccc(N(c5ccc(-c6cccc7ccccc67)cc5)c5ccc(-c6cccc7ccccc67)cc5)cc4)cc3)cccc2c1 ; c1ccc2cc(-c3ccc(N(c4ccc(-c5ccc6ccccc6c5)cc4)c4ccc(-c5cccc6ccccc56)c5ccccc45)cc3)ccc2c1]

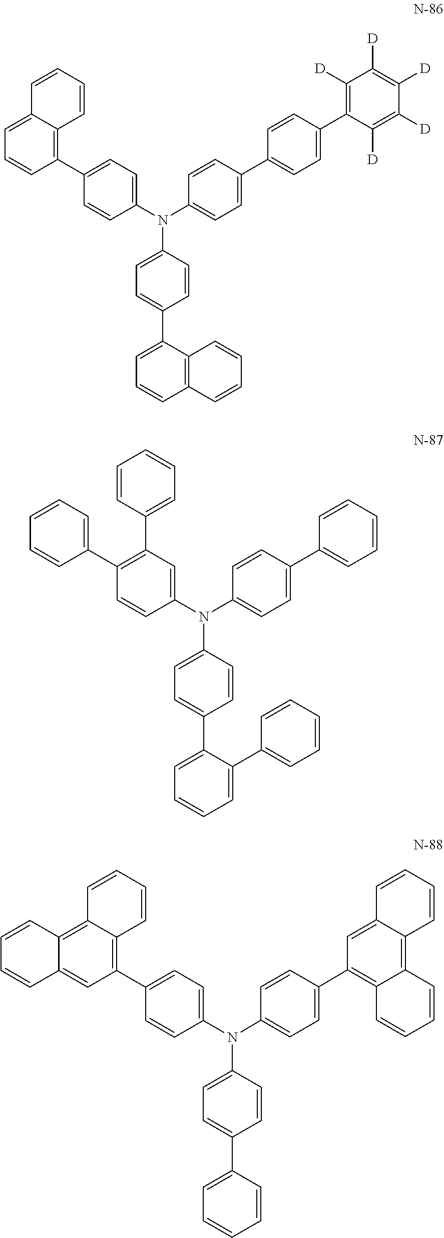 [SMILES: [2H]c1c([2H])c([2H])c(-c2ccc(-c3ccc(N(c4ccc(-c5cccc6ccccc56)cc4)c4ccc(-c5cccc6ccccc56)cc4)cc3)cc2)c([2H])c1[2H] ; c1ccc(-c2ccc(N(c3ccc(-c4ccccc4-c4ccccc4)cc3)c3ccc(-c4ccccc4)c(-c4ccccc4)c3)cc2)cc1 ; c1ccc(-c2ccc(N(c3ccc(-c4cc5ccccc5c5ccccc45)cc3)c3ccc(-c4cc5ccccc5c5ccccc45)cc3)cc2)cc1]

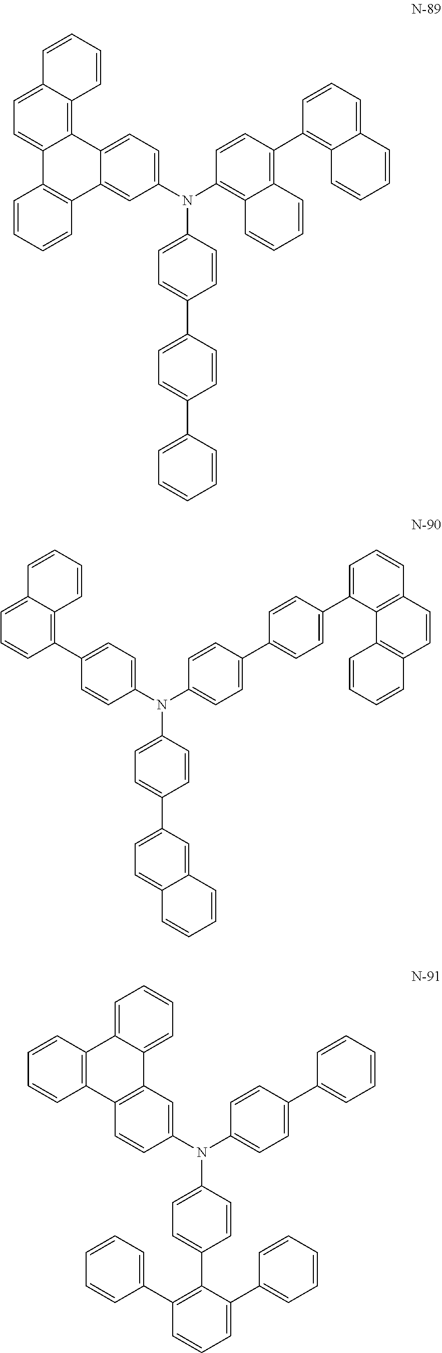 [SMILES: c1ccc(-c2ccc(-c3ccc(N(c4ccc5c(c4)c4ccccc4c4ccc6ccccc6c45)c4ccc(-c5cccc6ccccc56)c5ccccc45)cc3)cc2)cc1 ; c1ccc2cc(-c3ccc(N(c4ccc(-c5ccc(-c6cccc7ccc8ccccc8c67)cc5)cc4)c4ccc(-c5cccc6ccccc56)cc4)cc3)ccc2c1 ; c1ccc(-c2ccc(N(c3ccc(-c4c(-c5ccccc5)cccc4-c4ccccc4)cc3)c3ccc4c5ccccc5c5ccccc5c4c3)cc2)cc1]

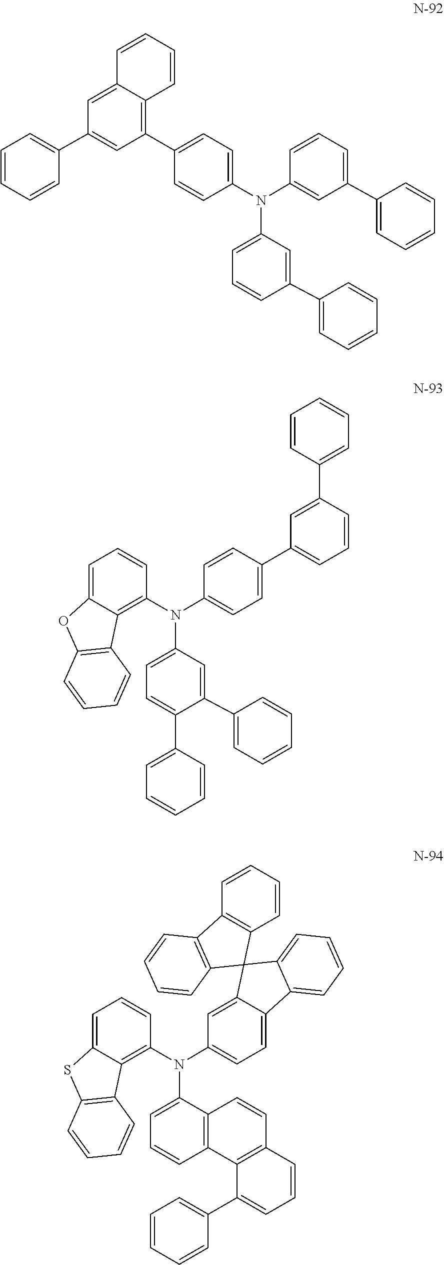 [SMILES: c1ccc(-c2cccc(N(c3ccc(-c4cc(-c5ccccc5)cc5ccccc45)cc3)c3cccc(-c4ccccc4)c3)c2)cc1 ; c1ccc(-c2cccc(-c3ccc(N(c4ccc(-c5ccccc5)c(-c5ccccc5)c4)c4cccc5oc6ccccc6c45)cc3)c2)cc1 ; c1ccc(-c2cccc3ccc4c(N(c5ccc6c(c5)C5(c7ccccc7-c7ccccc75)c5ccccc5-6)c5cccc6sc7ccccc7c56)cccc4c23)cc1]

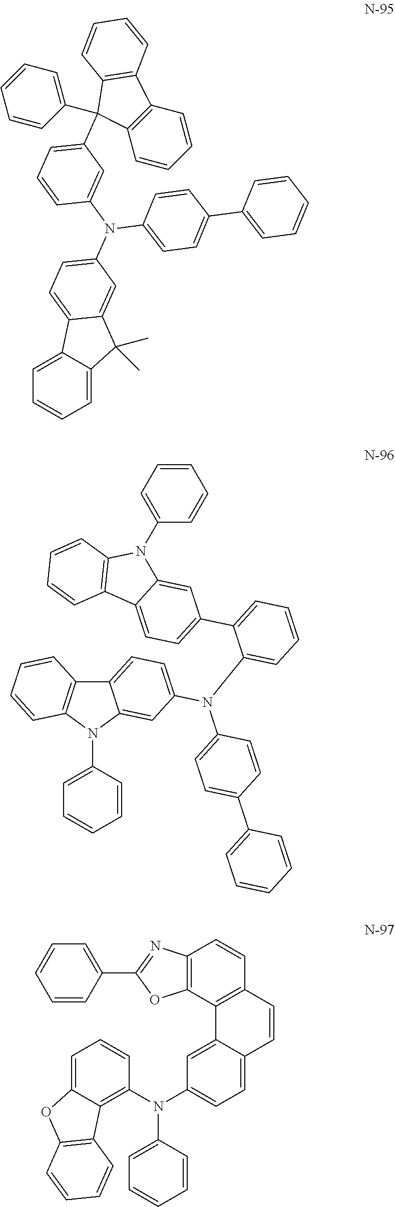 [SMILES: CC1(C)c2ccccc2-c2ccc(N(c3ccc(-c4ccccc4)cc3)c3cccc(C4(c5ccccc5)c5ccccc5-c5ccccc54)c3)cc21 ; c1ccc(-c2ccc(N(c3ccc4c5ccccc5n(-c5ccccc5)c4c3)c3ccccc3-c3ccc4c5ccccc5n(-c5ccccc5)c4c3)cc2)cc1 ; c1ccc(-c2nc3ccc4ccc5ccc(N(c6ccccc6)c6cccc7oc8ccccc8c67)cc5c4c3o2)cc1]

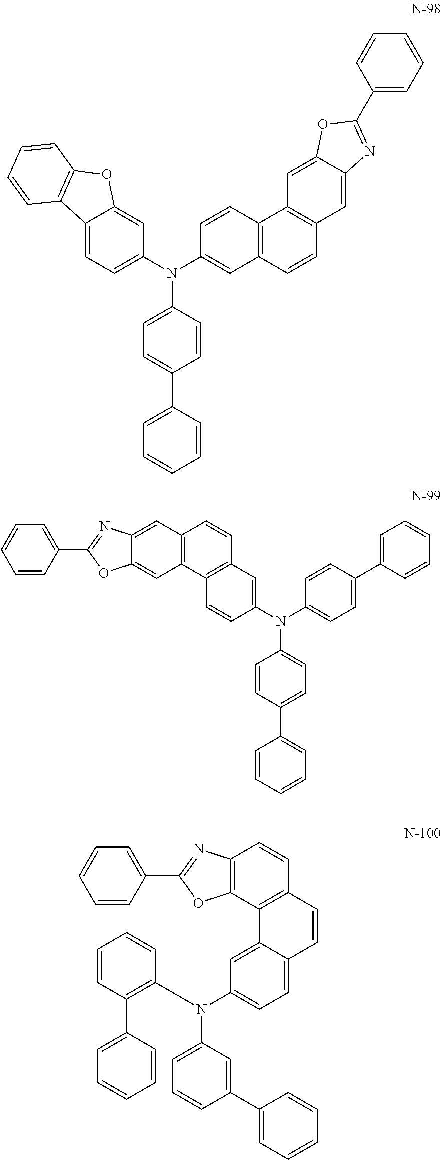 [SMILES: c1ccc(-c2ccc(N(c3ccc4c(ccc5cc6nc(-c7ccccc7)oc6cc54)c3)c3ccc4c(c3)oc3ccccc34)cc2)cc1 ; c1ccc(-c2ccc(N(c3ccc(-c4ccccc4)cc3)c3ccc4c(ccc5cc6nc(-c7ccccc7)oc6cc54)c3)cc2)cc1 ; c1ccc(-c2cccc(N(c3ccc4ccc5ccc6nc(-c7ccccc7)oc6c5c4c3)c3ccccc3-c3ccccc3)c2)cc1]

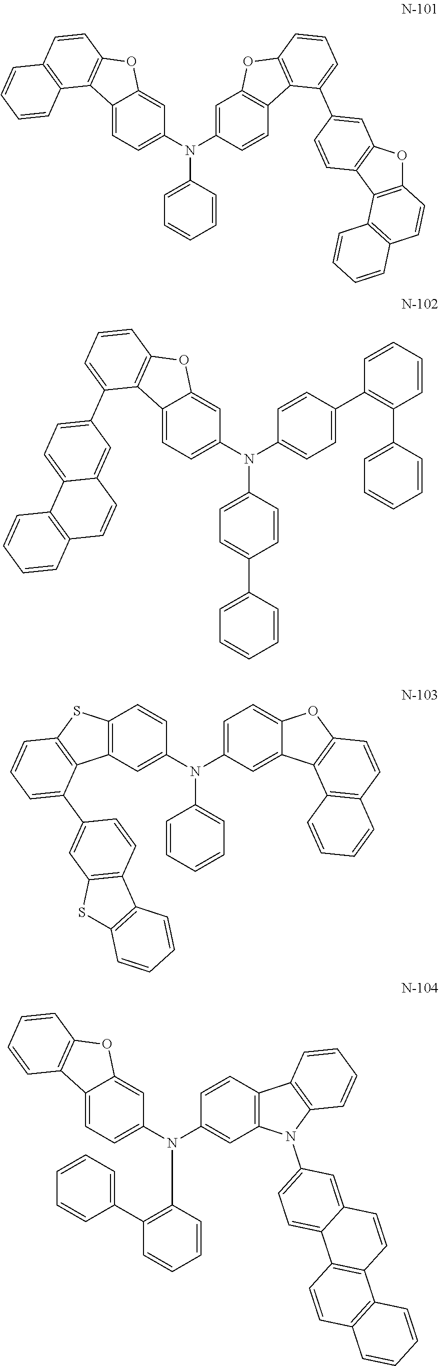

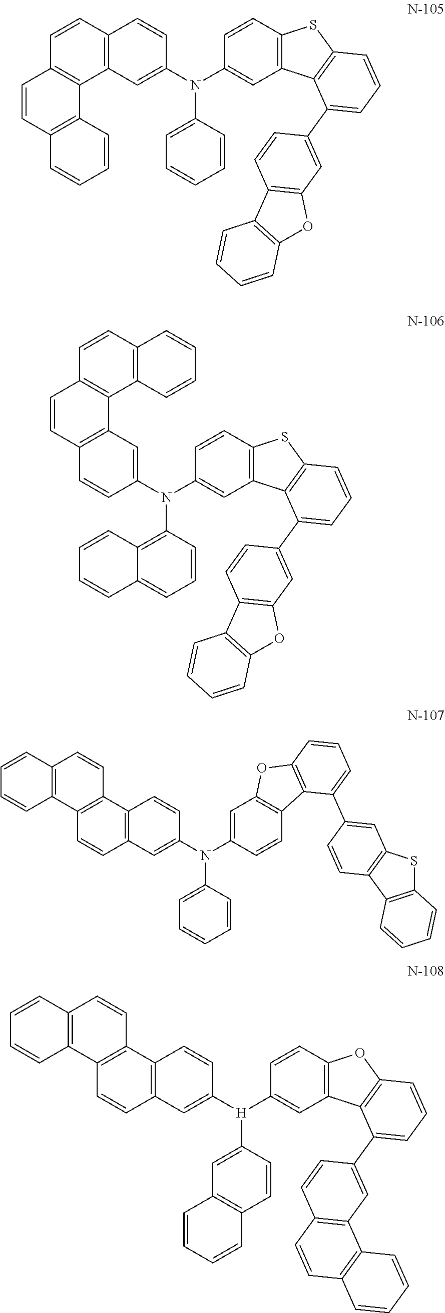

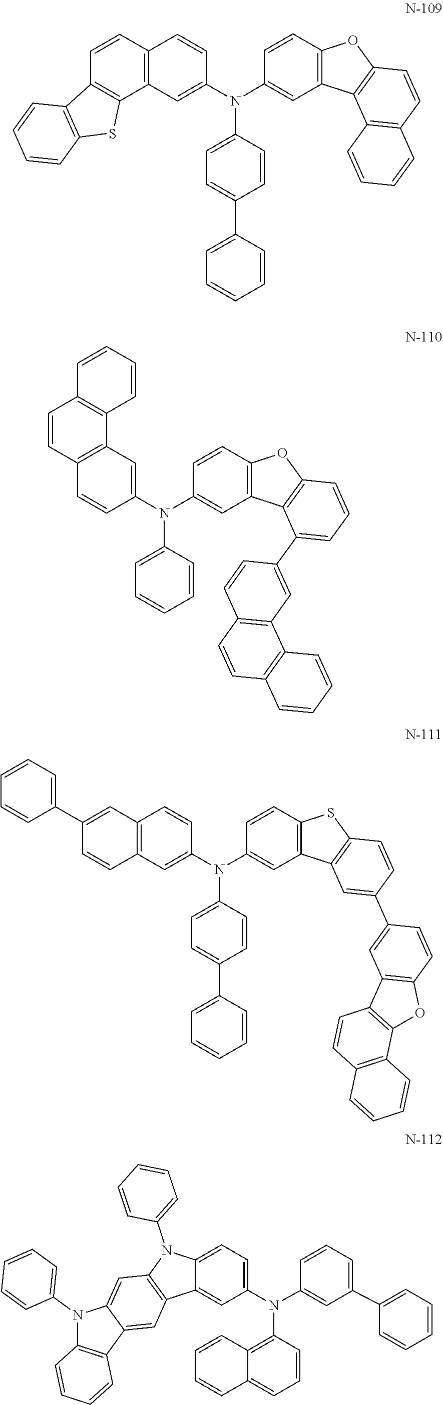 [SMILES: c1ccc(-c2ccc(N(c3ccc4ccc5c6ccccc6sc5c4c3)c3ccc4oc5ccc6ccccc6c5c4c3)cc2)cc1 ; c1ccc(N(c2ccc3ccc4ccccc4c3c2)c2ccc3oc4cccc(-c5ccc6ccc7ccccc7c6c5)c4c3c2)cc1 ; c1ccc(-c2ccc(N(c3ccc4cc(-c5ccccc5)ccc4c3)c3ccc4sc5ccc(-c6ccc7oc8c9ccccc9ccc8c7c6)cc5c4c3)cc2)cc1 ; c1ccc(-c2cccc(N(c3ccc4c(c3)c3cc5c6ccccc6n(-c6ccccc6)c5cc3n4-c3ccccc3)c3cccc4ccccc34)c2)cc1]

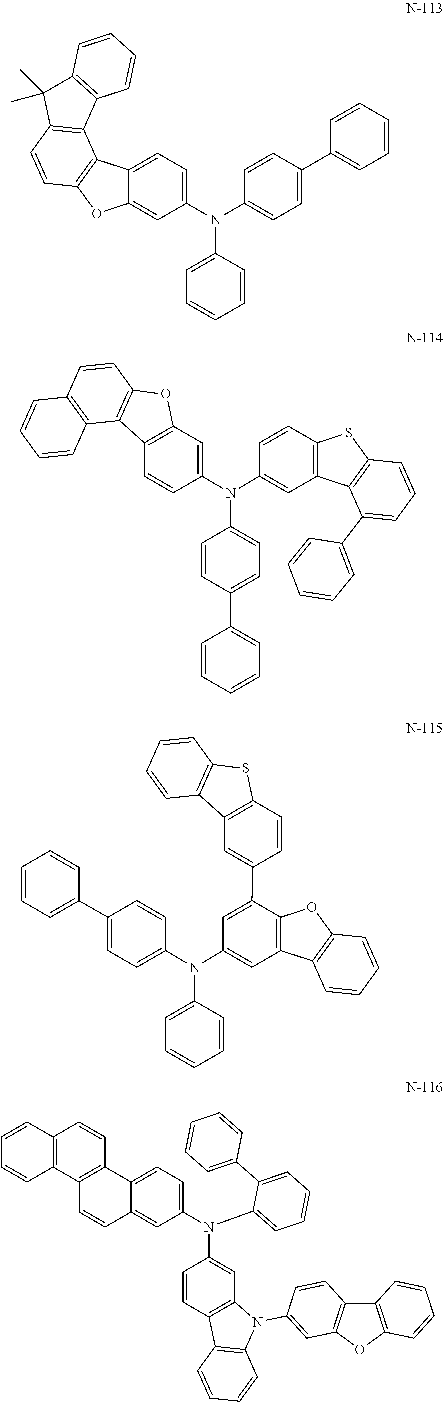 [SMILES: CC1(C)c2ccccc2-c2c1ccc1oc3cc(N(c4ccccc4)c4ccc(-c5ccccc5)cc4)ccc3c21 ; c1ccc(-c2ccc(N(c3ccc4c(c3)oc3ccc5ccccc5c34)c3ccc4sc5cccc(-c6ccccc6)c5c4c3)cc2)cc1 ; c1ccc(-c2ccc(N(c3ccccc3)c3cc(-c4ccc5sc6ccccc6c5c4)c4oc5ccccc5c4c3)cc2)cc1 ; c1ccc(-c2ccccc2N(c2ccc3c(ccc4c5ccccc5ccc34)c2)c2ccc3c4ccccc4n(-c4ccc5c(c4)oc4ccccc45)c3c2)cc1]

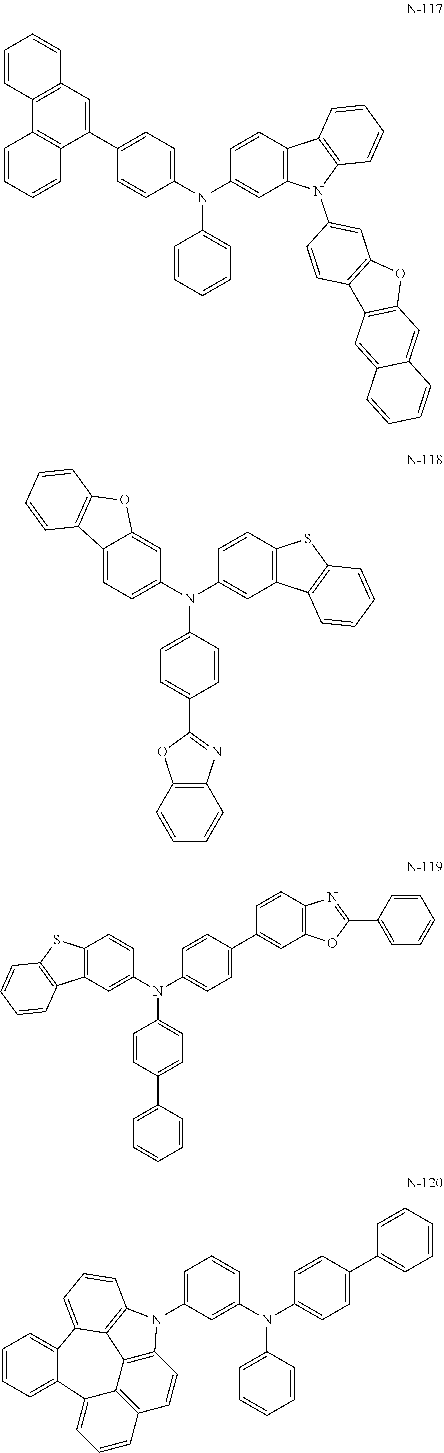 [SMILES: c1ccc(N(c2ccc(-c3cc4ccccc4c4ccccc34)cc2)c2ccc3c4ccccc4n(-c4ccc5c(c4)oc4cc6ccccc6cc45)c3c2)cc1 ; c1ccc2oc(-c3ccc(N(c4ccc5c(c4)oc4ccccc45)c4ccc5sc6ccccc6c5c4)cc3)nc2c1 ; c1ccc(-c2ccc(N(c3ccc(-c4ccc5nc(-c6ccccc6)oc5c4)cc3)c3ccc4sc5ccccc5c4c3)cc2)cc1 ; c1ccc(-c2ccc(N(c3ccccc3)c3cccc(-n4c5cccc6c5c5c7c(cccc7ccc54)-c4ccccc4-6)c3)cc2)cc1]

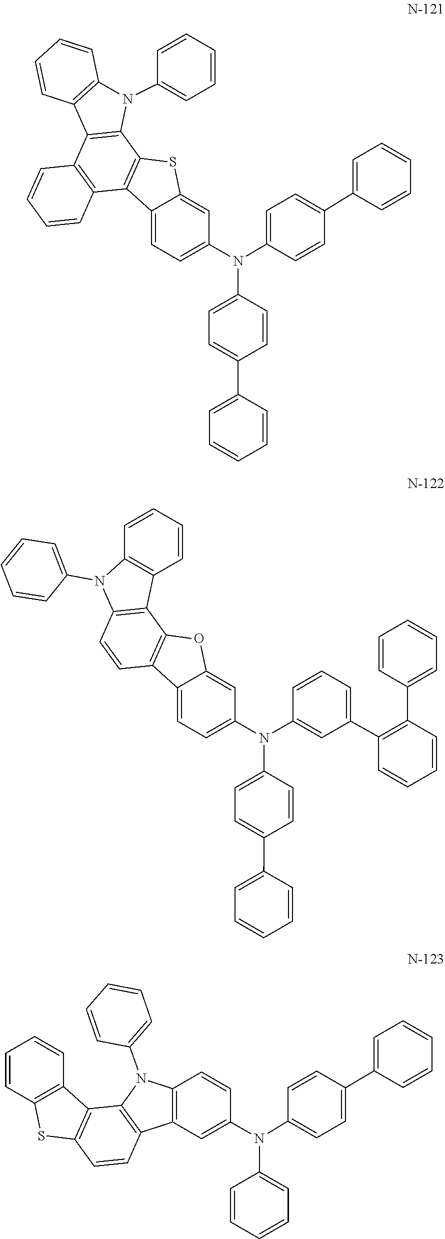 [SMILES: c1ccc(-c2ccc(N(c3ccc(-c4ccccc4)cc3)c3ccc4c(c3)sc3c4c4ccccc4c4c5ccccc5n(-c5ccccc5)c34)cc2)cc1 ; c1ccc(-c2ccc(N(c3cccc(-c4ccccc4-c4ccccc4)c3)c3ccc4c(c3)oc3c4ccc4c3c3ccccc3n4-c3ccccc3)cc2)cc1 ; c1ccc(-c2ccc(N(c3ccccc3)c3ccc4c(c3)c3ccc5sc6ccccc6c5c3n4-c3ccccc3)cc2)cc1]

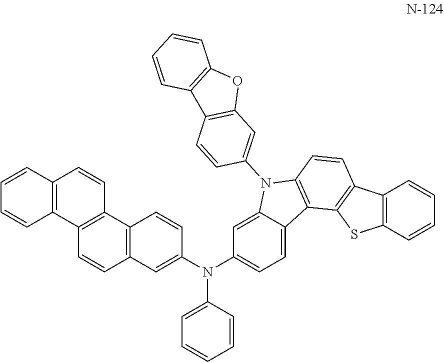 [SMILES: c1ccc(N(c2ccc3c(ccc4c5ccccc5ccc34)c2)c2ccc3c4c5sc6ccccc6c5ccc4n(-c4ccc5c(c4)oc4ccccc45)c3c2)cc1]

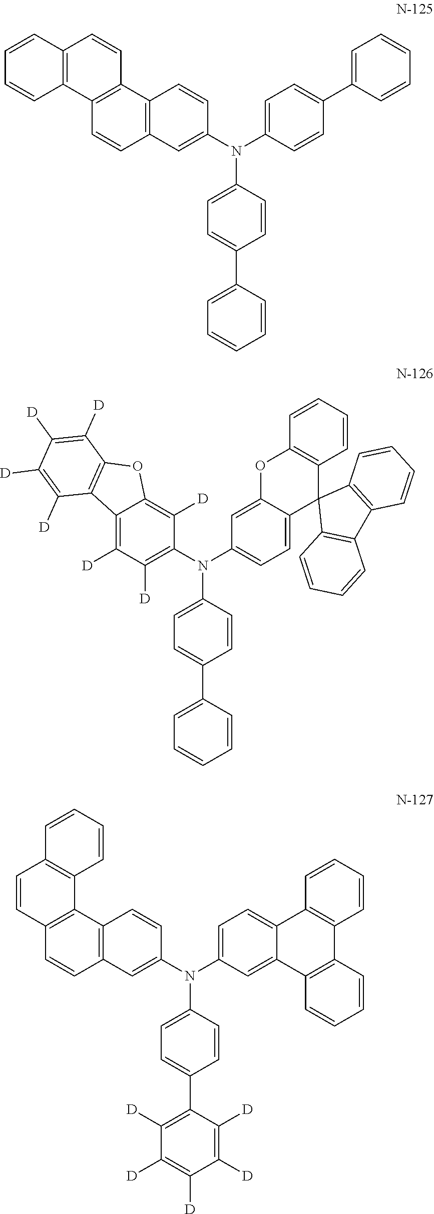 [SMILES: c1ccc(-c2ccc(N(c3ccc(-c4ccccc4)cc3)c3ccc4c(ccc5c6ccccc6ccc45)c3)cc2)cc1 ; [2H]c1c([2H])c([2H])c2c(oc3c([2H])c(N(c4ccc(-c5ccccc5)cc4)c4ccc5c(c4)Oc4ccccc4C54c5ccccc5-c5ccccc54)c([2H])c([2H])c32)c1[2H] ; [2H]c1c([2H])c([2H])c(-c2ccc(N(c3ccc4c(ccc5ccc6ccccc6c54)c3)c3ccc4c5ccccc5c5ccccc5c4c3)cc2)c([2H])c1[2H]]

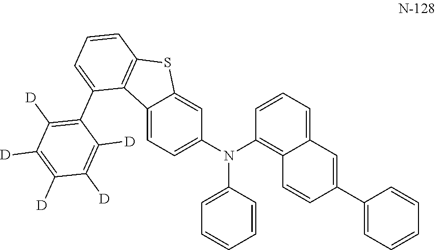 [SMILES: [2H]c1c([2H])c([2H])c(-c2cccc3sc4cc(N(c5ccccc5)c5cccc6cc(-c7ccccc7)ccc56)ccc4c23)c([2H])c1[2H]]

Specifically, the compound represented by Formula 3 may be a compound represented by any one of the compounds S-1 to S-120, but is not limited thereto.

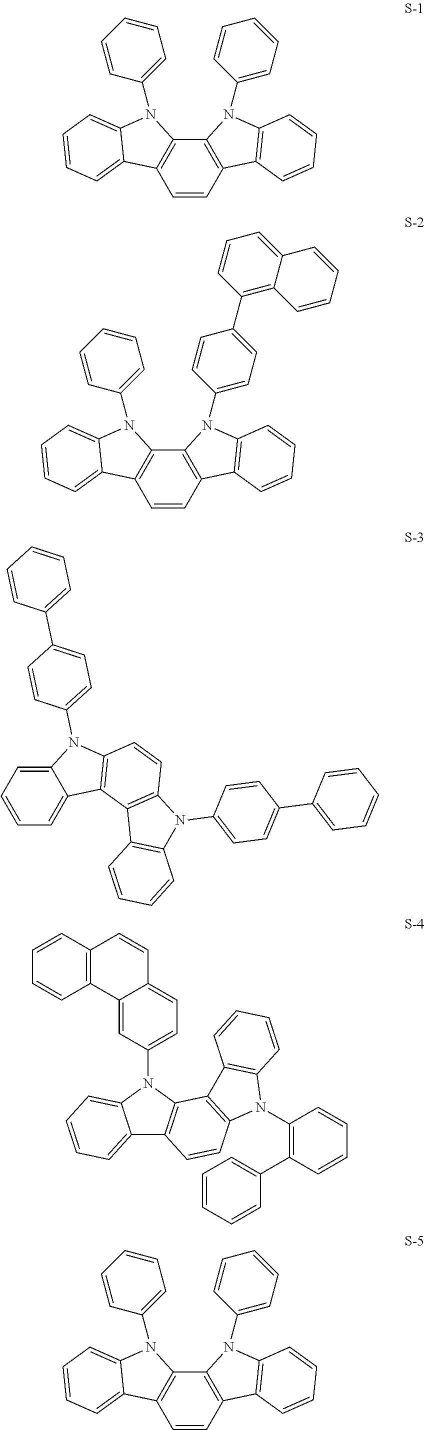

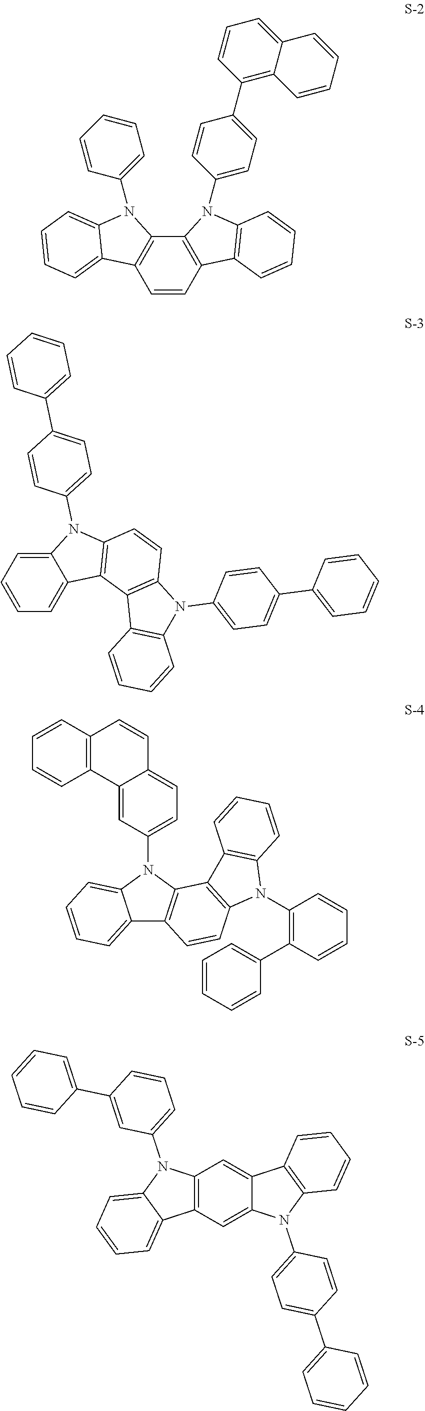

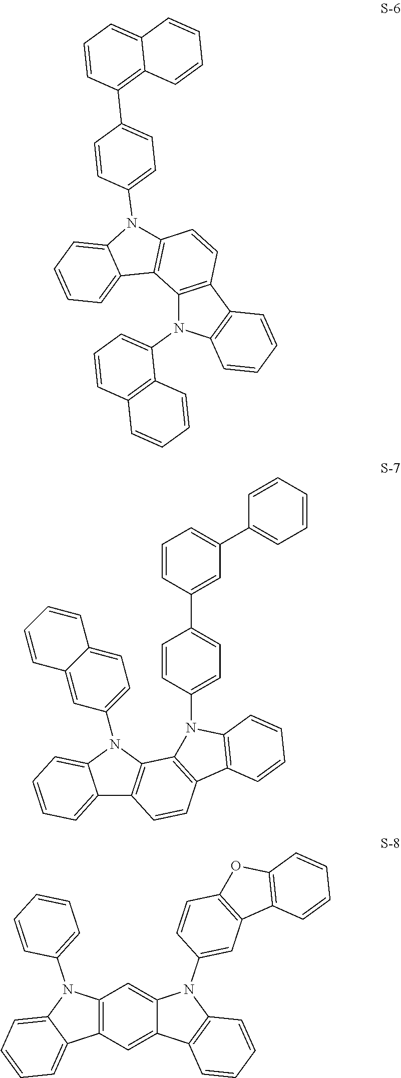 [SMILES: c1ccc2c(-c3ccc(-n4c5ccccc5c5c4ccc4c6ccccc6n(-c6cccc7ccccc67)c45)cc3)cccc2c1 ; c1ccc(-c2cccc(-c3ccc(-n4c5ccccc5c5ccc6c7ccccc7n(-c7ccc8ccccc8c7)c6c54)cc3)c2)cc1 ; c1ccc(-n2c3ccccc3c3cc4c5ccccc5n(-c5ccc6oc7ccccc7c6c5)c4cc32)cc1]

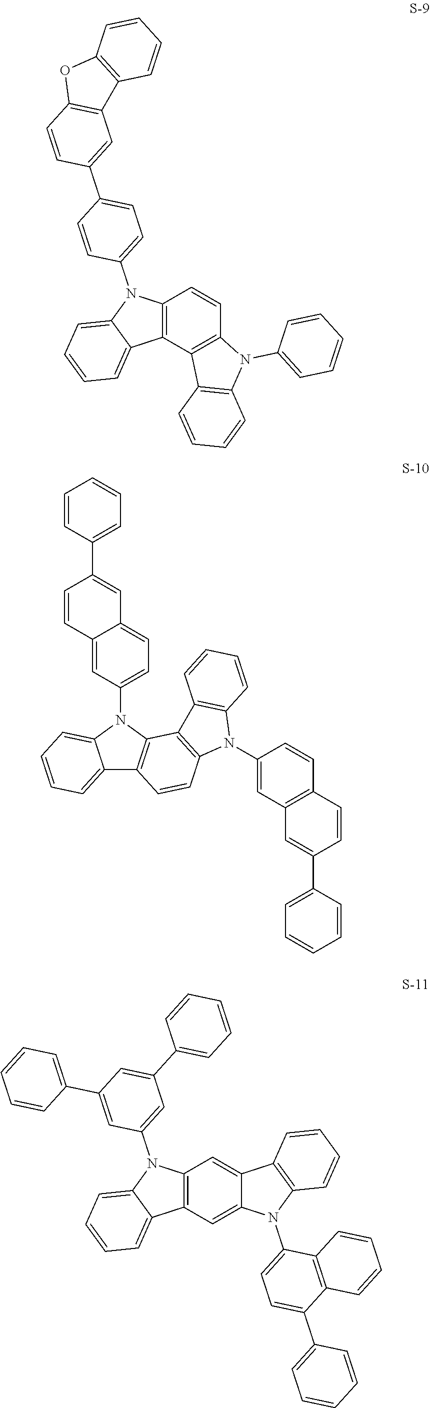 [SMILES: c1ccc(-n2c3ccccc3c3c4c5ccccc5n(-c5ccc(-c6ccc7oc8ccccc8c7c6)cc5)c4ccc32)cc1 ; c1ccc(-c2ccc3cc(-n4c5ccccc5c5ccc6c(c7ccccc7n6-c6ccc7ccc(-c8ccccc8)cc7c6)c54)ccc3c2)cc1 ; c1ccc(-c2cc(-c3ccccc3)cc(-n3c4ccccc4c4cc5c(cc43)c3ccccc3n5-c3ccc(-c4ccccc4)c4ccccc34)c2)cc1]

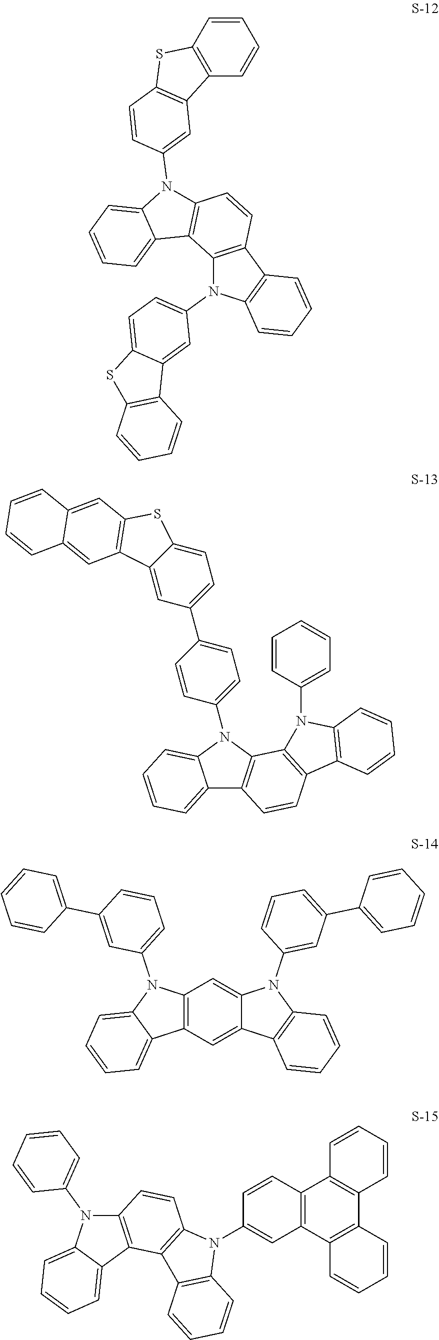 [SMILES: c1ccc2c(c1)sc1ccc(-n3c4ccccc4c4c3ccc3c5ccccc5n(-c5ccc6sc7ccccc7c6c5)c34)cc12 ; c1ccc(-n2c3ccccc3c3ccc4c5ccccc5n(-c5ccc(-c6ccc7sc8cc9ccccc9cc8c7c6)cc5)c4c32)cc1 ; c1ccc(-c2cccc(-n3c4ccccc4c4cc5c6ccccc6n(-c6cccc(-c7ccccc7)c6)c5cc43)c2)cc1 ; c1ccc(-n2c3ccccc3c3c4c5ccccc5n(-c5ccc6c7ccccc7c7ccccc7c6c5)c4ccc32)cc1]

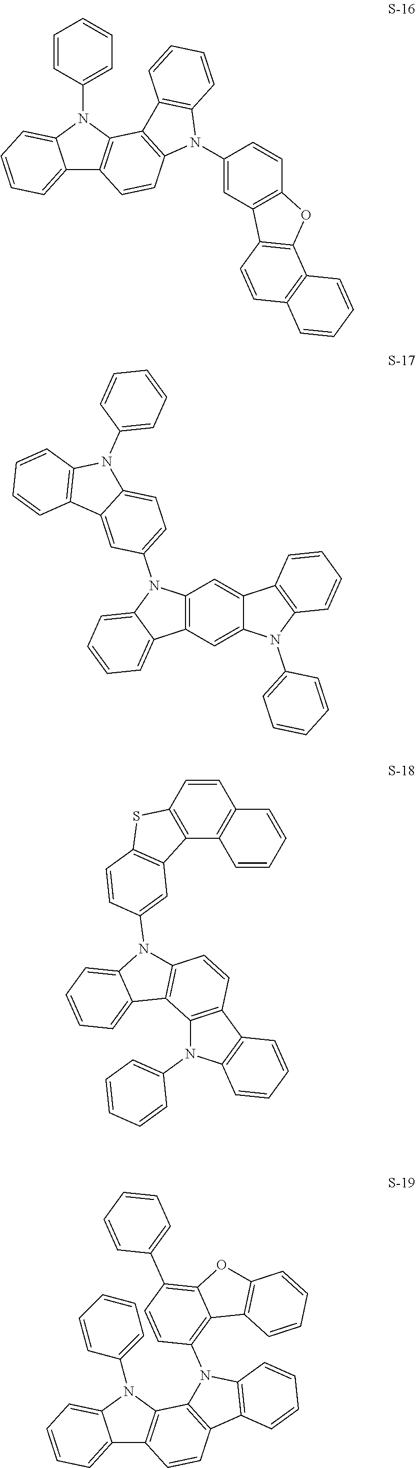 [SMILES: c1ccc(-n2c3ccccc3c3ccc4c(c5ccccc5n4-c4ccc5oc6c7ccccc7ccc6c5c4)c32)cc1 ; c1ccc(-n2c3ccccc3c3cc(-n4c5ccccc5c5cc6c(cc54)c4ccccc4n6-c4ccccc4)ccc32)cc1 ; c1ccc(-n2c3ccccc3c3ccc4c(c5ccccc5n4-c4ccc5sc6ccc7ccccc7c6c5c4)c32)cc1 ; c1ccc(-c2ccc(-n3c4ccccc4c4ccc5c6ccccc6n(-c6ccccc6)c5c43)c3c2oc2ccccc23)cc1]

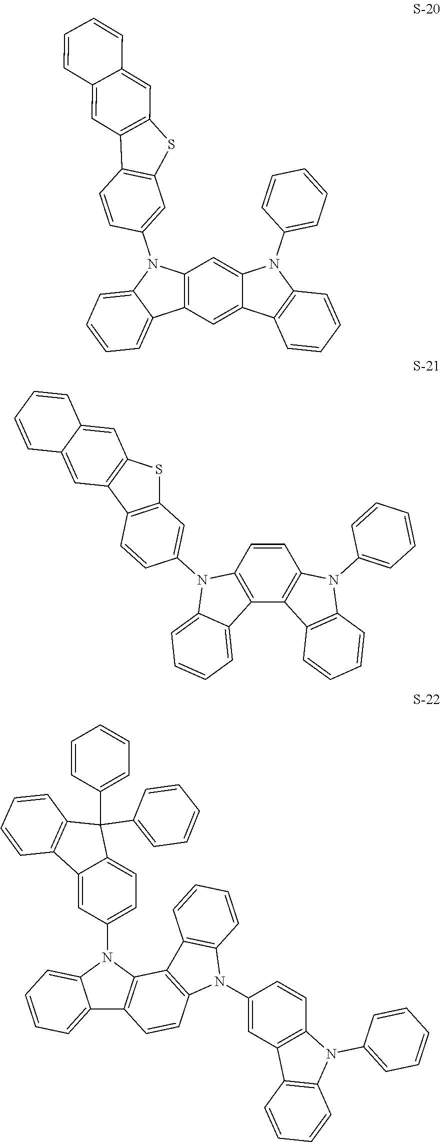 [SMILES: c1ccc(-n2c3ccccc3c3cc4c5ccccc5n(-c5ccc6c(c5)sc5cc7ccccc7cc56)c4cc32)cc1 ; c1ccc(-n2c3ccccc3c3c4c5ccccc5n(-c5ccc6c(c5)sc5cc7ccccc7cc56)c4ccc32)cc1 ; c1ccc(-n2c3ccccc3c3cc(-n4c5ccccc5c5c4ccc4c6ccccc6n(-c6ccc7c(c6)-c6ccccc6C7(c6ccccc6)c6ccccc6)c45)ccc32)cc1]

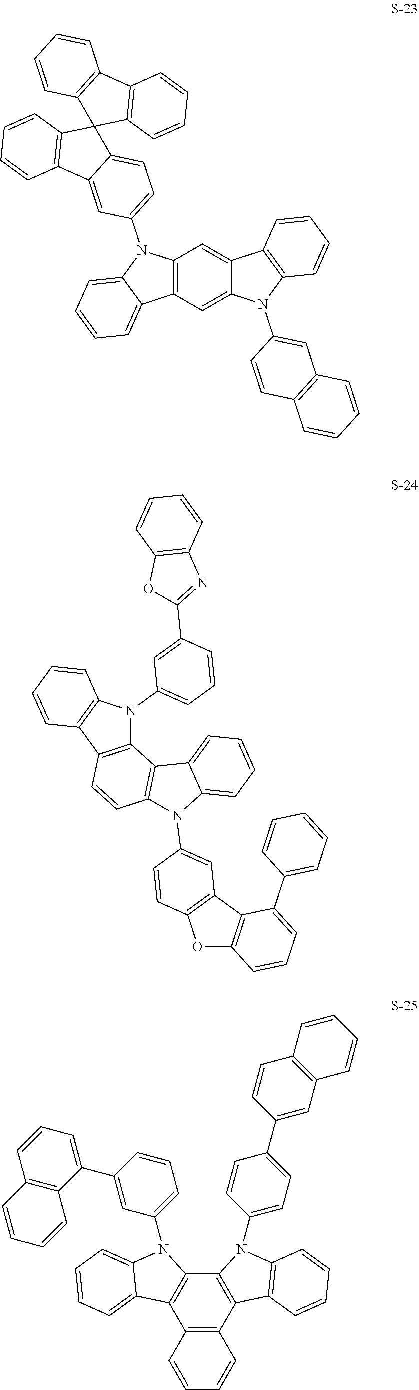 [SMILES: c1ccc2c(c1)-c1ccccc1C21c2ccccc2-c2cc(-n3c4ccccc4c4cc5c(cc43)c3ccccc3n5-c3ccc4ccccc4c3)ccc21 ; c1ccc(-c2cccc3oc4ccc(-n5c6ccccc6c6c5ccc5c7ccccc7n(-c7cccc(-c8nc9ccccc9o8)c7)c56)cc4c23)cc1 ; c1cc(-c2cccc3ccccc23)cc(-n2c3ccccc3c3c4ccccc4c4c5ccccc5n(-c5ccc(-c6ccc7ccccc7c6)cc5)c4c32)c1]

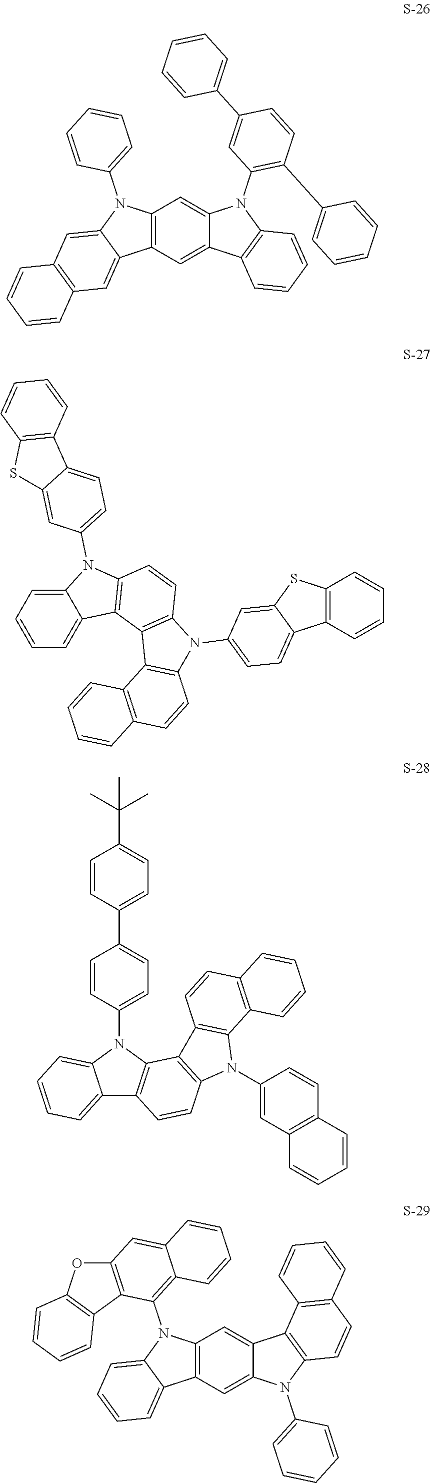

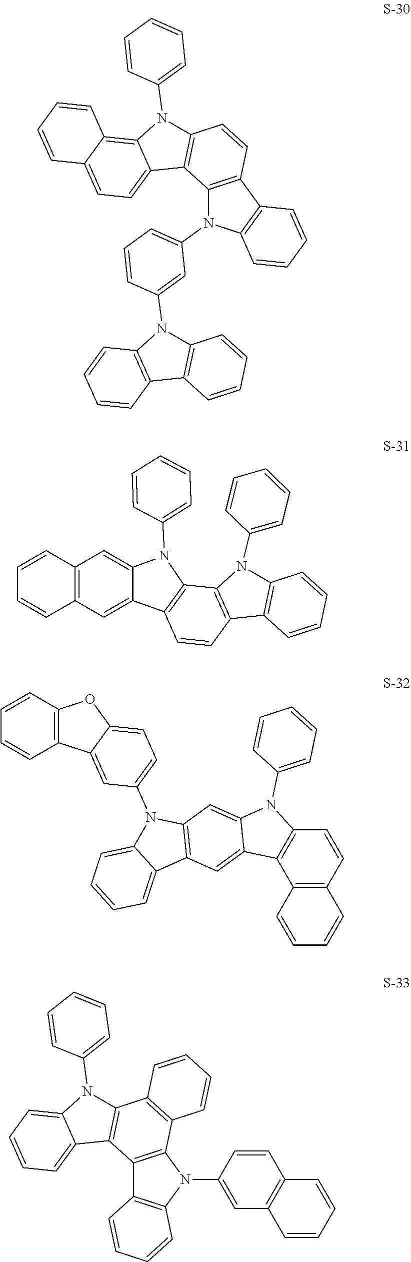 [SMILES: c1ccc(-n2c3ccc4c5ccccc5n(-c5cccc(-n6c7ccccc7c7ccccc76)c5)c4c3c3ccc4ccccc4c32)cc1 ; c1ccc(-n2c3ccccc3c3ccc4c5cc6ccccc6cc5n(-c5ccccc5)c4c32)cc1 ; c1ccc(-n2c3cc4c(cc3c3c5ccccc5ccc32)c2ccccc2n4-c2ccc3oc4ccccc4c3c2)cc1 ; c1ccc(-n2c3ccccc3c3c4c5ccccc5n(-c5ccc6ccccc6c5)c4c4ccccc4c32)cc1]

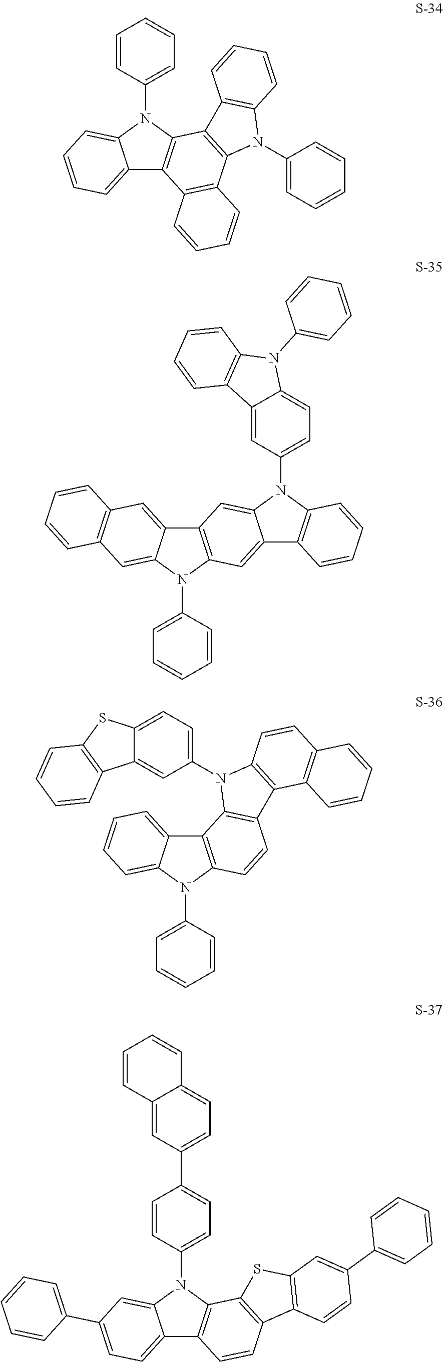

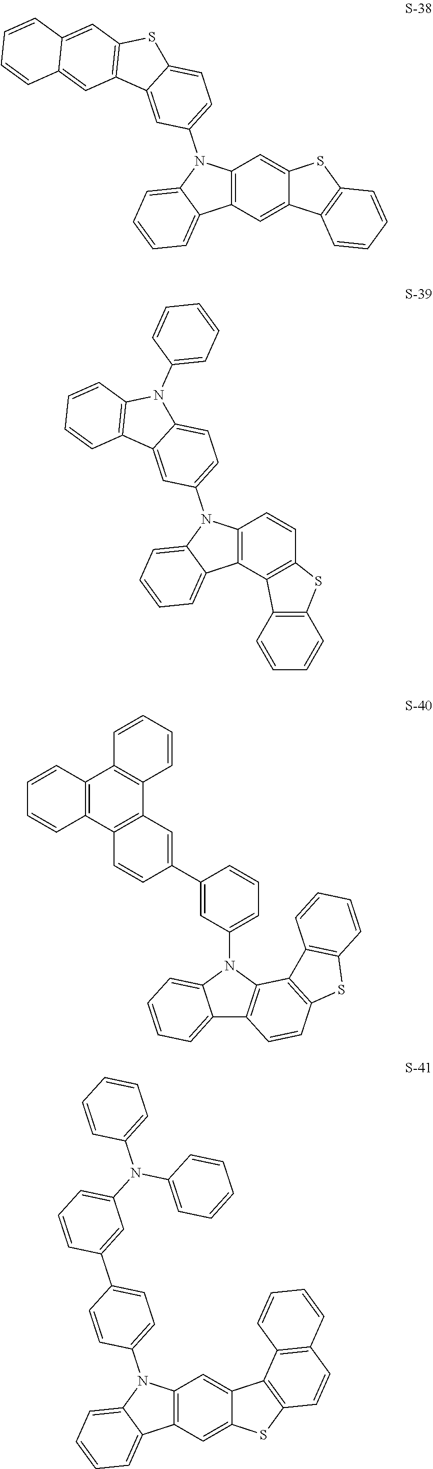 [SMILES: c1ccc2cc3c(cc2c1)sc1ccc(-n2c4ccccc4c4cc5c(cc42)sc2ccccc25)cc13 ; c1ccc(-n2c3ccccc3c3cc(-n4c5ccccc5c5c6c(ccc54)sc4ccccc46)ccc32)cc1 ; c1cc(-c2ccc3c4ccccc4c4ccccc4c3c2)cc(-n2c3ccccc3c3ccc4sc5ccccc5c4c32)c1 ; c1ccc(N(c2ccccc2)c2cccc(-c3ccc(-n4c5ccccc5c5cc6sc7ccc8ccccc8c7c6cc54)cc3)c2)cc1]

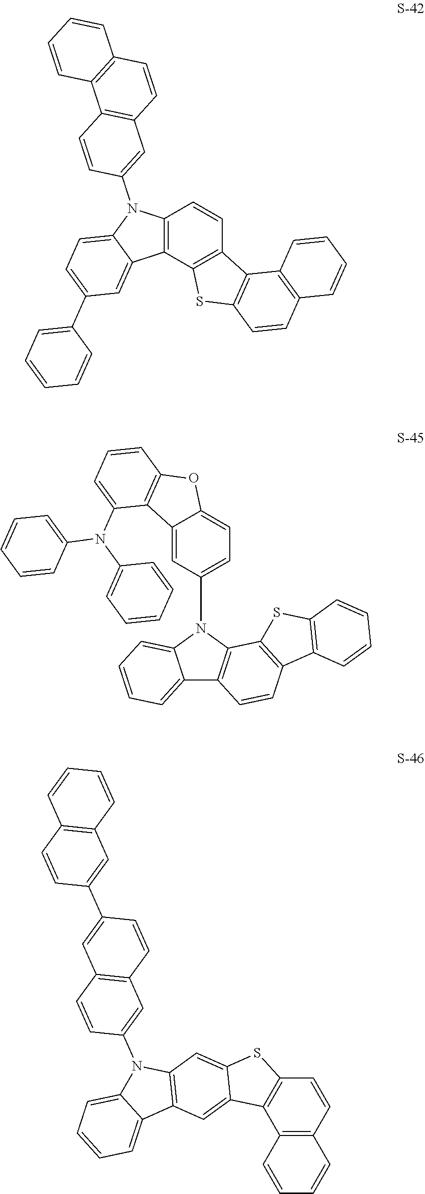 [SMILES: c1ccc(-c2ccc3c(c2)c2c4sc5ccc6ccccc6c5c4ccc2n3-c2ccc3c(ccc4ccccc43)c2)cc1 ; c1ccc(N(c2ccccc2)c2cccc3oc4ccc(-n5c6ccccc6c6ccc7c8ccccc8sc7c65)cc4c23)cc1 ; c1ccc2cc(-c3ccc4cc(-n5c6ccccc6c6cc7c(cc65)sc5ccc6ccccc6c57)ccc4c3)ccc2c1]

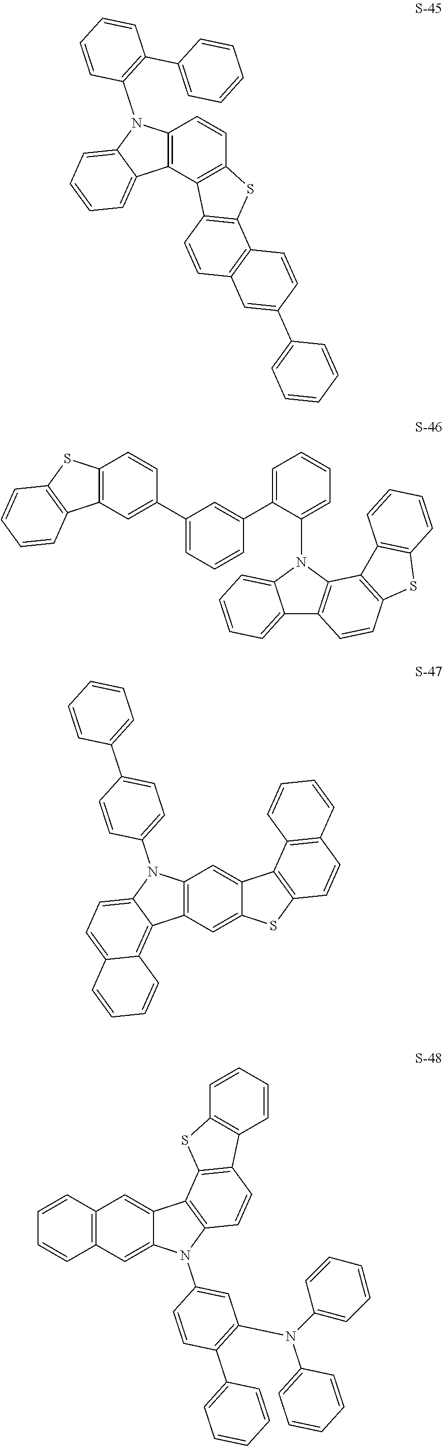 [SMILES: c1ccc(-c2ccc3c(ccc4c3sc3ccc5c(c6ccccc6n5-c5ccccc5-c5ccccc5)c34)c2)cc1 ; c1cc(-c2ccc3sc4ccccc4c3c2)cc(-c2ccccc2-n2c3ccccc3c3ccc4sc5ccccc5c4c32)c1 ; c1ccc(-c2ccc(-n3c4cc5c(cc4c4c6ccccc6ccc43)sc3ccc4ccccc4c35)cc2)cc1 ; c1ccc(-c2ccc(-n3c4cc5ccccc5cc4c4c5sc6ccccc6c5ccc43)cc2N(c2ccccc2)c2ccccc2)cc1]

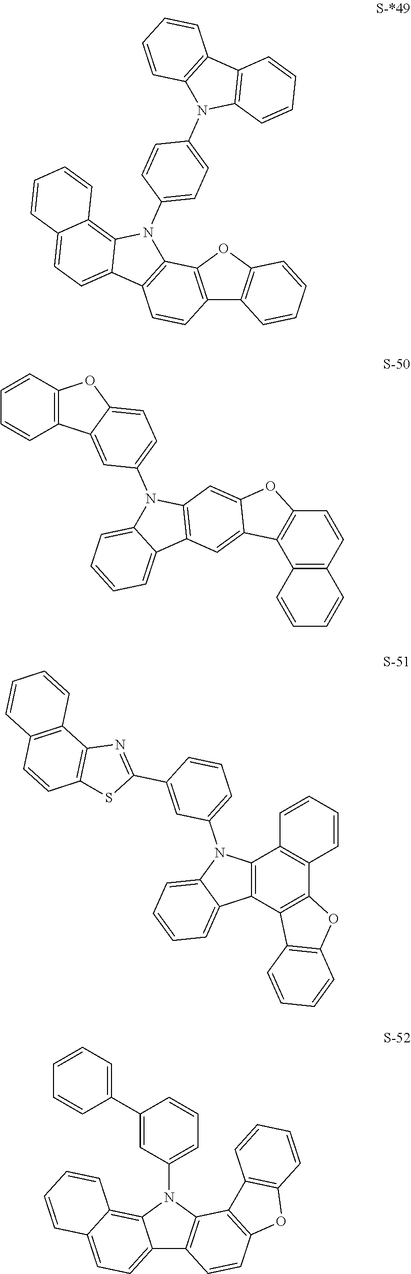 [SMILES: c1ccc2c(c1)ccc1c3ccc4c5ccccc5oc4c3n(-c3ccc(-n4c5ccccc5c5ccccc54)cc3)c21 ; c1ccc2c(c1)ccc1oc3cc4c(cc3c12)c1ccccc1n4-c1ccc2oc3ccccc3c2c1 ; c1cc(-c2nc3c(ccc4ccccc43)s2)cc(-n2c3ccccc3c3c4c5ccccc5oc4c4ccccc4c32)c1 ; c1ccc(-c2cccc(-n3c4c5ccccc5ccc4c4ccc5oc6ccccc6c5c43)c2)cc1]

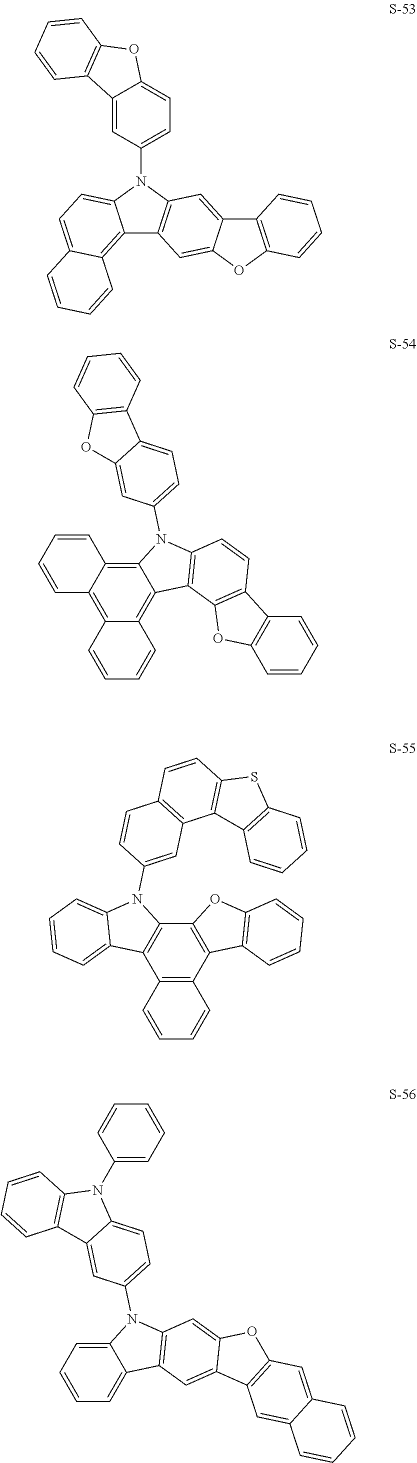

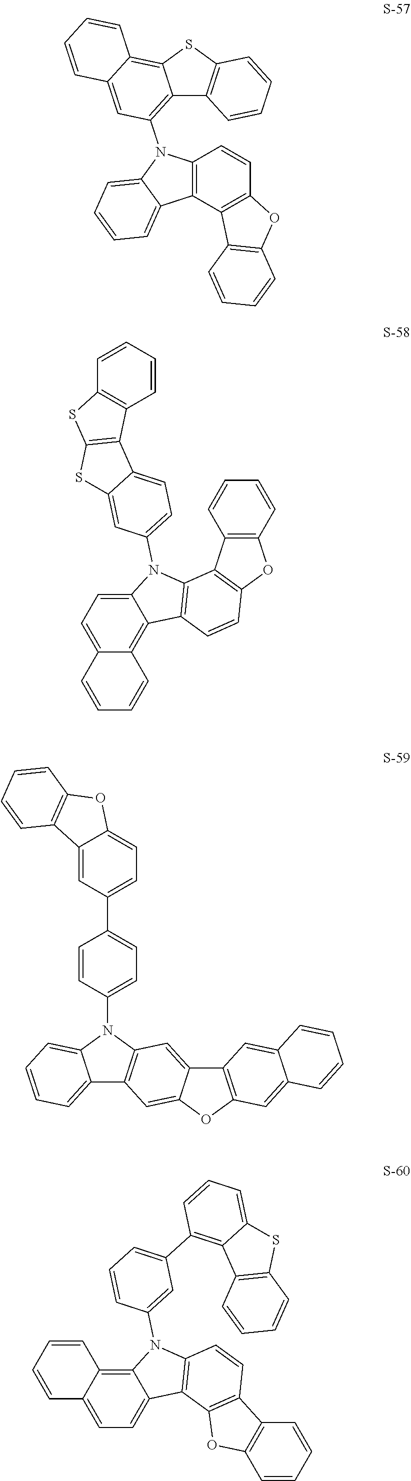 [SMILES: c1ccc2c(c1)cc(-n1c3ccccc3c3c4c(ccc31)oc1ccccc14)c1c3ccccc3sc21 ; c1ccc2c(c1)ccc1c2c2ccc3oc4ccccc4c3c2n1-c1ccc2c(c1)sc1sc3ccccc3c12 ; c1ccc2cc3c(cc2c1)oc1cc2c4ccccc4n(-c4ccc(-c5ccc6oc7ccccc7c6c5)cc4)c2cc13 ; c1cc(-c2cccc3sc4ccccc4c23)cc(-n2c3ccc4c5ccccc5oc4c3c3ccc4ccccc4c32)c1]

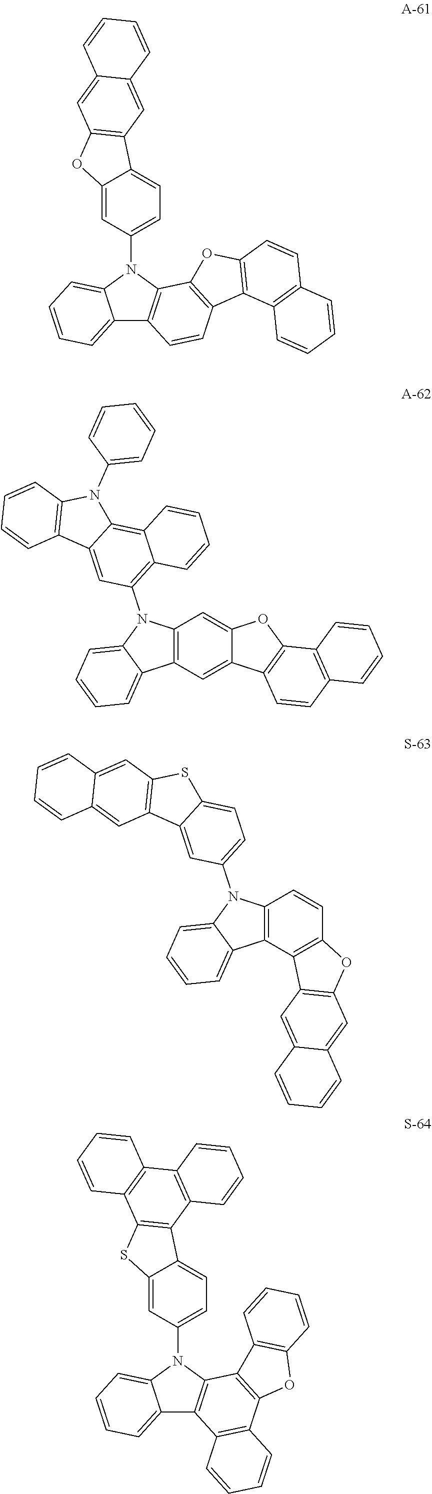 [SMILES: c1ccc2cc3c(cc2c1)oc1cc(-n2c4ccccc4c4ccc5c(oc6ccc7ccccc7c65)c42)ccc13 ; c1ccc(-n2c3ccccc3c3cc(-n4c5ccccc5c5cc6c(cc54)oc4c5ccccc5ccc64)c4ccccc4c32)cc1 ; c1ccc2cc3c(cc2c1)oc1ccc2c(c4ccccc4n2-c2ccc4sc5cc6ccccc6cc5c4c2)c13 ; c1ccc2c(c1)oc1c3ccccc3c3c4ccccc4n(-c4ccc5c(c4)sc4c6ccccc6c6ccccc6c54)c3c21]

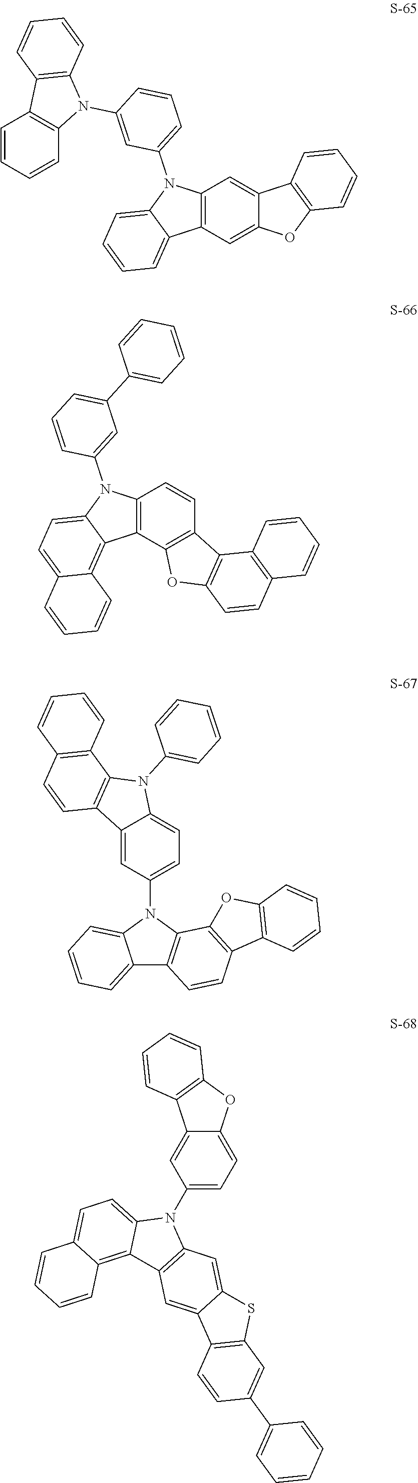 [SMILES: c1cc(-n2c3ccccc3c3ccccc32)cc(-n2c3ccccc3c3cc4oc5ccccc5c4cc32)c1 ; c1ccc(-c2cccc(-n3c4ccc5ccccc5c4c4c5oc6ccc7ccccc7c6c5ccc43)c2)cc1 ; c1ccc(-n2c3ccc(-n4c5ccccc5c5ccc6c7ccccc7oc6c54)cc3c3ccc4ccccc4c32)cc1 ; c1ccc(-c2ccc3c(c2)sc2cc4c(cc23)c2c3ccccc3ccc2n4-c2ccc3oc4ccccc4c3c2)cc1]

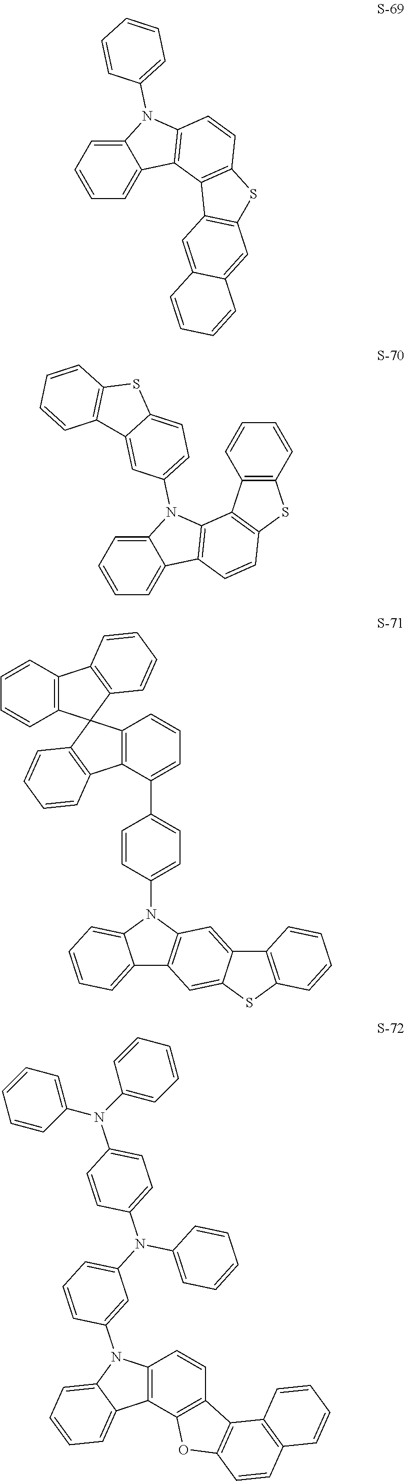

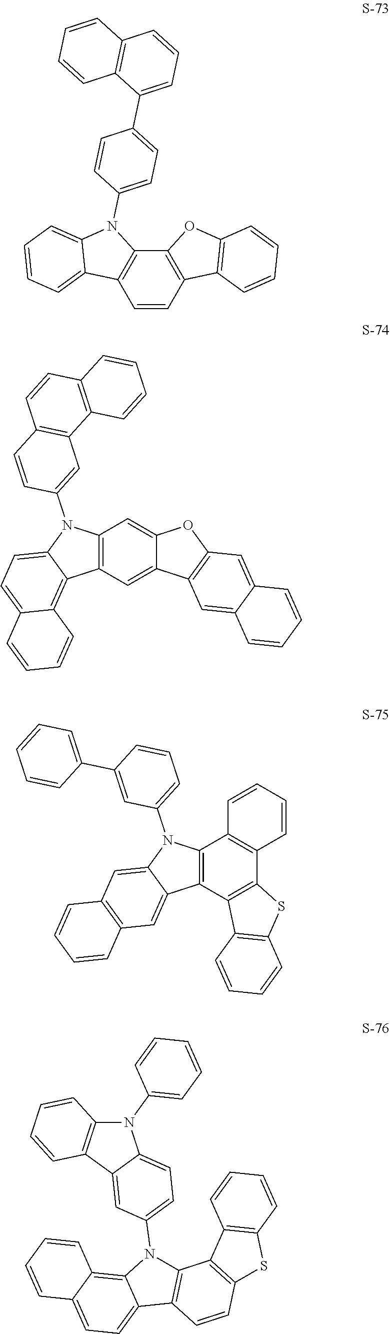 [SMILES: c1ccc2c(-c3ccc(-n4c5ccccc5c5ccc6c7ccccc7oc6c54)cc3)cccc2c1 ; c1ccc2cc3c(cc2c1)oc1cc2c(cc13)c1c3ccccc3ccc1n2-c1ccc2ccc3ccccc3c2c1 ; c1ccc(-c2cccc(-n3c4cc5ccccc5cc4c4c5c6ccccc6sc5c5ccccc5c43)c2)cc1 ; c1ccc(-n2c3ccccc3c3cc(-n4c5c6ccccc6ccc5c5ccc6sc7ccccc7c6c54)ccc32)cc1]

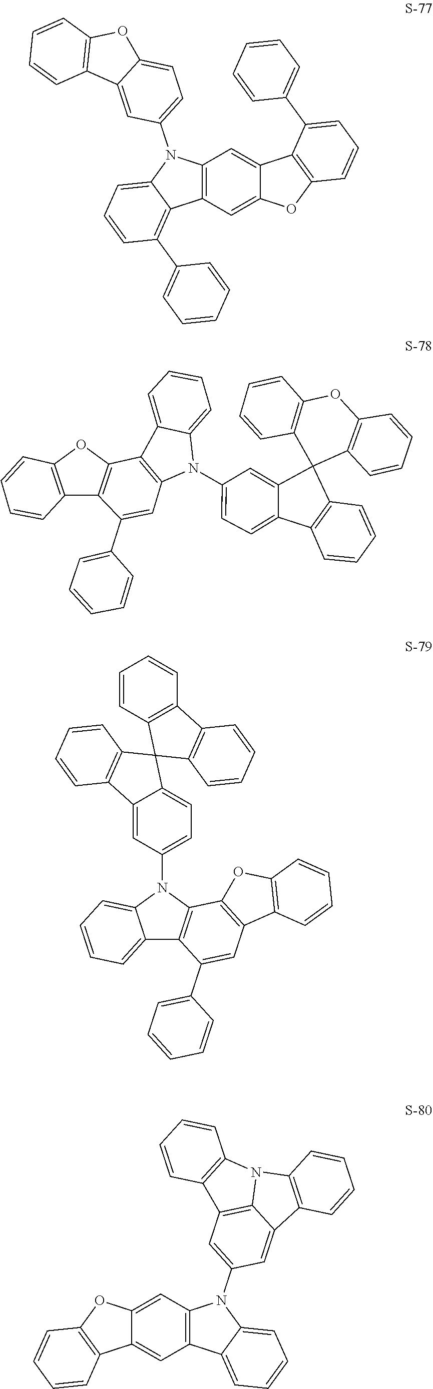 [SMILES: c1ccc(-c2cccc3oc4cc5c6c(-c7ccccc7)cccc6n(-c6ccc7oc8ccccc8c7c6)c5cc4c23)cc1 ; c1ccc(-c2cc3c(c4ccccc4n3-c3ccc4c(c3)C3(c5ccccc5Oc5ccccc53)c3ccccc3-4)c3oc4ccccc4c23)cc1 ; c1ccc(-c2cc3c4ccccc4oc3c3c2c2ccccc2n3-c2ccc3c(c2)-c2ccccc2C32c3ccccc3-c3ccccc32)cc1 ; c1ccc2c(c1)oc1cc3c(cc12)c1ccccc1n3-c1cc2c3ccccc3n3c4ccccc4c(c1)c23]

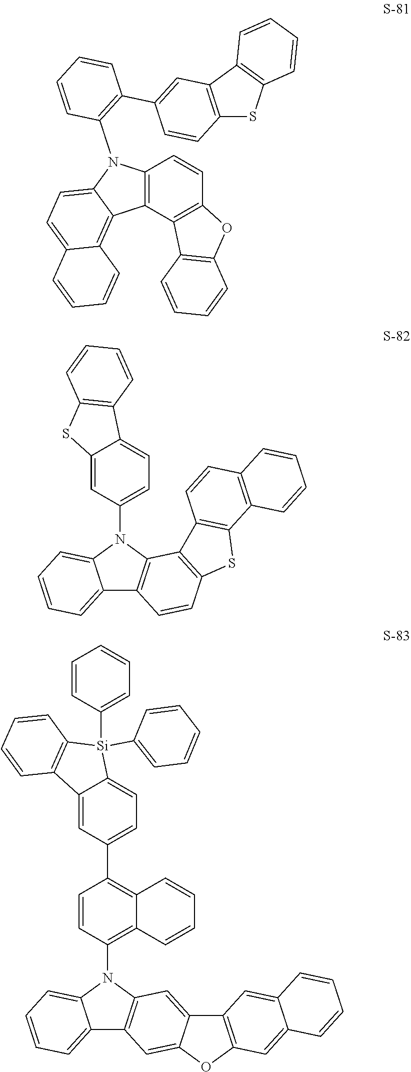

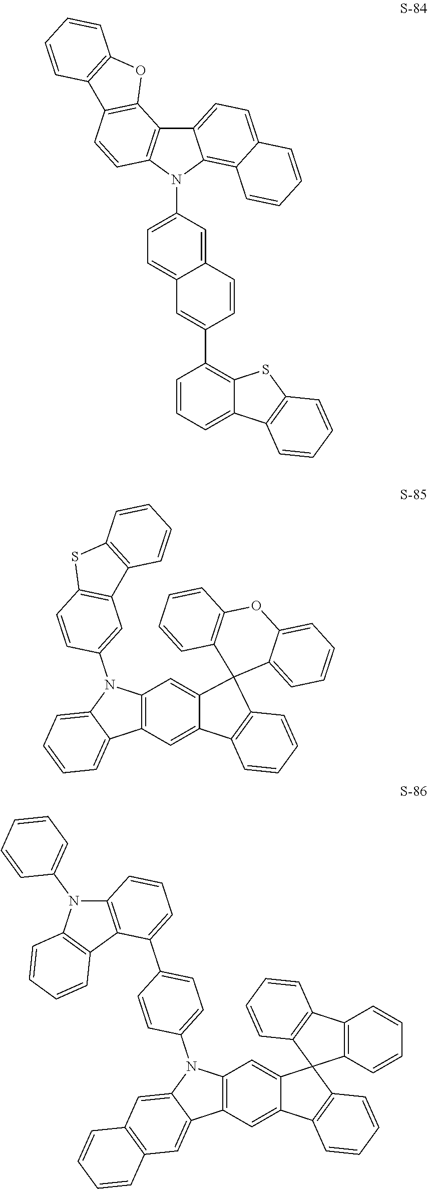

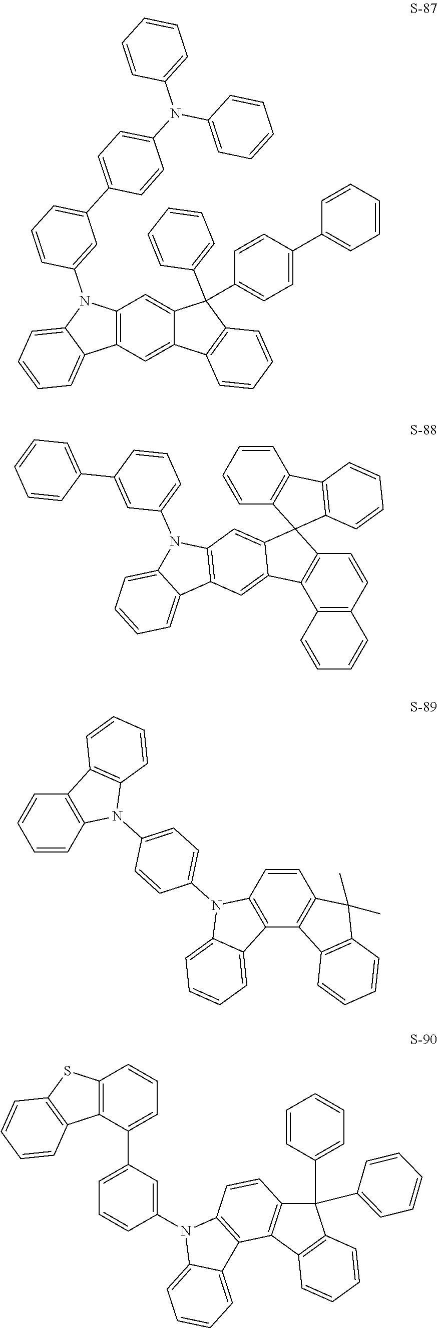 [SMILES: c1ccc(-c2ccc(C3(c4ccccc4)c4ccccc4-c4cc5c6ccccc6n(-c6cccc(-c7ccc(N(c8ccccc8)c8ccccc8)cc7)c6)c5cc43)cc2)cc1 ; c1ccc(-c2cccc(-n3c4ccccc4c4cc5c(cc43)C3(c4ccccc4-c4ccccc43)c3ccc4ccccc4c3-5)c2)cc1 ; CC1(C)c2ccccc2-c2c1ccc1c2c2ccccc2n1-c1ccc(-n2c3ccccc3c3ccccc32)cc1 ; c1ccc(C2(c3ccccc3)c3ccccc3-c3c2ccc2c3c3ccccc3n2-c2cccc(-c3cccc4sc5ccccc5c34)c2)cc1]

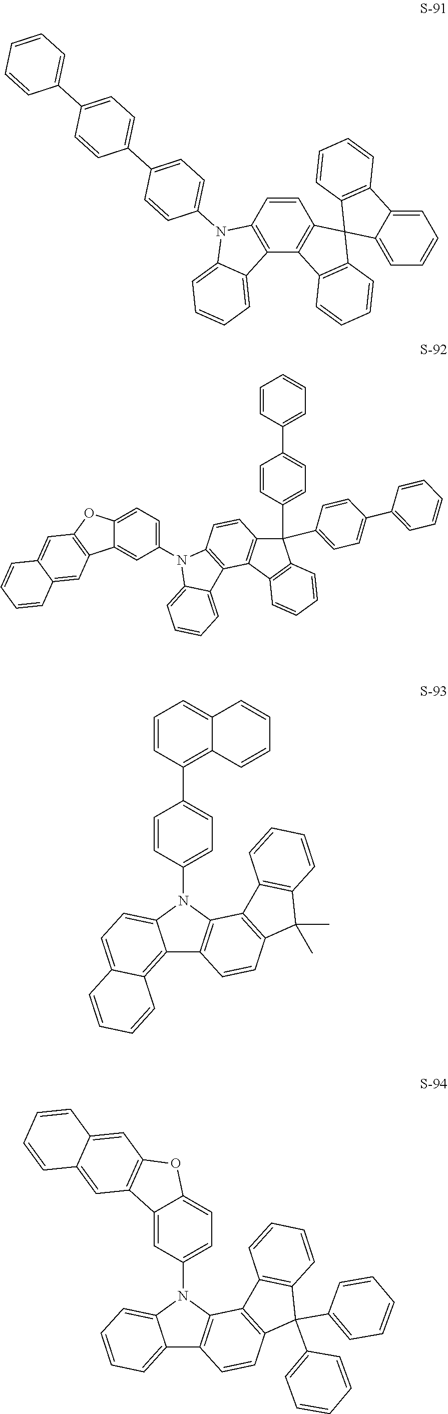 [SMILES: c1ccc(-c2ccc(-c3ccc(-n4c5ccccc5c5c6c(ccc54)C4(c5ccccc5-c5ccccc54)c4ccccc4-6)cc3)cc2)cc1 ; c1ccc(-c2ccc(C3(c4ccc(-c5ccccc5)cc4)c4ccccc4-c4c3ccc3c4c4ccccc4n3-c3ccc4oc5cc6ccccc6cc5c4c3)cc2)cc1 ; CC1(C)c2ccccc2-c2c1ccc1c3c4ccccc4ccc3n(-c3ccc(-c4cccc5ccccc45)cc3)c21 ; c1ccc(C2(c3ccccc3)c3ccccc3-c3c2ccc2c4ccccc4n(-c4ccc5oc6cc7ccccc7cc6c5c4)c32)cc1]

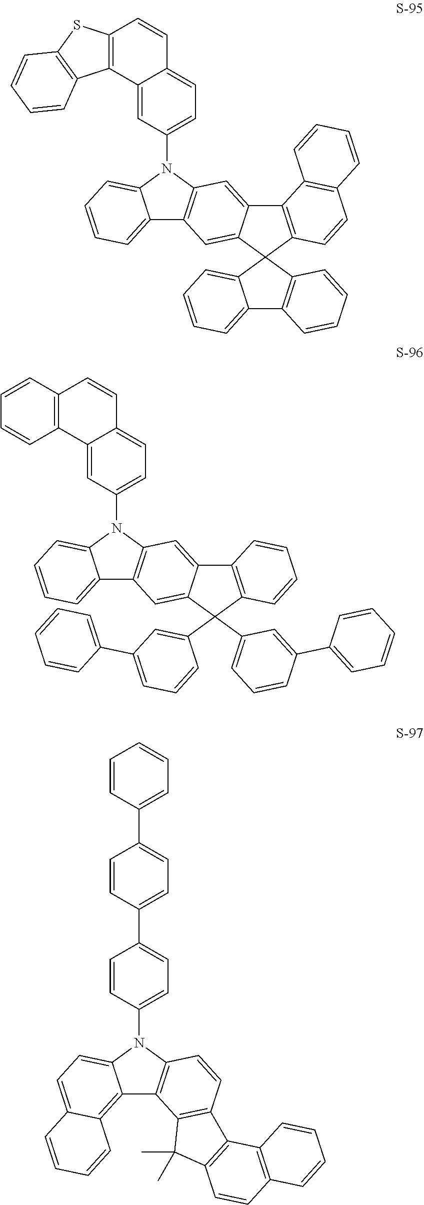 [SMILES: c1ccc2c(c1)-c1ccccc1C21c2cc3c4ccccc4n(-c4ccc5ccc6sc7ccccc7c6c5c4)c3cc2-c2c1ccc1ccccc21 ; c1ccc(-c2cccc(C3(c4cccc(-c5ccccc5)c4)c4ccccc4-c4cc5c(cc43)c3ccccc3n5-c3ccc4ccc5ccccc5c4c3)c2)cc1 ; CC1(C)c2ccc3ccccc3c2-c2ccc3c(c21)c1c2ccccc2ccc1n3-c1ccc(-c2ccc(-c3ccccc3)cc2)cc1]

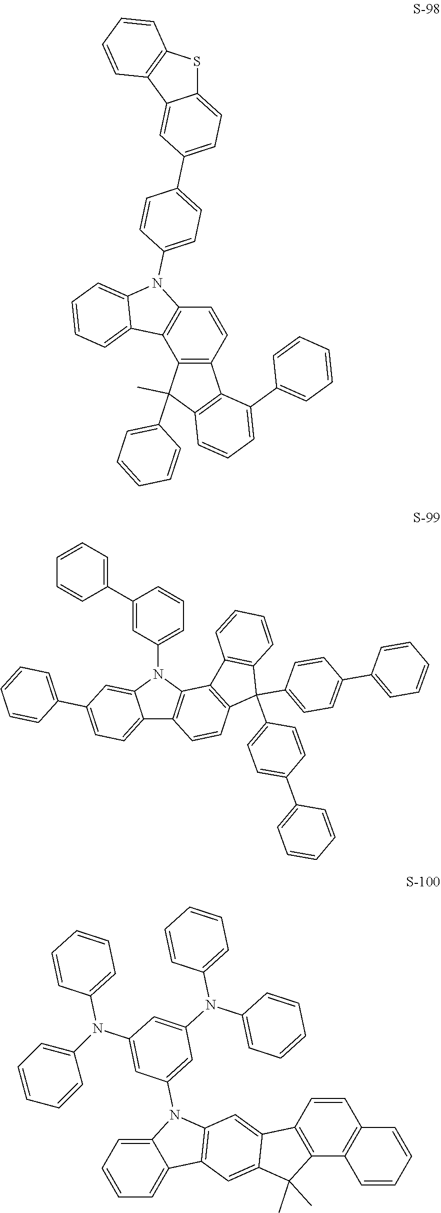 [SMILES: CC1(c2ccccc2)c2cccc(-c3ccccc3)c2-c2ccc3c(c21)c1ccccc1n3-c1ccc(-c2ccc3sc4ccccc4c3c2)cc1 ; c1ccc(-c2ccc(C3(c4ccc(-c5ccccc5)cc4)c4ccccc4-c4c3ccc3c5ccc(-c6ccccc6)cc5n(-c5cccc(-c6ccccc6)c5)c43)cc2)cc1 ; CC1(C)c2cc3c4ccccc4n(-c4cc(N(c5ccccc5)c5ccccc5)cc(N(c5ccccc5)c5ccccc5)c4)c3cc2-c2ccc3ccccc3c21]

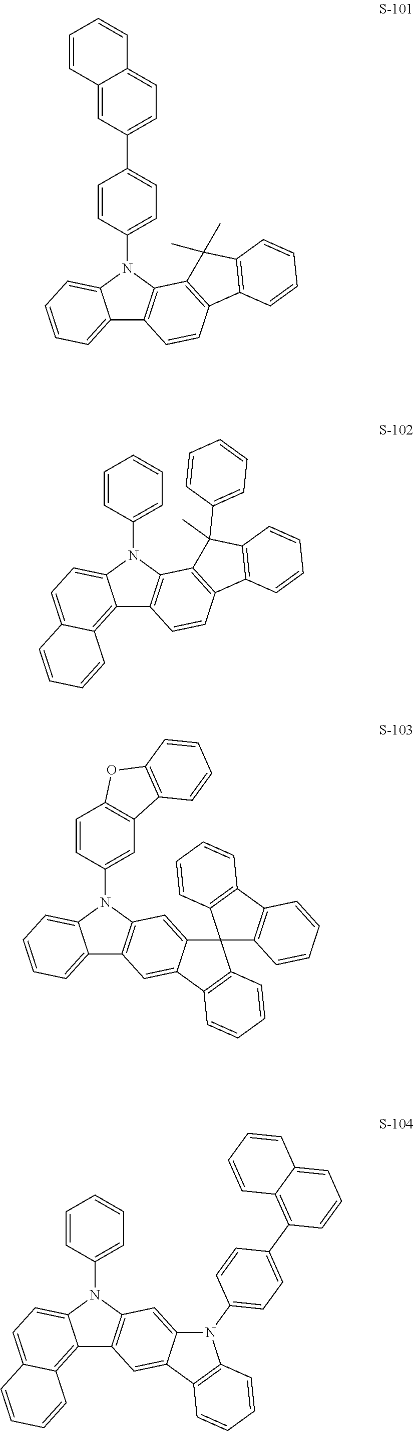 [SMILES: CC1(C)c2ccccc2-c2ccc3c4ccccc4n(-c4ccc(-c5ccc6ccccc6c5)cc4)c3c21 ; CC1(c2ccccc2)c2ccccc2-c2ccc3c4c5ccccc5ccc4n(-c4ccccc4)c3c21 ; c1ccc2c(c1)-c1ccccc1C21c2ccccc2-c2cc3c4ccccc4n(-c4ccc5oc6ccccc6c5c4)c3cc21 ; c1ccc(-n2c3cc4c(cc3c3c5ccccc5ccc32)c2ccccc2n4-c2ccc(-c3cccc4ccccc34)cc2)cc1]

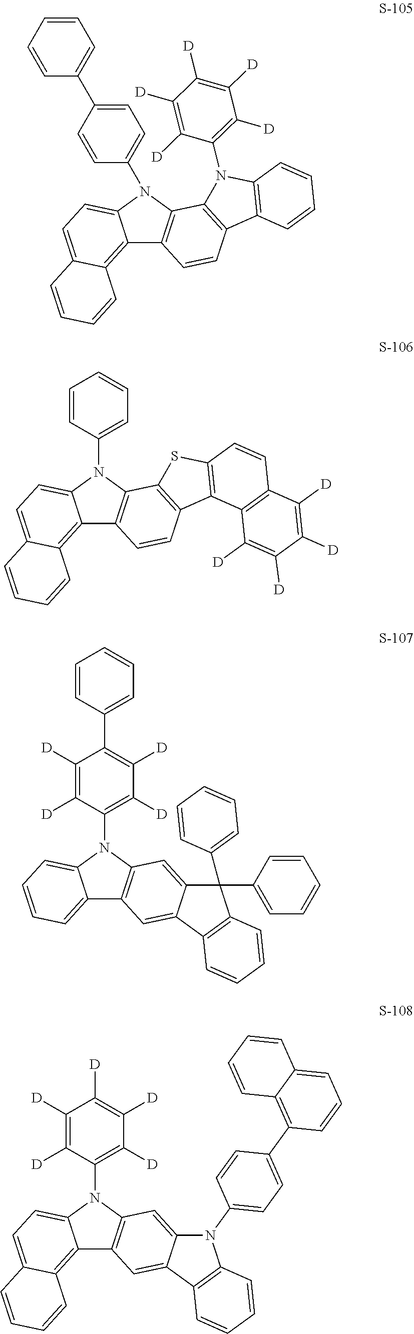 [SMILES: [2H]c1c([2H])c([2H])c(-n2c3ccccc3c3ccc4c5c6ccccc6ccc5n(-c5ccc(-c6ccccc6)cc5)c4c32)c([2H])c1[2H] ; [2H]c1c([2H])c([2H])c2c(ccc3sc4c(ccc5c6c7ccccc7ccc6n(-c6ccccc6)c54)c32)c1[2H] ; [2H]c1c([2H])c(-n2c3ccccc3c3cc4c(cc32)C(c2ccccc2)(c2ccccc2)c2ccccc2-4)c([2H])c([2H])c1-c1ccccc1 ; [2H]c1c([2H])c([2H])c(-n2c3cc4c(cc3c3c5ccccc5ccc32)c2ccccc2n4-c2ccc(-c3cccc4ccccc34)cc2)c([2H])c1[2H]]

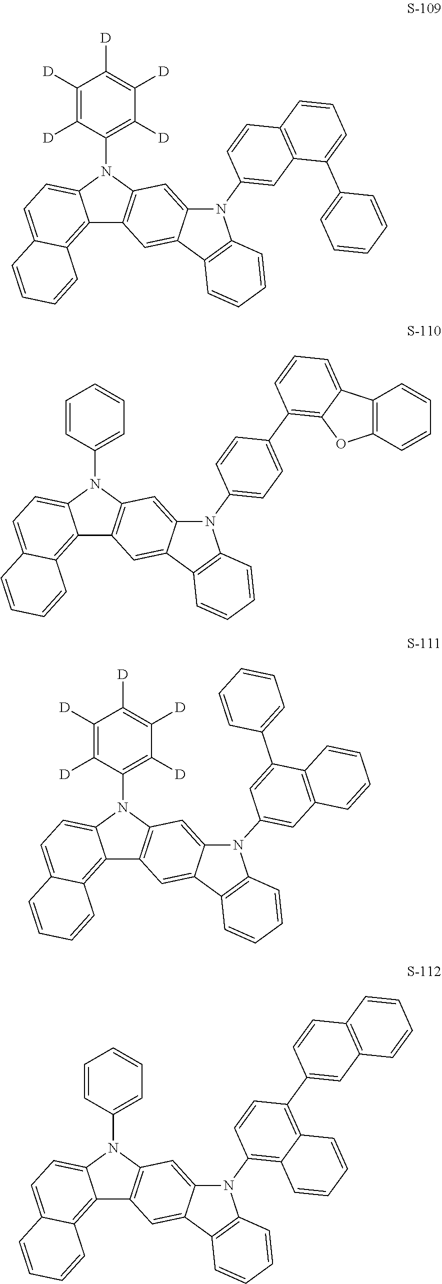 [SMILES: [2H]c1c([2H])c([2H])c(-n2c3cc4c(cc3c3c5ccccc5ccc32)c2ccccc2n4-c2ccc3cccc(-c4ccccc4)c3c2)c([2H])c1[2H] ; c1ccc(-n2c3cc4c(cc3c3c5ccccc5ccc32)c2ccccc2n4-c2ccc(-c3cccc4c3oc3ccccc34)cc2)cc1 ; [2H]c1c([2H])c([2H])c(-n2c3cc4c(cc3c3c5ccccc5ccc32)c2ccccc2n4-c2cc(-c3ccccc3)c3ccccc3c2)c([2H])c1[2H] ; c1ccc(-n2c3cc4c(cc3c3c5ccccc5ccc32)c2ccccc2n4-c2ccc(-c3ccc4ccccc4c3)c3ccccc23)cc1]

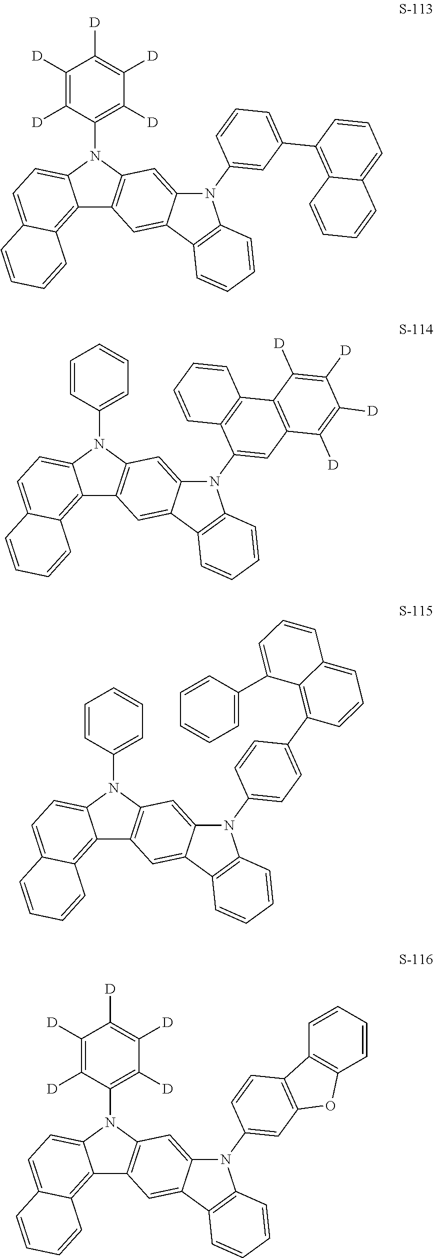 [SMILES: [2H]c1c([2H])c([2H])c(-n2c3cc4c(cc3c3c5ccccc5ccc32)c2ccccc2n4-c2cccc(-c3cccc4ccccc34)c2)c([2H])c1[2H] ; [2H]c1c([2H])c([2H])c2c(cc(-n3c4ccccc4c4cc5c6c7ccccc7ccc6n(-c6ccccc6)c5cc43)c3ccccc32)c1[2H] ; c1ccc(-c2cccc3cccc(-c4ccc(-n5c6ccccc6c6cc7c8c9ccccc9ccc8n(-c8ccccc8)c7cc65)cc4)c23)cc1 ; [2H]c1c([2H])c([2H])c(-n2c3cc4c(cc3c3c5ccccc5ccc32)c2ccccc2n4-c2ccc3c(c2)oc2ccccc23)c([2H])c1[2H]]

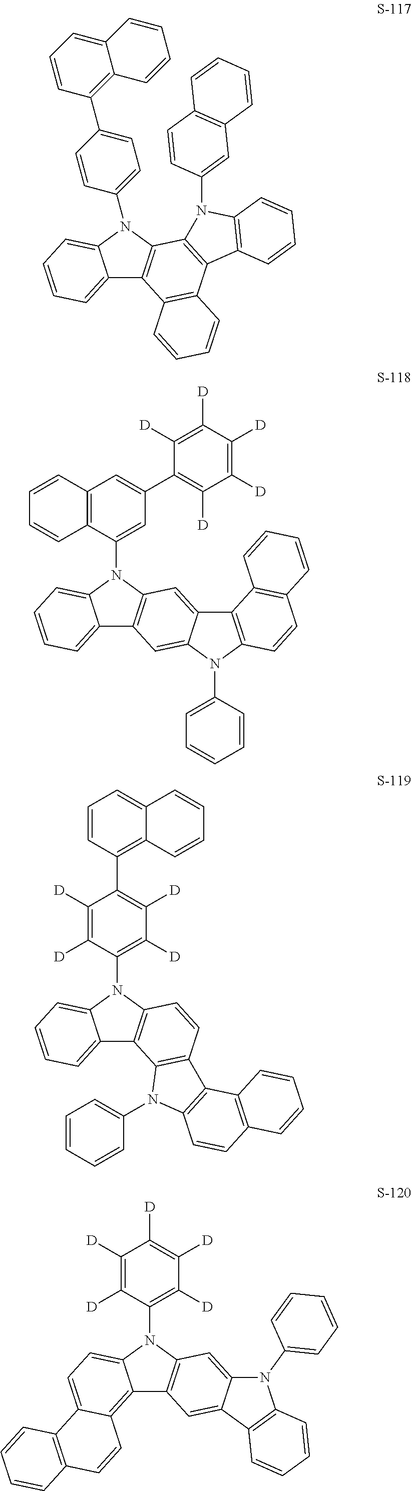

Also, in another aspect, the present invention provides an organic electronic element comprising a first electrode; a second electrode; and an organic material layer formed between the first electrode and the second electrode; wherein the organic material layer comprises a compound represented by Formula 1 or the composition for the organic electronic element.

In another aspect, the present invention provides a method for reusing a compound of Formula 1 comprising:
recovering a crude organic light emitting material comprising the compound of Formula 1 from a deposition apparatus used in the process for depositing the organic emitting material to prepare an organic light emitting device;
removing impurities from the crude organic light emitting material;
recovering the organic light emitting material after the impurities are removed; and
purifying the recovered organic light emitting material to have a purity of 99.9% or higher.

The step of removing impurities from the crude organic light emitting material recovered from the deposition apparatus may preferably comprise performing a pre-purification process to obtain a purity of 98% or more by recrystallization in a recrystallization solvent.

The recrystallization solvent may be preferably a polar solvent having a polarity index (PI) of 5.5 to 7.2.

The recrystallization solvent may preferably be used by mixing a polar solvent having a polarity value of 5.5 to 7.2 and a non-polar solvent having a polarity value of 2.0 to 4.7.

When a mixture of a polar solvent and a non-polar solvent is used, the recrystallization solvent may be used in an amount of 15% (v/v) or less of the non-polar solvent compared to the polar solvent.

The recrystallization solvent is preferably a single solvent of N-Methylpyrrolidone (NMP); or a polar solvent mixed any one selected from the group consisting of 1,3-Dimethyl-2-imidazolidinone, 2-pyrrolidone, N,N-Dimethyl formamide, Dimethyl acetamide, and Dimethyl sulfoxide to the N-Methylpyrrolidone; or alone; or mixed non-polar solvents; selected from the group consisting of Toluene, Dichloromethane (DCM), Dichloroethane (DCE), Tetrahydrofuran (THF), Chloroform, Ethyl acetate and Butanone; or a mixture of a polar solvent and a non-polar solvent.

The pre-purification process may comprise a step of precipitating crystals of by cooling to 0° C. to 5° C. after dissolving the crude organic light emitting material recovered from the deposition apparatus in a polar solvent at 90° C. to 120° C.

The pre-purification process may comprise a step of precipitating crystals by cooling to 35° C. to 40° C., adding a non-polar solvent, and then cooling to 0° C. to 5° C. after dissolving the crude organic light emitting material recovered from the deposition apparatus in a polar solvent at 90° C. to 120° C.

The pre-purification process may comprise a step of precipitating crystals while concentrating the solvent and removing the non-polar solvent, after dissolving the crude organic light emitting material recovered from the deposition apparatus in a non-polar solvent.

The pre-purification process may comprise a step of recrystallizing again with a non-polar solvent after recrystallizing first with a polar solvent.

The step of purifying the recovered impurities to a purity of 99.9% or higher may comprise performing an adsorption separation process to adsorb and remove impurities by adsorbing on the adsorbent.

The adsorbent may be activated carbon, silica gel, alumina, or a material for known adsorption purposes.

The step of purifying the recovered impurities to a purity of 99.9% or higher may comprise performing sublimation purification.

Referring to FIG. 1, the organic electronic element (100) according to the present invention comprises a first electrode (110), a second electrode (170), an organic material layer comprising single compound or 2 or more compounds represented by Formula 1 between the first electrode (110) and the second electrode (170). Wherein, the first electrode (110) may be an anode or a positive electrode, and the second electrode (170) may be a cathode or a negative electrode. In the case of an inverted organic electronic element, the first electrode may be a cathode, and the second electrode may be an anode.

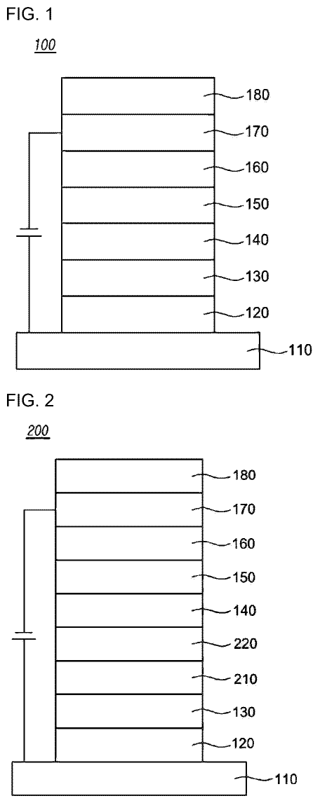

The organic material layer may sequentially comprise a hole injection layer (120), a hole transport layer (130), an emitting layer (140), an electron transport layer (150), and an electron injection layer (160) on the first electrode (110). Here, the remaining layers except the emitting layer (140) may not be formed. The organic material layer may further comprise a hole blocking layer, an electron blocking layer, an emitting-auxiliary layer (220), a buffer layer (210), etc., and the electron transport layer (150), etc. may serve as a hole blocking layer (see FIG. 2).

Also, the organic electronic element according to an embodiment of the present invention may further include a protective layer or a light efficiency enhancing layer (180). Wherein the light efficiency enhancing layer is formed on one of both surfaces of the first electrode that is not in contact with the organic material layer or on one of both surfaces of the second electrode that is not in contact with the organic material layer. The compound or materials for organic electronic element according to an embodiment of the present invention applied to the organic material layer may be used as a material for a hole injection layer (120), a hole transport layer (130), an emitting-auxiliary layer (220), an electron transport auxiliary layer, an electron transport layer (150), an electron injection layer (160), a host or dopant of an emitting layer (140), or the light efficiency enhancing layer. Preferably, for example, a composition for an organic electronic element comprising a compound according to Formula 1 of the present invention, or a mixture of a compound represented by Formula 1 and a compound represented by Formula 2 or 3, can be used as a host material of an emitting layer.

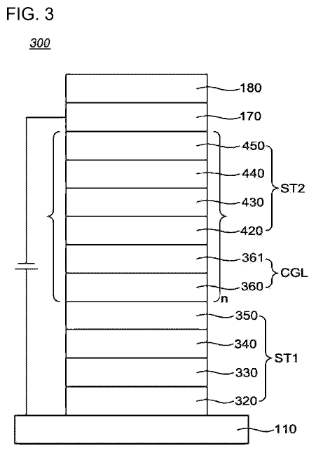

The organic material layer may comprise 2 or more stacks comprising a hole transport layer, an emitting layer and an electron transport layer sequentially formed on the anode, and may further comprise a charge generation layer formed between the 2 or more stacks (see FIG. 3).

Otherwise, even if the same core is used, the band gap, the electrical characteristics, the interface characteristics, etc. may vary depending on which substituent is bonded at which position, therefore the choice of core and the combination of sub-substituents associated therewith is also very important, and in particular, when the optimal combination of energy levels and T1 values, and unique properties of materials (mobility, interfacial characteristics, etc.) of each organic material layer is achieved, a long life span and high efficiency can be achieved at the same time.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer including the hole injection layer (120), the hole transport layer (130), the emitting layer (140), the electron transport layer (150), and the electron injection layer (160) is formed thereon, and then depositing a material usable as a cathode thereon can manufacture an organic electroluminescent device according to an embodiment of the present invention.

Also, the present invention provides the organic electronic element wherein the organic material layer is formed by one of a spin coating process, a nozzle printing process, an inkjet printing process, a slot coating process, a dip coating process or a roll-to-roll process, and the organic material layer provides an organic electronic element comprising the compound or a composition for an organic electronic element as an electron transport material.

As another specific example, the present invention provides an organic electronic element used by mixing the same or different compounds of the compound represented by Formula 1 to the organic material layer. Preferably, the organic material layer comprises an emitting layer, wherein the emitting layer comprises a composition for an organic electronic element comprising a compound represented by Formula 1 or a mixture of a compound represented by Formula 1 and a compound represented by Formula 2 or Formula 3.

Also, the present invention provides a composition for an organic electronic element comprising a compound represented by Formula 1, or a mixture of a compound represented by Formula 1 and a compound represented by Formula 2 or Formula 3, and provides an organic electronic element comprising the composition.

Also, the present invention also provides an electronic device comprising a display device comprising the organic electronic element; and a control unit for driving the display device.

According to another aspect, the present invention provides a display device wherein the organic electronic element is at least one of an OLED, an organic solar cell, an organic photo conductor, an organic transistor (organic TFT) and an element for monochromic or white illumination. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, Synthesis examples of the compound represented by Formula 1, Formula 2 and Formula 3 according to the present invention and preparation examples of the organic electronic element of the present invention will be described in detail by way of example, but are not limited to the following examples.

### Synthesis Example 1

The compound (final products) represented by Formula 1 according to the present invention can be synthesized by reacting Sub 1 and Sub 2 as shown in Reaction Scheme 1, but is not limited thereto.

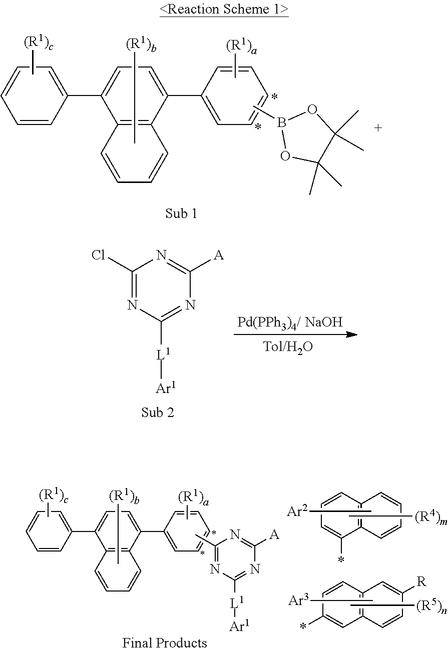

### I. Synthesis of Final Product

### 1. Synthesis Example of P-1

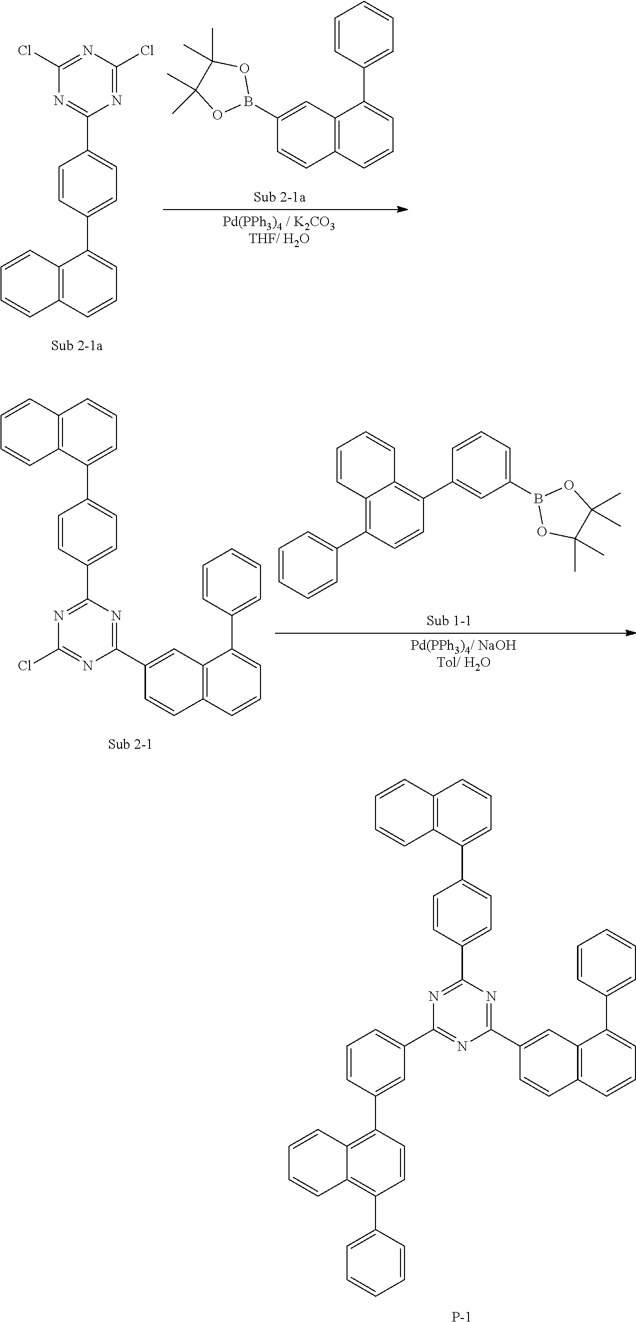

### 1) Synthesis of Sub 2-1

Sub 2-1a (106.66 g, 302.82 mmol), Sub 2-1b (50.00 g, 151.41 mmol), Pd(PPh₃)₄ (5.25 g, 4.54 mmol), K₂CO₃ (41.85 g, 302.82 mmol) were placed in a round-bottom flask, dissolved in THF (504 mL) and water (168 mL) and refluxed at 75° C. for 12 hours. When the reaction was complete, the reaction product was cooled to room temperature, extracted with CH₂Cl₂ and water, and then treated with MgSO₄. The product was produced by concentrating the organic solvent and recrystallizing using a silica gel column to obtain 32.28 g (41%) of Sub 2-1.

### 2) Synthesis of P-1

Sub 2-1 (30.00 g, 57.69 mmol), Sub 1-1 (23.44 g, 57.69 mmol), Pd(PPh₃)₄ (2.00 g, 1.73 mmol), NaOH (4.62 g, 115.38 mmol) and Toluene (192 mL) and water (64 mL) were added in a round-bottom flask, and the mixture was refluxed at 110° C. for 12 hours. When the reaction was complete, the reaction product was cooled to room temperature, extracted with CH₂Cl₂ and water, and then treated with MgSO₄. The product was produced by concentrating the organic solvent and recrystallizing using a silica gel column to obtain 31.29 g (71%) of P-1.

### 2. Synthesis Example of P-18

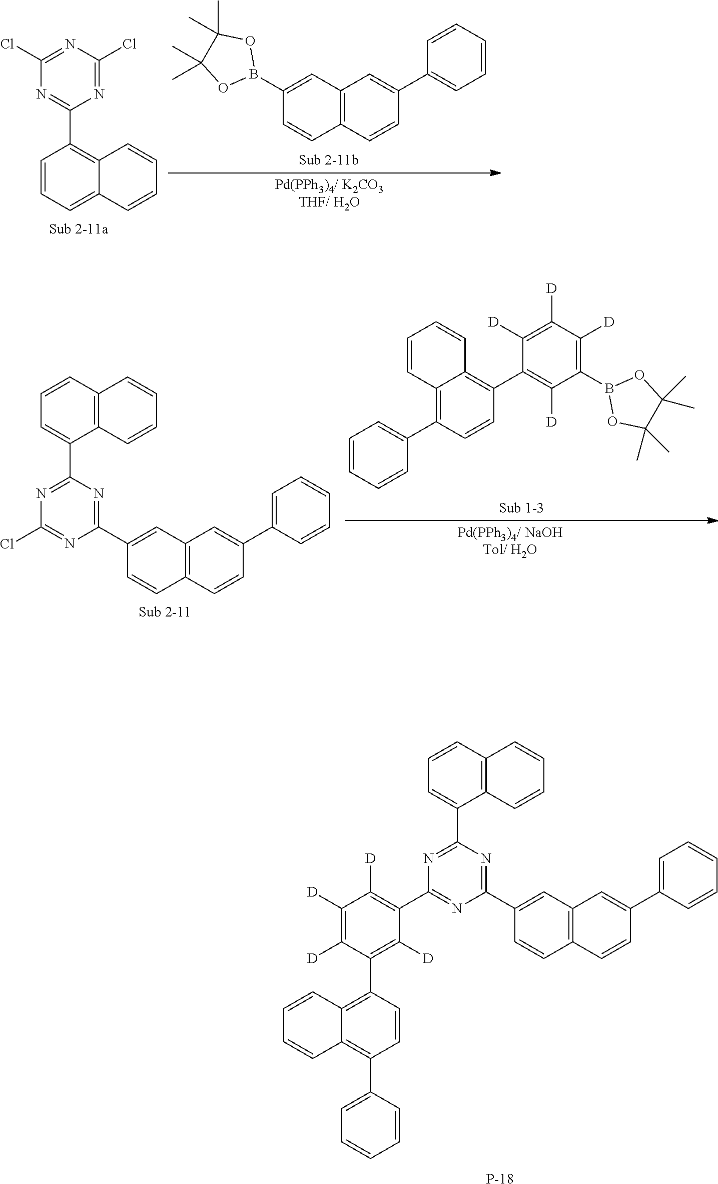

### 1) Synthesis of Sub 2-11

Sub 2-11a (66.89 g, 242.26 mmol), Sub 2-11b (40.00 g, 121.13 mmol), Pd(PPh₃)₄ (4.20 g, 3.63 mmol), K₂CO₃ (33.48 g, 242.26 mmol) were added in a round-bottom flask, and dissolved in THF (404 mL) and water (135 mL), and 20.43 g (38%) of Sub 2-11 was obtained using the synthetic method of Sub 2-1.

### 2) Synthesis of P-18

Sub 2-11 (20.00 g, 45.05 mmol), Sub 1-3 (18.49 g, 45.05 mmol), Pd(PPh₃)₄ (1.56 g, 1.35 mmol), NaOH (3.60 g, 90.10 mmol) were added in a round-bottom flask, and dissolved in Toluene (150 mL) and water (50 mL), and 21.20 g (68%) of P-18 was obtained using the synthetic method of P-1.

### 3. Synthesis Example of P-23

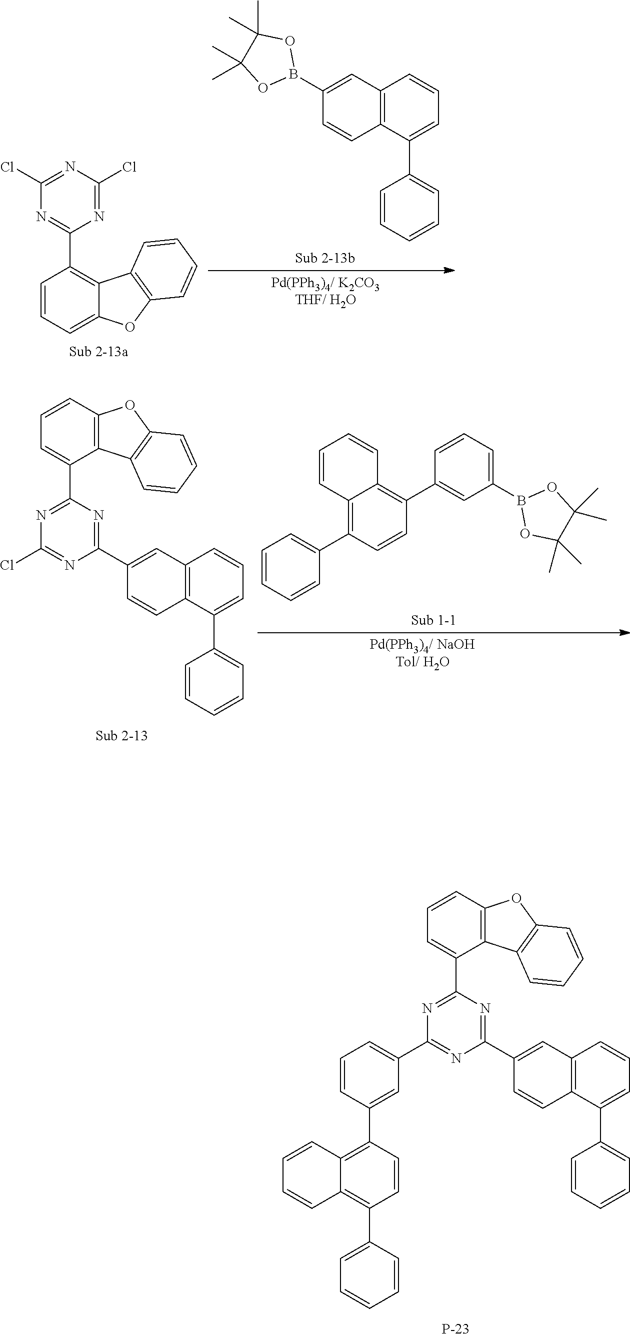

### 1) Synthesis of Sub 2-13

Sub 2-13a (72.76 g, 230.14 mmol), Sub 2-13b (38.00 g, 115.07 mmol), Pd(PPh₃)₄ (3.99 g, 3.45 mmol), K₂CO₃ (31.81 g, 230.14 mmol) were added in a round bottom flask and after dissolving in THF (384 mL) and water (128 mL), 25.06 g (45%) of Sub 2-13 was obtained using the synthetic method of Sub 2-1.

### 2) Synthesis of P-23

Sub 2-13 (24.06 g, 49.72 mmol), Sub 1-1 (20.20 g, 49.72 mmol), Pd(PPh₃)₄ (1.72 g, 1.49 mmol), NaOH (3.98 g, 99.43 mmol) were added in a round-bottom flask, and dissolved in Toluene (166 mL) and water (55 mL), and 26.42 g (73%) of product P-23 was obtained using the synthetic method of P-1.

### 4. Synthesis Example of P-29

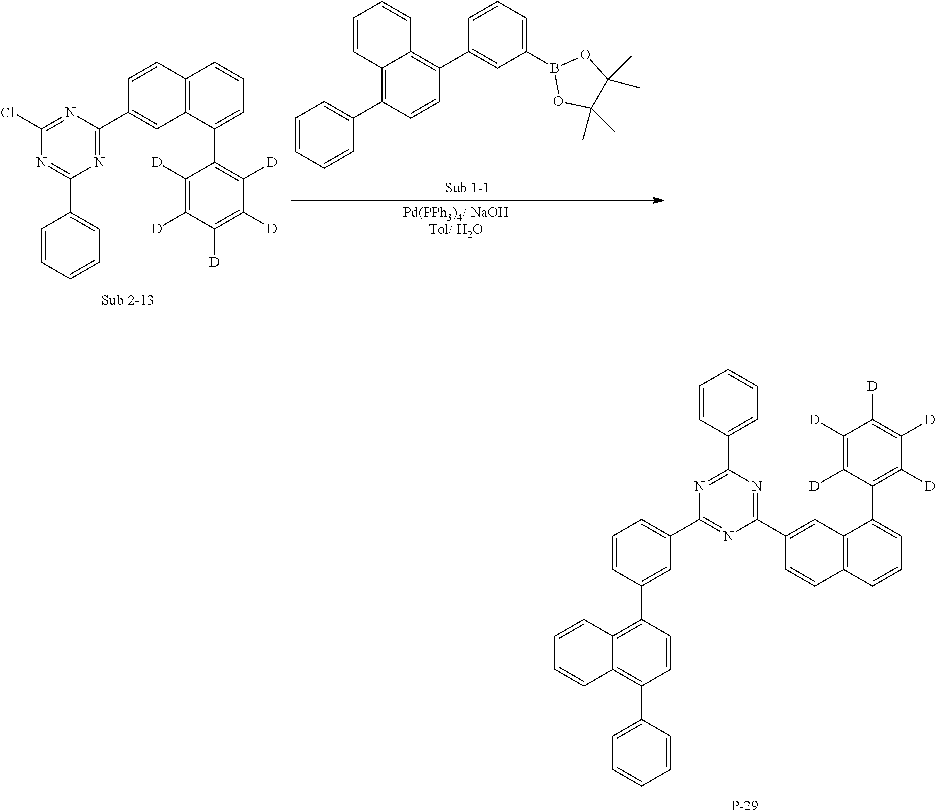

Sub 2-10 (19.66 g, 49.28 mmol), Sub 1-1 (20.02 g, 49.28 mmol), Pd(PPh₃)₄ (1.71 g, 1.48 mmol), NaOH (3.94 g, 98.55 mmol) were added in a round-bottom flask, and dissolved in Toluene (164 mL) and water (54 mL), and 23.76 g (75%) of product P-29 was obtained using the synthetic method of P-1.

### 5. Synthesis Example of P-44

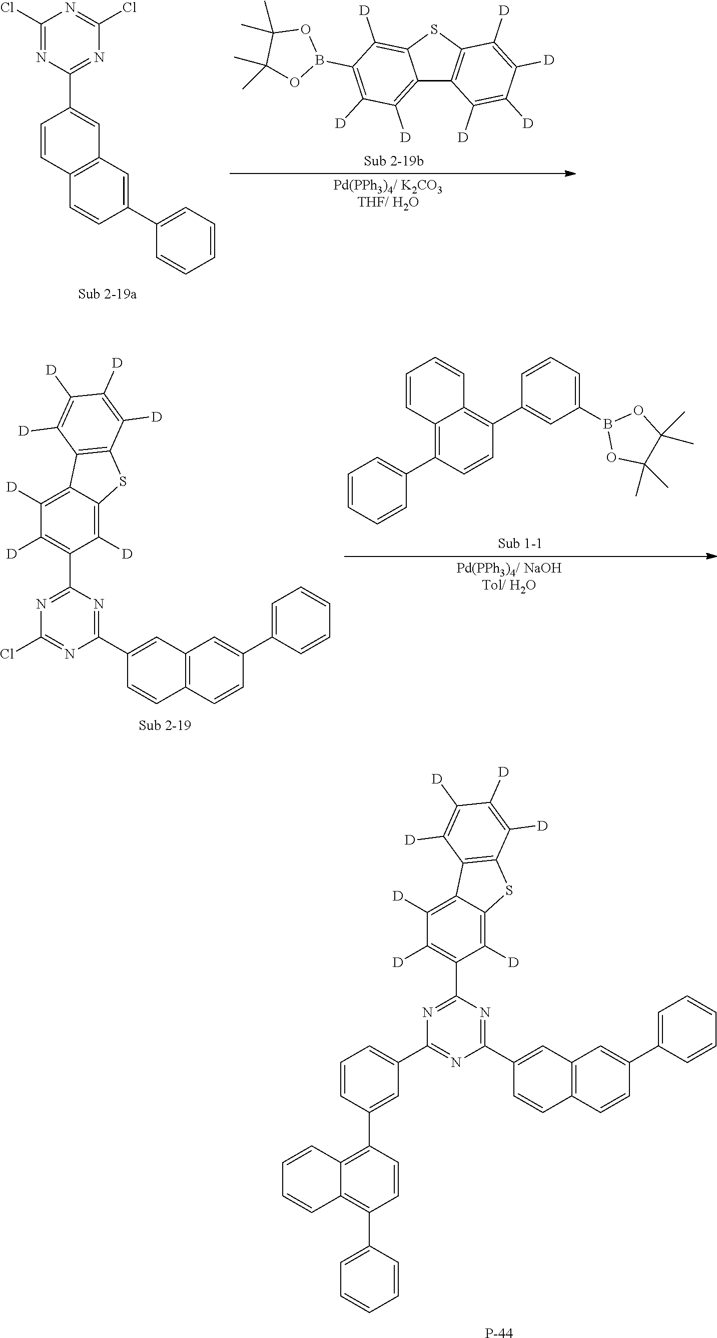

### 1) Synthesis of Sub 2-19

Sub 2-19a (99.92 g, 283.68 mmol), Sub 2-19b (45.00 g, 141.84 mmol), Pd(PPh₃)₄ (4.92 g, 4.26 mmol), K₂CO₃ (39.21 g, 283.68 mmol) were added in a round bottom flask and after dissolving in THF (473 mL) and water (158 mL), 26.61 g (37%) of Sub 2-19 was obtained using the synthetic method of Sub 2-1.

### 2) Synthesis of P-44

Sub 2-19 (25.61 g, 50.51 mmol), Sub 1-1 (20.52 g, 50.51 mmol), Pd(PPh₃)₄ (1.75 g, 1.52 mmol), NaOH (4.04 g, 101.02 mmol) were added in a round-bottom flask, and dissolved in Toluene (168 mL) and water (56 mL), and 29.59 g (78%) of P-44 was obtained using the synthetic method of P-1.

### 6. Synthesis Example of P-64

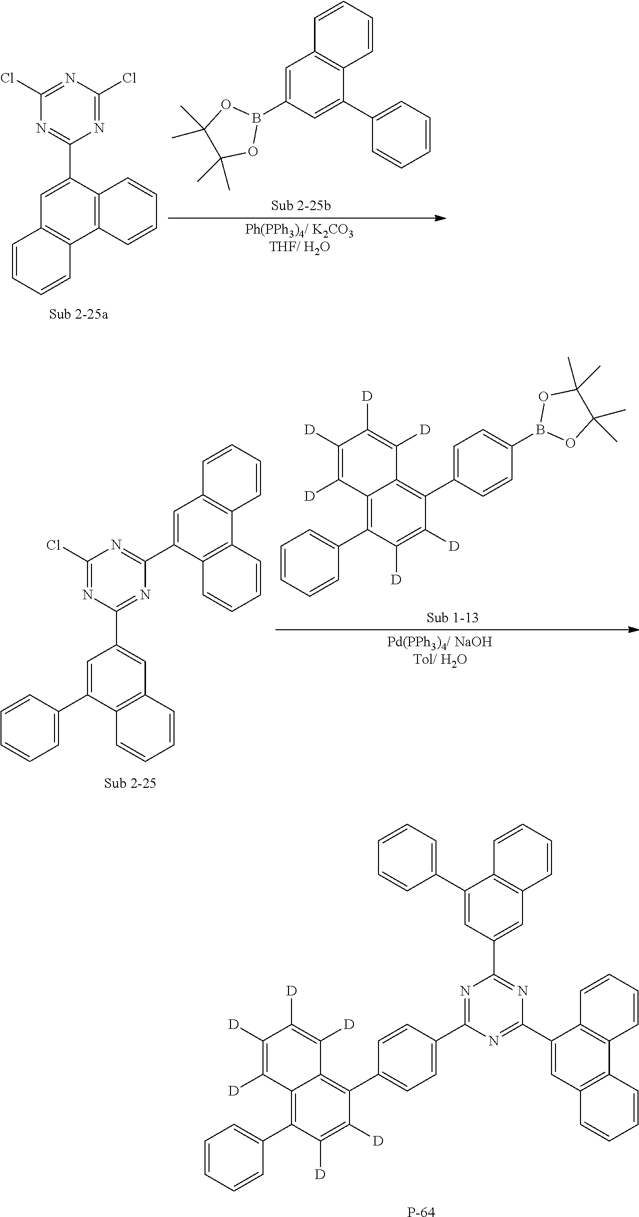

### 1) Synthesis of Sub 2-25

Sub 2-25a (98.77 g, 302.82 mmol), Sub 2-25b (50.00 g, 151.41 mmol), Pd(PPh₃)₄ (5.25 g, 4.54 mmol), K₂CO₃ (41.85 g, 302.82 mmol) were added in a round bottom flask and after dissolving in THF (505 mL) and water (168 mL), 31.41 g (42%) of Sub 2-25 was obtained using the synthetic method of Sub 2-1.

### 2) Synthesis of P-64

Sub 2-25 (30.41 g, 61.57 mmol), Sub 1-13 (25.39 g, 61.57 mmol), Pd(PPh₃)₄ (2.14 g, 1.85 mmol), NaOH (4.93 g, 123.14 mmol) were added in a round-bottom flask, and dissolved in Toluene (205 mL) and water (68 mL), and 40.31 g (88%) of P-64 was obtained using the synthetic method of P-1.

### 7. Synthesis Example of P-86

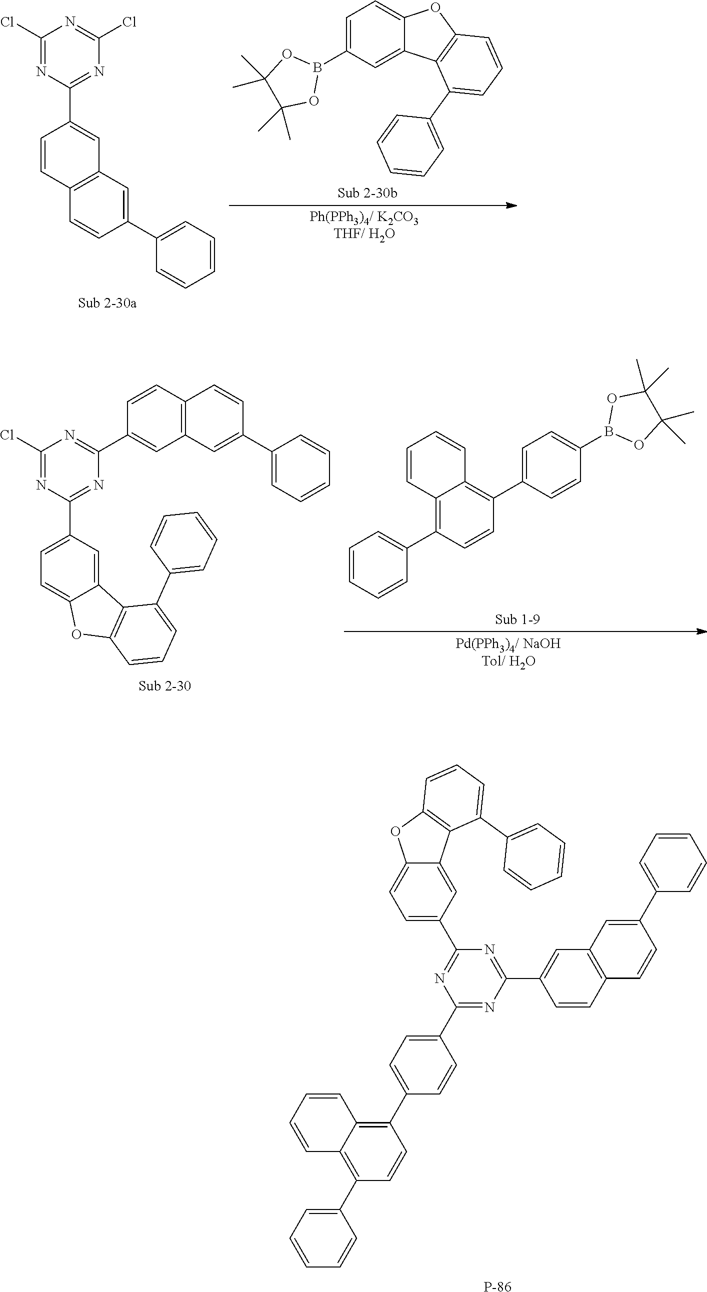

### 1) Synthesis of Sub 2-30

Sub 2-19a (57.08 g, 162.05 mmol), Sub 2-30b (30.00 g, 81.02 mmol), Pd(PPh₃)₄ (2.81 g, 2.43 mmol), K₂CO₃ (22.40 g, 162.05 mmol) were added in a round bottom flask and after dissolving in THF (270 mL) and water (90 mL), 24.96 g (55%) of Sub 2-30 was obtained using the synthetic method of Sub 2-1.

### 2) Synthesis of P-86

Sub 2-30 (23.96 g, 42.78 mmol), Sub 1-9 (17.38 g, 42.78 mmol), Pd(PPh₃)₄ (1.48 g, 1.28 mmol), NaOH (3.42 g, 85.56 mmol) were added in a round-bottom flask, and dissolved in Toluene (143 mL) and water (48 mL), and 29.58 g (86%) of P-86 was obtained using the synthetic method of P-1.

### 8. Synthesis Example of P-88

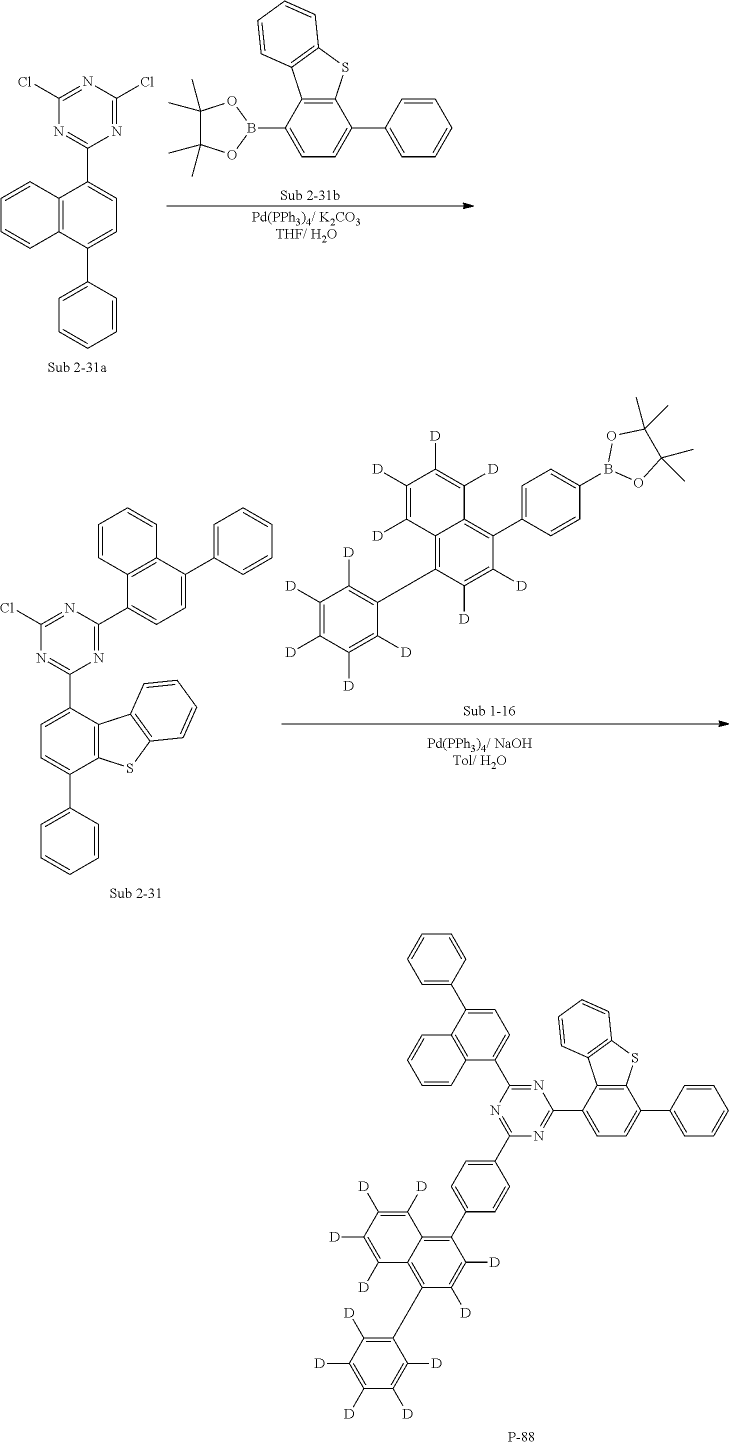

### 1) Synthesis of Sub 2-31

Sub 2-31a (63.82 g, 181.20 mmol), Sub 2-31b (35.00 g, 90.60 mmol), Pd(PPh₃)₄ (3.14 g, 2.72 mmol), K₂CO₃ (25.04 g, 181.20 mmol) were added in a round bottom flask and after dissolving in THF (302 mL) and water (101 mL), 25.05 g (48%) of Sub 2-31 was obtained using the synthetic method of Sub 2-1.

### 2) Synthesis of P-88

Sub 2-31 (24.05 g, 41.75 mmol), Sub 1-16 (17.43 g, 41.75 mmol), Pd(PPh₃)₄ (1.45 g, 1.25 mmol), NaOH (3.34 g, 83.50 mmol) were added in a round-bottom flask, and dissolved in Toluene (140 mL) and water (45 mL), and 27.07 g (78%) of product P-88 was obtained using the synthetic method of P-1.

### 9. Synthesis Example of P-95

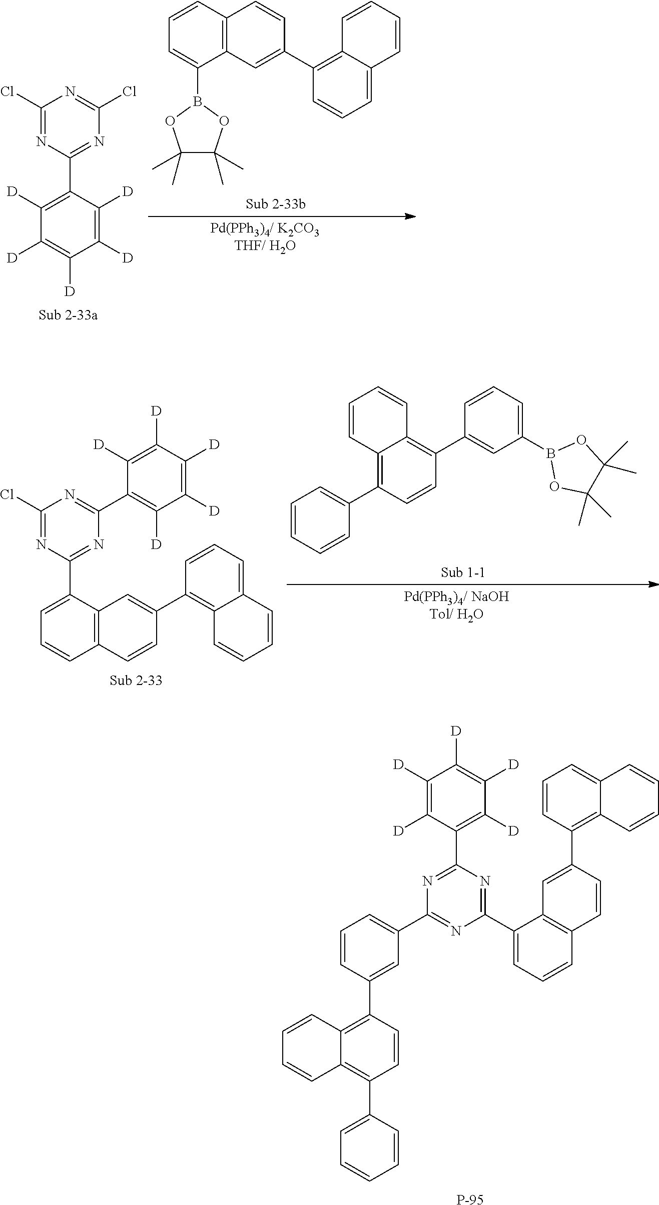

### 1) Synthesis of Sub 2-33

Sub 2-33a (48.61 g, 210.37 mmol), Sub 2-33b (40.00 g, 105.18 mmol), Pd(PPh₃)₄ (3.65 g, 3.16 mmol), K₂CO₃ (29.07 g, 210.37 mmol) were added in a round bottom flask and after dissolving in THF (351 mL) and water (117 mL), 16.53 g (35%) of Sub 2-33 was obtained using the synthetic method of Sub 2-1.

### 2) Synthesis of P-95

Sub 2-33 (15.53 g, 34.59 mmol), Sub 1-1 (14.05 g, 34.59 mmol), Pd(PPh₃)₄ (1.20 g, 1.04 mmol), NaOH (2.77 g, 69.17 mmol) were added in a round-bottom flask, and dissolved in Toluene (115 mL) and water (38 mL), and 20.37 g (85%) of P-95 was obtained using the synthetic method of P-1.

### 10. Synthesis Example of P-100

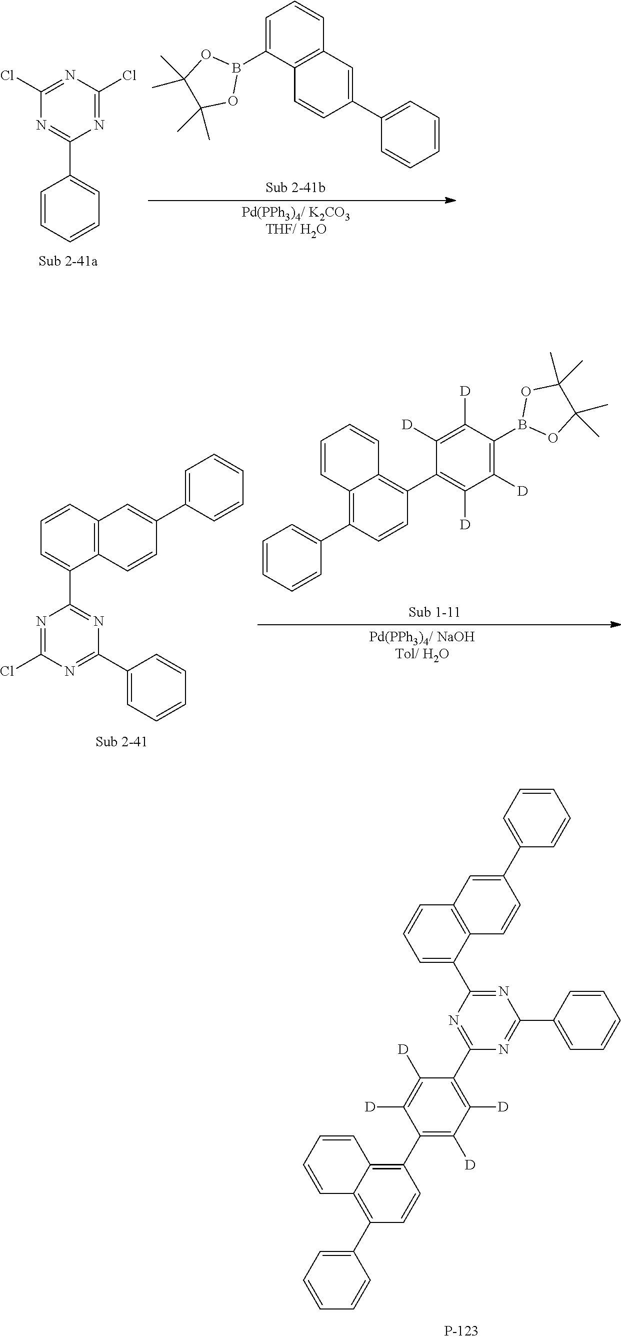

### 1) Synthesis of Sub 2-41

Sub 2-41a (41 g, 181.69 mmol), Sub 2-41b (30.00 g, 90.85 mmol), Pd(PPh₃)₄ (3.15 g, 2.73 mmol), K₂CO₃ (25.11 g, 181.69 mmol) were added in a round bottom flask and after dissolving in THF (303 mL) and water (101 mL), 23.26 g (65%) of Sub 2-41 was obtained using the synthetic method of Sub 2-1.

### 2) Synthesis of P-123

Sub 2-41 (20.00 g, 50.78 mmol), Sub 1-11 (20.84 g, 50.78 mmol), Pd(PPh₃)₄ (1.76 g, 1.52 mmol), NaOH (4.06 g, 101.56 mmol) were added in a round-bottom flask, and dissolved in Toluene (170 mL) and water (56 mL), and 33.79 g (73%) of P-123 was obtained using the synthetic method of P-1.

Sub 1 of the reaction scheme 1 may be, but is not limited to, the compounds below, and the FD-MS (Field Desorption-Mass Spectrometry) values of compounds belonging to Sub 1 are as shown in Table 1.

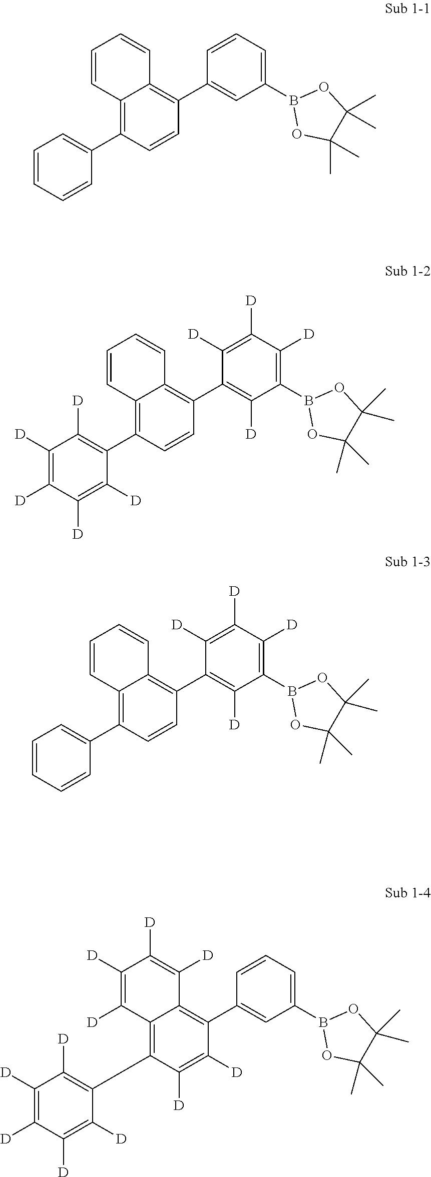 [SMILES: CC1(C)OB(c2cccc(-c3ccc(-c4ccccc4)c4ccccc34)c2)OC1(C)C ; [2H]c1c([2H])c([2H])c(-c2ccc(-c3c([2H])c([2H])c([2H])c(B4OC(C)(C)C(C)(C)O4)c3[2H])c3ccccc23)c([2H])c1[2H] ; [2H]c1c([2H])c(B2OC(C)(C)C(C)(C)O2)c([2H])c(-c2ccc(-c3ccccc3)c3ccccc23)c1[2H] ; [2H]c1c([2H])c([2H])c(Cc2c([2H])c([2H])c(-c3cccc(B4OC(C)(C)C(C)(C)O4)c3)c3c([2H])c([2H])c([2H])c([2H])c23)c([2H])c1[2H]]

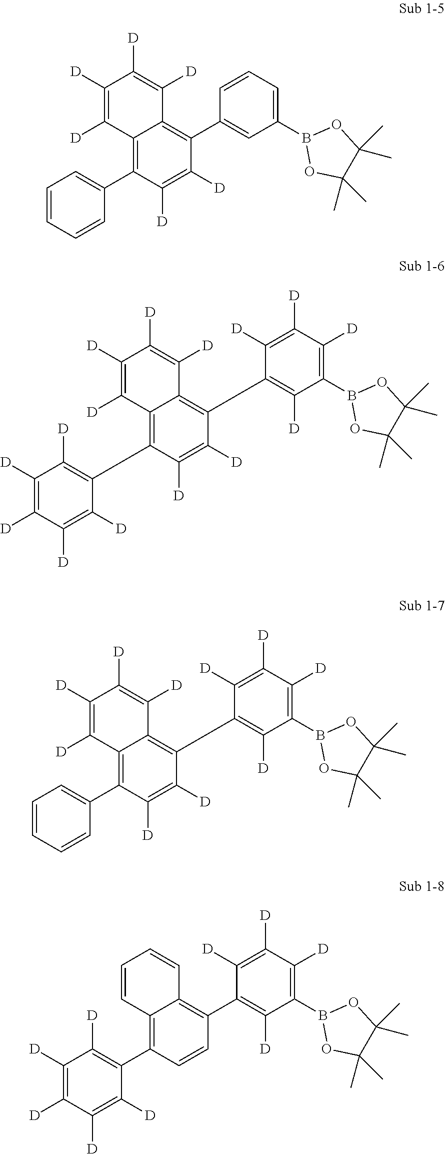 [SMILES: [2H]c1c([2H])c([2H])c2c(-c3cccc(B4OC(C)(C)C(C)(C)O4)c3)c([2H])c([2H])c(-c3ccccc3)c2c1[2H] ; [2H]c1c([2H])c([2H])c(Cc2c([2H])c([2H])c(Cc3c([2H])c([2H])c([2H])c(B4OC(C)(C)C(C)(C)O4)c3[2H])c3c([2H])c([2H])c([2H])c([2H])c23)c([2H])c1[2H] ; [2H]c1c([2H])c(Cc2c([2H])c([2H])c(-c3ccccc3)c3c([2H])c([2H])c([2H])c([2H])c23)c([2H])c(B2OC(C)(C)C(C)(C)O2)c1[2H] ; [2H]c1c([2H])c([2H])c(-c2ccc(-c3c([2H])c([2H])c([2H])c(B4OC(C)(C)C(C)(C)O4)c3[2H])c3ccccc23)c([2H])c1[2H]]

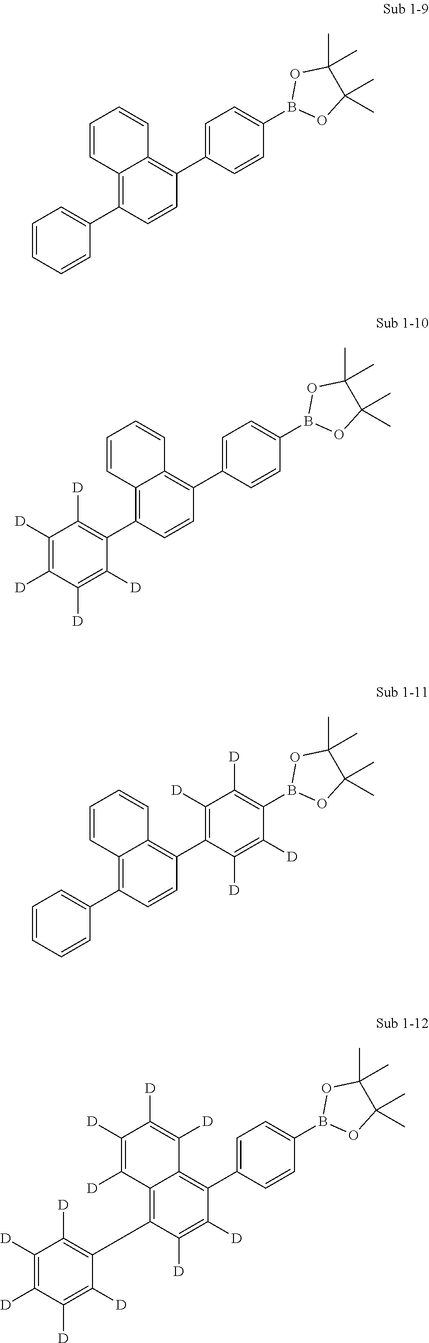 [SMILES: CC1(C)OB(c2ccc(-c3ccc(-c4ccccc4)c4ccccc34)cc2)OC1(C)C ; [2H]c1c([2H])c([2H])c(-c2ccc(-c3ccc(B4OC(C)(C)C(C)(C)O4)cc3)c3ccccc23)c([2H])c1[2H] ; [2H]c1c([2H])c(-c2ccc(-c3ccccc3)c3ccccc23)c([2H])c([2H])c1B1OC(C)(C)C(C)(C)O1 ; [2H]c1c([2H])c([2H])c(Cc2c([2H])c([2H])c(-c3ccc(B4OC(C)(C)C(C)(C)O4)cc3)c3c([2H])c([2H])c([2H])c([2H])c23)c([2H])c1[2H]]

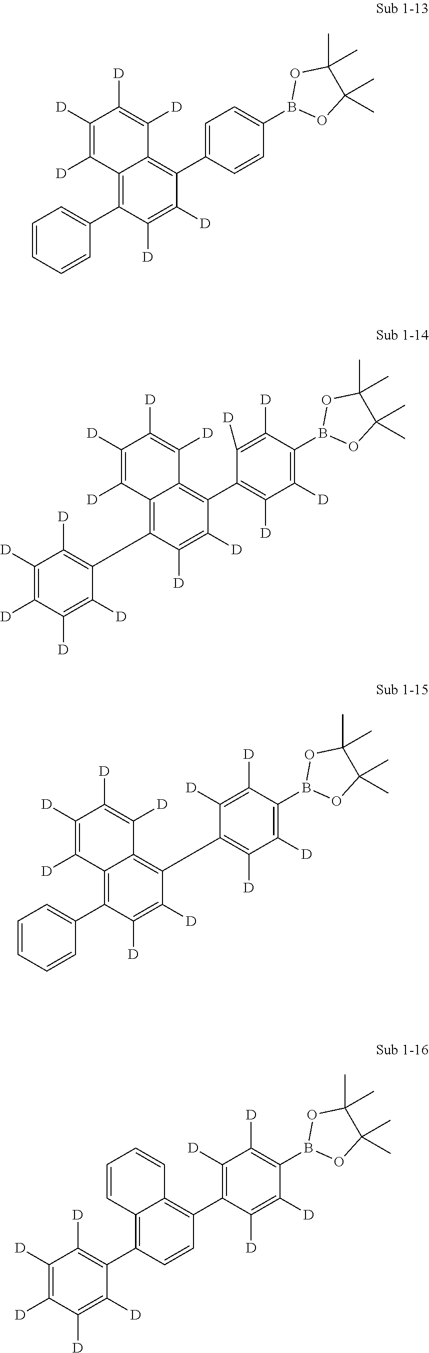 [SMILES: [2H]c1c([2H])c([2H])c2c(-c3ccc(B4OC(C)(C)C(C)(C)O4)cc3)c([2H])c([2H])c(-c3ccccc3)c2c1[2H] ; [2H]c1c([2H])c([2H])c(Cc2c([2H])c([2H])c(-c3c([2H])c([2H])c(B4OC(C)(C)C(C)(C)O4)c([2H])c3[2H])c3c([2H])c([2H])c([2H])c([2H])c23)c([2H])c1[2H] ; [2H]c1c([2H])c(B2OC(C)(C)C(C)(C)O2)c([2H])c([2H])c1Cc1c([2H])c([2H])c(-c2ccccc2)c2c([2H])c([2H])c([2H])c([2H])c12 ; [2H]c1c([2H])c([2H])c(-c2ccc(-c3c([2H])c([2H])c(B4OC(C)(C)C(C)(C)O4)c([2H])c3[2H])c3ccccc23)c([2H])c1[2H]]

TABLE 1

| compound | FD-MS | compound | FD-MS |
|---|---|---|---|
| Sub1-1 | m/z = 406.21(C₂₈H₂₇BO₂ = 406.33) | Sub1-2 | m/z = 411.24(C₂₈H₂₂D₅BO₂ = 411.36) |
| Sub1-3 | m/z = 410.24(C₂₈H₂₃D₄BO₂ = 410.36) | Sub1-4 | m/z = 417.28(C₂₈H₁₆D₁₁BO₂ = 417.40) |
| Sub1-5 | m/z = 412.25(C₂₈H₂₁D₆BO₂ = 412.37) | Sub1-6 | m/z = 421.30(C₂₈H₁₂D₁₅BO₂ = 421.42) |
| Sub1-7 | m/z = 416.27(C₂₈H₁₇D₁₀BO₂ = 416.39) | Sub1-8 | m/z = 415.27(C₂₈H₁₈D₉BO₂ = 415.39) |
| Sub1-9 | m/z = 406.21(C₂₈H₂₇BO₂ = 406.33) | Sub1-10 | m/z = 411.24(C₂₈H₂₂D₅BO₂ = 411.36) |
| Sub1-11 | m/z = 410.24(C₂₈H₂₃D₄BO₂ = 410.36) | Sub1-12 | m/z = 417.28(C₂₈H₁₆D₁₁BO₂ = 417.40) |
| Sub1-13 | m/z = 412.25(C₂₈H₂₁D₆BO₂ = 412.37) | Sub1-14 | m/z = 421.30(C₂₈H₁₂D₁₅BO₂ = 421.42) |
| Sub1-15 | m/z = 416.27(C₂₈H₁₇D₁₀BO₂ = 416.39) | Sub1-16 | m/z = 415.27(C₂₈H₁₈D₉BO₂ = 415.39) |

Sub 2 of the reaction scheme 2 may be, but is not limited to, the compounds below, and the FD-MS (Field Desorption-Mass Spectrometry) values of compounds belonging to Sub 2 are as shown in Table 2.

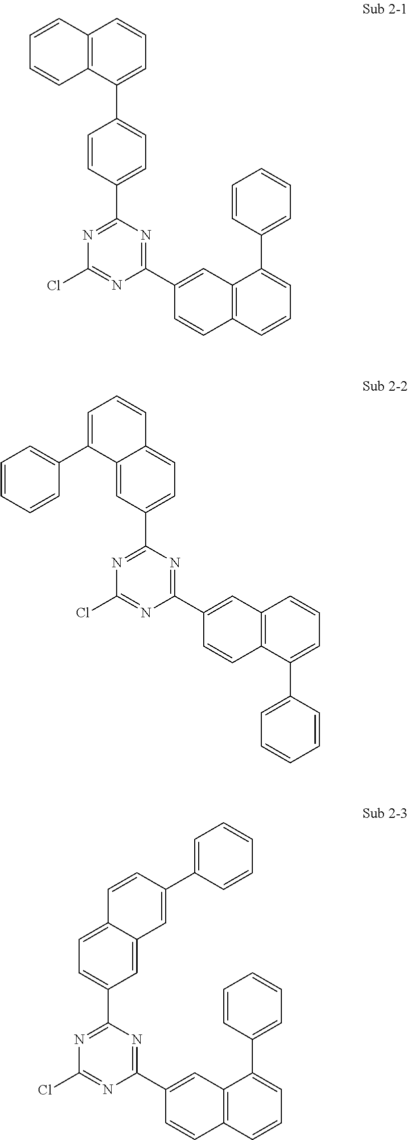 [SMILES: Clc1nc(-c2ccc(-c3cccc4ccccc34)cc2)nc(-c2ccc3cccc(-c4ccccc4)c3c2)n1 ; Clc1nc(-c2ccc3c(-c4ccccc4)cccc3c2)nc(-c2ccc3cccc(-c4ccccc4)c3c2)n1 ; Clc1nc(-c2ccc3ccc(-c4ccccc4)cc3c2)nc(-c2ccc3cccc(-c4ccccc4)c3c2)n1]

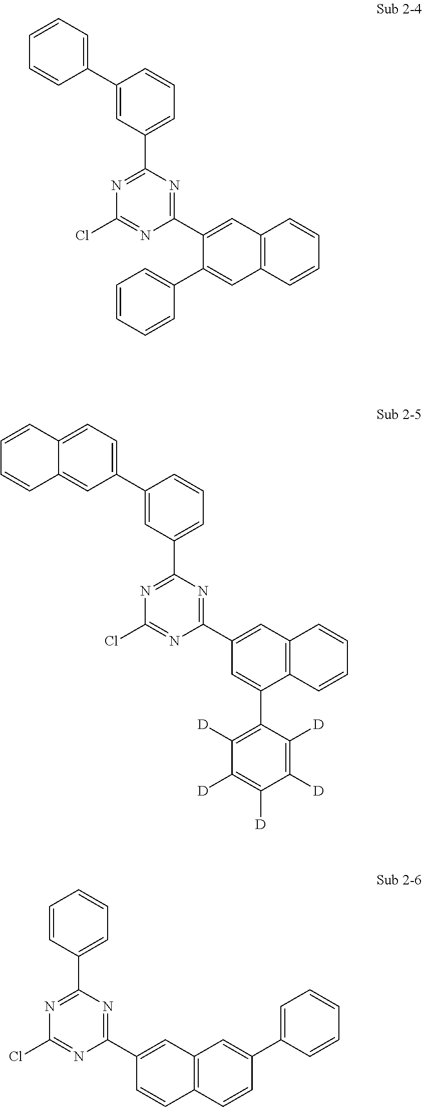 [SMILES: Clc1nc(-c2cccc(-c3ccccc3)c2)nc(-c2cc3ccccc3cc2-c2ccccc2)n1 ; [2H]c1c([2H])c([2H])c(-c2cc(-c3nc(Cl)nc(-c4cccc(-c5ccc6ccccc6c5)c4)n3)cc3ccccc23)c([2H])c1[2H] ; Clc1nc(-c2ccccc2)nc(-c2ccc3ccc(-c4ccccc4)cc3c2)n1]

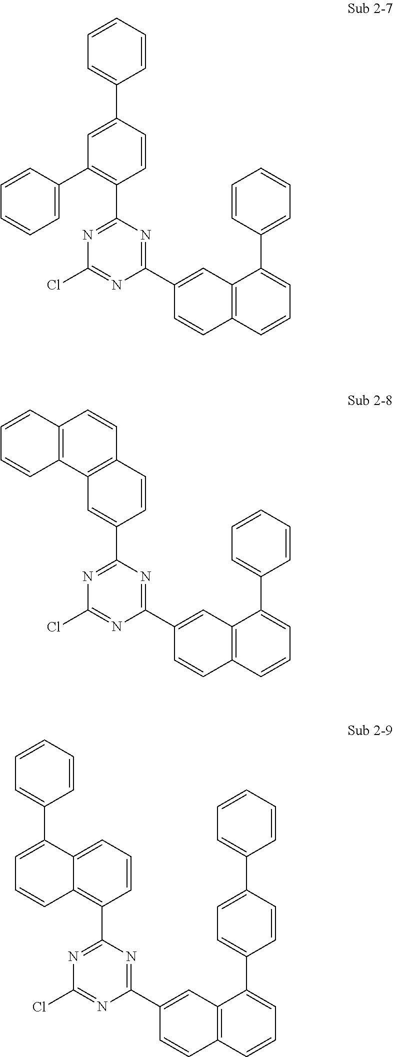 [SMILES: Clc1nc(-c2ccc3cccc(-c4ccccc4)c3c2)nc(-c2ccc(-c3ccccc3)cc2-c2ccccc2)n1 ; Clc1nc(-c2ccc3cccc(-c4ccccc4)c3c2)nc(-c2ccc3ccc4ccccc4c3c2)n1 ; Clc1nc(-c2ccc3cccc(-c4ccc(-c5ccccc5)cc4)c3c2)nc(-c2cccc3c(-c4ccccc4)cccc23)n1]

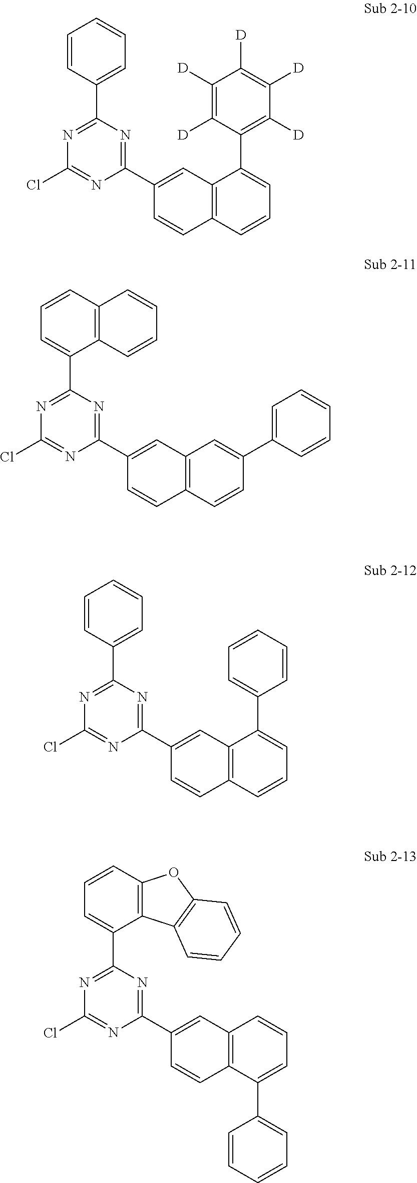 [SMILES: [2H]c1c([2H])c([2H])c(-c2cccc3ccc(-c4nc(Cl)nc(-c5ccccc5)n4)cc23)c([2H])c1[2H] ; Clc1nc(-c2ccc3ccc(-c4ccccc4)cc3c2)nc(-c2cccc3ccccc23)n1 ; Clc1nc(-c2ccccc2)nc(-c2ccc3cccc(-c4ccccc4)c3c2)n1 ; Clc1nc(-c2ccc3c(-c4ccccc4)cccc3c2)nc(-c2cccc3oc4ccccc4c23)n1]

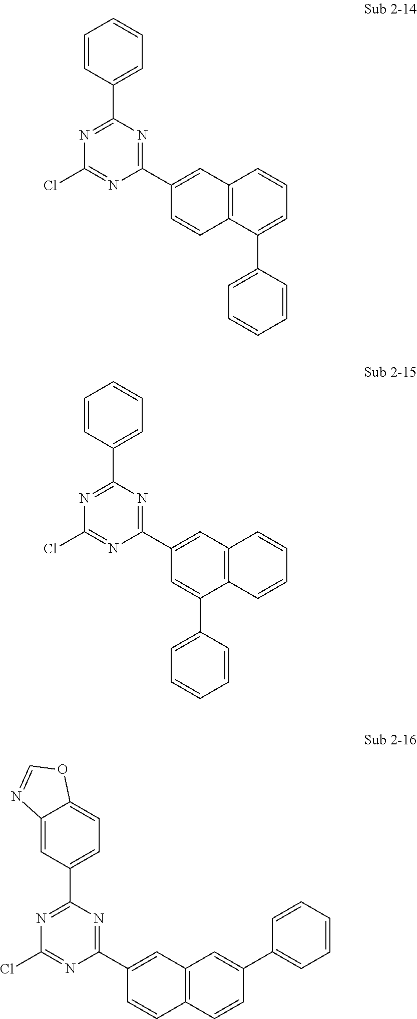 [SMILES: Clc1nc(-c2ccccc2)nc(-c2ccc3c(-c4ccccc4)cccc3c2)n1 ; Clc1nc(-c2ccccc2)nc(-c2cc(-c3ccccc3)c3ccccc3c2)n1 ; Clc1nc(-c2ccc3ccc(-c4ccccc4)cc3c2)nc(-c2ccc3ocnc3c2)n1]

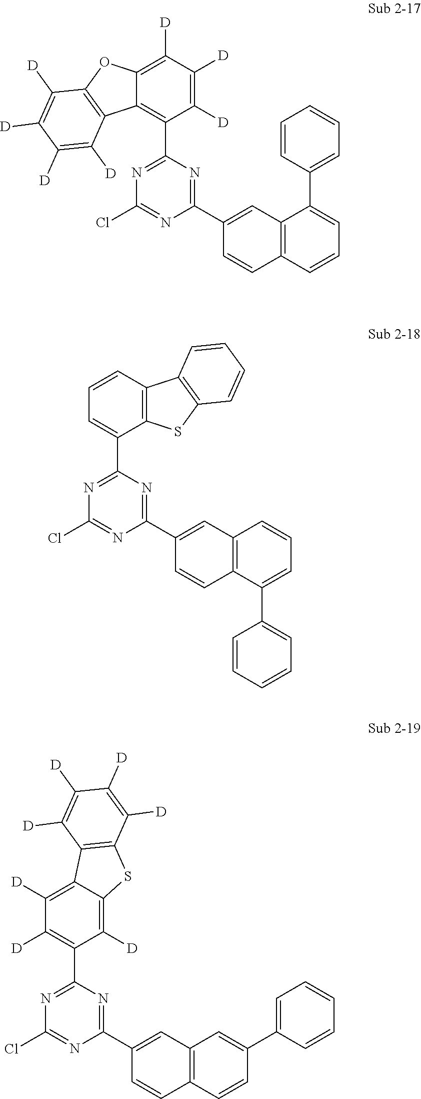 [SMILES: [2H]c1c([2H])c([2H])c2c(oc3c([2H])c([2H])c([2H])c(-c4nc(Cl)nc(-c5ccc6cccc(-c7ccccc7)c6c5)n4)c32)c1[2H] ; Clc1nc(-c2ccc3c(-c4ccccc4)cccc3c2)nc(-c2cccc3c2sc2ccccc23)n1 ; [2H]c1c([2H])c([2H])c2c(sc3c([2H])c(-c4nc(Cl)nc(-c5ccc6ccc(-c7ccccc7)cc6c5)n4)c([2H])c([2H])c32)c1[2H]]

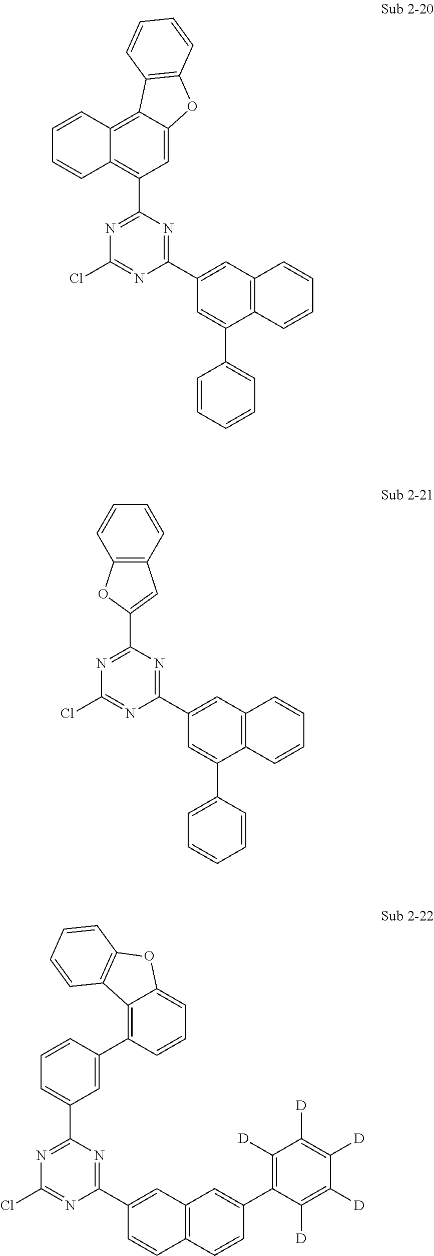 [SMILES: Clc1nc(-c2cc(-c3ccccc3)c3ccccc3c2)nc(-c2cc3oc4ccccc4c3c3ccccc23)n1 ; Clc1nc(-c2cc(-c3ccccc3)c3ccccc3c2)nc(-c2cc3ccccc3o2)n1 ; [2H]c1c([2H])c([2H])c(-c2ccc3ccc(-c4nc(Cl)nc(-c5cccc(-c6cccc7oc8ccccc8c67)c5)n4)cc3c2)c([2H])c1[2H]]

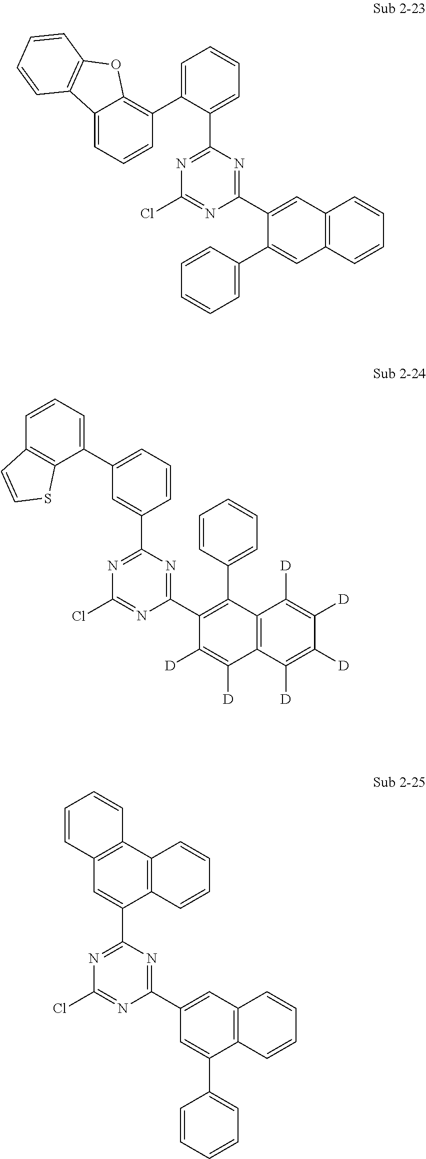 [SMILES: Clc1nc(-c2cc3ccccc3cc2-c2ccccc2)nc(-c2ccccc2-c2cccc3c2oc2ccccc23)n1 ; [2H]c1c([2H])c([2H])c2c(-c3ccccc3)c(-c3nc(Cl)nc(-c4cccc(-c5cccc6ccsc56)c4)n3)c([2H])c([2H])c2c1[2H] ; Clc1nc(-c2cc(-c3ccccc3)c3ccccc3c2)nc(-c2cc3ccccc3c3ccccc23)n1]

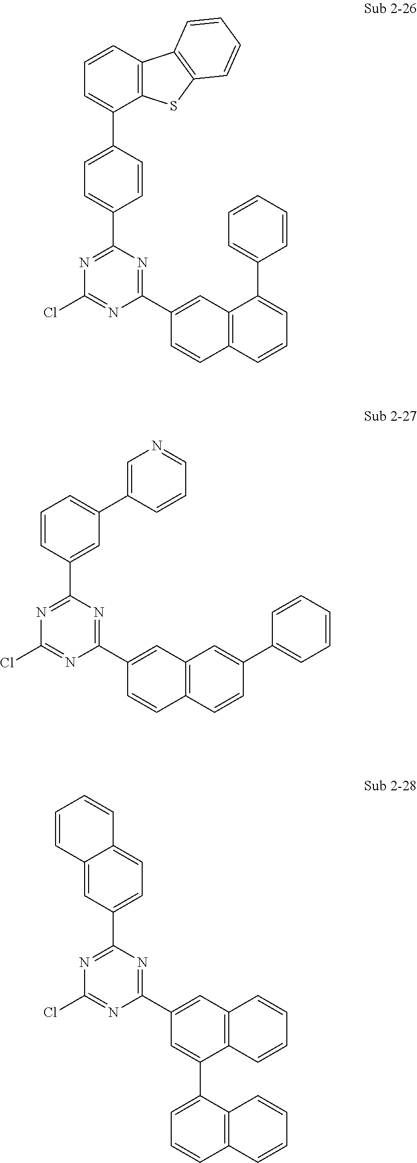 [SMILES: Clc1nc(-c2ccc(-c3cccc4c3sc3ccccc34)cc2)nc(-c2ccc3cccc(-c4ccccc4)c3c2)n1 ; Clc1nc(-c2cccc(-c3cccnc3)c2)nc(-c2ccc3ccc(-c4ccccc4)cc3c2)n1 ; Clc1nc(-c2ccc3ccccc3c2)nc(-c2cc(-c3cccc4ccccc34)c3ccccc3c2)n1]

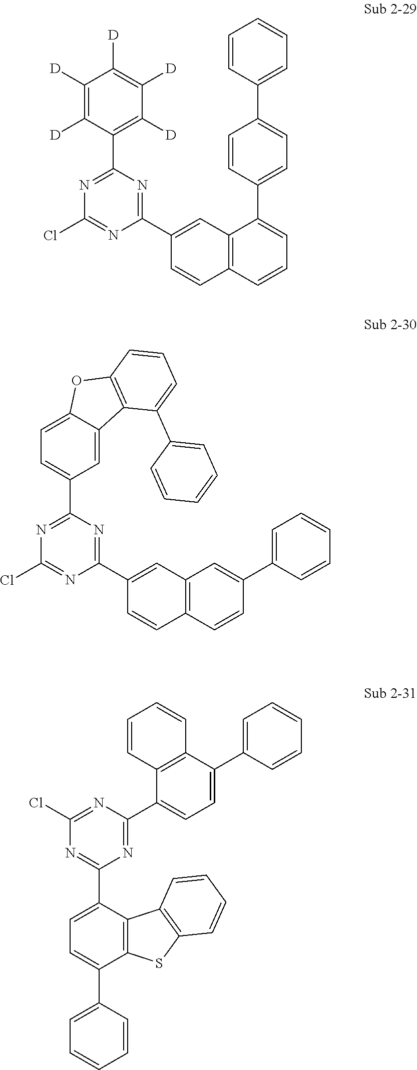 [SMILES: [2H]c1c([2H])c([2H])c(-c2nc(Cl)nc(-c3ccc4cccc(-c5ccc(-c6ccccc6)cc5)c4c3)n2)c([2H])c1[2H] ; Clc1nc(-c2ccc3ccc(-c4ccccc4)cc3c2)nc(-c2ccc3oc4cccc(-c5ccccc5)c4c3c2)n1 ; Clc1nc(-c2ccc(-c3ccccc3)c3ccccc23)nc(-c2ccc(-c3ccccc3)c3sc4ccccc4c23)n1]

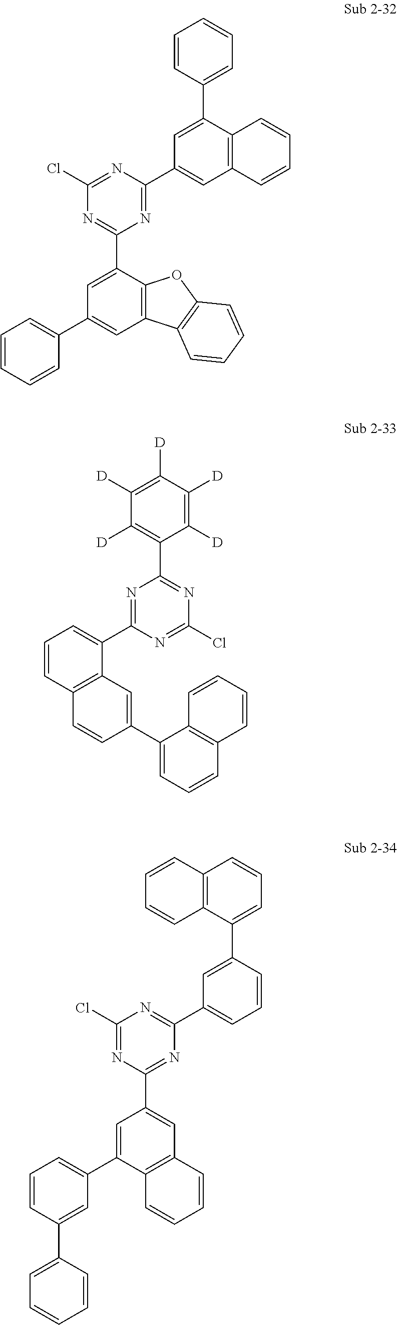 [SMILES: Clc1nc(-c2cc(-c3ccccc3)c3ccccc3c2)nc(-c2cc(-c3ccccc3)cc3c2oc2ccccc23)n1 ; [2H]c1c([2H])c([2H])c(-c2nc(Cl)nc(-c3cccc4ccc(-c5cccc6ccccc56)cc34)n2)c([2H])c1[2H] ; Clc1nc(-c2cccc(-c3cccc4ccccc34)c2)nc(-c2cc(-c3cccc(-c4ccccc4)c3)c3ccccc3c2)n1]

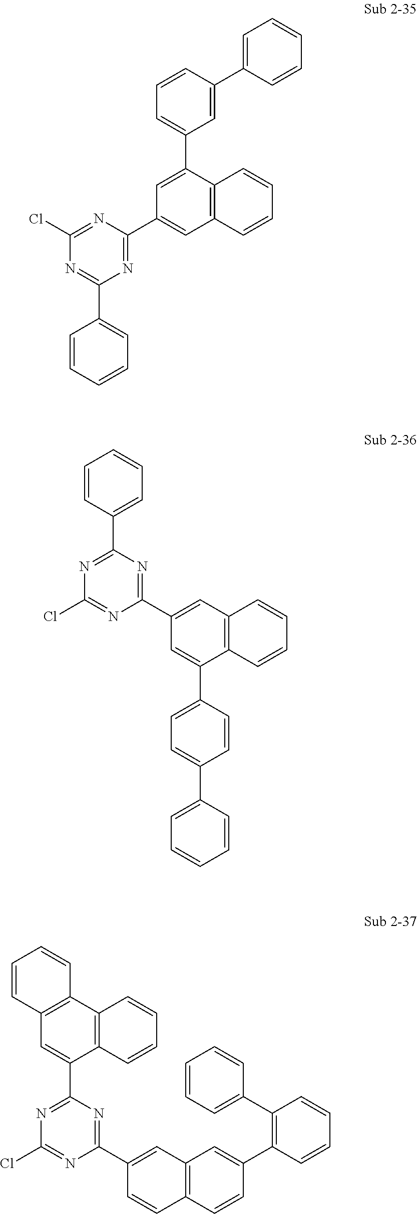 [SMILES: Clc1nc(-c2ccccc2)nc(-c2cc(-c3cccc(-c4ccccc4)c3)c3ccccc3c2)n1 ; Clc1nc(-c2ccccc2)nc(-c2cc(-c3ccc(-c4ccccc4)cc3)c3ccccc3c2)n1 ; Clc1nc(-c2ccc3ccc(-c4ccccc4-c4ccccc4)cc3c2)nc(-c2cc3ccccc3c3ccccc23)n1]

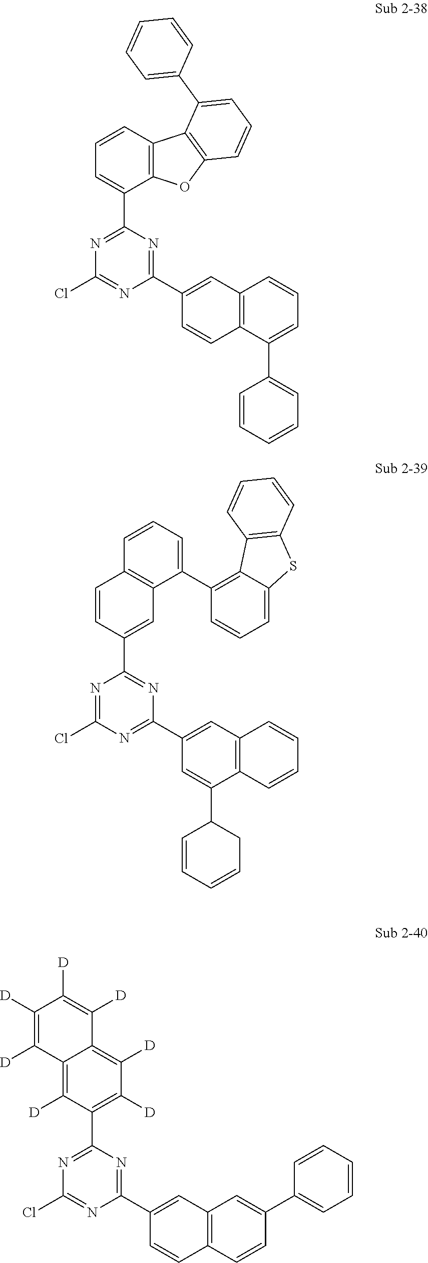

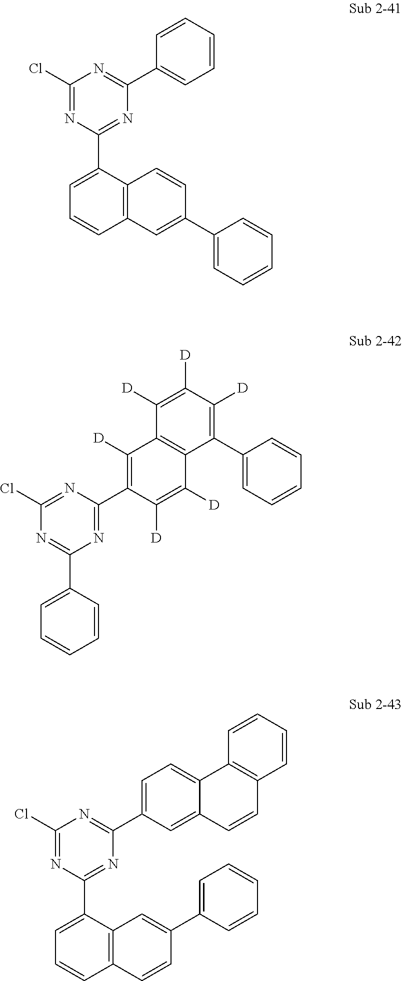 [SMILES: Clc1nc(-c2ccccc2)nc(-c2cccc3cc(-c4ccccc4)ccc23)n1 ; [2H]c1c([2H])c(-c2ccccc2)c2c([2H])c([2H])c(-c3nc(Cl)nc(-c4ccccc4)n3)c([2H])c2c1[2H] ; Clc1nc(-c2ccc3c(ccc4ccccc43)c2)nc(-c2cccc3ccc(-c4ccccc4)cc23)n1]

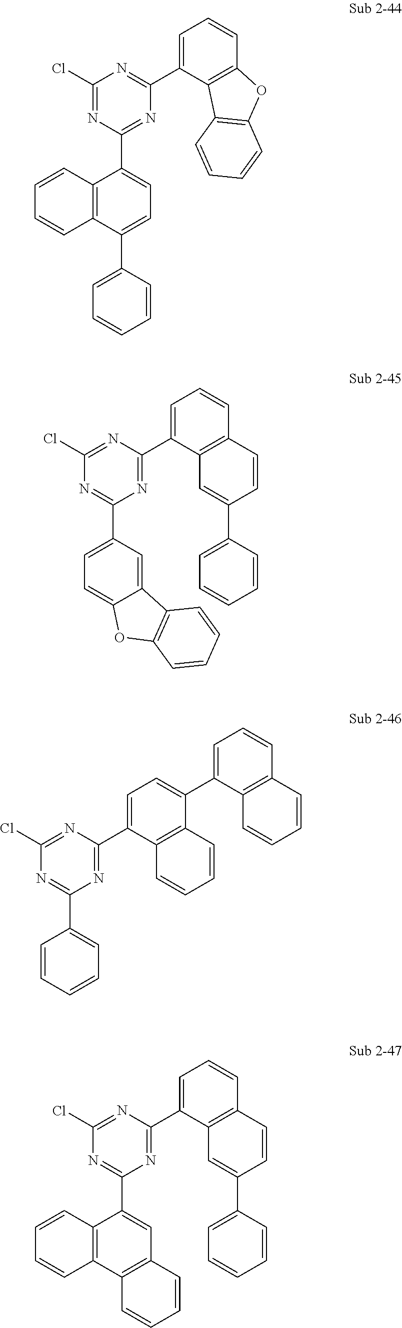 [SMILES: Clc1nc(-c2ccc(-c3ccccc3)c3ccccc23)nc(-c2cccc3oc4ccccc4c23)n1 ; Clc1nc(-c2ccc3oc4ccccc4c3c2)nc(-c2cccc3ccc(-c4ccccc4)cc23)n1 ; Clc1nc(-c2ccccc2)nc(-c2ccc(-c3cccc4ccccc34)c3ccccc23)n1 ; Clc1nc(-c2cccc3ccc(-c4ccccc4)cc23)nc(-c2cc3ccccc3c3ccccc23)n1]

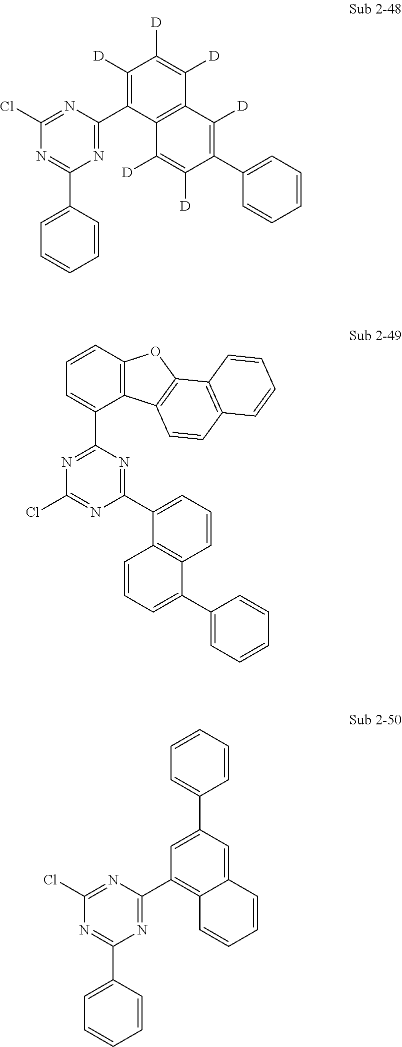 [SMILES: [2H]c1c([2H])c(-c2nc(Cl)nc(-c3ccccc3)n2)c2c([2H])c([2H])c(-c3ccccc3)c([2H])c2c1[2H] ; Clc1nc(-c2cccc3c(-c4ccccc4)cccc23)nc(-c2cccc3oc4c5ccccc5ccc4c23)n1 ; Clc1nc(-c2ccccc2)nc(-c2cc(-c3ccccc3)cc3ccccc23)n1]

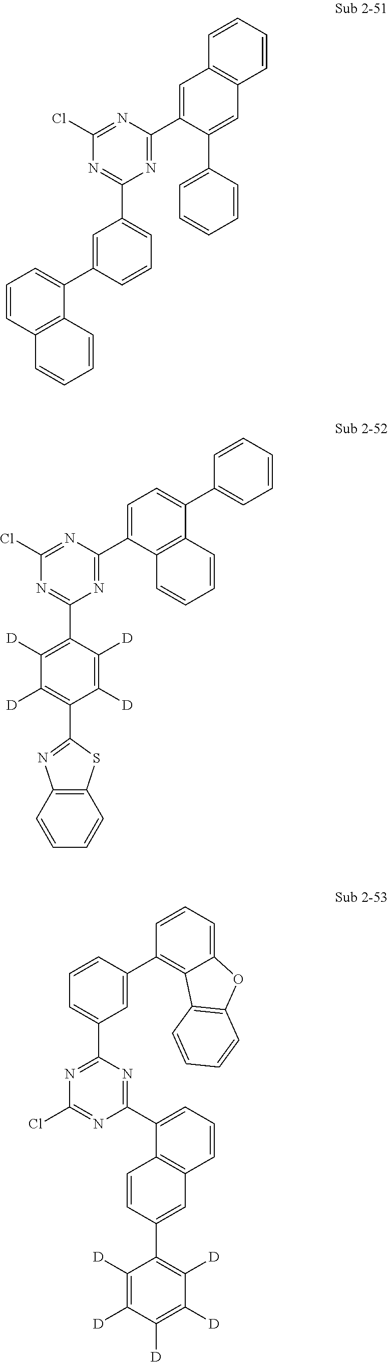 [SMILES: Clc1nc(-c2cccc(-c3cccc4ccccc34)c2)nc(-c2cc3ccccc3cc2-c2ccccc2)n1 ; [2H]c1c([2H])c(-c2nc3ccccc3s2)c([2H])c([2H])c1-c1nc(Cl)nc(-c2ccc(-c3ccccc3)c3ccccc23)n1 ; [2H]c1c([2H])c([2H])c(-c2ccc3c(-c4nc(Cl)nc(-c5cccc(-c6cccc7oc8ccccc8c67)c5)n4)cccc3c2)c([2H])c1[2H]]

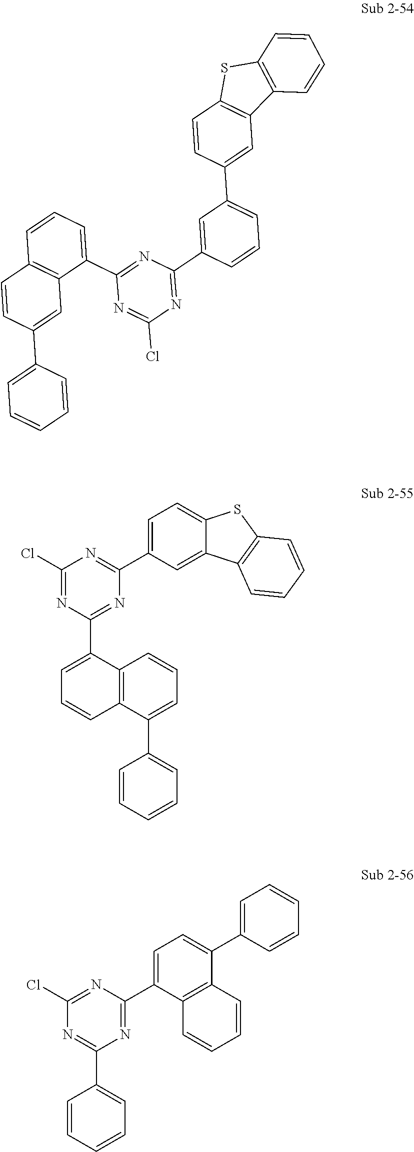 [SMILES: Clc1nc(-c2cccc(-c3ccc4sc5ccccc5c4c3)c2)nc(-c2cccc3ccc(-c4ccccc4)cc23)n1 ; Clc1nc(-c2ccc3sc4ccccc4c3c2)nc(-c2cccc3c(-c4ccccc4)cccc23)n1 ; Clc1nc(-c2ccccc2)nc(-c2ccc(-c3ccccc3)c3ccccc23)n1]

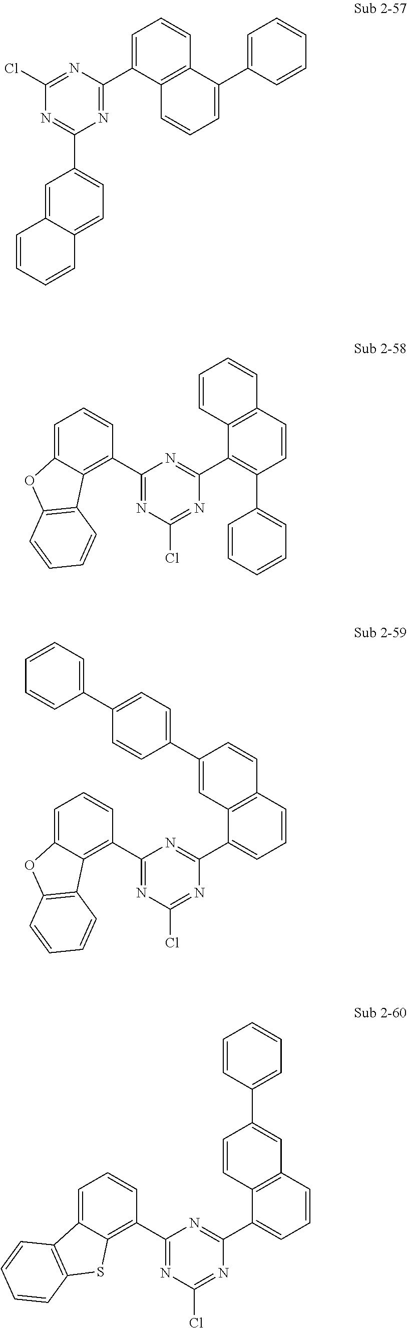 [SMILES: Clc1nc(-c2ccc3ccccc3c2)nc(-c2cccc3c(-c4ccccc4)cccc23)n1 ; Clc1nc(-c2c(-c3ccccc3)ccc3ccccc23)nc(-c2cccc3oc4ccccc4c23)n1 ; Clc1nc(-c2cccc3ccc(-c4ccc(-c5ccccc5)cc4)cc23)nc(-c2cccc3oc4ccccc4c23)n1 ; Clc1nc(-c2cccc3cc(-c4ccccc4)ccc23)nc(-c2cccc3c2sc2ccccc23)n1]

TABLE 2

| Compound | FD-MS | Compound | FD-MS |
|---|---|---|---|
| Sub2-1 | m/z = 519.15(C₃₅H₂₂ClN₃ = 520.03) | Sub2-2 | m/z = 519.15(C₃₅H₂₂ClN₃ = 520.03) |
| Sub2-3 | m/z = 519.15(C₃₅H₂₂ClN₃ = 520.03) | Sub2-4 | m/z = 469.13(C₃₁H₂₀ClN₃ = 469.97) |
| Sub2-5 | m/z = 524.18(C₃₅H₁₇D₅ClN₃ = 525.06) | Sub2-6 | m/z = 393.1(C₂₅H₁₆ClN₃ = 393.87) |
| Sub2-7 | m/z = 545.17(C₃₇H₂₄ClN₃ = 546.07) | Sub2-8 | m/z = 493.13(C₃₃H₂₀ClN₃ = 493.99) |
| Sub2-9 | m/z = 595.18(C₄₁H₂₆ClN₃ = 596.13) | Sub2-10 | m/z = 398.13(C₂₅H₁₁D₅ClN₃ = 398.9) |
| Sub2-11 | m/z = 443.12(C₂₉H₁₈ClN₃ = 443.93) | Sub2-12 | m/z = 393.1(C₂₅H₁₆ClN₃ = 393.87) |
| Sub2-13 | m/z = 483.11(C₃₁H₁₈ClN₃O = 483.96) | Sub2-14 | m/z = 393.1(C₂₅H₁₆ClN₃ = 393.87) |
| Sub2-15 | m/z = 393.1(C₂₅H₁₆ClN₃ = 393.87) | Sub2-16 | m/z = 434.09(C₂₆H₁₅ClN₄O = 434.88) |
| Sub2-17 | m/z = 490.16(C₃₁H₁₁D₇ClN₃O = 491) | Sub2-18 | m/z = 499.09(C₃₁H₁₈ClN₃S = 500.02) |
| Sub2-19 | m/z = 506.13(C₃₁H₁₁D₇ClN₃S = 507.06) | Sub2-20 | m/z = 533.13(C₃₅H₂₀ClN₃O = 534.02) |
| Sub2-21 | m/z = 433.1(C₂₇H₁₆ClN₃O = 433.9) | Sub2-22 | m/z = 564.18(C₃₇H₁₇D₅ClN₃O = 565.08) |
| Sub2-23 | m/z = 559.15(C₃₇H₂₂ClN₃O = 560.05) | Sub2-24 | m/z = 531.14(C₃₃H₁₄D₆ClN₃S = 532.09) |
| Sub2-25 | m/z = 493.13(C₃₃H₂₀ClN₃ = 493.99) | Sub2-26 | m/z = 575.12(C₃₇H₂₂ClN₃S = 576.11) |
| Sub2-27 | m/z = 470.13(C₃₀H₁₉ClN₄ = 470.96) | Sub2-28 | m/z = 493.13(C₃₃H₂₀ClN₃ = 493.99) |
| Sub2-29 | m/z = 474.17(C₃₁H₁₅D₅ClN₃ = 475) | Sub2-30 | m/z = 559.15(C₃₇H₂₂ClN₃O = 560.05) |
| Sub2-31 | m/z = 575.12(C₃₇H₂₂ClN₃S = 576.11) | Sub2-32 | m/z = 559.15(C₃₇H₂₂ClN₃O = 560.05) |
| Sub2-33 | m/z = 448.15(C₂₉H₁₃D₅ClN₃ = 448.96) | Sub2-34 | m/z = 595.18(C₄₁H₂₆ClN₃ = 596.13) |
| Sub2-35 | m/z = 469.13(C₃₁H₂₀ClN₃ = 469.97) | Sub2-36 | m/z = 469.13(C₃₁H₂₀ClN₃ = 469.97) |
| Sub2-37 | m/z = 569.17(C₃₉H₂₄ClN₃ = 570.09) | Sub2-38 | m/z = 559.15(C₃₇H₂₂ClN₃O = 560.05) |
| Sub2-39 | m/z = 625.14(C₄₁H₂₄ClN₃S = 626.17) | Sub2-40 | m/z = 450.16(C₂₉H₁₁D₇ClN₃ = 450.98) |
| Sub2-41 | m/z = 393.10(C₂₅H₁₆ClN₃ = 393.87) | Sub2-42 | m/z = 399.14(C₂₅H₁₀D₆ClN₃ = 399.91) |
| Sub2-43 | m/z = 493.13(C₃₃H₂₀ClN₃ = 493.99) | Sub2-44 | m/z = 483.11(C₃₁H₁₈ClN₃O = 483.96) |
| Sub2-45 | m/z = 483.11(C₃₁H₁₈ClN₃O = 483.96) | Sub2-46 | m/z = 443.12(C₂₉H₁₈ClN₃ = 443.93) |
| Sub2-47 | m/z = 493.13(C₃₃H₂₀ClN₃S = 493.99) | Sub2-48 | m/z = 399.14(C₂₅H₁₀D₆ClN₃S = 399.91) |
| Sub2-49 | m/z = 533.13(C₃₅H₂₀ClN₃ = 534.02) | Sub2-50 | m/z = 393.10(C₂₅H₁₆ClN₃ = 393.87) |
| Sub2-51 | m/z = 519.15(C₃₅H₂₂ClN₃ = 520.03) | Sub2-52 | m/z = 530.13(C₃₂H₁₄D₄ClN₄S = 531.07) |
| Sub2-53 | m/z = 564.18(C₃₇H₁₇D₅ClN₃O = 565.08) | Sub2-54 | m/z = 575.12(C₃₇H₂₂ClN₃S = 576.11) |
| Sub2-55 | m/z = 499.09(C₃₁H₁₈ClN₃S = 500.02) | Sub2-56 | m/z = 393.10(C₂₅H₁₆ClN₃ = 393.87) |
| Sub2-57 | m/z = 443.12(C₂₉H₁₈ClN₃ = 443.93) | Sub2-58 | m/z = 483.11(C₃₁H₁₈ClN₃O = 483.96) |
| Sub2-59 | m/z = 559.15(C₃₇H₂₂ClN₃O = 560.05) | Sub2-60 | m/z = 499.09(C₃₁H₁₈ClN₃S = 500.02) |

The FD-MS (Field Desorption-Mass Spectrometry) values of compounds P-1 to P-132 of the present invention manufactured according to the synthetic examples are as shown in Table 3.

TABLE 3

| Compound | FD-MS | Compound | FD-MS |
|---|---|---|---|
| P-1 | m/z = 763.3(C₅₇H₃₇N₃ = 763.94) | P-2 | m/z = 763.3(C₅₇H₃₇N₃ = 763.94) |
| P-3 | m/z = 763.3(C₅₇H₃₇N₃ = 763.94) | P-4 | m/z = 763.3(C₅₇H₃₇N₃ = 763.94) |
| P-5 | m/z = 713.28(C₅₃H₃₅N₃ = 713.88) | P-6 | m/z = 763.3(C₅₇H₃₇N₃ = 763.94) |
| P-7 | m/z = 768.33(C₅₇H₃₂D₅N₃ = 768.97) | P-8 | m/z = 789.31(C₅₉H₃₉N₃ = 789.98) |
| P-9 | m/z = 763.3(C₅₇H₃₇N₃ = 763.94) | P-10 | m/z = 737.28(C₅₅H₃₅N₃ = 737.91) |
| P-11 | m/z = 763.3(C₅₇H₃₇N₃ = 763.94) | P-12 | m/z = 737.28(C₅₅H₃₅N₃ = 737.91) |
| P-13 | m/z = 763.3(C₅₇H₃₇N₃ = 763.94) | P-14 | m/z = 713.28(C₅₃H₃₅N₃ = 713.88) |
| P-15 | m/z = 839.33(C₆₃H₄₁N₃ = 840.04) | P-16 | m/z = 713.28(C₅₃H₃₅N₃ = 713.88) |
| P-17 | m/z = 687.27(C₅₁H₃₃N₃ = 687.85) | P-18 | m/z = 691.29(C₅₁H₂₉D₄N₃ = 691.87) |
| P-19 | m/z = 687.27(C₅₁H₃₃N₃ = 687.85) | P-20 | m/z = 694.31(C₅₁H₂₆DN₃ = 694.89) |
| P-21 | m/z = 687.27(C₅₁H₃₃N₃ = 687.85) | P-22 | m/z = 687.27(C₅₁H₃₃N₃ = 687.85) |
| P-23 | m/z = 727.26(C₅₃H₃₃N₃O = 727.87) | P-24 | m/z = 727.26(C₅₃H₃₃N₃O = 727.87) |
| P-25 | m/z = 637.25(C₄₇H₃₁N₃ = 637.79) | P-26 | m/z = 637.25(C₄₇H₃₁N₃ = 637.79) |
| P-27 | m/z = 637.25(C₄₇H₃₁N₃ = 637.79) | P-28 | m/z = 642.28(C₄₇H₂₆D₅N₃ = 642.82) |
| P-29 | m/z = 642.28(C₄₇H₂₆D₅N₃ = 642.82) | P-30 | m/z = 637.25(C₄₇H₃₁N₃ = 637.79) |
| P-31 | m/z = 687.27(C₅₁H₃₃N₃ = 687.85) | P-32 | m/z = 687.27(C₅₁H₃₃N₃ = 687.85) |
| P-33 | m/z = 678.24(C₄₈H₃₀N₄O = 678.8) | P-34 | m/z = 734.31(C₅₃H₂₆D₇N₃O = 734.91) |
| P-35 | m/z = 743.24(C₅₃H₃₃N₃S = 743.93) | P-36 | m/z = 727.26(C₅₃H₃₃N₃O = 727.87) |
| P-37 | m/z = 743.24(C₅₃H₃₃N₃S = 743.93) | P-38 | m/z = 727.26(C₅₃H₃₃N₃O = 727.87) |
| P-39 | m/z = 758.33(C₅₃H₁₈D₁₅N₃S = 759.02) | P-40 | m/z = 733.3(C₅₃H₂₇D₆N₃O = 733.9) |
| P-41 | m/z = 727.26(C₅₃H₃₃N₃O = 727.87) | P-42 | m/z = 743.24(C₅₃H₃₃N₃S = 743.93) |
| P-43 | m/z = 732.29(C₅₃H₂₈D₅N₃O = 732.9) | P-44 | m/z = 750.28(C₅₃H₂₆D₇N₃S = 750.97) |
| P-45 | m/z = 727.26(C₅₃H₃₃N₃O = 727.87) | P-46 | m/z = 793.26(C₅₇H₃₅N₃S = 793.99) |
| P-47 | m/z = 786.33(C₅₇H₂₆D₉N₃O = 786.98) | P-48 | m/z = 743.24(C₅₃H₃₃N₃S = 743.93) |
| P-49 | m/z = 693.22(C₄₉H₃₁N₃S = 693.87) | P-50 | m/z = 681.27(C₄₉H₂₇D₄N₃O = 681.83) |
| P-51 | m/z = 677.25(C₄₉H₃₁N₃O = 677.81) | P-52 | m/z = 693.22(C₄₉H₃₁N₃S = 693.87) |
| P-53 | m/z = 819.27(C₅₉H₃₇N₃S = 820.03) | P-54 | m/z = 777.28(C₅₇H₃₅N₃O = 777.93) |
| P-55 | m/z = 774.28(C₅₄H₃₀D₄N₄S = 774.98) | P-56 | m/z = 808.33(C₅₉H₃₂D₅N₃O = 809) |
| P-57 | m/z = 803.29(C₅₉H₃₇N₃O = 803.97) | P-58 | m/z = 824.3(C₅₉H₃₂D₅N₃S = 825.06) |
| P-59 | m/z = 819.27(C₅₉H₃₇N₃S = 820.03) | P-60 | m/z = 754.27(C₅₄H₃₄N₄O = 754.89) |
| P-61 | m/z = 753.28(C₅₅H₃₅N₃O = 753.9) | P-62 | m/z = 775.29(C₅₅H₂₉D₆N₃S = 776) |
| P-63 | m/z = 810.34(C₅₉H₃₀D₇N₃O = 811.01) | P-64 | m/z = 743.32(C₅₅H₂₉D₆N₃ = 743.94) |
| P-65 | m/z = 819.27(C₅₉H₃₇N₃S = 820.03) | P-66 | m/z = 751.3(C₅₆H₃₇N₃ = 751.93) |
| P-67 | m/z = 819.27(C₅₉H₃₇N₃S = 820.03) | P-68 | m/z = 819.27(C₅₉H₃₇N₃S = 820.03) |
| P-69 | m/z = 753.28(C₅₅H₃₅N₃O = 753.9) | P-70 | m/z = 765.34(C₅₄H₂₃D₁₁N₄O = 765.96) |
| P-71 | m/z = 714.28(C₅₂H₃₄N₄ = 714.87) | P-72 | m/z = 714.28(C₅₂H₃₄N₄ = 714.87) |
| P-73 | m/z = 763.3(C₅₇H₃₇N₃ = 763.94) | P-74 | m/z = 737.28(C₅₅H₃₅N₃ = 737.91) |
| P-75 | m/z = 769.26(C₅₅H₃₅N₃S = 769.97) | P-76 | m/z = 737.28(C₅₅H₃₅N₃ = 737.91) |
| P-77 | m/z = 763.3(C₅₇H₃₇N₃ = 763.94) | P-78 | m/z = 737.28(C₅₅H₃₅N₃ = 737.91) |
| P-79 | m/z = 768.33(C₅₇H₃₂D₅N₃ = 768.97) | P-80 | m/z = 839.33(C₆₃H₄₁N₃ = 840.04) |
| P-81 | m/z = 803.29(C₅₉H₃₇N₃O = 803.97) | P-82 | m/z = 804.29(C₅₈H₃₆N₄O = 804.95) |
| P-83 | m/z = 692.3(C₅₁H₂₈D₅N₃ = 692.88) | P-84 | m/z = 713.28(C₅₃H₃₅N₃ = 713.88) |
| P-85 | m/z = 803.29(C₅₉H₃₇N₃O = 803.97) | P-86 | m/z = 803.29(C₅₉H₃₇N₃O = 803.97) |
| P-87 | m/z = 895.3(C₆₅H₄₁N₃S = 896.12) | P-88 | m/z = 830.34(C₅₉H₂₆D₁₁N₃S = 831.09) |
| P-89 | m/z = 803.29(C₅₉H₃₇N₃O = 803.97) | P-90 | m/z = 803.29(C₅₉H₃₇N₃O = 803.97) |
| P-91 | m/z = 869.29(C₆₃H₃₉N₃S = 870.09) | P-92 | m/z = 803.29(C₅₉H₃₇N₃O = 803.97) |
| P-93 | m/z = 687.27(C₅₁H₃₃N₃ = 687.85) | P-94 | m/z = 813.31(C₆₁H₃₉N₃ = 814) |
| P-95 | m/z = 687.27(C₅₁H₃₃N₃ = 687.85) | P-96 | m/z = 718.31(C₅₃H₃₀D₅N₃ = 718.91) |
| P-97 | m/z = 713.28(C₅₃H₃₅N₃ = 713.88) | P-98 | m/z = 718.31(C₅₃H₃₀D₅N₃ = 718.91) |
| P-99 | m/z = 687.27(C₅₁H₃₃N₃ = 687.85) | P-100 | m/z = 713.28(C₅₃H₃₅N₃ = 713.88) |
| P-101 | m/z = 648.32(C₄₇H₂₀D₁₁N₃ = 648.85) | P-102 | m/z = 643.29(C₄₇H₂₅D₆N₃ = 643.82) |
| P-103 | m/z = 637.25(C₄₇H₃₁N₃ = 637.79) | P-104 | m/z = 641.28(C₄₇H₂₇D₄N₃ = 641.81) |
| P-105 | m/z = 643.29(C₄₇H₂₅D₆N₃ = 643.82) | P-106 | m/z = 642.28(C₄₇H₂₆D₅N₃ = 642.82) |
| P-107 | m/z = 687.27(C₅₁H₃₃N₃ = 687.85) | P-108 | m/z = 713.28(C₅₃H₃₅N₃ = 713.88) |
| P-109 | m/z = 713.28(C₅₃H₃₅N₃ = 713.88) | P-110 | m/z = 718.31(C₅₃H₃₀D₅N₃ = 718.91) |
| P-111 | m/z = 722.34(C₅₃H₂₆D₉N₃ = 722.94) | P-112 | m/z = 643.29(C₄₇H₂₅D₆N₃ = 643.82) |
| P-113 | m/z = 637.25(C₄₇H₃₁N₃ = 637.79) | P-114 | m/z = 637.25(C₄₇H₃₁N₃ = 637.79) |
| P-115 | m/z = 643.29(C₄₇H₂₅D₆N₃ = 643.82) | P-116 | m/z = 637.25(C₄₇H₃₁N₃ = 637.79) |
| P-117 | m/z = 637.25(C₄₇H₃₁N₃ = 637.79) | P-118 | m/z = 693.31(C₅₁H₂₇D₆N₃ = 693.88) |
| P-119 | m/z = 643.29(C₄₇H₂₅D₆N₃ = 643.82) | P-120 | m/z = 642.28(C₄₇H₂₆D₅N₃ = 642.82) |
| P-121 | m/z = 643.29(C₄₇H₂₅D₆N₃ = 643.82) | P-122 | m/z = 637.25(C₄₇H₃₁N₃ = 637.79) |
| P-123 | m/z = 641.28(C₄₇H₂₇D₄N₃ = 641.81) | P-124 | m/z = 637.25(C₄₇H₃₁N₃ = 637.79) |
| P-125 | m/z = 693.31(C₅₁H₂₇D₆N₃ = 693.88) | P-126 | m/z = 641.28(C₄₇H₂₇D₄N₃ = 641.81) |
| P-127 | m/z = 643.29(C₄₇H₂₅D₆N₃ = 643.82) | P-128 | m/z = 642.28(C₄₇H₂₆D₅N₃ = 642.82) |
| P-129 | m/z = 643.29(C₄₇H₂₅D₆N₃ = 643.82) | P-130 | m/z = 643.29(C₄₇H₂₅D₆N₃ = 643.82) |
| P-131 | m/z = 643.29(C₄₇H₂₅D₆N₃ = 643.82) | P-132 | m/z = 642.28(C₄₇H₂₆D₅N₃ = 642.82) |

### Synthesis Example 2

The compound represented by Formula 2 or Formula 3 can be prepared by a known synthetic method (named reaction) or by referring to published patent publications, such as Korean Patent Registration No. 10-2395819 and U.S. Patent Publication No. 2023-0129535, but is not limited thereto.

Meanwhile, exemplary synthesis examples of the present invention represented by Formula 1 have been described, but these are all based on the Buchwald-Hartwig cross coupling reaction, Miyaura boration reaction, Suzuki cross-coupling reaction, Intramolecular acid-induced cyclization reaction (J. mater. Chem. 1999, 9, 2095.), Pd(II)-catalyzed oxidative cyclization reaction (Org. Lett. 2011, 13, 5504), and PPh₃-mediated reductive cyclization reaction (J. Org. Chem. 2005, 70, 5014.), and it will be easily understood by those skilled in the art that the reaction proceeds even when other substituents defined in Formula 1 are bonded in addition to the substituents specified in the specific synthesis examples.

The FD-MS values of compounds N-1 to N-128 and S-1 to S-120 of the present invention manufactured according to Synthetic Example 2 as described above are as shown in Tables 4 and 5.

TABLE 4

| Compound | FD-MS | Compound | FD-MS |
|---|---|---|---|
| N-1 | m/z = 487.19(C₃₆H₂₅NO = 487.60) | N-2 | m/z = 553.19(C₄₀H₂₇NS = 553.72) |
| N-3 | m/z = 563.26(C₄₃H₃₃N = 563.74) | N-4 | m/z = 602.27(C₄₅H₃₄N₂ = 602.78) |
| N-5 | m/z = 517.15(C₃₆H₂₃NOS = 517.65) | N-6 | m/z = 603.20(C₄₄H₂₉NS = 603.78) |
| N-7 | m/z = 735.29(C₅₇H₃₇N = 735.93) | N-8 | m/z = 562.24(C₄₂H₃₀N₂ = 562.72) |
| N-9 | m/z = 565.17(C₄₀H₂₃NO₃ = 565.63) | N-10 | m/z = 581.14(C₄₀H₂₃NO₂S = 581.69) |
| N-11 | m/z = 823.24(C₅₉H₃₇NS₂ = 824.07) | N-12 | m/z = 727.30(C₅₄H₃₇N₃ = 727.91) |
| N-13 | m/z = 627.22(C₄₆H₂₉NO₂ = 627.74) | N-14 | m/z = 633.16(C₄₄H₂₇NS₂ = 633.83) |
| N-15 | m/z = 675.29(C₅₂H₃₇N = 675.88) | N-16 | m/z = 678.30(C₅₁H₃₈N₂ = 678.88) |
| N-17 | m/z = 669.21(C₄₈H₃₁NOS = 669.84) | N-18 | m/z = 785.22(C₅₆H₃₅NS₂ = 786.02) |
| N-19 | m/z = 617.18(C₄₄H₂₇NOS = 617.77) | N-20 | m/z = 601.20(C₄₄H₂₇NO₂ = 601.71) |
| N-21 | m/z = 779.32(C₅₉H₄₁NO = 779.98) | N-22 | m/z = 583.23(C₄₂H₃₃NS = 583.79) |
| N-23 | m/z = 679.32(C₅₂H₄₁N = 679.91) | N-24 | m/z = 726.27(C₅₄H₃₄N₂O = 726.88) |
| N-25 | m/z = 593.18(C₄₂H₂₇NOS = 593.74) | N-26 | m/z = 774.22(C₅₄H₃₄N₂S₂ = 775.00) |
| N-27 | m/z = 557.24(C₄₀H₃₁NO₂ = 557.69) | N-28 | m/z = 652.25(C₄₈H₃₂N₂O = 652.80) |
| N-29 | m/z = 619.29(C₄₆H₃₇NO = 619.81) | N-30 | m/z = 603.20(C₄₄H₂₉NS = 603.78) |
| N-31 | m/z = 813.30(C₆₂H₃₉NO = 814.00) | N-32 | m/z = 784.29(C₅₇H₄₀N₂S = 785.02) |
| N-33 | m/z = 577.20(C₄₂H₂₇NO₂ = 577.68) | N-34 | m/z = 607.14(C₄₂H₂₅NS₂ = 607.79) |
| N-35 | m/z = 801.34(C₆₂H₄₃N = 802.03) | N-36 | m/z = 575.24(C₄₂H₂₉N₃ = 575.72) |
| N-37 | m/z = 577.20(C₄₂H₂₇NO₂ = 577.68) | N-38 | m/z = 607.14(C₄₂H₂₅NS₂ = 607.79) |
| N-39 | m/z = 801.34(C₆₂H₄₃N = 802.03) | N-40 | m/z = 575.24(C₄₂H₂₉N₃ = 575.72) |
| N-41 | m/z = 601.20(C₄₄H₂₇NO₂ = 601.71) | N-42 | m/z = 471.11(C₃₁H₂₁NS₂ = 471.64) |
| N-43 | m/z = 675.29(C₅₂H₃₇N = 675.88) | N-44 | m/z = 727.30(C₅₄H₃₇N₃ = 727.91) |
| N-45 | m/z = 603.20(C₄₄H₂₉NS = 603.78) | N-46 | m/z = 561.16(C₃₈H₂₇NS₂ = 561.76) |
| N-47 | m/z = 799.32(C₆₂H₄₁N = 800.02) | N-48 | m/z = 702.27(C₅₂H₃₄N₂O = 702.86) |
| N-49 | m/z = 729.27(C₅₄H₃₅NO₂ = 729.88) | N-50 | m/z = 785.22(C₅₆H₃₅NS₂ = 786.02) |
| N-51 | m/z = 812.32(C₆₂H₄₀N₂ = 813.02) | N-52 | m/z = 681.22(C₄₈H₃₁N₃S = 681.86) |
| N-53 | m/z = 615.18(C₄₄H₂₅NO₃ = 615.69) | N-54 | m/z = 763.15(C₅₂H₂₉NS₃ = 763.99) |
| N-55 | m/z = 593.31(C₄₅H₃₉N = 593.81) | N-56 | m/z = 840.33(C₆₂H₄₀N₄ = 841.03) |
| N-57 | m/z = 657.18(C₄₆H₂₇NO₂S = 657.79) | N-58 | m/z = 824.23(C₅₈H₃₆N₂S₂ = 825.06) |
| N-59 | m/z = 1195.42(C₉₁H₅₇NS = 1196.52) | N-60 | m/z = 656.19(C₄₆H₂₈N₂OS = 656.80) |
| N-61 | m/z = 607.16(C₄₂H₂₅NO₂S = 607.73) | N-62 | m/z = 773.20(C₅₄H₃₁NO₃S = 773.91) |
| N-63 | m/z = 1013.40(C₇₉H₅₁N = 1014.28) | N-64 | m/z = 758.24(C₅₄H₃₄N₂OS = 758.94) |
| N-65 | m/z = 623.14(C₄₂H₂₅NOS₂ = 623.79) | N-66 | m/z = 763.16(C₅₂H₂₉NO₂S₂ = 763.93) |
| N-67 | m/z = 799.20(C₅₆H₃₃NOS₂ = 800.01) | N-68 | m/z = 743.23(C₅₄H₃₃NOS = 743.92) |
| N-69 | m/z = 872.25(C₆₂H₃₆N₂O₂S = 873.04) | N-70 | m/z = 772.22(C₅₄H₃₂N₂O₂S = 772.92) |
| N-71 | m/z = 830.28(C₆₁H₃₈N₂S = 831.05) | N-72 | m/z = 808.25(C₅₈H₃₃FN₂O₂ = 808.91) |
| N-73 | m/z = 929.21(C₆₄H₃₅NO₃S₂ = 930.11) | N-74 | m/z = 963.27(C₆₈H₄₁N₃S₂ = 964.22) |
| N-75 | m/z = 809.24(C₅₈H₃₅NO₂S = 809.98) | N-76 | m/z = 893.29(C₆₆H₃₉NO₃ = 894.04) |
| N-77 | m/z = 794.28(C₅₈H₃₈N₂S = 795.02) | N-78 | m/z = 900.26(C₆₄H₄₀N₂S₂ = 901.16) |
| N-79 | m/z = 758.28(C₅₅H₃₈N₂S = 758.98) | N-80 | m/z = 1082.37(C₈₁H₅₀N₂S = 1083.37) |
| N-81 | m/z = 573.25(C₄₄H₃₁N = 573.74) | N-82 | m/z = 649.28(C₅₀H₃₅N = 649.84) |
| N-83 | m/z = 699.29(C₅₄H₃₇N = 699.90) | N-84 | m/z = 699.29(C₅₄H₃₇N = 699.90) |
| N-85 | m/z = 673.28(C₅₂H₃₅N = 673.86) | N-86 | m/z = 649.28(C₅₀H₃₅N = 649.84) |
| N-87 | m/z = 625.28(C₄₈H₃₅N = 625.82) | N-88 | m/z = 673.28(C₅₂H₃₅N = 673.86) |
| N-89 | m/z = 773.31(C₆₀H₃₉N = 773.98) | N-90 | m/z = 749.31(C₅₈H₃₉N = 749.96) |
| N-91 | m/z = 699.29(C₅₄H₃₇N = 699.90) | N-92 | m/z = 599.26(C₄₆H₃₃N = 599.78) |
| N-93 | m/z = 639.26(C₄₈H₃₃NO = 639.80) | N-94 | m/z = 765.25(C₅₇H₃₅NS = 765.97) |
| N-95 | m/z = 677.31(C₅₂H₃₉N = 677.89) | N-96 | m/z = 727.30(C₅₄H₃₇N₃ = 727.91) |
| N-97 | m/z = 552.18(C₃₉H₂₄N₂O₂ = 552.63) | N-98 | m/z = 628.22(C₄₅H₂₈N₂O₂ = 628.73) |
| N-99 | m/z = 614.24(C₄₅H₃₀N₂O = 614.75) | N-100 | m/z = 614.24(C₄₅H₃₀N₂O = 614.75) |
| N-101 | m/z = 691.21(C₅₀H₂₉NO₃ = 691.79) | N-102 | m/z = 739.29(C₅₆H₃₇NO = 739.92) |
| N-103 | m/z = 673.15(C₄₆H₂₇NOS₂ = 673.85) | N-104 | m/z = 726.27(C₅₄H₃₄N₂O = 726.88) |
| N-105 | m/z = 667.2(C₄₈H₂₉NOS = 667.83) | N-106 | m/z = 717.21(C₅₂H₃₁NOS = 717.89) |
| N-107 | m/z = 667.2(C₄₈H₂₉NOS = 667.83) | N-108 | m/z = 711.26(C₅₄H₃₃NO = 711.86) |
| N-109 | m/z = 617.18(C₄₄H₂₇NOS = 617.77) | N-110 | m/z = 611.22(C₄₆H₂₉NO = 611.74) |
| N-111 | m/z = 769.24(C₅₆H₃₅NOS = 769.96) | N-112 | m/z = 701.28(C₅₂H₃₅N3 = 701.87) |
| N-113 | m/z = 527.22(C₃₉H₂₉NO = 527.67) | N-114 | m/z = 643.2(C₄₆H₂₉NOS = 643.8) |
| N-115 | m/z = 593.18(C₄₂H₂₇NOS = 593.74) | N-116 | m/z = 726.27(C₅₄H₃₄N₂O = 726.88) |
| N-117 | m/z = 726.27(C₅₄H₃₄N₂O = 726.88) | N-118 | m/z = 558.14(C₃₇H₂₂N₂O₂S = 558.66) |
| N-119 | m/z = 620.19(C₄₃H₂₈N₂OS = 620.77) | N-120 | m/z = 610.24(C₄₆H₃₀N₂ = 610.76) |
| N-121 | m/z = 718.24(C₅₂H₃₄N₂S = 718.92) | N-122 | m/z = 728.28(C₅₄H₃₆N₂O = 728.9) |
| N-123 | m/z = 592.2(C₄₂H₂₈N₂S = 592.76) | N-124 | m/z = 756.22(C₅₄H₃₂N₂OS = 756.92) |
| N-125 | m/z = 547.23(C₄₂H₂₉N = 547.7) | N-126 | m/z = 672.28(C₄₉H₂₄D₇NO₂ = 672.83) |
| N-127 | m/z = 626.28(C₄₈H₂₆D₅N = 626.81) | N-128 | m/z = 558.22(C₄₀H₂₂D₅NS = 558.75) |

TABLE 5

| Compound | FD-MS | Compound | FD-MS |
|---|---|---|---|
| S-1 | m/z = 408.16(C₃₀H₂₀N₂ = 408.50) | S-2 | m/z = 534.21(C₄₀H₂₆N₂ = 534.66) |
| S-3 | m/z = 560.23(C₄₂H₂₈N₂ = 560.70) | S-4 | m/z = 584.23(C₄₄H₂₈N₂ = 584.72) |
| S-5 | m/z = 560.23(C₄₂H₂₈N₂ = 560.70) | S-6 | m/z = 634.24(C₄₈H₃₀N₂ = 634.78) |
| S-7 | m/z = 610.24(C₄₆H₃₀N₂ = 610.76) | S-8 | m/z = 498.17(C₃₆H₂₂N₂O = 498.59) |
| S-9 | m/z = 574.20(C₄₂H₂₆N₂O = 574.68) | S-10 | m/z = 660.26(C₅₀H₃₂N₂ = 660.82) |
| S-11 | m/z = 686.27(C₅₂H₃₄N₂ = 686.86) | S-12 | m/z = 620.14(C₄₂H₂₄N₂S₂ = 620.79) |
| S-13 | m/z = 640.20(C₄₆H₂₈N₂S = 640.80) | S-14 | m/z = 560.23(C₄₂H₂₈N₂ = 560.70) |
| S-15 | m/z = 558.21(C₄₂H₂₆N₂ = 558.68) | S-16 | m/z = 548.19(C₄₀H₂₄N₂O = 548.65) |
| S-17 | m/z = 573.22(C₄₂H₂₇N₃ = 573.70) | S-18 | m/z = 564.17(C₄₀H₂₄N₂S = 564.71) |
| S-19 | m/z = 574.20(C₄₂H₂₆N₂O = 574.68) | S-20 | m/z = 564.17(C₄₀H₂₄N₂S = 564.71) |
| S-21 | m/z = 564.17(C₄₀H₂₄N₂S = 564.71) | S-22 | m/z = 813.31(C₆₁H₃₉N₃ = 814.00) |
| S-23 | m/z = 696.26(C₅₃H₃₂N₂ = 696.85) | S-24 | m/z = 691.23(C₄₉H₂₉N₃O₂ = 691.79) |
| S-25 | m/z = 710.27(C₅₄H₃₄N₂ = 710.88) | S-26 | m/z = 610.24(C₄₆H₃₀N₂ = 610.76) |
| S-27 | m/z = 670.15(C₄₆H₂₆N₂S₂ = 670.85) | S-28 | m/z = 640.29(C₄₈H₃₆N₂ = 640.83) |
| S-29 | m/z = 598.20(C₄₄H₂₆N₂O = 598.71) | S-30 | m/z = 623.24(C₄₆H₂₉N₃ = 623.76) |
| S-31 | m/z = 458.18(C₃₄H₂₂N₂ = 458.56) | S-32 | m/z = 548.19(C₄₀H₂₄N₂O = 548.65) |
| S-33 | m/z = 508.19(C₃₈H₂₄N₂ = 508.62) | S-34 | m/z = 508.19(C₃₈H₂₄N₂ = 508.62) |
| S-35 | m/z = 623.24(C₄₆H₂₉N₃ = 623.76) | S-36 | m/z = 564.17(C₄₀H₂₄N₂S = 564.71) |
| S-37 | m/z = 627.20(C₄₆H₂₉NS = 627.81) | S-38 | m/z = 505.10(C₃₄H₁₉NS₂ = 505.65) |
| S-39 | m/z = 514.15(C₃₆H₂₂N₂S = 514.65) | S-40 | m/z = 575.17(C₄₂H₂₅NS = 575.73) |
| S-41 | m/z = 642.21(C₄₆H₃₀N₂S = 642.82) | S-42 | m/z = 575.17(C₄₂H₂₅NS = 575.73) |
| S-43 | m/z = 606.18(C₄₂H₂₆N₂OS = 606.74) | S-44 | m/z = 575.17(C₄₂H₂₅NS = 575.73) |
| S-45 | m/z = 551.17(C₄₀H₂₅NS = 551.71) | S-46 | m/z = 607.14(C₄₂H₂₅NS₂ = 607.79) |
| S-47 | m/z = 525.16(C₃₈H₂₃NS = 525.67) | S-48 | m/z = 642.21(C₄₆H₃₀N₂S = 642.82) |
| S-49 | m/z = 548.19(C₄₀H₂₄N₂O = 548.65) | S-50 | m/z = 473.14(C₃₄H₁₉NO₂ = 473.53) |
| S-51 | m/z = 566.15(C₃₉H₂₂N₂OS = 566.68) | S-52 | m/z = 459.16(C₃₄H₂₁NO = 459.55) |
| S-53 | m/z = 473.14(C₃₄H₁₉NO₂ = 473.53) | S-54 | m/z = 523.16(C₃₈H₂₁NO₂ = 523.59) |
| S-55 | m/z = 539.13(C₃₈H₂₁NOS = 539.65) | S-56 | m/z = 548.19(C₄₀H₂₄N₂O = 548.65) |
| S-57 | m/z = 489.12(C₃₄H₁₉NOS = 489.59) | S-58 | m/z = 545.09(C₃₆H₁₉NOS₂ = 545.67) |
| S-59 | m/z = 549.17(C₄₀H₂₃NO₂ = 549.63) | S-60 | m/z = 565.15(C₄₀H₂₃NOS = 565.69) |
| S-61 | m/z = 523.16(C₃₈H₂₁NO₂ = 523.59) | S-62 | m/z = 598.20(C₄₄H₂₆N₂O = 598.71) |
| S-63 | m/z = 539.13(C₃₈H₂₁NOS = 539.65) | S-64 | m/z = 589.15(C₄₂H₂₃NOS = 589.71) |
| S-65 | m/z = 498.17(C₃₆H₂₂N₂O = 498.59) | S-66 | m/z = 509.18(C₃₈H₂₃NO = 509.61) |
| S-67 | m/z = 548.19(C₄₀H₂₄N₂O = 548.65) | S-68 | m/z = 549.17(C₄₀H₂₃NO₂ = 549.63) |
| S-69 | m/z = 449.12(C₃₂H₁₉NS = 449.57) | S-70 | m/z = 439.10(C₃₀H₁₇NOS = 439.53) |
| S-71 | m/z = 647.22(C₄₉H₂₉NO = 647.78) | S-72 | m/z = 717.28(C₅₂H₃₅N₃O = 717.87) |
| S-73 | m/z = 459.16(C₃₄H₂₁NO = 459.55) | S-74 | m/z = 533.18(C₄₀H₂₃NO = 533.63) |
| S-75 | m/z = 525.16(C₃₈H₂₃NS = 525.67) | S-76 | m/z = 564.17(C₄₀H₂₄N₂S = 564.71) |
| S-77 | m/z = 575.19(C₄₂H₂₅NO₂ = 575.67) | S-78 | m/z = 663.22(C₄₉H₂₉NO₂ = 663.78) |
| S-79 | m/z = 647.22(C₄₉H₂₉NO = 647.78) | S-80 | m/z = 496.16(C₃₆H₂₀N₂O = 496.57) |
| S-81 | m/z = 565.15(C₄₀H₂₃NOS = 565.69) | S-82 | m/z = 505.10(C₃₄H₁₉NS₂ = 505.65) |
| S-83 | m/z = 765.25(C₅₆H₃₅NOSi = 765.99) | S-84 | m/z = 615.17(C₄₄H₂₅NOS = 615.75) |
| S-85 | m/z = 603.17(C₄₃H₂₅NOS = 603.74) | S-86 | m/z = 772.29(C₅₉H₃₆N₂ = 772.95) |
| S-87 | m/z = 802.33(C₆₁H₄₂N₂ = 803.02) | S-88 | m/z = 607.23(C₄₇H₂₉N = 607.76) |
| S-89 | m/z = 524.23(C₃₉H₂₈N₂ = 524.67) | S-90 | m/z = 665.22(C₄₉H₃₁NS = 665.85) |
| S-91 | m/z = 633.25(C₄₉H₃₁N = 633.79) | S-92 | m/z = 775.29(C₅₉H₃₇NO = 775.95) |
| S-93 | m/z = 535.23(C₄₁H₂₉N = 535.69) | S-94 | m/z = 623.22(C₄₇H₂₉NO = 623.76) |
| S-95 | m/z = 687.20(C₅₁H₂₉NS = 687.86) | S-96 | m/z = 735.29(C₅₇H₃₇N = 735.93) |
| S-97 | m/z = 611.26(C₄₇H₃₃N = 611.79) | S-98 | m/z = 679.23(C₅₀H₃₃NS = 679.88) |
| S-99 | m/z = 787.32(C₆₁H₄₁N = 788.01) | S-100 | m/z = 743.33(C₅₅H₄₁N₃ = 743.95) |
| S-101 | m/z = 485.21(C₃₇H₂₇N = 485.63) | S-102 | m/z = 471.20(C₃₆H₂₅N = 471.60) |
| S-103 | m/z = 571.19(C₄₃H₂₅NO = 571.68) | S-104 | m/z = 584.23(C₄₄H₂₈N₂ = 584.72) |
| S-105 | m/z = 539.24(C₄₀H₂₁D₅N₂ = 539.69) | S-106 | m/z = 453.15(C₃₂H₁₅NS = 471.60) |
| S-107 | m/z = 563.26(C₄₃H₂₆D₄NO = 563.74) | S-108 | m/z = 589.26(C₄₄H₂₃D₅N₂ = 584.72) |
| S-109 | m/z = 589.26(C₄₄H₂₃D₅N₂ = 589.75) | S-110 | m/z = 624.22(C₄₆H₂₈N₂O = 624.74) |
| S-111 | m/z = 589.26(C₄₄H₂₃D₅N₂ = 589.75) | S-112 | m/z = 634.24(C₄₈H₃₀N₂ = 634.78) |
| S-113 | m/z = 589.26(C₄₄H₂₃D₅N₂ = 589.75) | S-114 | m/z = 562.23(C₄₂H₂₂D₄N₂ = 562.71) |
| S-115 | m/z = 660.26(C₅₀H₃₂N₂ = 660.82) | S-116 | m/z = 553.22(C₄₀H₁₉D₅N₂O = 553.68) |
| S-117 | m/z = 634.24(C₄₈H₃₀N₂ = 634.78) | S-118 | m/z = 589.26(C₄₄H₂₃D₅N₂ = 589.75) |
| S-119 | m/z = 588.25(C₄₄H₂₄D₄N₂ = 588.75) | S-120 | m/z = 513.23(C₃₈H₁₉D₅N₂ = 513.65) |

### [Example 1] Red Organic Light Emitting Device (Phosphorescent Host)

Compound A and Compound B were used on the ITO layer (anode) formed on a glass substrate, and Compound B was doped at a weight ratio of 98:2 to form a hole injection layer with a thickness of 10 nm. Then, Compound A was vacuum-deposited on the hole injection layer with a thickness of 110 nm to form a hole transport layer.

Next, compound C-R was vacuum-deposited on the hole transport layer to a thickness of 10 nm to form an emitting auxiliary layer. Thereafter, the host material of the emitting layer uses compound P-5, a compound of the present invention, as a first host, and compound N-123, a compound of the present invention, as a second host, and uses a mixture in which the first host and the second host are mixed at a weight ratio of 5:5, and bis-(1-phenylisoquinolyl) iridium (III) acetylacetonate (hereinafter abbreviated as ‘(piq)₂Ir(acac)’) was used as the dopant material, and the dopant was doped so that the weight ratio of the host and the dopant was 95:5 to form an emitting layer with a thickness of 30 nm.

Next, compound E was vacuum-deposited on the emitting layer to form a hole-blocking layer with a thickness of 10 nm, and a mixture of compound F and compound G at a weight ratio of 5:5 was used to form an electron transport layer with a thickness of 30 nm on the hole-blocking layer. Afterwards, compound G was deposited on the electron transport layer to form an electron injection layer with a thickness of 0.2 nm, and then Al was deposited to form a cathode with a thickness of 150 nm.
Compound A: N-([1,1′-biphenyl]-4-yl)-9,9-dimethyl-N-(4-(9-phenyl-9H-carbazol-3-yl)phenyl)-9H-fluoren-2-amine
Compound B: 4,4′,4″-((1E,1′E,1″E)-cyclopropane-1,2,3-triylidenetris(cyanomethaneylylidene))tris(2,3,5,6-tetrafluorobenzonitrile)
Compound C-R N⁷-(dibenzo[b,d]thiophen-2-yl)-N²,N²,N⁷-triphenyldibenzo[b,d]thiophene-2,7-diamine
Compound E: 2-(4′-(9,9-dimethyl-9H-fluoren-2-yl)-[1,1′-biphenyl]-3-yl)-4,6-diphenyl-1,3,5-triazine
Compound F: 2,7-bis(4-(4,6-diphenyl-1,3,5-triazin-2-yl)phenyl) naphthalene
Compound G: (8-quinolinolato) lithium

### [Example 2] to [Example 34]

An organic light emitting device was manufactured in the same manner as in Example 1, except that the compounds described in Table 6 were used as the first host and second host of the emitting layer.

### [Comparative Example 1] to [Comparative Example 5]

An organic light emitting device was manufactured in the same manner as in Example 1, except that Comparative Compounds A to E were used as the first host material of the emitting layer.

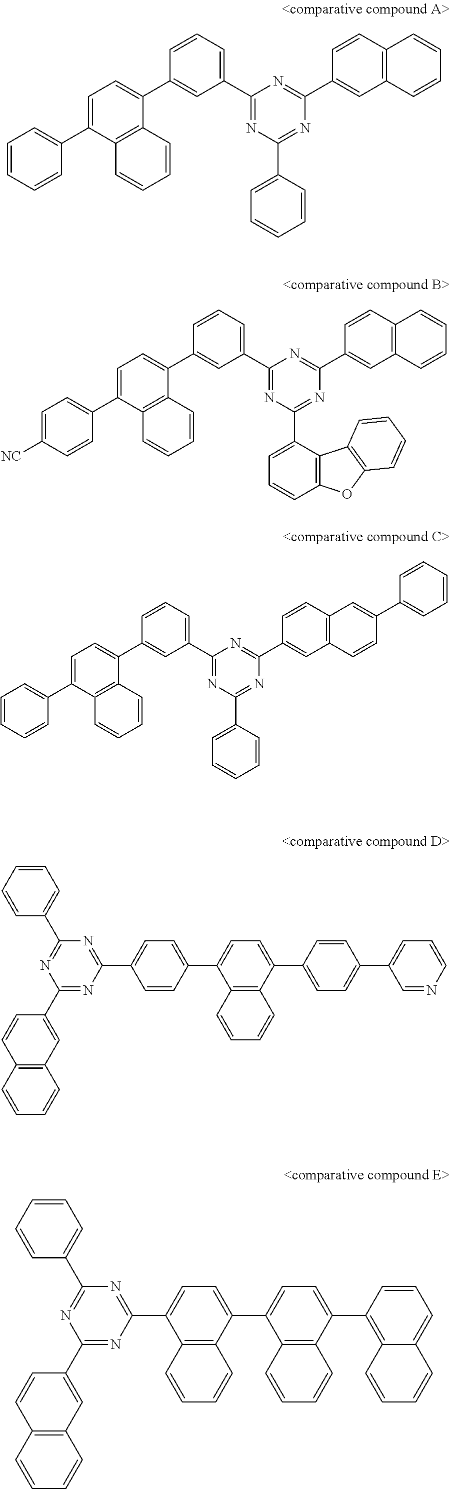 [SMILES: c1ccc(-c2nc(-c3cccc(-c4ccc(-c5ccccc5)c5ccccc45)c3)nc(-c3ccc4ccccc4c3)n2)cc1 ; N#Cc1ccc(-c2ccc(-c3cccc(-c4nc(-c5ccc6ccccc6c5)nc(-c5cccc6oc7ccccc7c56)n4)c3)c3ccccc23)cc1 ; c1ccc(-c2ccc3cc(-c4nc(-c5ccccc5)nc(-c5cccc(-c6ccc(-c7ccccc7)c7ccccc67)c5)n4)ccc3c2)cc1 ; c1ccc(-c2nc(-c3ccc(-c4ccc(-c5ccc(-c6cccnc6)cc5)c5ccccc45)cc3)nc(-c3ccc4ccccc4c3)n2)cc1 ; c1ccc(-c2nc(-c3ccc4ccccc4c3)nc(-c3ccc(-c4ccc(-c5cccc6ccccc56)c5ccccc45)c4ccccc34)n2)cc1]

The electroluminescence (EL) characteristics were measured using PR-650 from Photoresearch by applying a forward bias DC voltage to the organic electroluminescence devices manufactured by the examples and comparative examples manufactured in this way. As a result of the measurement, the T95 lifetime was measured using a lifetime measuring device manufactured by Max Science at a standard brightness of 2500 cd/m². Table 6 shows the results of the device fabrication and evaluation.

The measuring apparatus can evaluate the performance of new materials compared to comparative compounds under identical conditions, without being affected by possible daily fluctuations in deposition rate, vacuum quality or other parameters.

During the evaluation, one batch contains 4 identically prepared OLEDs including a comparative compound, and the performance of a total of 12 OLEDs is evaluated in 3 batches, so the value of the experimental results obtained in this way indicates statistical significance.

TABLE 6

| | | | Driving | Current | | |
|---|---|---|---|---|---|---|
| | First | Second | Voltage | Density | Efficiency | |
| | compound | compound | (V) | (mA/cm²) | (cd/A) | T(95) |
| Comparative | Comparative | N-123 | 5.2 | 10.4 | 24.1 | 98.6 |
| Example (1) | compound A | | | | | |
| Comparative | Comparative | N-123 | 5.4 | 11.2 | 22.3 | 92.7 |
| Example (2) | compound B | | | | | |
| Comparative | Comparative | N-123 | 5.1 | 10.6 | 23.6 | 101.5 |
| Example (3) | compound C | | | | | |
| Comparative | Comparative | N-123 | 5.3 | 12.0 | 20.9 | 91.4 |
| Example (4) | compound D | | | | | |
| Comparative | Comparative | N-123 | 5.2 | 10.7 | 23.4 | 93.0 |
| Example (5) | compound E | | | | | |
| Example (1) | P-5 | N-123 | 4.2 | 6.6 | 37.7 | 130.3 |
| Example (2) | P-7 | N-123 | 4.3 | 6.4 | 39.1 | 129.5 |
| Example (3) | P-14 | N-123 | 4.3 | 6.1 | 41.3 | 132.1 |
| Example (4) | P-15 | N-123 | 4.3 | 5.9 | 42.3 | 137.9 |
| Example (5) | P-16 | N-123 | 4.4 | 5.8 | 42.8 | 129.7 |
| Example (6) | P-17 | N-123 | 4.1 | 6.3 | 39.4 | 129.1 |
| Example (7) | P-20 | N-123 | 4.4 | 5.8 | 42.8 | 141.9 |
| Example (8) | P-24 | N-123 | 4.4 | 6.2 | 40.2 | 136.5 |
| Example (9) | P-26 | N-123 | 4.4 | 6.6 | 38.0 | 133.7 |
| Example (10) | P-27 | N-123 | 4.2 | 6.4 | 39.1 | 130.0 |
| Example (11) | P-28 | N-123 | 4.2 | 6.8 | 36.9 | 141.4 |
| Example (12) | P-29 | N-123 | 4.4 | 6.4 | 39.3 | 143.2 |
| Example (13) | P-30 | N-123 | 4.2 | 7.0 | 35.6 | 127.1 |
| Example (14) | P-37 | N-123 | 4.5 | 6.7 | 37.2 | 131.2 |
| Example (15) | P-46 | N-123 | 4.4 | 7.0 | 35.7 | 126.1 |
| Example (16) | P-54 | N-123 | 4.5 | 6.8 | 36.5 | 125.2 |
| Example (17) | P-60 | N-123 | 4.3 | 5.7 | 43.6 | 136.1 |
| Example (18) | P-64 | N-123 | 4.2 | 5.9 | 42.1 | 141.0 |
| Example (19) | P-86 | N-123 | 4.5 | 6.0 | 41.7 | 134.3 |
| Example (20) | P-94 | N-123 | 4.2 | 6.3 | 39.8 | 130.6 |
| Example (21) | P-101 | N-123 | 4.3 | 6.2 | 40.2 | 142.9 |
| Example (22) | P-107 | N-123 | 4.3 | 5.8 | 43.2 | 137.8 |
| Example (23) | P-111 | N-123 | 4.2 | 5.8 | 43.4 | 142.7 |
| Example (24) | P-114 | N-123 | 4.2 | 6.3 | 40.0 | 138.3 |
| Example (25) | P-124 | N-123 | 4.2 | 6.1 | 40.7 | 138.7 |
| Example (26) | P-5 | S-104 | 4.3 | 6.5 | 38.6 | 127.8 |
| Example (27) | P-7 | S-104 | 4.2 | 6.4 | 39.0 | 127.6 |
| Example (28) | P-15 | S-104 | 4.2 | 6.0 | 41.8 | 138.6 |
| Example (29) | P-18 | S-104 | 4.3 | 7.0 | 35.6 | 135.4 |
| Example (30) | P-24 | S-104 | 4.5 | 6.0 | 41.8 | 132.8 |
| Example (31) | P-27 | S-104 | 4.3 | 6.4 | 38.9 | 129.5 |
| Example (32) | P-29 | S-104 | 4.5 | 6.3 | 40.0 | 133.0 |
| Example (33) | P-111 | S-104 | 4.5 | 5.7 | 43.6 | 144.7 |
| Example (34) | P-124 | S-104 | 4.3 | 5.7 | 44.0 | 138.9 |

As can be seen from the results in Table 6, when a red organic light-emitting device was manufactured using the compound of the present invention as a phosphorescent host material, not only was the driving voltage of the organic light-emitting device lowered compared to when comparative compounds A to E were used, but the efficiency and lifetime were also significantly improved.

As can be seen from the above, when a plurality of compounds are mixed to form a host for the emitting layer, the characteristics differ depending on the type of the first compound and the second compound, and when the same compound is applied to the second compound, it can be confirmed that there is a significant difference in the characteristics depending on the type of the first compound. Similarly, it shows differences in driving voltage, efficiency, and lifetime depending on the type of second compound.

Comparative compounds A to E have a similar skeleton to the compound of the present invention, but differ in the substitution position and type of substituent.

The compound of the present invention is a compound in which one of the substituents of the triazine is a specific aryl moiety limited to a bonding position of [phenyl-naphthyl-phenyl] configuration, and the specific aryl moiety must be bonded in a para or meta position with respect to the phenyl base bonded to the triazine.

Additionally, another one of the substituents of the triazine must be a naphthyl group further substituted with an additional aryl group, that is, Formula 1-1 and Formula 1-2 must be bonded to the triazine, and it is characterized in that a structure in which an aryl group is substituted at the 6th position of the 2nd naphthyl group among the bonding positions is excluded.

Comparative Compound A, Comparative Compound B, Comparative Compound D and Comparative Compound E differs from the compound of the present invention in that no additional substituent is substituted on the naphthyl group directly bonded to the triazine, and Comparative Compound C differs from the compound of the present invention in that phenyl is substituted at the 6th position of the 2nd naphthyl group directly bonded to triazine.

In particular, Comparative Compound B has a structure in which a cyano group is further substituted at the R³ position of the present invention, and Comparative Compound D has a structure in which a pyridine group is further substituted at the R³ position of the present invention, which are structurally further different from the compound of the present invention in which no substituent other than hydrogen or deuterium is substituted at R¹ to R³.

As a result, it can be seen that the device performance using the compound of the present invention is superior to the device performance using comparative compounds A to E having these differences, and this result can be explained by the LUMO values of comparative compounds B to E and the compound of the present invention. Referring to Table 7, it can be seen that the compound of the present invention has a higher LUMO energy level than the comparative compounds.

TABLE 7

| | comparative | comparative | comparative | comparative | | |
|---|---|---|---|---|---|---|
| | compound B | compound C | compound D | compound E | P-114 | P-124 |
| LUMO(eV) | −2.072 | −1.959 | −1.939 | −1.924 | −1.846 | −1.902 |

The present invention shall be construed on the basis of the accompanying claims. In the case of the compound of the present invention, it participates in electron transport within the emitting layer as a first host compound, and plays a role in accepting and moving electrons entering from the electron transport layer to the emitting layer. At this time, if the LUMO energy level of the electron-transporting host is too low, excessive electrons may be injected from the electron transport layer to the emitting layer, which may break the charge balance between electrons and holes within the emitting layer.

That is, the compound of the present invention, which forms an appropriate LUMO energy level between the electron transport layer and the dopant in the emitting layer, appears to improve efficiency and lifetime by controlling electron injection into the emitting layer and effectively forming excitons within the emitting layer by preventing direct electron transfer from the electron transport layer to the dopant.

From the Tables 6 and 7, it can be confirmed that the compound of the present invention, which satisfies all complex elements such as the type of substituent and the substitution position of the substituent, exhibits a remarkable effect in organic electronic elements compared to other comparative compounds, even though the compounds have similar configurations. Through this, it can be seen that the compound of the present invention, which satisfies all specific configurations, exhibits a remarkable effect in organic electronic elements compared to other comparative compounds not described in this specification.

These results show that even in compounds with similar molecular components, depending on the type and substitution position of the substituent, the properties of compounds such as the hole characteristics, light efficiency characteristics, energy level, hole injection and mobility characteristics, charge balance of holes and electrons, volume density, and intermolecular distance of the molecule may vary significantly enough to be difficult to predict, additionally, it suggests that rather than one configuration affecting the overall results of the element, the performance of the element may vary due to complex factors.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of claims, and it shall be construed that all of the technical ideas included within the scope equivalent to the claims belong to the present invention.

1. A compound represented by Formula 1: 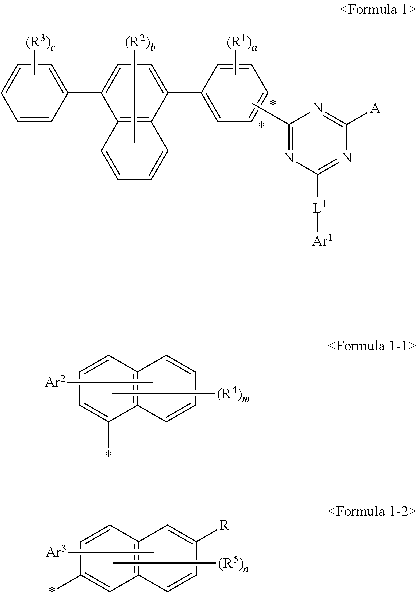
wherein:
A is represented by Formula 1-1 or Formula 1-2,
R¹, R² and R³ are, independently from each other, hydrogen or deuterium,
a is an integer of 0 to 4, b is an integer of 0 to 6, c is an integer of 0 to 5,
L¹ is a single bond or a C₆-C₆₀ arylene group,
Ar¹ is a C₆-C₆₀ aryl group or a C₂-C₆₀ heterocyclic group including at least one heteroatom of O, N, S, Si and P,
* indicates the binding position,
R⁴ and R⁵ are, independently from each other, hydrogen, deuterium or a C₆-C₆₀ aryl group,
a plurality of adjacent groups of R⁴ or a plurality of adjacent groups of R⁵ does not form a ring by bonding with each other,
R is hydrogen or deuterium,
m is an integer of 0 to 6, and n is an integer of 0 to 5,
Ar² and Ar³ are, independently from each other, a C₆-C₆₀ aryl group,
wherein the aryl group, arylene group and heterocyclic group may be substituted with one or more substituents selected from the group consisting of deuterium; a C₁-C₂₀ alkyl group; a C₆-C₂₀ aryl group; a C₆-C₂₀ aryl group substituted with deuterium; and a C₃-C₆₀ aliphatic ring, and the hydrogen(s) of the substituents may be further substituted with one or more deuterium.

2. The compound according to claim 1, wherein Ar¹ is represented by one of Formulas (A-1) to (A-5): 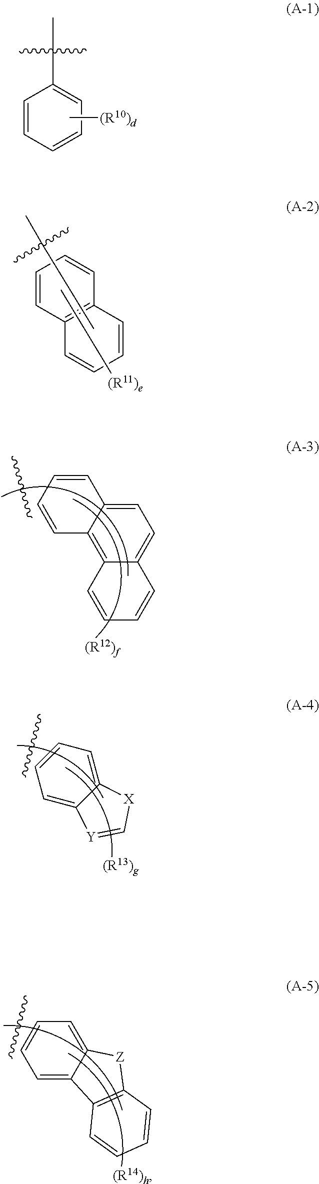
wherein:
R¹⁰, R¹¹, R¹², R¹³ and R¹⁴ are, independently from each other, selected from the group consisting of hydroge; deuterium; a C₆-C₂₀ aryl group; and a C₆-C₂₀ aryl group substituted with deuterium,
an adjacent plurality of R¹⁰s or a plurality of R¹¹s or a plurality of R¹²s or a plurality of R¹³s or a plurality of R¹⁴s may be bonded to each other to form a ring,
d is an integer of 0 to 5,
e and h are an integer of 0 to 7, f is an integer of 0 to 9, g is an integer of 0 to 4,
X and Z are independently O or S,
Y is N or C, and [symbol]
indicates a position to be bonded.

3. The compound according to claim 1, wherein L¹ is represented by one of Formulas (L-1) to (L-19): 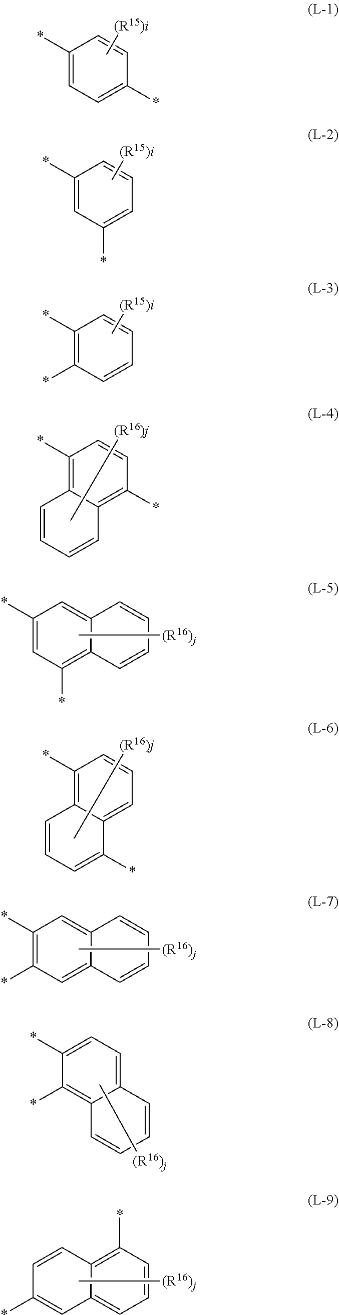 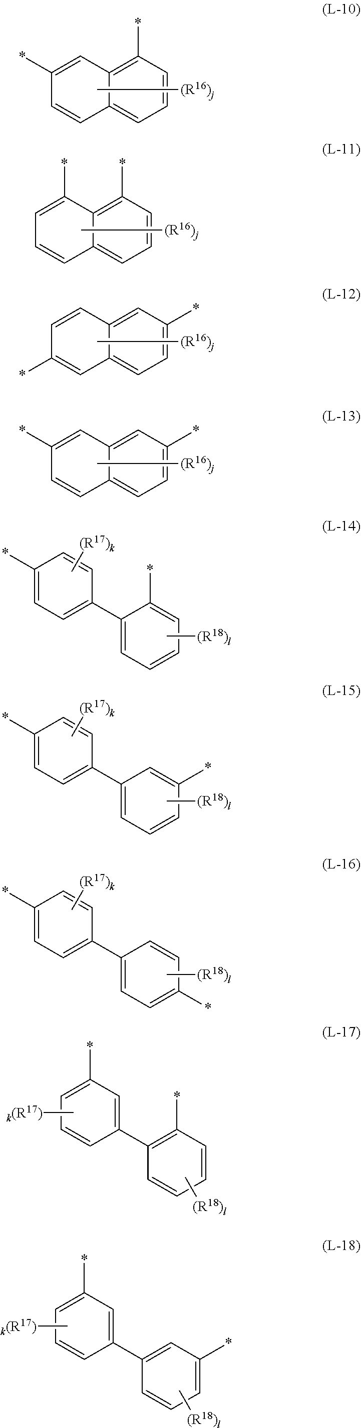 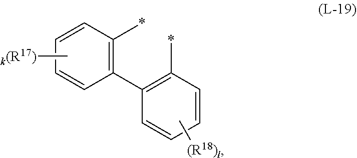
wherein:
R¹⁵, R¹⁶, R¹⁷ and R¹⁸ are, independently from each other, hydrogen or deuterium,
i, k and l are each independently an integer of 0 to 4, and j is an integer of 0 to 6.

4. The compound according to claim 1, selected from the group consisting of the following compounds:

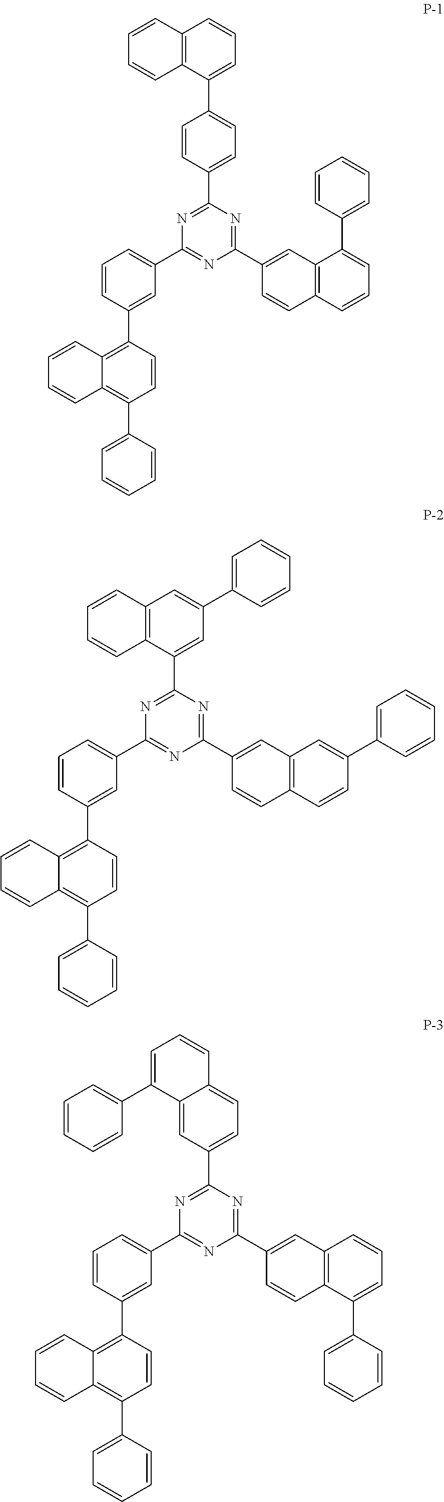 [SMILES: c1ccc(-c2cccc3ccc(-c4nc(-c5ccc(-c6cccc7ccccc67)cc5)nc(-c5cccc(-c6ccc(-c7ccccc7)c7ccccc67)c5)n4)cc23)cc1 ; c1ccc(-c2ccc3ccc(-c4nc(-c5cccc(-c6ccc(-c7ccccc7)c7ccccc67)c5)nc(-c5cc(-c6ccccc6)cc6ccccc56)n4)cc3c2)cc1 ; c1ccc(-c2cccc3cc(-c4nc(-c5cccc(-c6ccc(-c7ccccc7)c7ccccc67)c5)nc(-c5ccc6cccc(-c7ccccc7)c6c5)n4)ccc23)cc1]

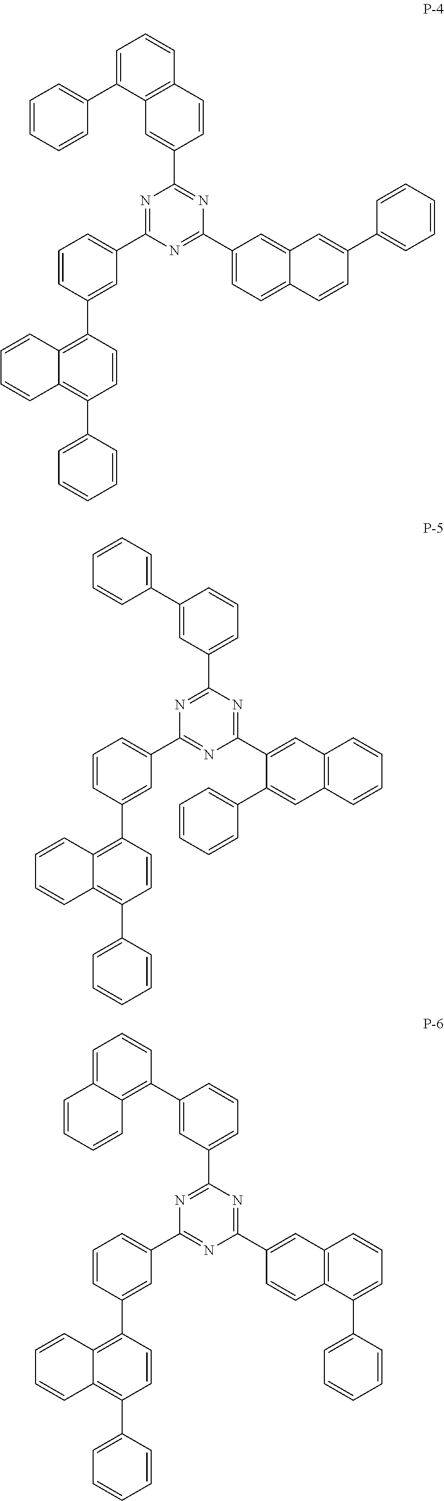

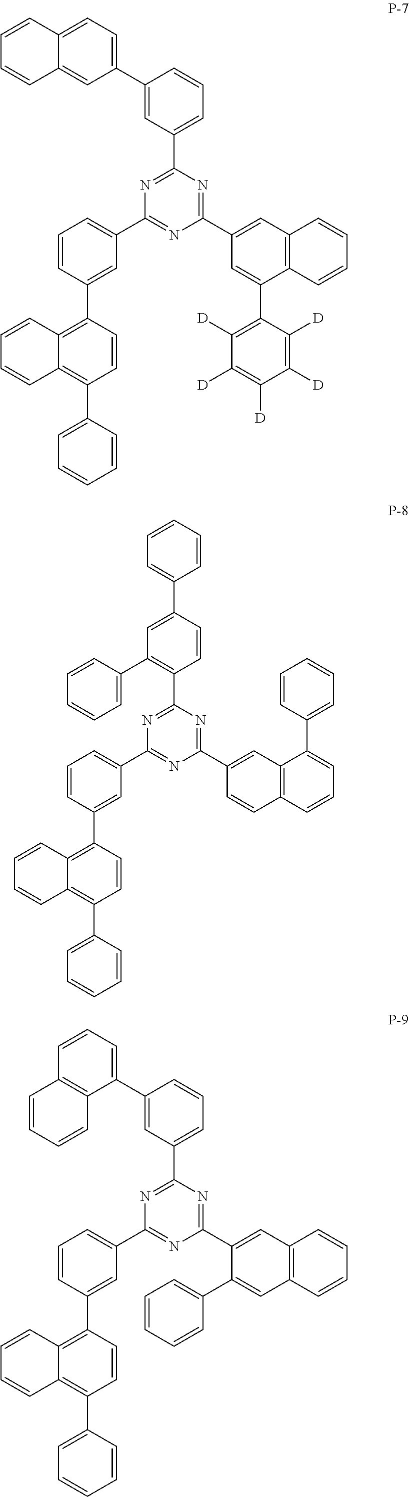

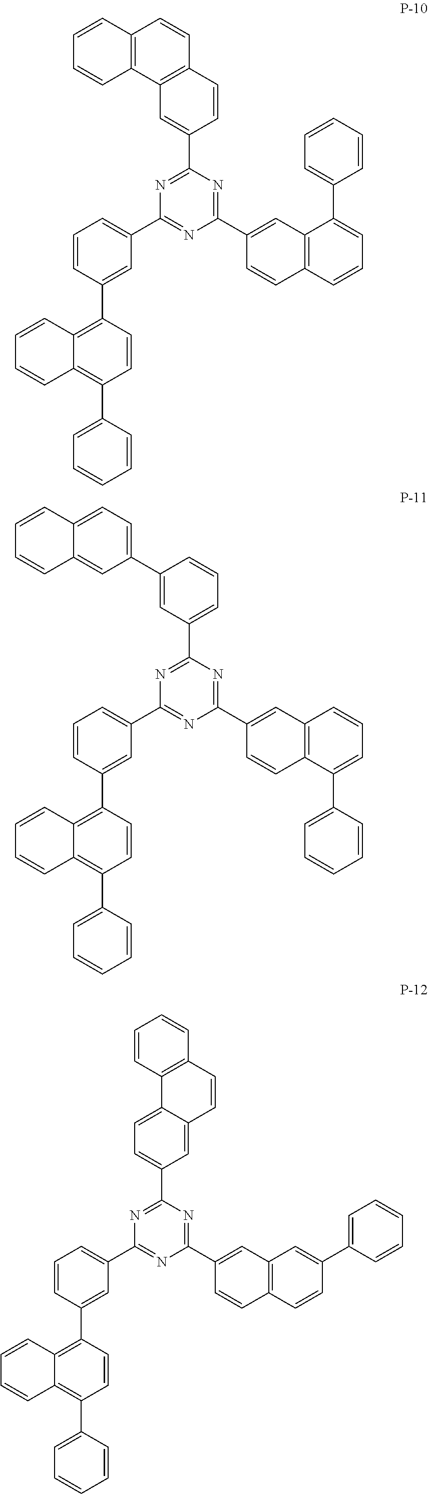

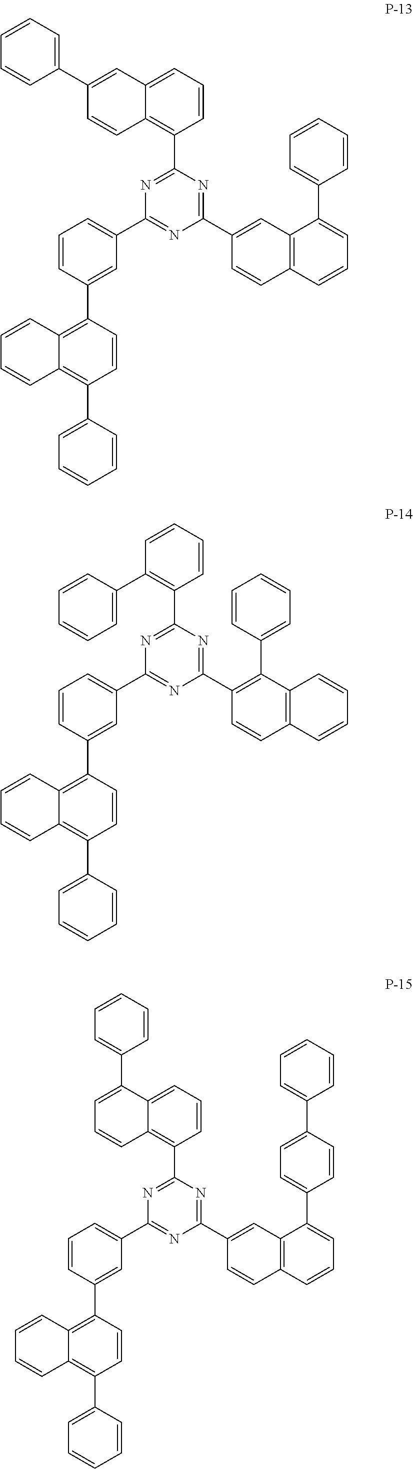 [SMILES: c1ccc(-c2ccc3c(-c4nc(-c5cccc(-c6ccc(-c7ccccc7)c7ccccc67)c5)nc(-c5ccc6cccc(-c7ccccc7)c6c5)n4)cccc3c2)cc1 ; c1ccc(-c2ccccc2-c2nc(-c3cccc(-c4ccc(-c5ccccc5)c5ccccc45)c3)nc(-c3ccc4ccccc4c3-c3ccccc3)n2)cc1 ; c1ccc(-c2ccc(-c3cccc4ccc(-c5nc(-c6cccc(-c7ccc(-c8ccccc8)c8ccccc78)c6)nc(-c6cccc7c(-c8ccccc8)cccc67)n5)cc34)cc2)cc1]

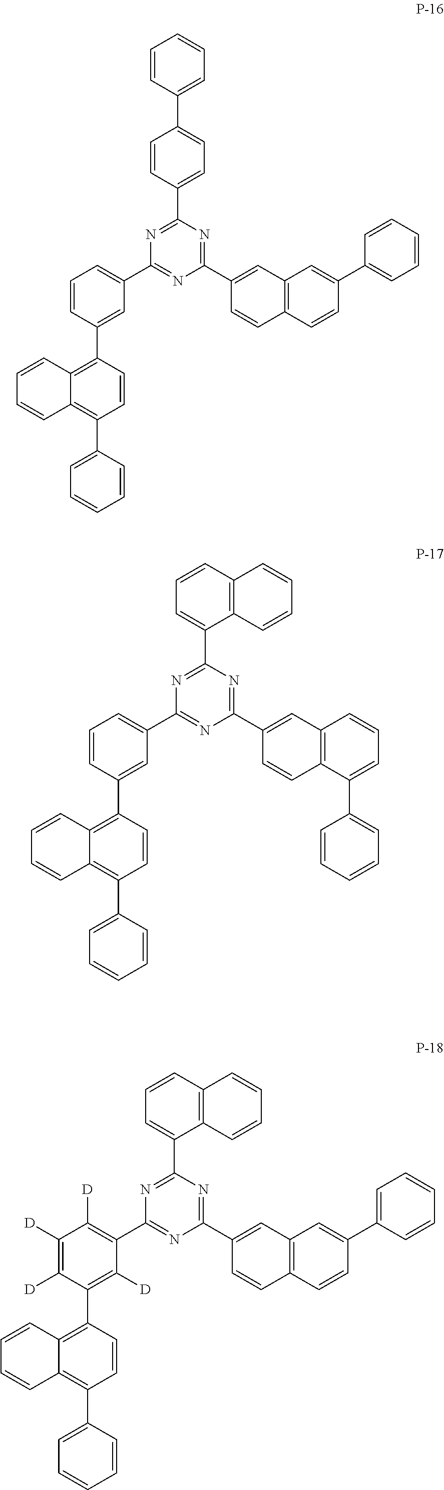 [SMILES: c1ccc(-c2ccc(-c3nc(-c4cccc(-c5ccc(-c6ccccc6)c6ccccc56)c4)nc(-c4ccc5ccc(-c6ccccc6)cc5c4)n3)cc2)cc1 ; c1ccc(-c2cccc3cc(-c4nc(-c5cccc(-c6ccc(-c7ccccc7)c7ccccc67)c5)nc(-c5cccc6ccccc56)n4)ccc23)cc1 ; [2H]c1c([2H])c(-c2nc(-c3ccc4ccc(-c5ccccc5)cc4c3)nc(-c3cccc4ccccc34)n2)c([2H])c(-c2ccc(-c3ccccc3)c3ccccc23)c1[2H]]

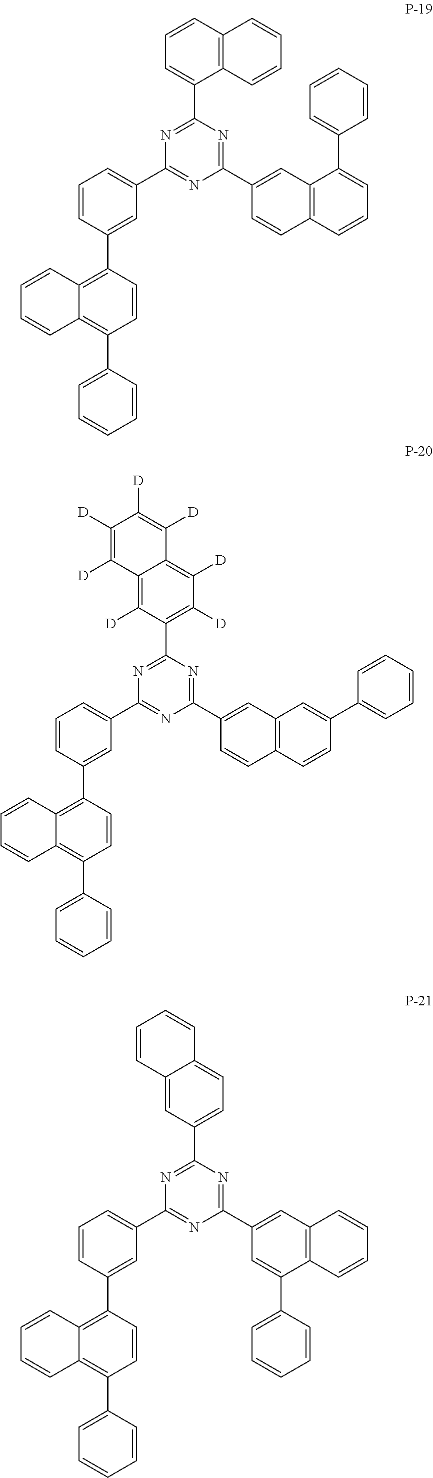 [SMILES: c1ccc(-c2cccc3ccc(-c4nc(-c5cccc(-c6ccc(-c7ccccc7)c7ccccc67)c5)nc(-c5cccc6ccccc56)n4)cc23)cc1 ; [2H]c1c([2H])c([2H])c2c([2H])c(-c3nc(-c4cccc(-c5ccc(-c6ccccc6)c6ccccc56)c4)nc(-c4ccc5ccc(-c6ccccc6)cc5c4)n3)c([2H])c([2H])c2c1[2H] ; c1ccc(-c2cc(-c3nc(-c4cccc(-c5ccc(-c6ccccc6)c6ccccc56)c4)nc(-c4ccc5ccccc5c4)n3)cc3ccccc23)cc1]

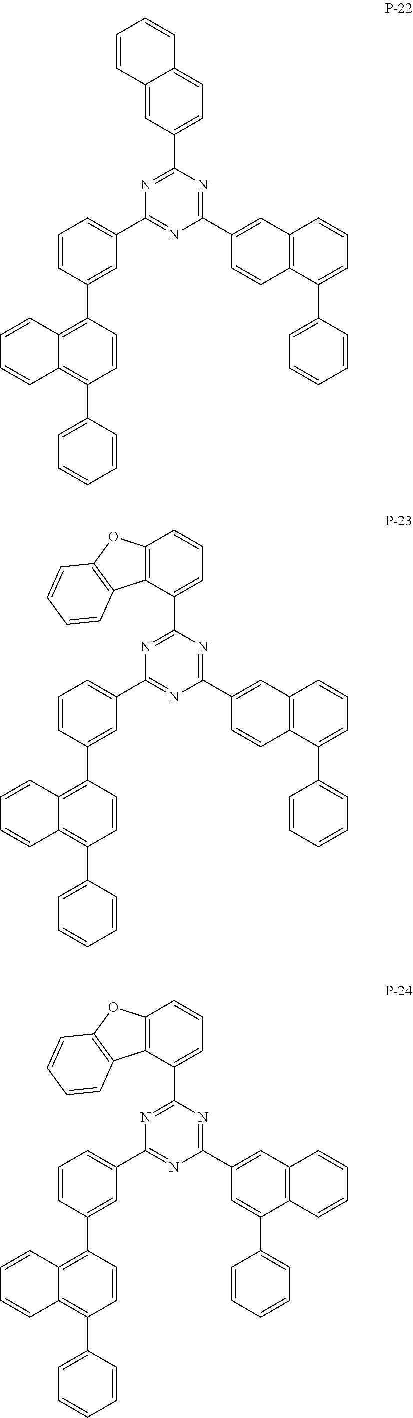 [SMILES: c1ccc(-c2cccc3cc(-c4nc(-c5cccc(-c6ccc(-c7ccccc7)c7ccccc67)c5)nc(-c5ccc6ccccc6c5)n4)ccc23)cc1 ; c1ccc(-c2cccc3cc(-c4nc(-c5cccc(-c6ccc(-c7ccccc7)c7ccccc67)c5)nc(-c5cccc6oc7ccccc7c56)n4)ccc23)cc1 ; c1ccc(-c2cc(-c3nc(-c4cccc(-c5ccc(-c6ccccc6)c6ccccc56)c4)nc(-c4cccc5oc6ccccc6c45)n3)cc3ccccc23)cc1]

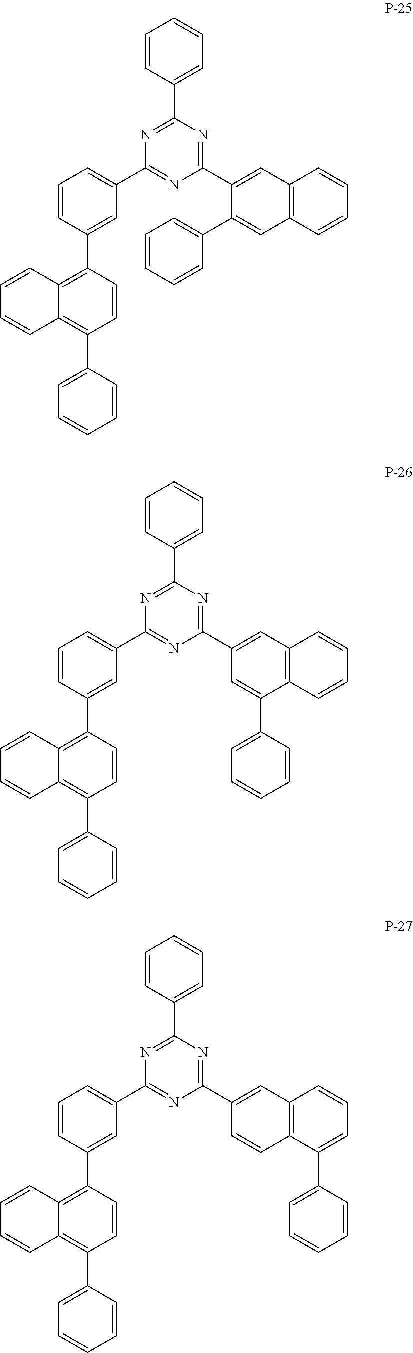 [SMILES: c1ccc(-c2nc(-c3cccc(-c4ccc(-c5ccccc5)c5ccccc45)c3)nc(-c3cc4ccccc4cc3-c3ccccc3)n2)cc1 ; c1ccc(-c2nc(-c3cccc(-c4ccc(-c5ccccc5)c5ccccc45)c3)nc(-c3cc(-c4ccccc4)c4ccccc4c3)n2)cc1 ; c1ccc(-c2nc(-c3cccc(-c4ccc(-c5ccccc5)c5ccccc45)c3)nc(-c3ccc4c(-c5ccccc5)cccc4c3)n2)cc1]

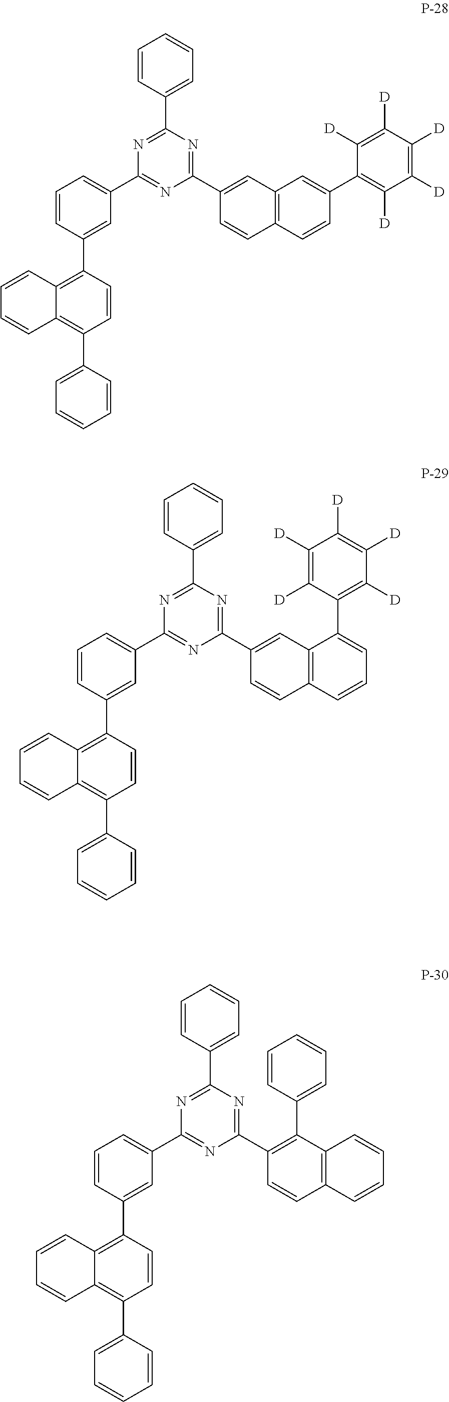 [SMILES: [2H]c1c([2H])c([2H])c(-c2ccc3ccc(-c4nc(-c5ccccc5)nc(-c5cccc(-c6ccc(-c7ccccc7)c7ccccc67)c5)n4)cc3c2)c([2H])c1[2H] ; [2H]c1c([2H])c([2H])c(-c2cccc3ccc(-c4nc(-c5ccccc5)nc(-c5cccc(-c6ccc(-c7ccccc7)c7ccccc67)c5)n4)cc23)c([2H])c1[2H] ; c1ccc(-c2nc(-c3cccc(-c4ccc(-c5ccccc5)c5ccccc45)c3)nc(-c3ccc4ccccc4c3-c3ccccc3)n2)cc1]

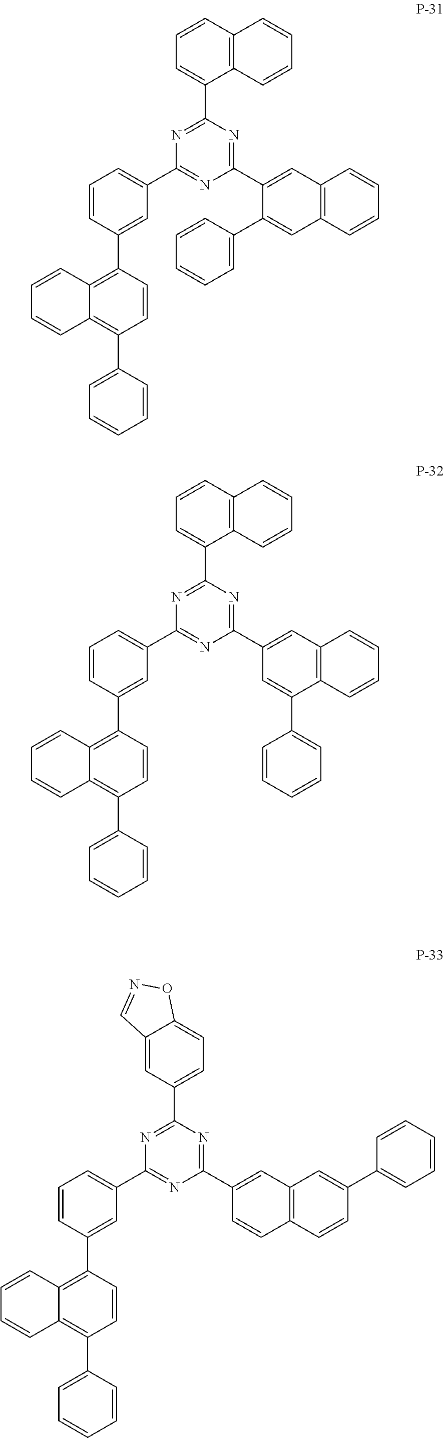 [SMILES: c1ccc(-c2cc3ccccc3cc2-c2nc(-c3cccc(-c4ccc(-c5ccccc5)c5ccccc45)c3)nc(-c3cccc4ccccc34)n2)cc1 ; c1ccc(-c2cc(-c3nc(-c4cccc(-c5ccc(-c6ccccc6)c6ccccc56)c4)nc(-c4cccc5ccccc45)n3)cc3ccccc23)cc1 ; c1ccc(-c2ccc3ccc(-c4nc(-c5cccc(-c6ccc(-c7ccccc7)c7ccccc67)c5)nc(-c5ccc6oncc6c5)n4)cc3c2)cc1]

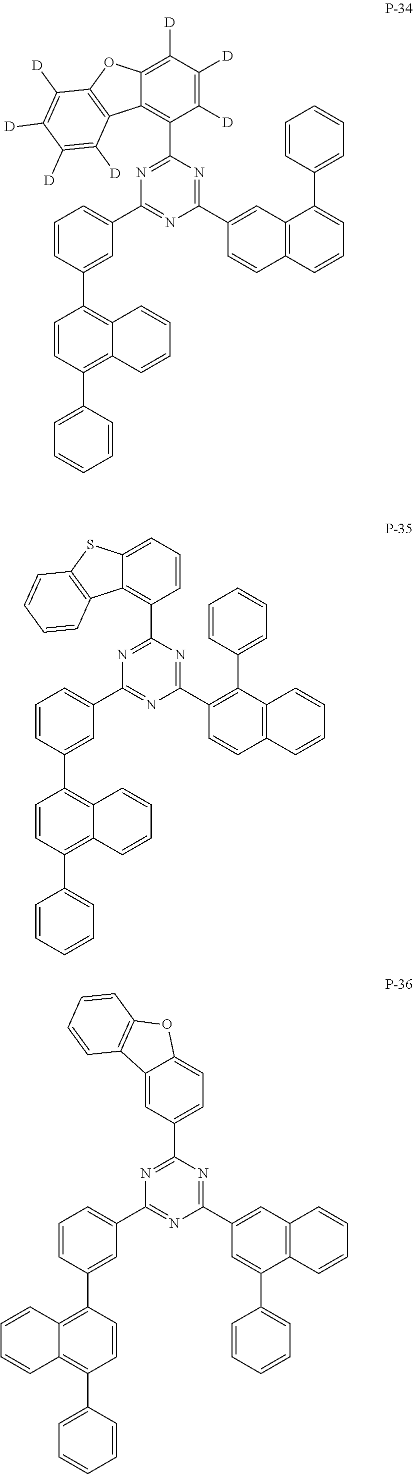

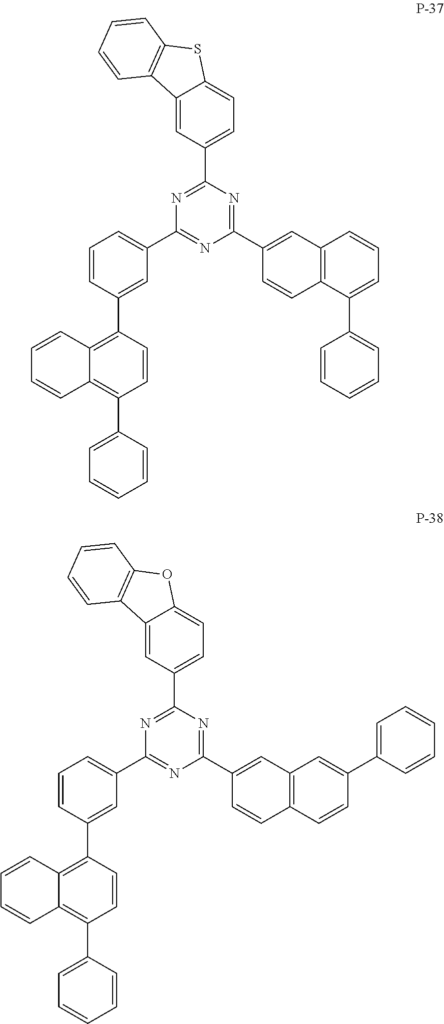

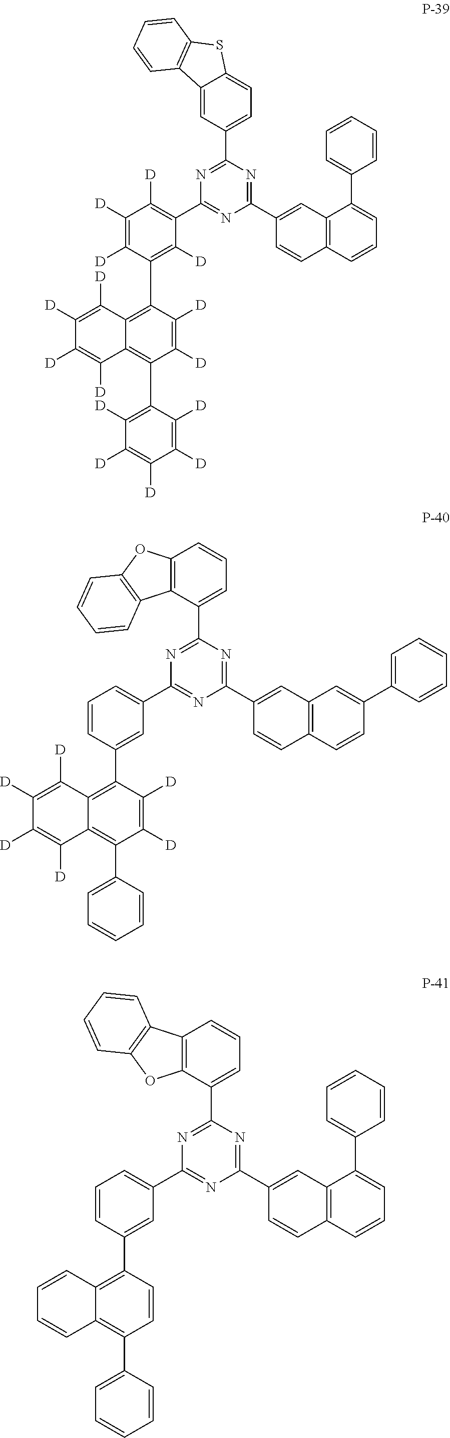 [SMILES: [2H]c1c([2H])c([2H])c(-c2c([2H])c([2H])c(-c3c([2H])c([2H])c([2H])c(-c4nc(-c5ccc6cccc(-c7ccccc7)c6c5)nc(-c5ccc6sc7ccccc7c6c5)n4)c3[2H])c3c([2H])c([2H])c([2H])c([2H])c23)c([2H])c1[2H] ; [2H]c1c([2H])c([2H])c2c(-c3cccc(-c4nc(-c5ccc6ccc(-c7ccccc7)cc6c5)nc(-c5cccc6oc7ccccc7c56)n4)c3)c([2H])c([2H])c(-c3ccccc3)c2c1[2H] ; c1ccc(-c2cccc3ccc(-c4nc(-c5cccc(-c6ccc(-c7ccccc7)c7ccccc67)c5)nc(-c5cccc6c5oc5ccccc56)n4)cc23)cc1]

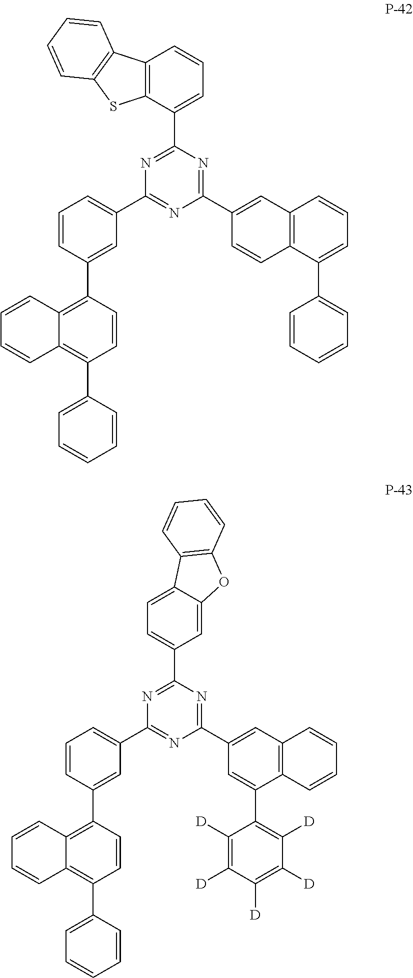 [SMILES: c1ccc(-c2cccc3cc(-c4nc(-c5cccc(-c6ccc(-c7ccccc7)c7ccccc67)c5)nc(-c5cccc6c5sc5ccccc56)n4)ccc23)cc1 ; [2H]c1c([2H])c([2H])c(-c2cc(-c3nc(-c4cccc(-c5ccc(-c6ccccc6)c6ccccc56)c4)nc(-c4ccc5c(c4)oc4ccccc45)n3)cc3ccccc23)c([2H])c1[2H]]

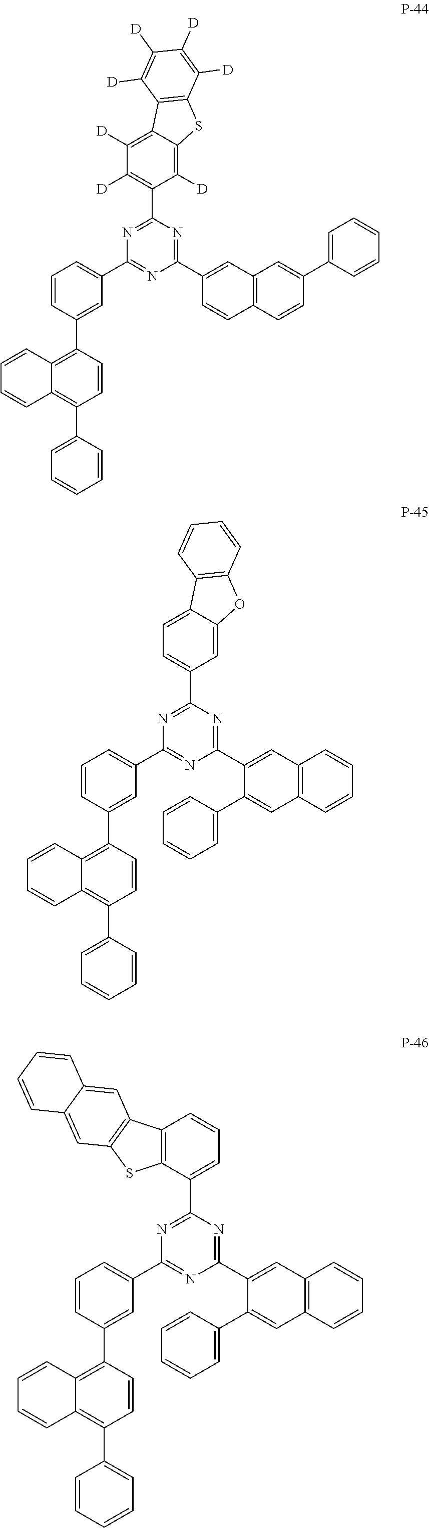 [SMILES: [2H]c1c([2H])c([2H])c2c(sc3c([2H])c(-c4nc(-c5cccc(-c6ccc(-c7ccccc7)c7ccccc67)c5)nc(-c5ccc6ccc(-c7ccccc7)cc6c5)n4)c([2H])c([2H])c32)c1[2H] ; c1ccc(-c2cc3ccccc3cc2-c2nc(-c3cccc(-c4ccc(-c5ccccc5)c5ccccc45)c3)nc(-c3ccc4c(c3)oc3ccccc34)n2)cc1 ; c1ccc(-c2cc3ccccc3cc2-c2nc(-c3cccc(-c4ccc(-c5ccccc5)c5ccccc45)c3)nc(-c3cccc4c3sc3cc5ccccc5cc34)n2)cc1]

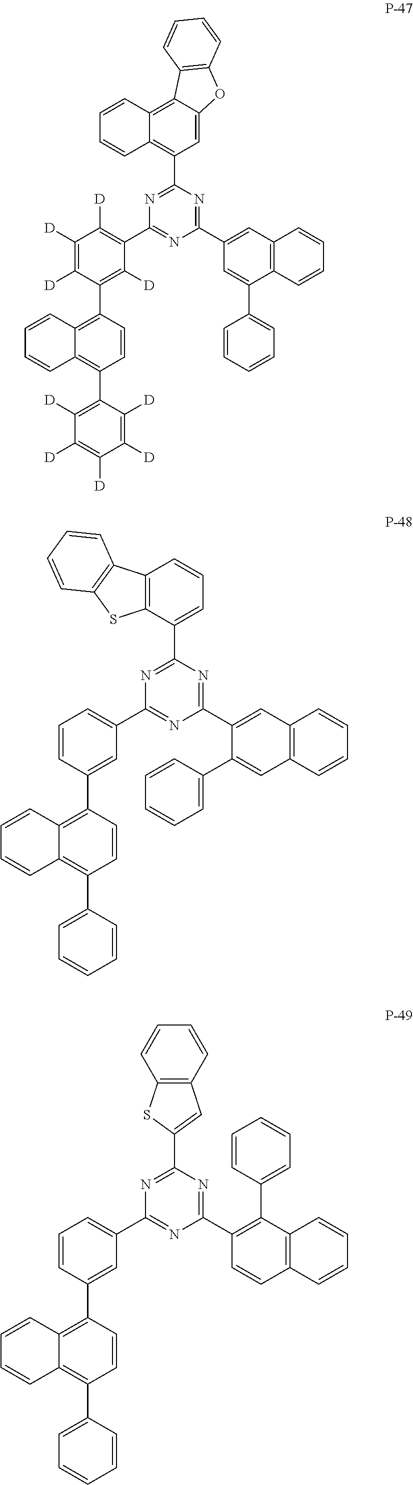 [SMILES: [2H]c1c([2H])c([2H])c(-c2ccc(-c3c([2H])c([2H])c([2H])c(-c4nc(-c5cc(-c6ccccc6)c6ccccc6c5)nc(-c5cc6oc7ccccc7c6c6ccccc56)n4)c3[2H])c3ccccc23)c([2H])c1[2H] ; c1ccc(-c2cc3ccccc3cc2-c2nc(-c3cccc(-c4ccc(-c5ccccc5)c5ccccc45)c3)nc(-c3cccc4c3sc3ccccc34)n2)cc1 ; c1ccc(-c2c(-c3nc(-c4cccc(-c5ccc(-c6ccccc6)c6ccccc56)c4)nc(-c4cc5ccccc5s4)n3)ccc3ccccc23)cc1]

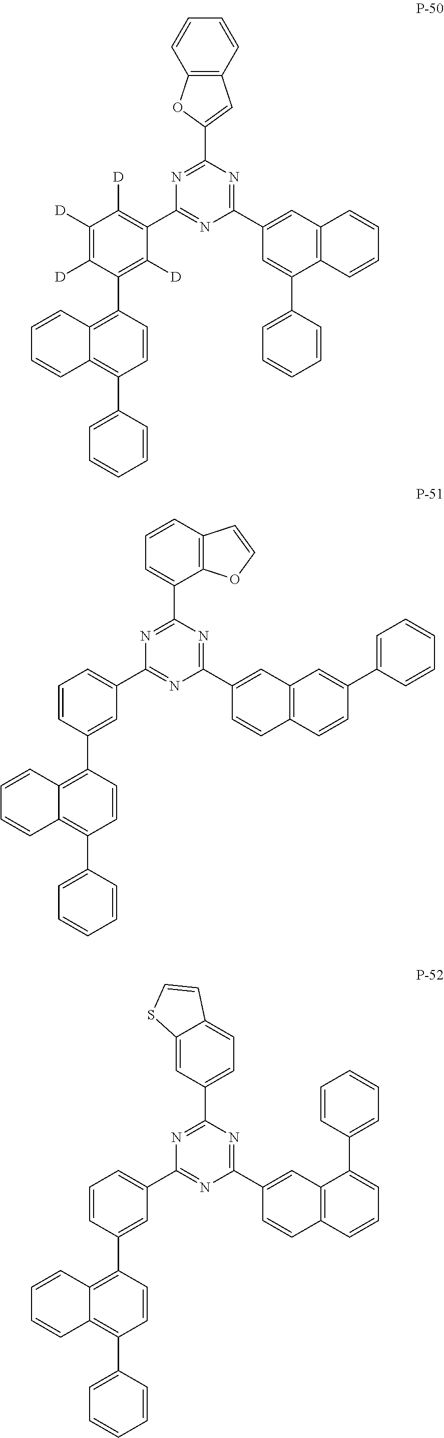 [SMILES: [2H]c1c([2H])c(-c2nc(-c3cc(-c4ccccc4)c4ccccc4c3)nc(-c3cc4ccccc4o3)n2)c([2H])c(-c2ccc(-c3ccccc3)c3ccccc23)c1[2H] ; c1ccc(-c2ccc3ccc(-c4nc(-c5cccc(-c6ccc(-c7ccccc7)c7ccccc67)c5)nc(-c5cccc6ccoc56)n4)cc3c2)cc1 ; c1ccc(-c2cccc3ccc(-c4nc(-c5cccc(-c6ccc(-c7ccccc7)c7ccccc67)c5)nc(-c5ccc6ccsc6c5)n4)cc23)cc1]

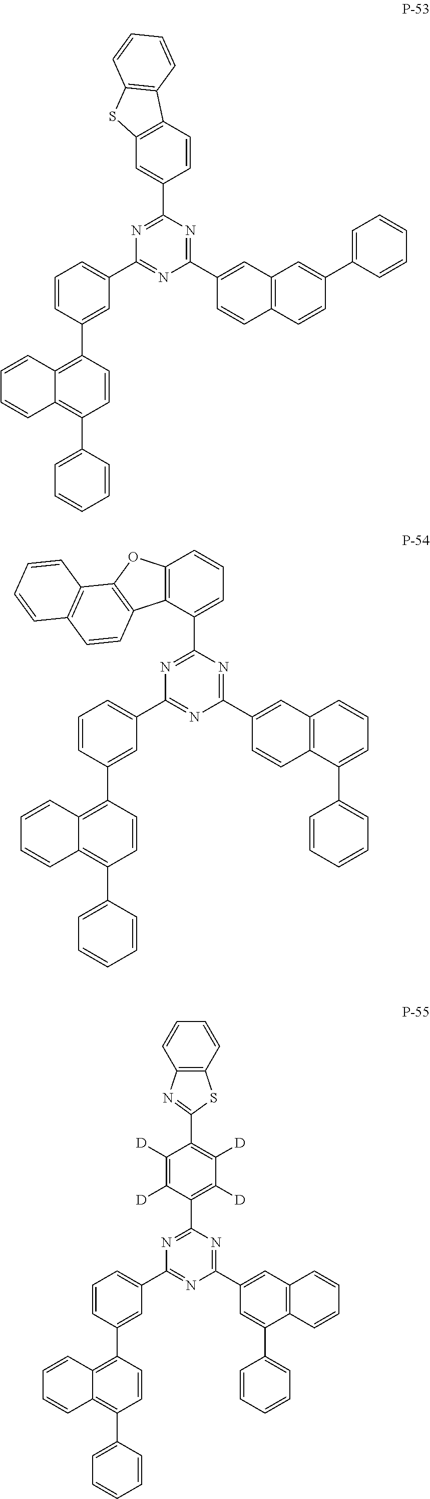 [SMILES: c1ccc(-c2ccc3ccc(-c4nc(-c5cccc(-c6ccc(-c7ccccc7)c7ccccc67)c5)nc(-c5ccc6c(c5)sc5ccccc56)n4)cc3c2)cc1 ; c1ccc(-c2cccc3cc(-c4nc(-c5cccc(-c6ccc(-c7ccccc7)c7ccccc67)c5)nc(-c5cccc6oc7c8ccccc8ccc7c56)n4)ccc23)cc1 ; [2H]c1c([2H])c(-c2nc3ccccc3s2)c([2H])c([2H])c1-c1nc(-c2cccc(-c3ccc(-c4ccccc4)c4ccccc34)c2)nc(-c2cc(-c3ccccc3)c3ccccc3c2)n1]

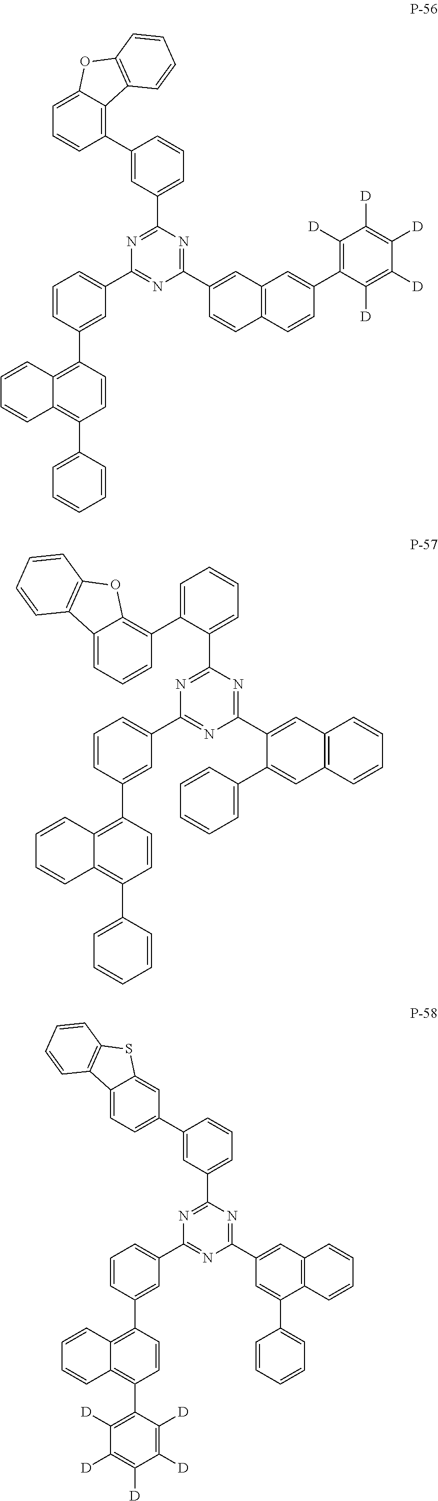 [SMILES: [2H]c1c([2H])c([2H])c(-c2ccc3ccc(-c4nc(-c5cccc(-c6ccc(-c7ccccc7)c7ccccc67)c5)nc(-c5cccc(-c6cccc7oc8ccccc8c67)c5)n4)cc3c2)c([2H])c1[2H] ; c1ccc(-c2cc3ccccc3cc2-c2nc(-c3cccc(-c4ccc(-c5ccccc5)c5ccccc45)c3)nc(-c3ccccc3-c3cccc4c3oc3ccccc34)n2)cc1 ; [2H]c1c([2H])c([2H])c(-c2ccc(-c3cccc(-c4nc(-c5cccc(-c6ccc7c(c6)sc6ccccc67)c5)nc(-c5cc(-c6ccccc6)c6ccccc6c5)n4)c3)c3ccccc23)c([2H])c1[2H]]

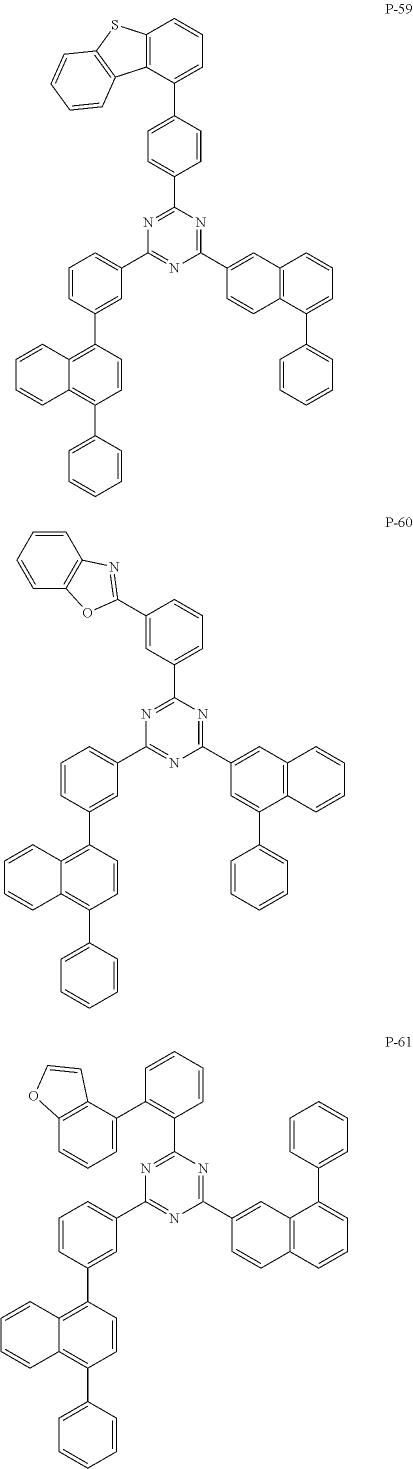 [SMILES: c1ccc(-c2cccc3cc(-c4nc(-c5ccc(-c6cccc7sc8ccccc8c67)cc5)nc(-c5cccc(-c6ccc(-c7ccccc7)c7ccccc67)c5)n4)ccc23)cc1 ; c1ccc(-c2cc(-c3nc(-c4cccc(-c5nc6ccccc6o5)c4)nc(-c4cccc(-c5ccc(-c6ccccc6)c6ccccc56)c4)n3)cc3ccccc23)cc1 ; c1ccc(-c2cccc3ccc(-c4nc(-c5cccc(-c6ccc(-c7ccccc7)c7ccccc67)c5)nc(-c5ccccc5-c5cccc6occc56)n4)cc23)cc1]

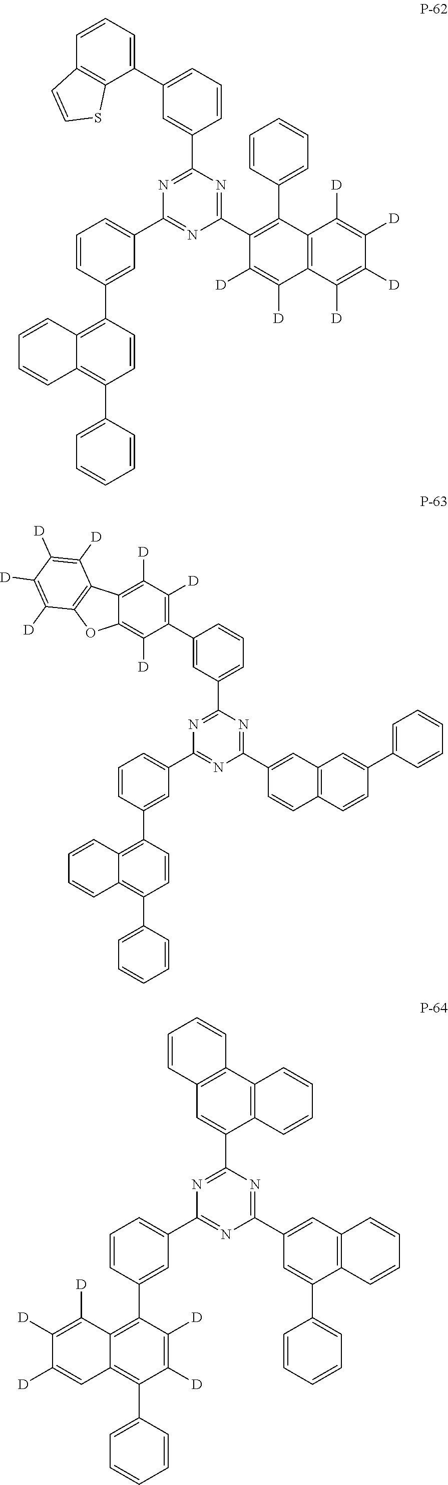 [SMILES: [2H]c1c([2H])c([2H])c2c(-c3ccccc3)c(-c3nc(-c4cccc(-c5ccc(-c6ccccc6)c6ccccc56)c4)nc(-c4cccc(-c5cccc6ccsc56)c4)n3)c([2H])c([2H])c2c1[2H] ; [2H]c1c([2H])c([2H])c2c(oc3c([2H])c(-c4cccc(-c5nc(-c6cccc(-c7ccc(-c8ccccc8)c8ccccc78)c6)nc(-c6ccc7ccc(-c8ccccc8)cc7c6)n5)c4)c([2H])c([2H])c32)c1[2H] ; [2H]c1cc2c(-c3ccccc3)c([2H])c([2H])c(-c3cccc(-c4nc(-c5cc(-c6ccccc6)c6ccccc6c5)nc(-c5cc6ccccc6c6ccccc56)n4)c3)c2c([2H])c1[2H]]

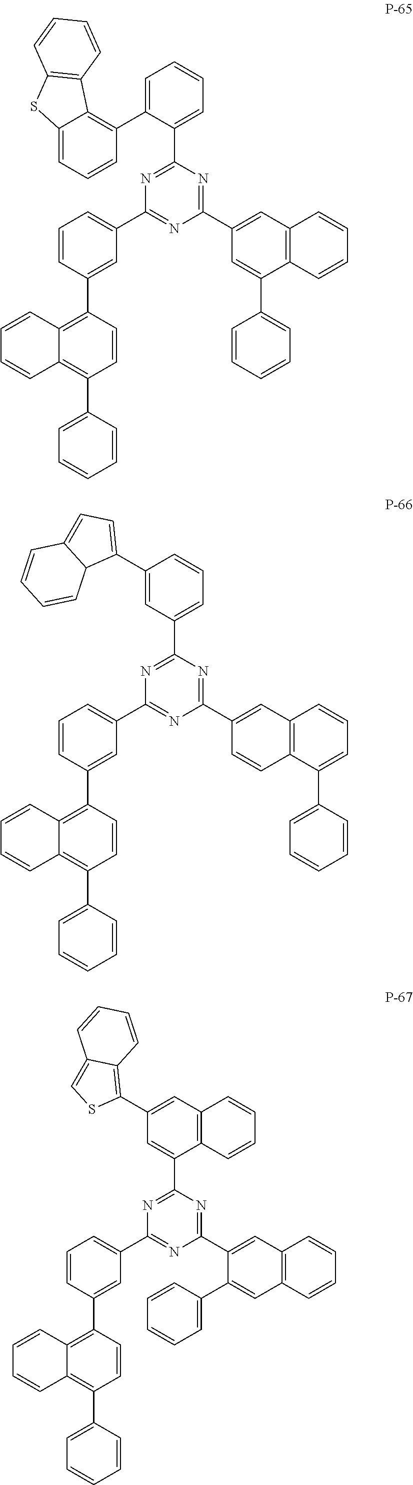

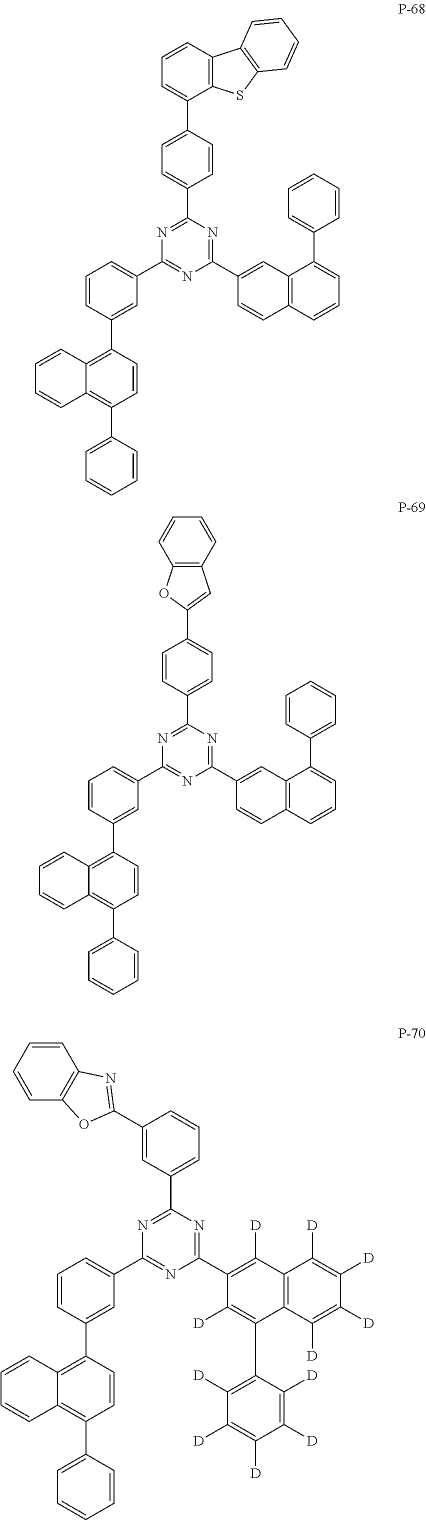 [SMILES: c1ccc(-c2cccc3ccc(-c4nc(-c5ccc(-c6cccc7c6sc6ccccc67)cc5)nc(-c5cccc(-c6ccc(-c7ccccc7)c7ccccc67)c5)n4)cc23)cc1 ; c1ccc(-c2cccc3ccc(-c4nc(-c5ccc(-c6cc7ccccc7o6)cc5)nc(-c5cccc(-c6ccc(-c7ccccc7)c7ccccc67)c5)n4)cc23)cc1 ; [2H]c1c([2H])c([2H])c(-c2c([2H])c(-c3nc(-c4cccc(-c5nc6ccccc6o5)c4)nc(-c4cccc(-c5ccc(-c6ccccc6)c6ccccc56)c4)n3)c([2H])c3c([2H])c([2H])c([2H])c([2H])c23)c([2H])c1[2H]]

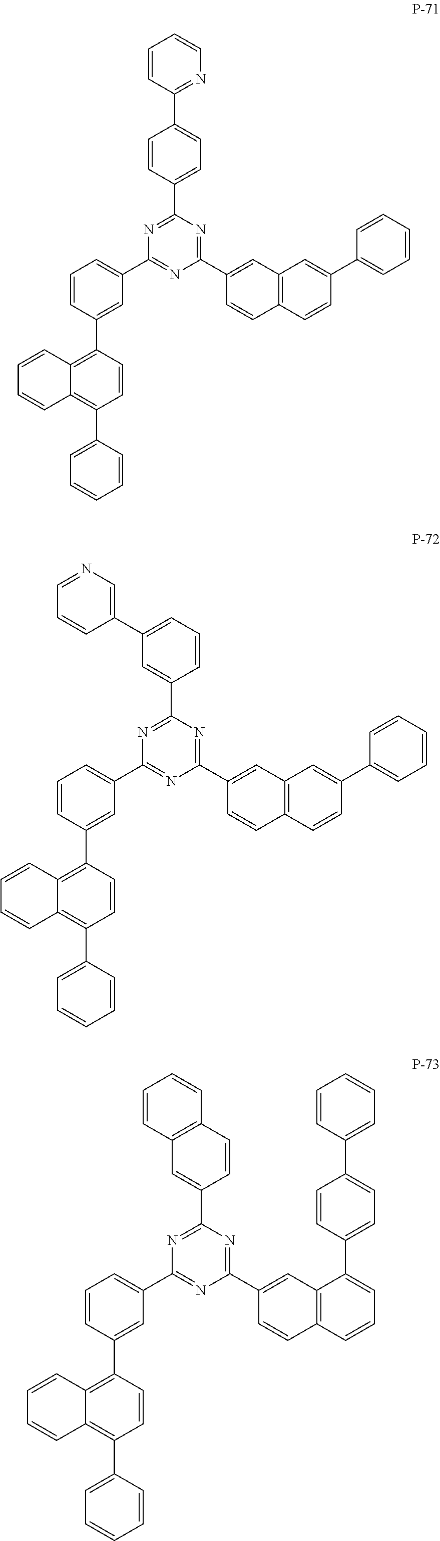 [SMILES: c1ccc(-c2ccc3ccc(-c4nc(-c5ccc(-c6ccccn6)cc5)nc(-c5cccc(-c6ccc(-c7ccccc7)c7ccccc67)c5)n4)cc3c2)cc1 ; c1ccc(-c2ccc3ccc(-c4nc(-c5cccc(-c6cccnc6)c5)nc(-c5cccc(-c6ccc(-c7ccccc7)c7ccccc67)c5)n4)cc3c2)cc1 ; c1ccc(-c2ccc(-c3cccc4ccc(-c5nc(-c6cccc(-c7ccc(-c8ccccc8)c8ccccc78)c6)nc(-c6ccc7ccccc7c6)n5)cc34)cc2)cc1]

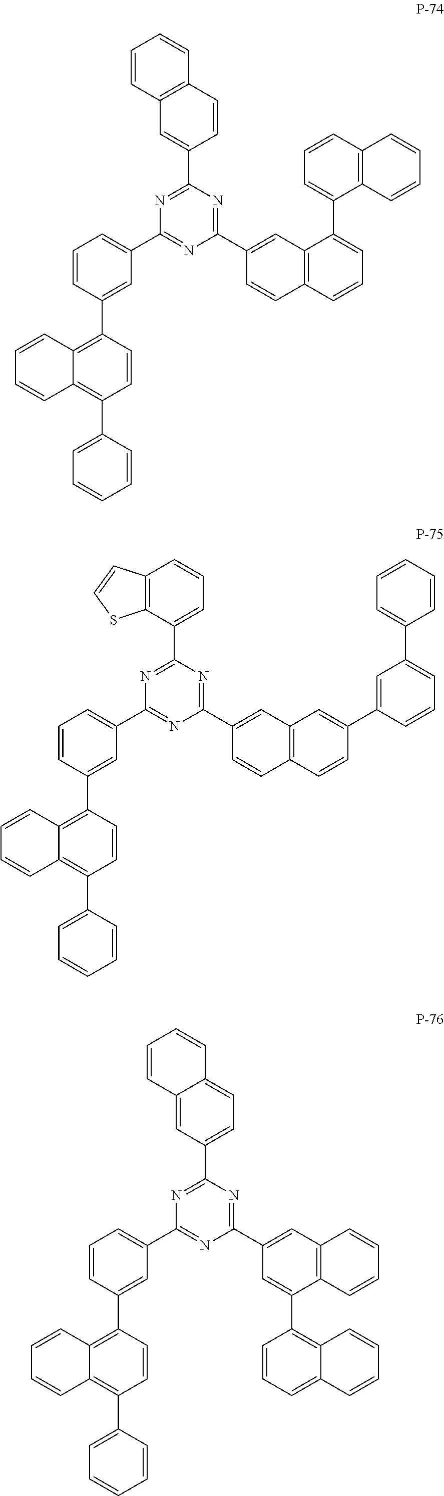 [SMILES: c1ccc(-c2ccc(-c3cccc(-c4nc(-c5ccc6ccccc6c5)nc(-c5ccc6cccc(-c7cccc8ccccc78)c6c5)n4)c3)c3ccccc23)cc1 ; c1ccc(-c2cccc(-c3ccc4ccc(-c5nc(-c6cccc(-c7ccc(-c8ccccc8)c8ccccc78)c6)nc(-c6cccc7ccsc67)n5)cc4c3)c2)cc1 ; c1ccc(-c2ccc(-c3cccc(-c4nc(-c5ccc6ccccc6c5)nc(-c5cc(-c6cccc7ccccc67)c6ccccc6c5)n4)c3)c3ccccc23)cc1]

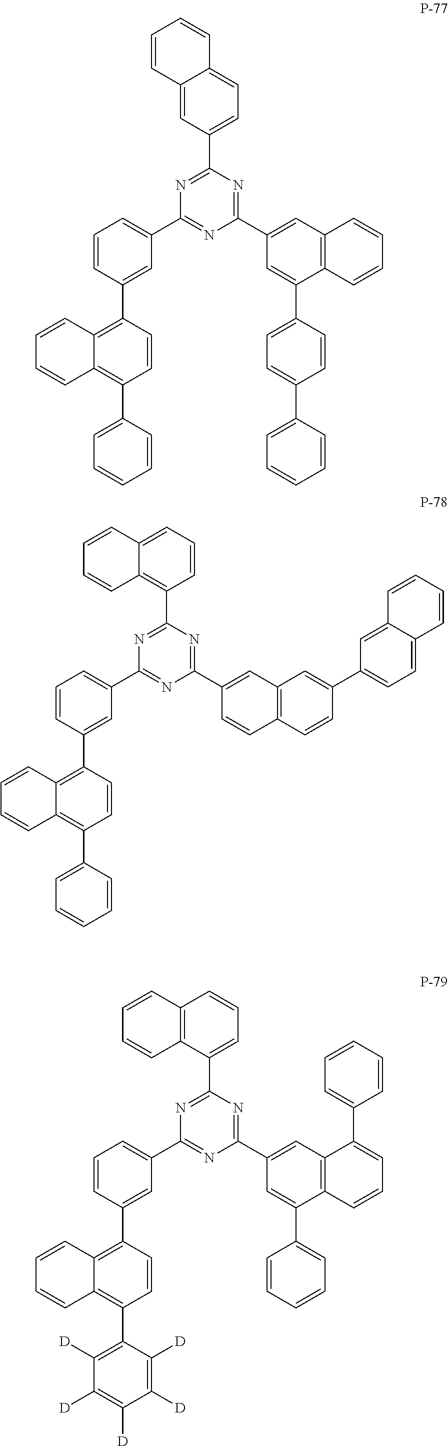

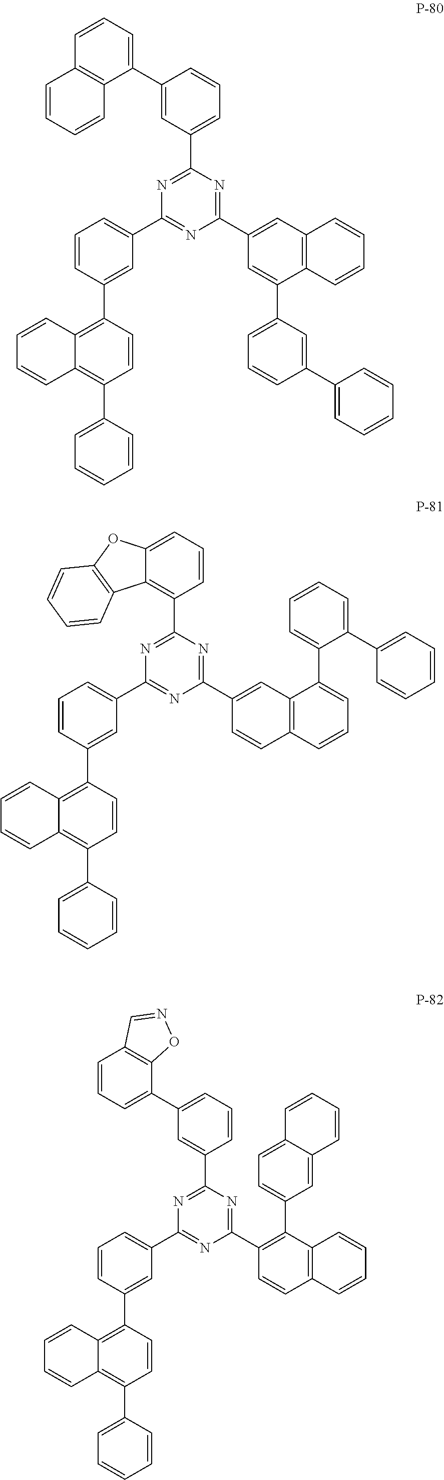 [SMILES: c1ccc(-c2cccc(-c3cc(-c4nc(-c5cccc(-c6cccc7ccccc67)c5)nc(-c5cccc(-c6ccc(-c7ccccc7)c7ccccc67)c5)n4)cc4ccccc34)c2)cc1 ; c1ccc(-c2ccccc2-c2cccc3ccc(-c4nc(-c5cccc(-c6ccc(-c7ccccc7)c7ccccc67)c5)nc(-c5cccc6oc7ccccc7c56)n4)cc23)cc1 ; c1ccc(-c2ccc(-c3cccc(-c4nc(-c5cccc(-c6cccc7cnoc67)c5)nc(-c5ccc6ccccc6c5-c5ccc6ccccc6c5)n4)c3)c3ccccc23)cc1]

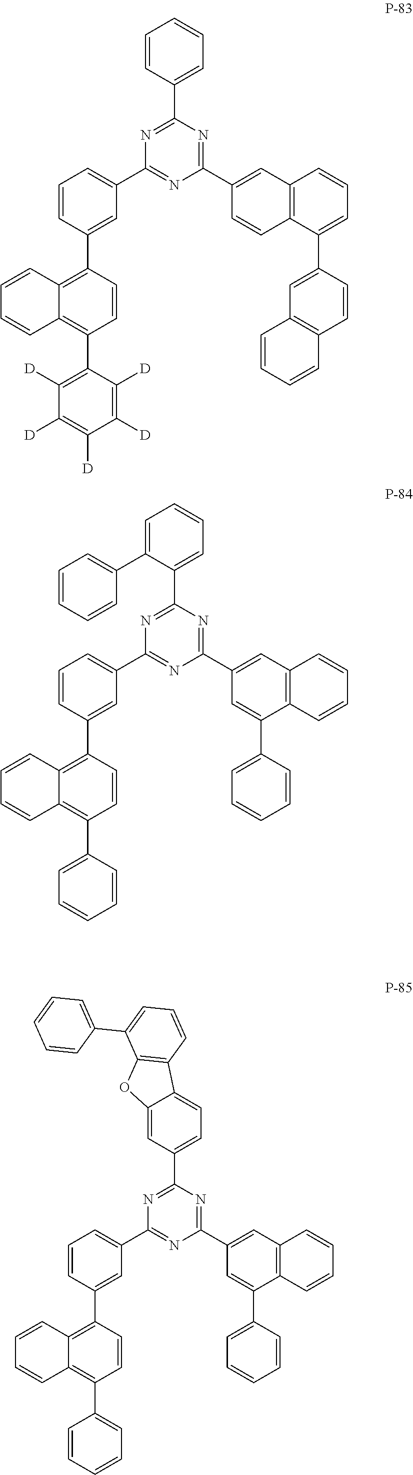 [SMILES: [2H]c1c([2H])c([2H])c(-c2ccc(-c3cccc(-c4nc(-c5ccccc5)nc(-c5ccc6c(-c7ccc8ccccc8c7)cccc6c5)n4)c3)c3ccccc23)c([2H])c1[2H] ; c1ccc(-c2ccccc2-c2nc(-c3cccc(-c4ccc(-c5ccccc5)c5ccccc45)c3)nc(-c3cc(-c4ccccc4)c4ccccc4c3)n2)cc1 ; c1ccc(-c2cc(-c3nc(-c4cccc(-c5ccc(-c6ccccc6)c6ccccc56)c4)nc(-c4ccc5c(c4)oc4c(-c6ccccc6)cccc45)n3)cc3ccccc23)cc1]

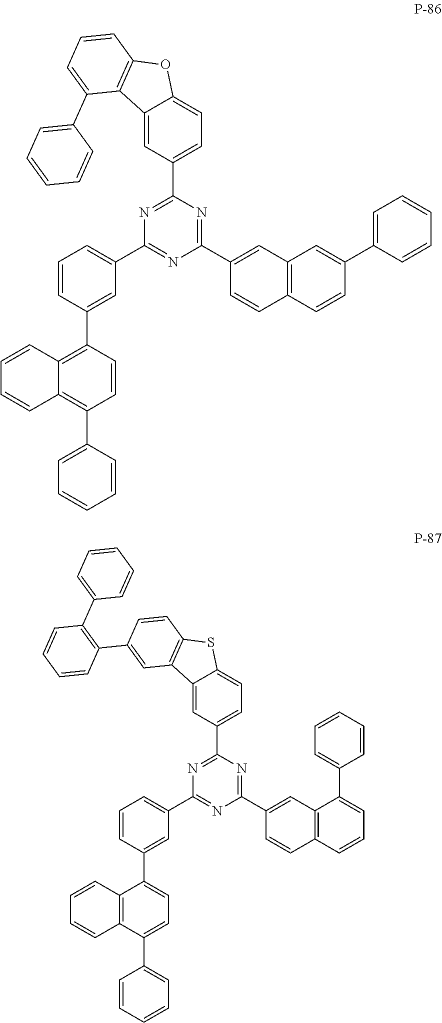 [SMILES: c1ccc(-c2ccc3ccc(-c4nc(-c5cccc(-c6ccc(-c7ccccc7)c7ccccc67)c5)nc(-c5ccc6oc7cccc(-c8ccccc8)c7c6c5)n4)cc3c2)cc1 ; c1ccc(-c2ccccc2-c2ccc3sc4ccc(-c5nc(-c6cccc(-c7ccc(-c8ccccc8)c8ccccc78)c6)nc(-c6ccc7cccc(-c8ccccc8)c7c6)n5)cc4c3c2)cc1]

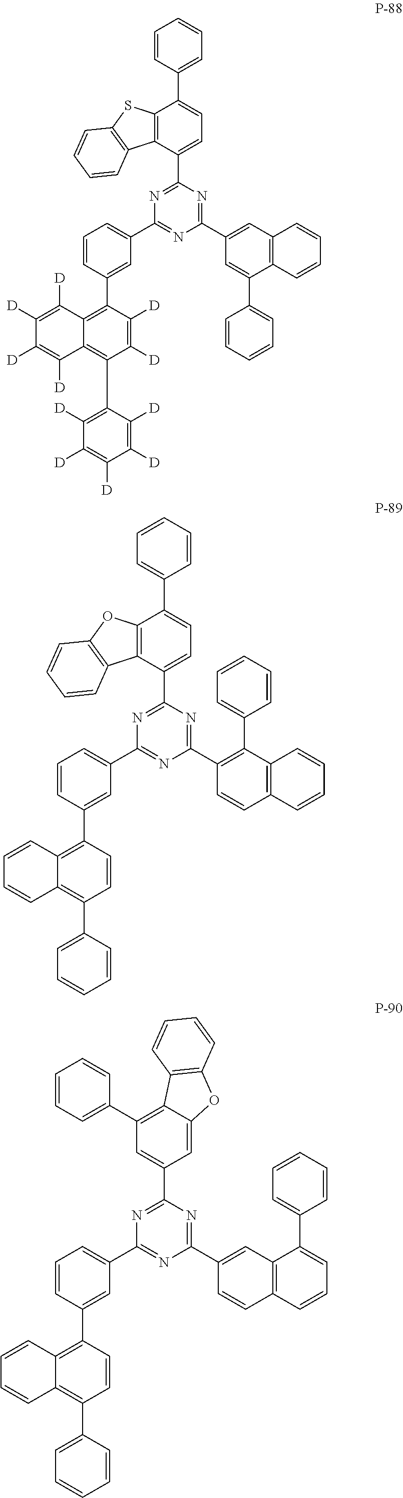 [SMILES: [2H]c1c([2H])c([2H])c(-c2c([2H])c([2H])c(-c3cccc(-c4nc(-c5cc(-c6ccccc6)c6ccccc6c5)nc(-c5ccc(-c6ccccc6)c6sc7ccccc7c56)n4)c3)c3c([2H])c([2H])c([2H])c([2H])c23)c([2H])c1[2H] ; c1ccc(-c2c(-c3nc(-c4cccc(-c5ccc(-c6ccccc6)c6ccccc56)c4)nc(-c4ccc(-c5ccccc5)c5oc6ccccc6c45)n3)ccc3ccccc23)cc1 ; c1ccc(-c2cccc3ccc(-c4nc(-c5cccc(-c6ccc(-c7ccccc7)c7ccccc67)c5)nc(-c5cc(-c6ccccc6)c6c(c5)oc5ccccc56)n4)cc23)cc1]

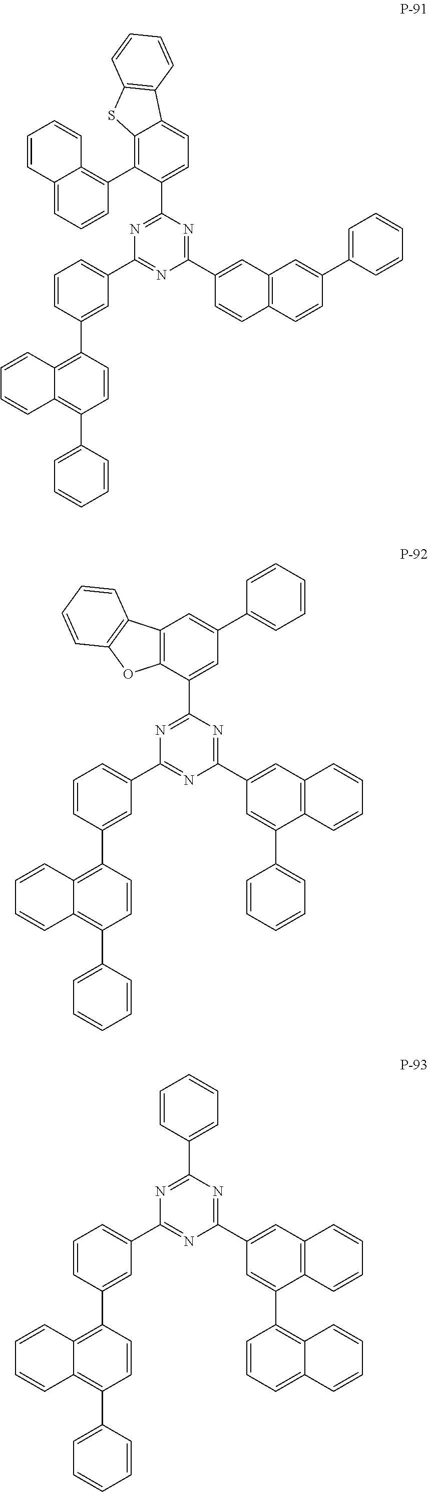 [SMILES: c1ccc(-c2ccc3ccc(-c4nc(-c5cccc(-c6ccc(-c7ccccc7)c7ccccc67)c5)nc(-c5ccc6c(sc7ccccc76)c5-c5cccc6ccccc56)n4)cc3c2)cc1 ; c1ccc(-c2cc(-c3nc(-c4cccc(-c5ccc(-c6ccccc6)c6ccccc56)c4)nc(-c4cc(-c5ccccc5)c5ccccc5c4)n3)c3oc4ccccc4c3c2)cc1 ; c1ccc(-c2nc(-c3cccc(-c4ccc(-c5ccccc5)c5ccccc45)c3)nc(-c3cc(-c4cccc5ccccc45)c4ccccc4c3)n2)cc1]

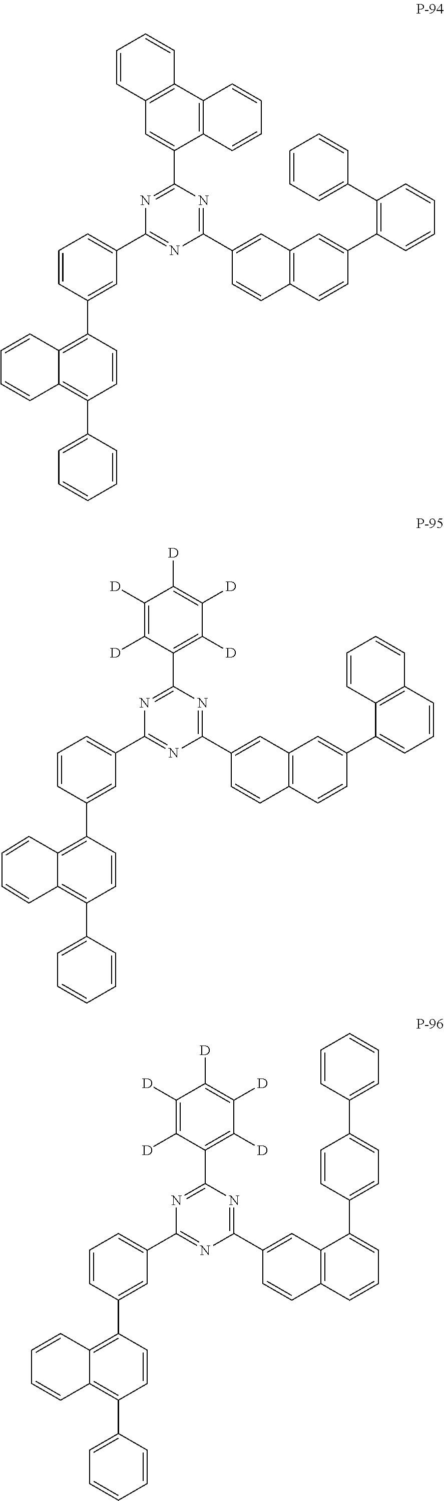 [SMILES: c1ccc(-c2ccccc2-c2ccc3ccc(-c4nc(-c5cccc(-c6ccc(-c7ccccc7)c7ccccc67)c5)nc(-c5cc6ccccc6c6ccccc56)n4)cc3c2)cc1 ; [2H]c1c([2H])c([2H])c(-c2nc(-c3cccc(-c4ccc(-c5ccccc5)c5ccccc45)c3)nc(-c3ccc4ccc(-c5cccc6ccccc56)cc4c3)n2)c([2H])c1[2H] ; [2H]c1c([2H])c([2H])c(-c2nc(-c3cccc(-c4ccc(-c5ccccc5)c5ccccc45)c3)nc(-c3ccc4cccc(-c5ccc(-c6ccccc6)cc5)c4c3)n2)c([2H])c1[2H]]

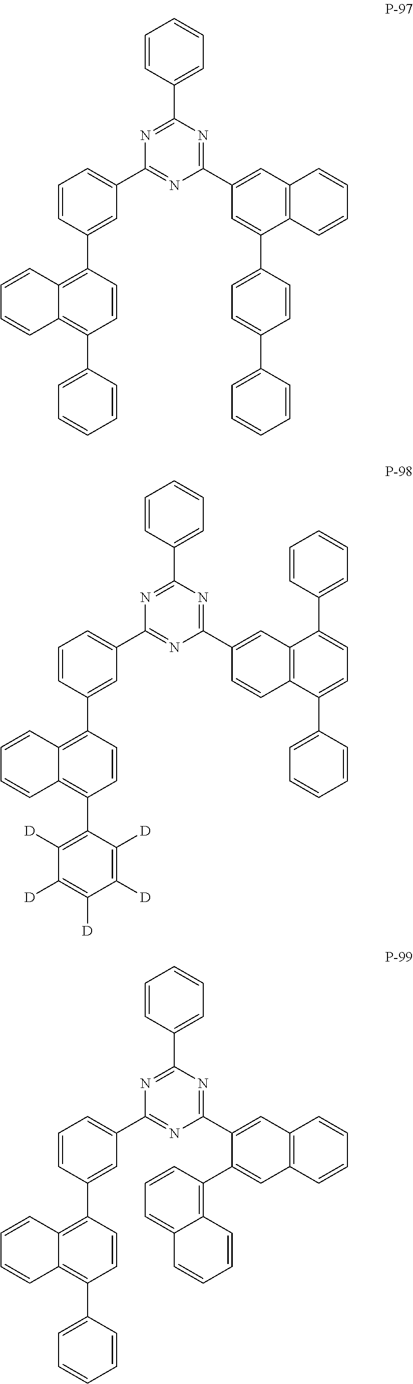 [SMILES: c1ccc(-c2ccc(-c3cc(-c4nc(-c5ccccc5)nc(-c5cccc(-c6ccc(-c7ccccc7)c7ccccc67)c5)n4)cc4ccccc34)cc2)cc1 ; [2H]c1c([2H])c([2H])c(-c2ccc(-c3cccc(-c4nc(-c5ccccc5)nc(-c5ccc6c(-c7ccccc7)ccc(-c7ccccc7)c6c5)n4)c3)c3ccccc23)c([2H])c1[2H] ; c1ccc(-c2nc(-c3cccc(-c4ccc(-c5ccccc5)c5ccccc45)c3)nc(-c3cc4ccccc4cc3-c3cccc4ccccc34)n2)cc1]

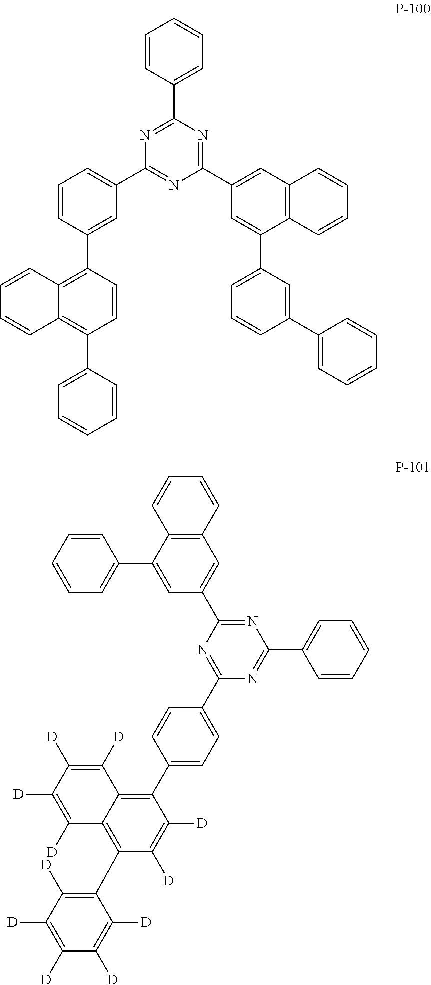 [SMILES: c1ccc(-c2cccc(-c3cc(-c4nc(-c5ccccc5)nc(-c5cccc(-c6ccc(-c7ccccc7)c7ccccc67)c5)n4)cc4ccccc34)c2)cc1 ; [2H]c1c([2H])c([2H])c(-c2c([2H])c([2H])c(-c3ccc(-c4nc(-c5ccccc5)nc(-c5cc(-c6ccccc6)c6ccccc6c5)n4)cc3)c3c([2H])c([2H])c([2H])c([2H])c23)c([2H])c1[2H]]

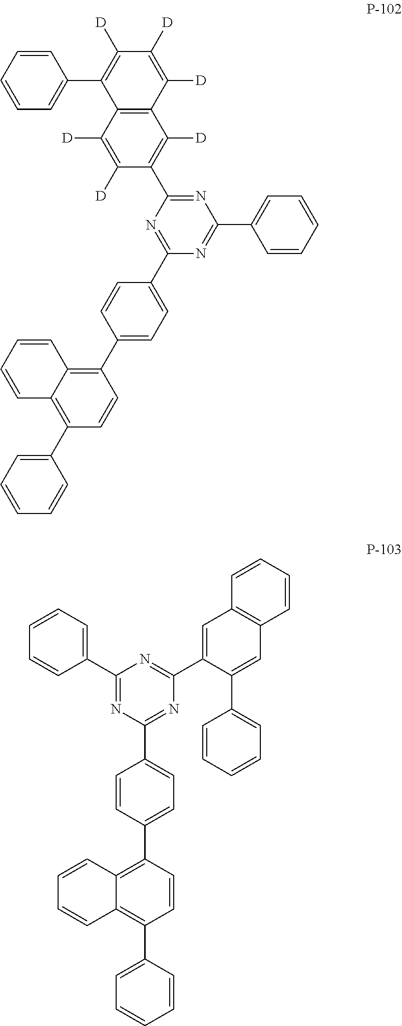 [SMILES: [2H]c1c([2H])c(-c2ccccc2)c2c([2H])c([2H])c(-c3nc(-c4ccccc4)nc(-c4ccc(-c5ccc(-c6ccccc6)c6ccccc56)cc4)n3)c([2H])c2c1[2H] ; c1ccc(-c2nc(-c3ccc(-c4ccc(-c5ccccc5)c5ccccc45)cc3)nc(-c3cc4ccccc4cc3-c3ccccc3)n2)cc1]

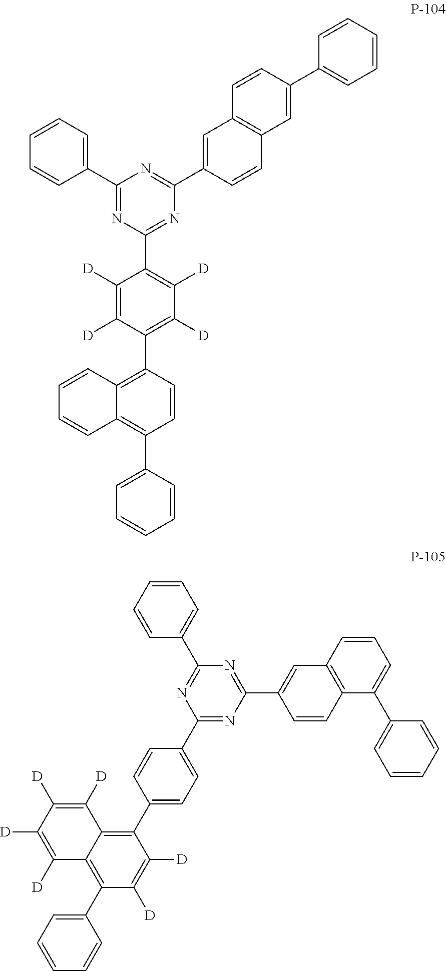 [SMILES: [2H]c1c([2H])c(-c2ccc(-c3ccccc3)c3ccccc23)c([2H])c([2H])c1-c1nc(-c2ccccc2)nc(-c2ccc3cc(-c4ccccc4)ccc3c2)n1 ; [2H]c1c([2H])c([2H])c2c(-c3ccc(-c4nc(-c5ccccc5)nc(-c5ccc6c(-c7ccccc7)cccc6c5)n4)cc3)c([2H])c([2H])c(-c3ccccc3)c2c1[2H]]

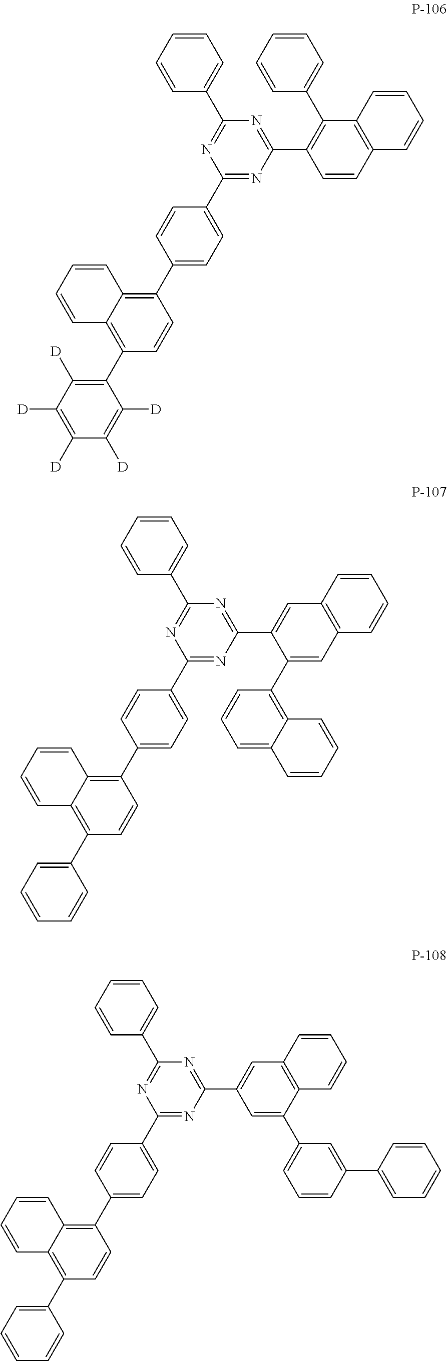 [SMILES: [2H]c1c([2H])c([2H])c(-c2ccc(-c3ccc(-c4nc(-c5ccccc5)nc(-c5ccc6ccccc6c5-c5ccccc5)n4)cc3)c3ccccc23)c([2H])c1[2H] ; c1ccc(-c2nc(-c3ccc(-c4ccc(-c5ccccc5)c5ccccc45)cc3)nc(-c3cc4ccccc4cc3-c3cccc4ccccc34)n2)cc1 ; c1ccc(-c2cccc(-c3cc(-c4nc(-c5ccccc5)nc(-c5ccc(-c6ccc(-c7ccccc7)c7ccccc67)cc5)n4)cc4ccccc34)c2)cc1]

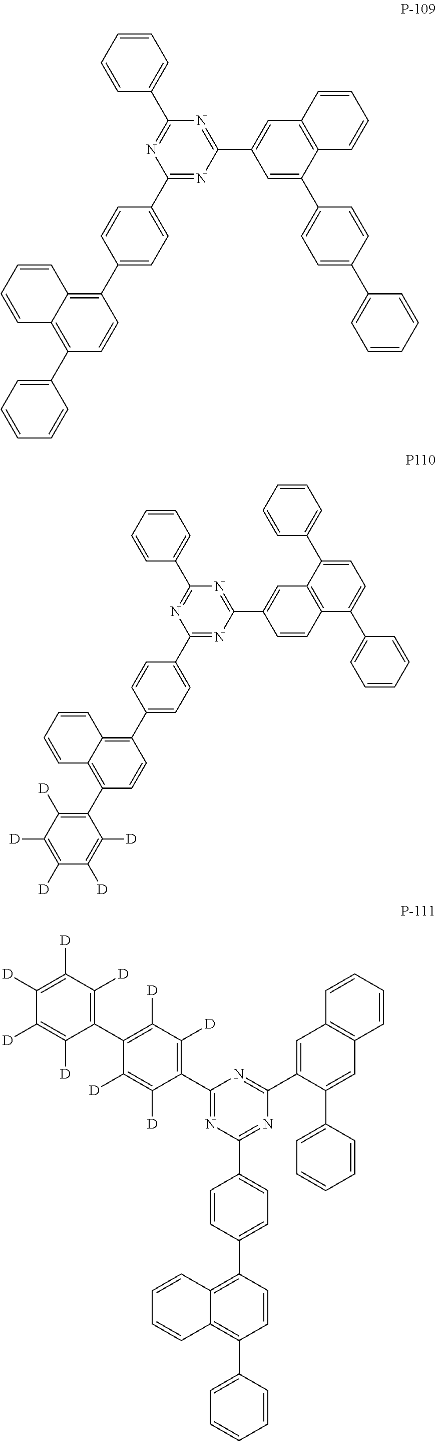 [SMILES: c1ccc(-c2ccc(-c3cc(-c4nc(-c5ccccc5)nc(-c5ccc(-c6ccc(-c7ccccc7)c7ccccc67)cc5)n4)cc4ccccc34)cc2)cc1 ; [2H]c1c([2H])c([2H])c(-c2ccc(-c3ccc(-c4nc(-c5ccccc5)nc(-c5ccc6c(-c7ccccc7)ccc(-c7ccccc7)c6c5)n4)cc3)c3ccccc23)c([2H])c1[2H] ; [2H]c1c([2H])c([2H])c(-c2c([2H])c([2H])c(-c3nc(-c4ccc(-c5ccc(-c6ccccc6)c6ccccc56)cc4)nc(-c4cc5ccccc5cc4-c4ccccc4)n3)c([2H])c2[2H])c([2H])c1[2H]]

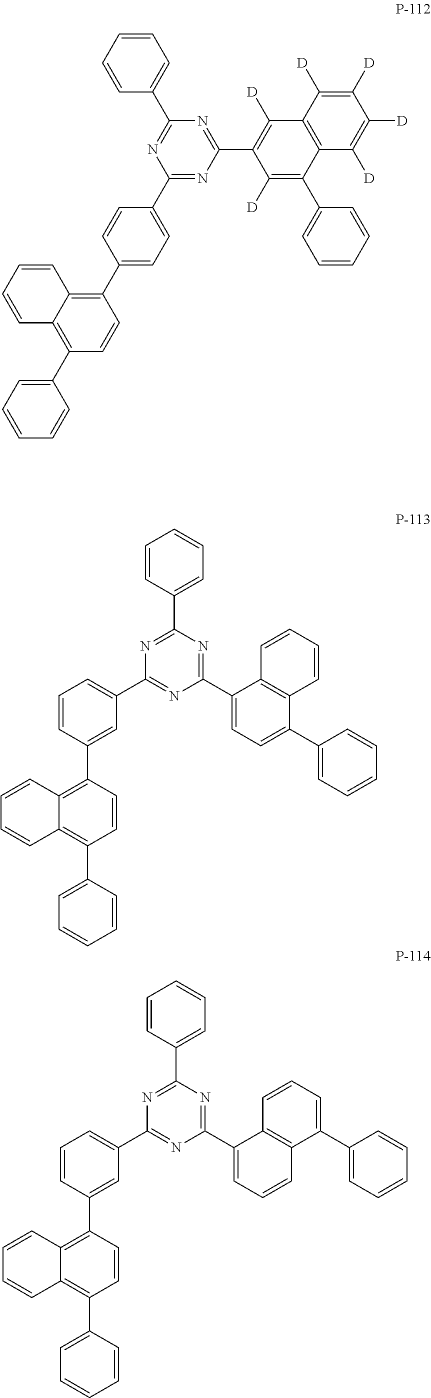 [SMILES: [2H]c1c([2H])c([2H])c2c(-c3ccccc3)c([2H])c(-c3nc(-c4ccccc4)nc(-c4ccc(-c5ccc(-c6ccccc6)c6ccccc56)cc4)n3)c([2H])c2c1[2H] ; c1ccc(-c2nc(-c3cccc(-c4ccc(-c5ccccc5)c5ccccc45)c3)nc(-c3ccc(-c4ccccc4)c4ccccc34)n2)cc1 ; c1ccc(-c2nc(-c3cccc(-c4ccc(-c5ccccc5)c5ccccc45)c3)nc(-c3cccc4c(-c5ccccc5)cccc34)n2)cc1]

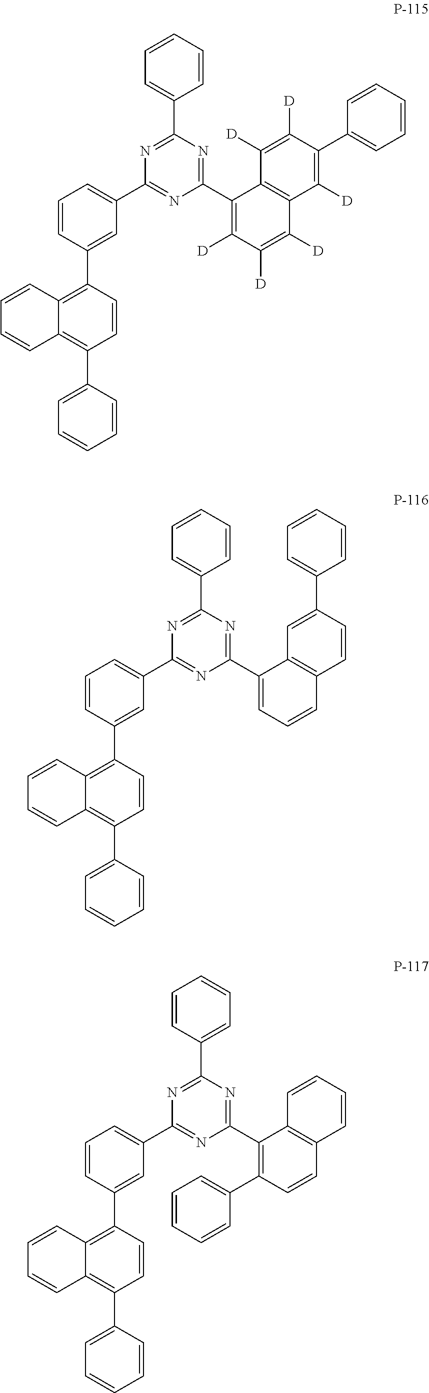 [SMILES: [2H]c1c([2H])c(-c2nc(-c3ccccc3)nc(-c3cccc(-c4ccc(-c5ccccc5)c5ccccc45)c3)n2)c2c([2H])c([2H])c(-c3ccccc3)c([2H])c2c1[2H] ; c1ccc(-c2ccc3cccc(-c4nc(-c5ccccc5)nc(-c5cccc(-c6ccc(-c7ccccc7)c7ccccc67)c5)n4)c3c2)cc1 ; c1ccc(-c2nc(-c3cccc(-c4ccc(-c5ccccc5)c5ccccc45)c3)nc(-c3c(-c4ccccc4)ccc4ccccc34)n2)cc1]

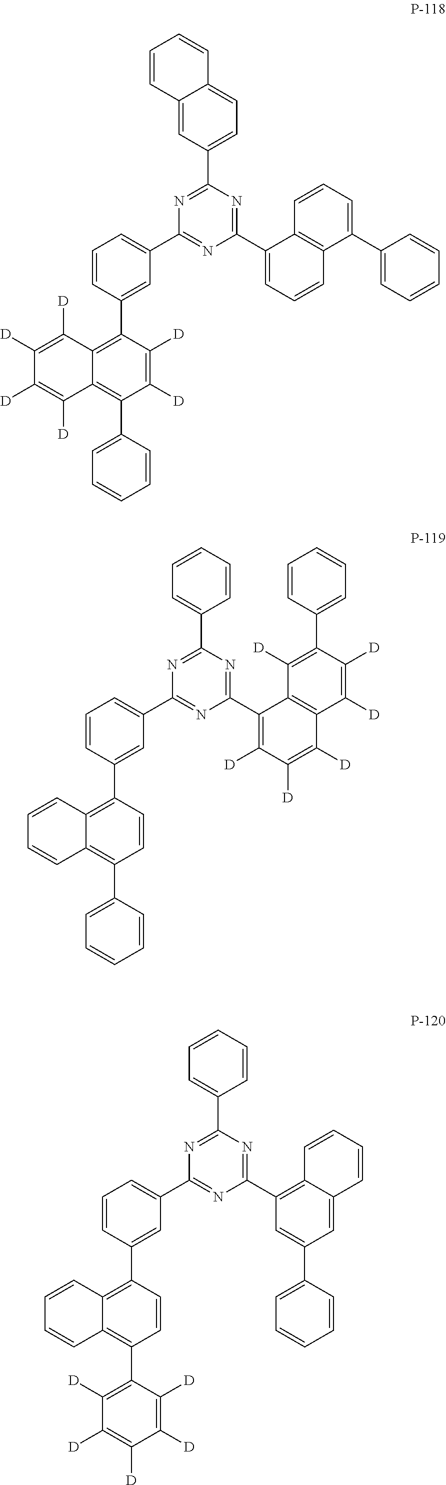 [SMILES: [2H]c1c([2H])c([2H])c2c(-c3cccc(-c4nc(-c5ccc6ccccc6c5)nc(-c5cccc6c(-c7ccccc7)cccc56)n4)c3)c([2H])c([2H])c(-c3ccccc3)c2c1[2H] ; [2H]c1c([2H])c(-c2nc(-c3ccccc3)nc(-c3cccc(-c4ccc(-c5ccccc5)c5ccccc45)c3)n2)c2c([2H])c(-c3ccccc3)c([2H])c([2H])c2c1[2H] ; [2H]c1c([2H])c([2H])c(-c2ccc(-c3cccc(-c4nc(-c5ccccc5)nc(-c5cc(-c6ccccc6)cc6ccccc56)n4)c3)c3ccccc23)c([2H])c1[2H]]

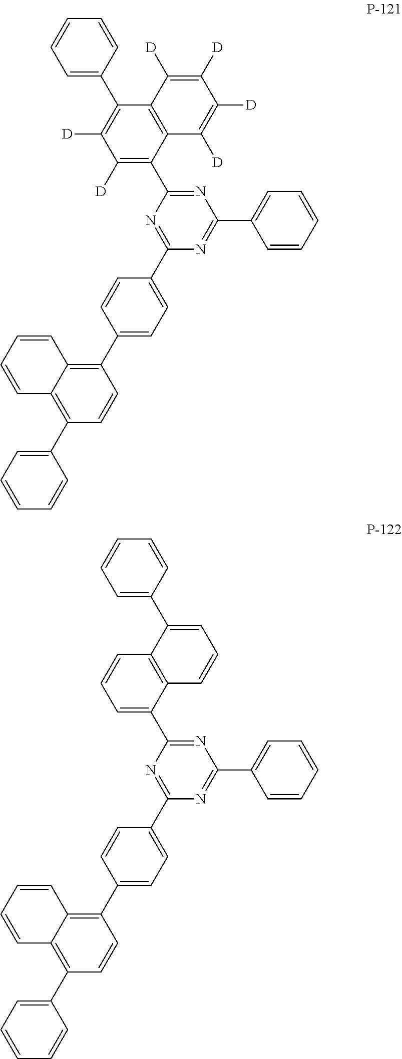 [SMILES: [2H]c1c([2H])c([2H])c2c(-c3nc(-c4ccccc4)nc(-c4ccc(-c5ccc(-c6ccccc6)c6ccccc56)cc4)n3)c([2H])c([2H])c(-c3ccccc3)c2c1[2H] ; c1ccc(-c2nc(-c3ccc(-c4ccc(-c5ccccc5)c5ccccc45)cc3)nc(-c3cccc4c(-c5ccccc5)cccc34)n2)cc1]

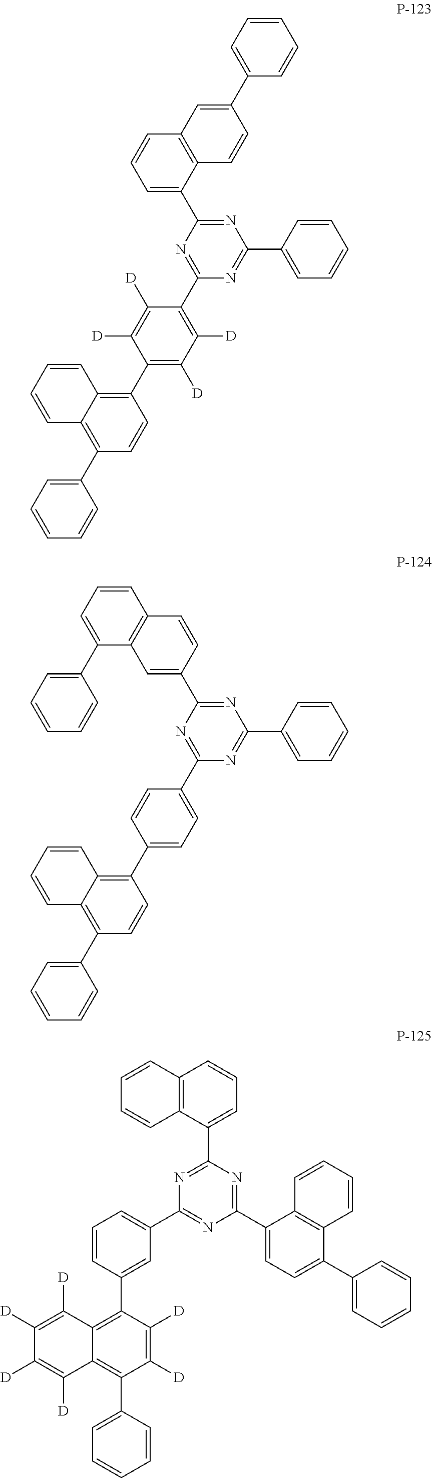 [SMILES: [2H]c1c([2H])c(-c2ccc(-c3ccccc3)c3ccccc23)c([2H])c([2H])c1-c1nc(-c2ccccc2)nc(-c2cccc3cc(-c4ccccc4)ccc23)n1 ; c1ccc(-c2nc(-c3ccc(-c4ccc(-c5ccccc5)c5ccccc45)cc3)nc(-c3ccc4cccc(-c5ccccc5)c4c3)n2)cc1 ; [2H]c1c([2H])c([2H])c2c(-c3cccc(-c4nc(-c5cccc6ccccc56)nc(-c5ccc(-c6ccccc6)c6ccccc56)n4)c3)c([2H])c([2H])c(-c3ccccc3)c2c1[2H]]

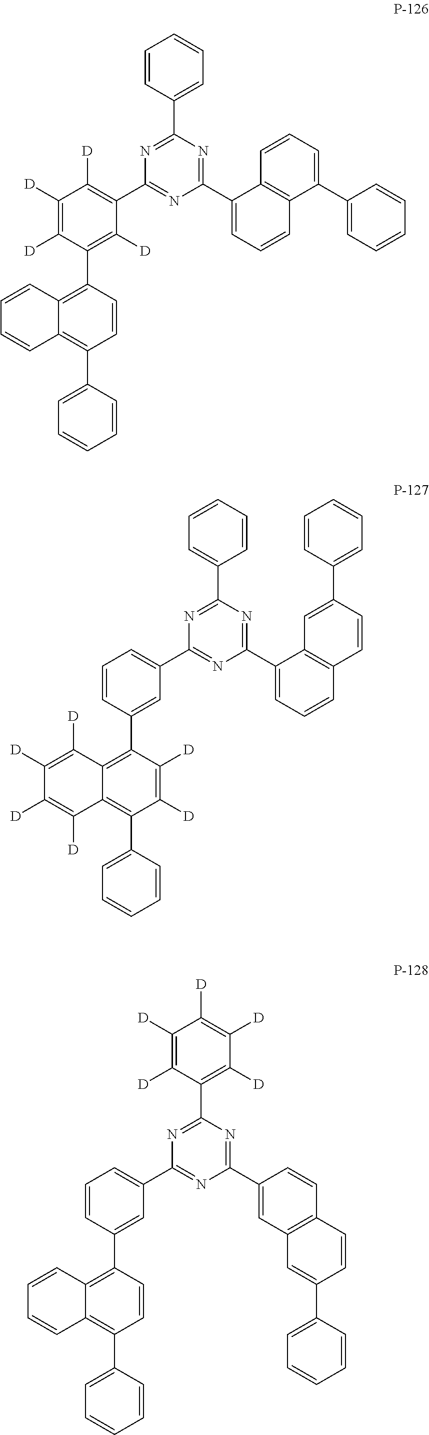 [SMILES: [2H]c1c([2H])c(-c2nc(-c3ccccc3)nc(-c3cccc4c(-c5ccccc5)cccc34)n2)c([2H])c(-c2ccc(-c3ccccc3)c3ccccc23)c1[2H] ; [2H]c1c([2H])c([2H])c2c(-c3cccc(-c4nc(-c5ccccc5)nc(-c5cccc6ccc(-c7ccccc7)cc56)n4)c3)c([2H])c([2H])c(-c3ccccc3)c2c1[2H] ; [2H]c1c([2H])c([2H])c(-c2nc(-c3cccc(-c4ccc(-c5ccccc5)c5ccccc45)c3)nc(-c3ccc4ccc(-c5ccccc5)cc4c3)n2)c([2H])c1[2H]]

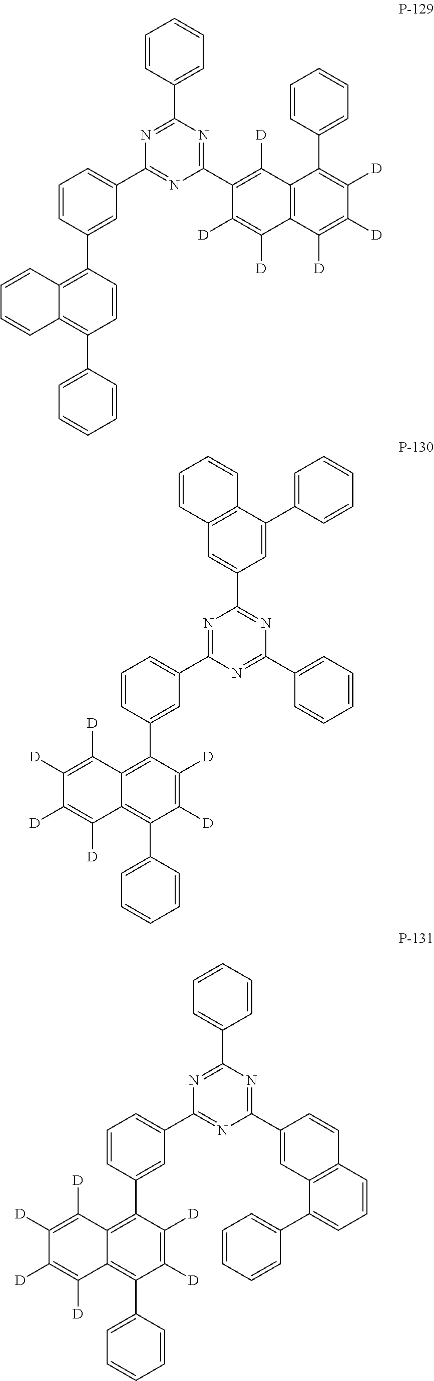 [SMILES: [2H]c1c([2H])c(-c2ccccc2)c2c([2H])c(-c3nc(-c4ccccc4)nc(-c4cccc(-c5ccc(-c6ccccc6)c6ccccc56)c4)n3)c([2H])c([2H])c2c1[2H] ; [2H]c1c([2H])c([2H])c2c(-c3cccc(-c4nc(-c5ccccc5)nc(-c5cc(-c6ccccc6)c6ccccc6c5)n4)c3)c([2H])c([2H])c(-c3ccccc3)c2c1[2H] ; [2H]c1c([2H])c([2H])c2c(-c3cccc(-c4nc(-c5ccccc5)nc(-c5ccc6cccc(-c7ccccc7)c6c5)n4)c3)c([2H])c([2H])c(-c3ccccc3)c2c1[2H]]

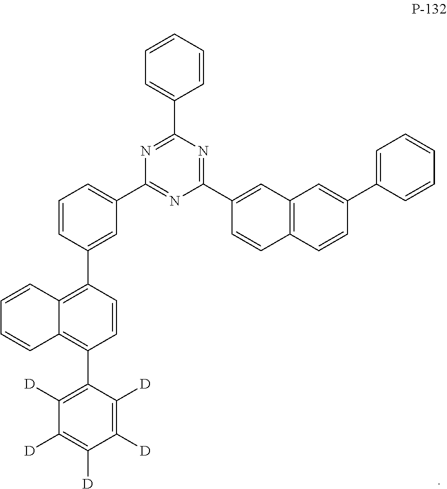 [SMILES: [2H]c1c([2H])c([2H])c(-c2ccc(-c3cccc(-c4nc(-c5ccccc5)nc(-c5ccc6ccc(-c7ccccc7)cc6c5)n4)c3)c3ccccc23)c([2H])c1[2H]]

5. A composition for an organic electronic element comprising a mixture of a compound of claim 1 and a compound represented by Formula 2 or 3: 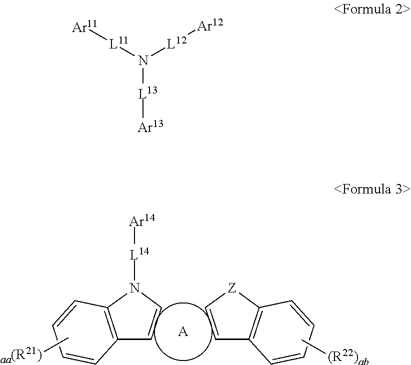
wherein:
L¹¹, L¹², L¹³ and L¹⁴ are each independently selected from the group consisting of a single bond; a C₆-C₆₀ arylene group; a fluorenylene group; a C₂-C₆₀ heterocyclic group including at least one heteroatom of O, N, S, Si and P; and a fused ring group of a C₃-C₆₀ aliphatic ring and a C₆-C₆₀ aromatic ring;
Ar¹¹, Ar¹² and Ar¹³ are each independently selected from the group consisting of a C₆-C₆₀ aryl group; a fluorenyl group; a C₂-C₆₀ heterocyclic group including at least one heteroatom of O, N, S, Si and P; a fused ring group of a C₈-C₆₀ aliphatic ring and a C₆-C₆₀ aromatic ring; a C₃-C₆₀ aliphatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group;
Ar¹⁴ is selected from the group consisting of a C₆-C₆₀ aryl group; a fluorenyl group; a C₂-C₆₀ heterocyclic group including at least one heteroatom of O, N, S, Si and P; a fused ring group of a C₃-C₆₀ aliphatic ring and a C₆-C₆₀ aromatic ring; and -L′-N(R^{a})(R^{b}), wherein L′ is selected from the group consisting of a single bond; a C₆-C₆₀ arylene group; a fluorenylene group; a C₂-C₆₀ heterocyclic group including at least one heteroatom of O, N, S, Si and P; and a C₃-C₆₀ aliphatic ring;
R^{a} and R^{b} are the same as the definition of Ar¹¹,
Z is O, S, C(R⁵¹)(R⁵²) or NR⁵³,
wherein R⁵¹, R⁵² and R⁵³ are the same as the definition of Ar¹¹, or R⁵¹ and R⁵² may be bonded to each other to form a spiro ring,
Ring A is a C₆-C₂₀ aryl group,
R²¹ and R²² are, independently from each other, selected from the group consisting of hydrogen; deuterium; halogen; a cyano group; a C₆-C₆₀ aryl group; a fluorenyl group;
a C₂-C₆₀ heterocyclic group including at least one hetero atom of O, N, S, Si and P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group; and a plurality of adjacent R²¹s or a plurality of adjacent R²²s may be bonded to each other to form a ring,
aa and ab are each independently an integer of 0 to 4,
wherein the aryl group, arylene group, heterocyclic group, fluorenyl group, fluorenylene group, fused ring group, aliphatic ring group, alkyl group, alkenyl group, alkynyl group, alkoxy group and aryloxy group may be substituted with one or more substituents selected from the group consisting of deuterium; halogen; a silane group; a siloxane group; a boron group; a germanium group; a cyano group; a nitro group; a C₁-C₂₀ alkylthio group; a C₁-C₂₀ alkoxyl group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ aliphatic ring; a C₇-C₂₀ arylalkyl group; a C₈-C₂₀ arylalkenyl group; a C₇-C₂₀ alkylaryl group; and -L′-N(R^{a})(R^{b}), and the hydrogen(s) of these substituents may be further substituted with one or more deuterium, and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

6. An organic electronic element comprising a first electrode; a second electrode; and an organic material layer formed between the first electrode and the second electrode, wherein the organic material layer comprises the compound of claim 1, or the composition of claim 5.

7. The organic electronic element according to claim 6, further comprising a light efficiency enhancing layer formed on at least one surface of the first electrode and the second electrode, the surface being opposite to the organic material layer.

8. The organic electronic element according to claim 6, wherein the organic material layer comprises 2 or more stacks comprising a hole transport layer, an emitting layer and an electron transport layer sequentially formed on the first electrode.

9. The organic electronic element according to claim 8, wherein the organic material layer further comprises a charge generation layer formed between the 2 or more stacks.

10. An electronic device comprising a display device comprising the organic electronic element of claim 6; and a control unit for driving the display device.

11. The electronic device according to claim 10, wherein the organic electronic element is at least one of an OLED, an organic solar cell, an organic photo conductor (OPC), organic transistor (organic TFT) and an element for monochromic or white illumination.

12. A method for reusing the compound of Formula 1 of claim 1 comprising:
recovering a crude organic light emitting material comprising the compound of Formula 1 from a deposition apparatus used in a process for depositing the organic emitting material to prepare an organic an organic light emitting device;
removing impurities from the crude organic light emitting material;
recovering the organic light emitting material after the impurities are removed; and
purifying the recovered organic light emitting material to have a purity of 99.9% or higher.
